(19) United States
(12) Patent Application Publication
(10) Pub. No.: US 2025/0108082 A1
(43) Pub. Date: Apr. 3, 2025

(54) THERAPEUTIC COMBINATIONS FOR INFLAMMATORY CONDITIONS AND DISORDERS

(71) Applicant: Native Code Bio, LLC, Bainbridge Island, WA (US)

(72) Inventors: Adam R. Nelson, PIEDMONT, CA (US); Stephen Y. Hung, BAINBRIDGE ISLAND, WA (US)

(21) Appl. No.: 18/840,761

(22) PCT Filed: Feb. 20, 2024
(86) PCT No.: PCT/US24/16584

§ 371 Date: Aug. 22, 2024

Related U.S. Application Data
(60) Provisional application No. 63/446,403, filed on Feb. 17, 2023.

(51) Int. Cl. A61K 36/07; A61K 31/00; A61K 31/37; A61K 31/4045; A61K 31/675; A61K 31/737; A61K 36/04; A61K 36/185; A61K 36/48; A61P 29/00

(52) U.S. Cl. CPC A61K 36/078; A61K 31/37; A61K 31/4045; A61K 31/658; A61K 31/675; A61K 31/737; A61K 36/04; A61K 36/3482; A61K 36/48; A61P 29/00

(57) ABSTRACT

Provided are therapeutic combinations, pharmaceutical compositions, and pharmaceutical kits comprising fungi (e.g., Psilocybe spp. fungi), plants (e.g., Cannabis spp., Dipteryx spp.), and algae (e.g., from the family Bangicaeae, including Pyropia spp. and Porphyra spp.), including extracts and bioactive molecules derived therefrom. Methods of producing the disclosed combinations, compositions, and kits are also provided, such as using natural, biosynthetic, or synthetic means. Further provided are methods of using the disclosed combinations, compositions, and kits in the treatment of medical conditions, and in particular for treating inflammatory diseases and conditions, for which the disclosed combinations are demonstrated to provide significant advantages.

### CROSS-REFERENCE

Priority is claimed under PCT Art. 8(1) and Rule 4.10 to U.S. Prov. App. No. 63/446,403, filed Feb. 17, 2023, and incorporated by reference for all purposes as if fully set forth herein.

### FIELD OF THE INVENTION

Provided are therapeutic combinations comprising fungi, plants, and algae, including extracts thereof and bioactive molecules therefrom. Also provided are pharmaceutical compositions and kits comprising them, and methods of their use in medicine, and in particular to treat inflammatory conditions and disorders.

### BACKGROUND OF THE INVENTION

Globally, approximately 50% of people die due to inflammatory disease (GBD 2017 Causes of Death Collaborators. Lancet. 2018; 392(10159):1736-1788). Inflammation may be acute, which typically is short lived and may result from, for example, an injury. Alternatively, inflammation may be chronic, which is associated with a wide range of diseases including asthma, autoimmune diseases like rheumatoid arthritis, cardiovascular diseases, metabolic diseases, neurodegenerative diseases, gastrointestinal diseases, respiratory diseases, and certain types of cancers.

Many inflammatory conditions or disorders are not curable, and treatment generally attempts only to ease symptoms or slow their progression. Novel treatments that can reduce the severity of symptoms, prevent the development of new symptoms, and reverse disease progression, are sorely needed.

Provided herein are therapeutic combinations, pharmaceutical compositions and kits, and methods of their use to treat inflammatory conditions and disorders, to meet these needs and others, and having such advantages and improvements over prior treatment options as will be readily appreciated.

### INCORPORATION BY REFERENCE

Each cited patent, publication, and non-patent literature is incorporated by reference in its entirety, as if each was incorporated individually, and as if each is fully set forth herein. However, no such citation should be construed as an admission that a cited reference comes from an area that is analogous or directly applicable to the invention, nor should a citation be construed as an admission that a document or underlying information, in any jurisdiction, is prior art or forms part of the common general knowledge in the art.

### BRIEF SUMMARY OF THE INVENTION

A simplified summary of some aspects and embodiments follows, to provide a basic understanding of the invention. This summary is not an extensive overview, nor is it intended to identify every key or critical element of the invention or to delineate the complete scope of the invention. Its sole purpose is to present some exemplary embodiments in a simplified form as a prelude to the more detailed description below.

In a first aspect, provided are methods of preventing or treating an inflammatory condition or disorder, comprising administering to a subject having the inflammatory condition or disorder a therapeutic combination comprising:
a. a fungal portion;
b. a first plant portion;
c. a second plant portion; and
d. an algal portion.

In another aspect, provided are uses of a therapeutic combination comprising:
a. a fungal portion;
b. a first plant portion;
c. a second plant portion; and
d. an algal portion;
for preventing or treating an inflammatory condition or disorder in a subject.

Also provided are method of preventing or treating an inflammatory condition or disorder, comprising administering to a subject having the inflammatory condition or disorder the therapeutic combination of any of the disclosed embodiments, or a therapeutic combination comprising:
a. a fungal portion;
b. a first plant portion;
c. optionally, a second plant portion; and
d. optionally, an algal portion.

In some embodiments, the fungal portion comprises any of a fungal extract, a primary bioactive molecule from a fungus, and a secondary bioactive molecule from a fungus.

In some embodiments, the first plant portion comprises any of a plant extract, a primary bioactive molecule from a plant, and a secondary bioactive molecule from a plant.

In some embodiments, the second plant portion comprises any of a plant extract, a primary bioactive molecule from a plant, and a secondary bioactive molecule from a plant.

In some embodiments, the algal portion comprises any of an algal extract, a primary bioactive molecule from an algae, and a secondary bioactive molecule from an algae.

In some embodiments, the fungal portion is from a psilocybin-producing species. In some embodiments, the psilocybin-producing species is from any of the genera Psilocybe, Athelia, Conocybe, Copelandia, Fibularhizoctonia, Galerina, Gymnopilus, Inocybe, Mycena, Panaeolus, Pholiotina, and Pluteus. In some embodiments, the psilocybin-producing species is from the genus Psilocybe. In some embodiments, the psilocybin-producing species from the genus Psilocybe is any of P. cubensis, P. azurescens, P. bohemica, P. semilanceata, P. baeocystis, P. cyanescens, P. tampanensis, P. weilii, P. hoogshagenii, P. stuntzii, P. cyanofibrillosa, and P. liniformans.

In embodiments, the first plant portion is from a species in the Cannabis genus. In embodiments, the species in the Cannabis genus is any of Cannabis sativa, Cannabis indica, and Cannabis uderalis.

In some embodiments, the second plant portion is from a species in the Dipteryx genus. In some embodiments, the species in the Dipteryx genus is Dipteryx odorata.

In some embodiments, the algal portion is from a species of marine algae. In some embodiments, the species of marine algae is from the family Bangiaceae. In some embodiments, the species of marine algae is from the genus Pyropia or Porphyra. In some embodiments, the species of marine algae is any of Pyropia yezoensis, Pyropia perforata, and Porphyra umbilicalis.

In some embodiments, any of the fungal portion, first plant portion, second plant portion, and algal portion are administered in separate compositions.

In some embodiments, any of the fungal portion, first plant portion, second plant portion, and algal portion are administered simultaneously. In some embodiments, any of the fungal portion, first plant portion, second plant portion, and algal portion are administered in a single composition.

In some embodiments:
a. the fungal portion comprises a Psilocybe cubensis extract;
b. the first plant portion comprises a Cannabis sativa extract;
c. the second plant portion comprises a Dipteryx odorata extract; and
d. the algal portion comprises a Pyropia yezoensis extract.

In some embodiments, the Psilocybe cubensis extract is obtained by ultrasonic extraction or Soxhlet extraction. In some embodiments, the Psilocybe cubensis extract comprises a mixture of a Psilocybe cubensis extract obtained by ultrasonic extraction and a Psilocybe cubensis extract obtained by Soxhlet extraction in a weight ratio from about 0.5:1 to about 5:1. In some embodiments, the weight ratio of the Psilocybe cubensis extract obtained by ultrasonic extraction and the Psilocybe cubensis extract obtained by Soxhlet extraction is 2:1.

In some embodiments, the Psilocybe cubensis extract comprises psilocybin and psilocin. In some embodiments, the Psilocybe cubensis extract comprises psilocybin and psilocin in a weight ratio from about 1:5 to about 5:1. In some embodiments, the Psilocybe cubensis extract comprises psilocybin and psilocin in a weight ratio of about 5:3. In some embodiments, the Psilocybe cubensis extract comprises from about 50 μg to about 500 μg of psilocybin and from about 20 μg to about 200 μg of psilocin. In some embodiments, the Psilocybe cubensis extract comprises about 250 μg of psilocybin and about 150 μg of psilocin. In some embodiments, the Cannabis sativa extract is obtained by Soxhlet extraction. In some embodiments, the Cannabis sativa extract comprises Δ⁹-tetrahydrocannabinol (THC) and cannabidiol (CBD). In some embodiments, the Cannabis sativa extract comprises THC and CBD in a weight ratio from about 1:5 to about 5:1. In some embodiments, the Cannabis sativa extract comprises THC and CBD in a weight ratio of about 1:1. In some embodiments, the Cannabis sativa extract comprises from about 0.1 mg to about 5 mg of THC and from about 0.1 to 5 mg of CBD. In some embodiments, the Cannabis sativa extract comprises about 1 mg of THC and about 1 mg of CBD.

In some embodiments, the Dipteryx odorata extract is obtained by anhydrous ethanol percolation. In some embodiments, the Dipteryx odorata extract comprises coumarin. In some embodiments, the Dipteryx odorata extract comprises from about 1 mg to about 10 mg of coumarin. In some embodiments, the Dipteryx odorata extract comprises about 1 mg of coumarin.

In some embodiments, the combination comprises from about 1 mg to about 20 mg of Pyropia yezoensis extract. In some embodiments, the combination comprises about 8 mg of Pyropia yezoensis extract. In some embodiments, the Pyropia yezoensis extract is obtained by ultrasonic extraction. In some embodiments, the Pyropia yezoensis extract comprises porphyran.

In some embodiments, any of the Psilocybe cubensis extract, Cannabis sativa extract, Dipteryx odorata extract, and Pyropia yezoensis extract are administered in separate compositions.

In some embodiments, any of the Psilocybe cubensis extract, Cannabis sativa extract, Dipteryx odorata extract, and Pyropia yezoensis extract are administered simultaneously. In some embodiments, any of the Psilocybe cubensis extract, Cannabis sativa extract, Dipteryx odorata extract, and Pyropia yezoensis extract are administered in a single composition.

In some embodiments, the Psilocybe cubensis extract constitutes from about 20% to about 60% by volume of the single composition. In some embodiments, the Psilocybe cubensis extract constitutes about 45% by volume of the single composition.

In some embodiments, the Cannabis sativa extract constitutes from about 5% to about 30% by volume of the single composition. In some embodiments, the Cannabis sativa extract constitutes about 15% by volume of the single composition.

In some embodiments, the Dipteryx odorata extract constitutes from about 1% to about 5% by volume of the single composition. In some embodiments, the Dipteryx odorata extract constitutes about 2% by volume of the single composition.

In some embodiments, the Pyropia yezoensis extract constitutes from about 5% to about 30% by volume of the single composition. In some embodiments, the Pyropia yezoensis extract constitutes about 15% by volume of the single composition.

In some embodiments, the single composition further comprises any of a flavorant, colorant, and diluent. In embodiments, the flavorant or colorant comprises ginger or bay laurel. In embodiments, the flavorant or colorant is ethanol infused with ginger and bay leaf. In embodiments, the flavorant or colorant constitutes from about 5% to about 30% by volume of the single composition. In embodiments, the flavorant or colorant constitutes about 15% by volume of the single composition. In embodiments, the diluent is water. In embodiments, the water constitutes from about 5% to about 15% by volume of the single composition. In embodiments, the water constitutes about 8% by volume of the single composition.

In some embodiments, the method or use comprises administering between about 10 and 200 mg of the single composition per single dose. In some embodiments, the method or use comprises administering between about 50 mg of the single composition per single dose. In some embodiments, the method or use comprises administering between 1 and 8 doses of the single composition per day. In some embodiments, the method or use comprises administering between 1 and 8 doses of the single composition per day for at least one week, two weeks, three weeks, 1 month, 2 months, or 3 months.

In some embodiments, the subject experiences a reduction in the severity of a symptom of the inflammatory condition or disorder. In some embodiments, the reduction in the severity of the symptom is durable for at least one week, two weeks, three weeks, 1 month, 2 months, 3 months, 6 months, 9 months, or at least 12 months, when measured from baseline.

In embodiments, the inflammatory condition or disorder is any of long COVID, rheumatoid arthritis, psoriatic arthritis, reactive arthritis, tendonitis, gout, scleroderma, systemic scleroderma, localized scleroderma, CREST syndrome, sciatica, neuropathy, peripheral neuropathy, hereditary neuropathy, acquired neuropathy, motor neuropathy, sensory neuropathy, autonomic neuropathy, combination neuropathies, sciatic neuritis, myalgic encephalomyelitis, chronic fatigue syndrome (CFS), fatty liver disease, endometriosis, type 1 diabetes mellitus, type 2 diabetes mellitus, inflammatory bowel disease, asthma, obesity, cancer, Kawasaki's disease, vasculitis, uveitis, Crohn's disease, ulcerative colitis, meningitis, allergies, psoriasis, Hashimoto's disease, Guillain Barre syndrome, hepatitis, celiac disease, multiple sclerosis, fibromyalgia, lupus, and Sjogren's syndrome. In some embodiments, the inflammatory condition or disorder is any of long COVID, CREST syndrome, psoriatic arthritis, Hashimoto's disease, multiple sclerosis, sciatica, peripheral neuritis, post-treatment Lyme disease/chronic Lyme disease (CLD) syndrome, chronic fatigue and immune dysfunction syndrome (CFIDS)/chronic fatigue syndrome (CFS), and inflammatory skin conditions.

In some embodiments, the inflammatory condition or disorder is long COVID. In some embodiments, the subject experiences a reduction in the severity of a long COVID symptom. In some embodiments, the symptom of long COVID is any of inflammation, fever, chills, cough, shortness of breath, difficulty breathing, fatigue, muscle ache, body ache, headache, loss of taste, loss of smell, sore throat, congestion, runny nose, nausea, vomiting, diarrhea, difficulty thinking or concentrating, sleep problems, dizziness, neuropathic pain, nerve sensitivity, change in taste, change in smell, depression, anxiety, confusion, poor sleep quality, heart palpitations, joint pain, chest pain, and stomach pain.

In some embodiments, the inflammatory condition or disorder is CREST syndrome. In some embodiments, the subject experiences a reduction in the severity of a CREST syndrome symptom. In some embodiments, the CREST syndrome symptom is any of inflammation, stiffness, decreased motor function, calcification, fatigue, depression, Raynaud's phenomenon, and poor sleep quality.

In some embodiments, the inflammatory condition or disorder is psoriatic arthritis. In some embodiments, the subject experiences a reduction in the severity of a psoriatic arthritis symptom. In some embodiments, the psoriatic arthritis symptom is any of joint swelling or inflammation, plaques, fatigue, itching, pitted fingernails, and red scaly rashes.

In some embodiments, the inflammatory condition or disorder is Hashimoto's disease. In some embodiments, the subject experiences a reduction in the severity of a Hashimoto's disease symptom. In some embodiments, the Hashimoto's disease symptom is any of inflammation, swelling, stiffness, reduced motor function, fatigue, reduced endurance, weight gain, muscle weakness, goiter, and pain.

In some embodiments, the inflammatory condition or disorder is multiple sclerosis. In embodiments, the subject experiences a reduction in the severity of a multiple sclerosis symptom. In embodiments, the multiple sclerosis symptom is any of inflammation, depression, poor sleep quality, and fatigue.

In some embodiments, the inflammatory condition or disorder is sciatica. In some embodiments, the subject experiences a reduction in the severity of a sciatica symptom. In some embodiments, the sciatica symptom is any of inflammation, nerve damage, pain, difficulty walking, muscle weakness, and numbness.

In some embodiments, the inflammatory condition or disorder is peripheral neuritis. In some embodiments, the subject experiences a reduction in the severity of a peripheral neuritis symptom. In some embodiments, the peripheral neuritis symptom is any of inflammation, nerve damage, pain, difficulty walking, muscle weakness, or numbness.

In some embodiments, the inflammatory condition or disorder is post-treatment Lyme disease (PTLD)/chronic Lyme disease (CLD) syndrome. In some embodiments, the subject experiences a reduction in the severity of a PTLD/CLD syndrome symptom. In embodiments, the PTLD/CLD syndrome symptom is any of difficulty thinking or concentrating, exhaustion, achiness, sensitivity, depression, and poor sleep quality.

In some embodiments, the inflammatory condition or disorder is chronic fatigue and immune dysfunction syndrome (CFIDS)/chronic fatigue syndrome (CFS). In some embodiments, the subject experiences a reduction in the severity of a chronic fatigue and immune dysfunction syndrome (CFIDS)/chronic fatigue syndrome (CFS) symptom. In some embodiments, the chronic fatigue and immune dysfunction syndrome (CFIDS)/chronic fatigue syndrome (CFS) symptom is any of difficulty thinking or concentrating and low post-exertion energy levels.

In some embodiments, the inflammatory condition or disorder is an inflammatory skin condition. In embodiments, the subject experiences a reduction in the severity of an inflammatory skin condition symptom. In embodiments, the inflammatory skin condition symptom is sensitivity to sunlight and allergen tolerance.

In some embodiments, the therapeutic combination further comprises an additional active agent.

The foregoing has outlined broadly and in summary certain pertinent features of the disclosure so that the detailed description of the invention that follows may be better understood, and so that the present contribution to the art can be more fully appreciated. Hence, this summary is to be considered as a brief and general synopsis of only some of the objects and embodiments disclosed herein, is provided solely for the benefit and convenience of the reader, and is not intended to limit in any manner the scope, or range of equivalents, to which the claims are lawfully entitled. Additional features of the invention are described hereinafter. It should be appreciated by those in the art that all disclosed specific compositions and methods are only exemplary, and may be readily utilized as a basis for modifying or designing other compositions and methods for carrying out the same purposes. Such equivalent compositions and methods will be appreciated to be also within the scope and spirit of the invention as set forth in the claims.

The headings within this document are being utilized only to expedite its review by a reader. They should not be construed as limiting the invention in any manner.

### BRIEF DESCRIPTION OF THE FIGURES

To further clarify various aspects of the invention, a more particular description is rendered by reference to certain exemplary embodiments illustrated in the figures. It will be appreciated that these figures depict only illustrated embodiments of the invention and should not be considered limiting of its scope. They are merely provided as exemplary illustrations of certain concepts of some embodiments of the invention. Certain aspects of the invention are therefore further described and explained with additional specificity and detail, but still by way of example only, with reference to the accompanying figures in which:

FIG. 1 illustrates certain synergistic effects of constituents of an exemplary therapeutic combination on NF-κB pathways in microglia.

FIG. 2 illustrates certain synergistic effects of constituents of an exemplary therapeutic combination on ROS/PPARy/Nrf2 crosstalk.

FIG. 3 illustrates the production of an exemplary composition according to the methods of certain described embodiments.

FIG. 4 illustrates an example analysis of a composition via liquid chromatography-mass spectrometry (LC-MS) testing, including chromatograms showing the various concentrations of bioactive molecules contained in a composition with parameters disclosed in TABLE 6.

FIG. 5 illustrates an example ¹H NMR spectrum of a disclosed composition having parameters described in TABLE 6.

FIG. 6 illustrates an example analysis of a composition via multiple reaction monitoring (MRM) LC-MS testing, including a chromatogram showing the concentration of bioactive molecules contained in a composition with parameters disclosed in TABLE 6.

FIG. 7 illustrates total antioxidant capacity of Cannabis Stock, measured via Folin-Ciocalteu assay, wherein the calculated Gallic Acid Equivalents (GAEs) of Cannabis Stock in milligrams (mg) are plotted per milliliter/liter (mL/L) of a given solution.

FIG. 8 illustrates total antioxidant capacity of Psilocybe Stock, using Folin-Ciocalteu assay, with the calculated GAEs of Psilocybe Stock in mg plotted per mL/L of a given solution.

FIG. 9 illustrates total antioxidant capacity of Pyropia Stock, using Folin-Ciocalteu assay, with the calculated GAEs of Pyropia Stock in mg plotted per mL/L of a given solution.

FIG. 10 illustrates total antioxidant capacity of Tonka Concentrate, using Folin-Ciocalteu assay, with calculated GAEs of Tonka Concentrate in mg plotted per mL/L of a given solution.

FIG. 11 illustrates the total antioxidant capacity of Cannabis Stock (Ca Stock), Psilocybe Stock (μs Stock), and the 2-ingredient blend of Cannabis/Psilocybe (Ca/Ps), using (i.e., obtained via) Folin-Ciocalteu assay. FIG. 11 shows calculated GAEs for Ca Stock, for Ps Stock, and for Ca/Ps in mg per mL/L of solution utilized. The table under the graph shown in FIG. 11 depicts the statistical significance of Ca Stock to Ca/Ps versus Ps Stock to Ca/Ps, and is indicated by asterisks (P<0.10: (*), P<0.05: *, P<0.01: **). The 2-ingredient blend of Cannabis and Psilocybe had a higher antioxidant capacity than each ingredient alone, and these data suggest an additive effect, where the 2 ingredients contributed to the capacity of the 2-ingredient blend.

FIG. 12 illustrates the total antioxidant capacity of Cannabis Stock (Ca Stock), Pyropia Stock (Py Stock), and the 2-ingredient blend of Cannabis/Pyropia (Ca/Py), obtained via Folin-Ciocalteu assay. FIG. 12 shows the calculated GAEs for Ca Stock, for Py Stock, and for Ca/Py in mg per mL/L of solution utilized. The table under the graph shown in FIG. 12 depicts the statistical significance of Ca Stock to Ca/Py versus Py Stock to Ca/Py, and is indicated by asterisks (as above). The 2-ingredient blend of Cannabis and Pyropia had a higher antioxidant capacity than each ingredient alone, and these data suggest a synergistic effect, since the antioxidant capacity of the 2-ingredient blend was more than what would be expected by adding the numbers for the two ingredients together.

FIG. 13 illustrates the total antioxidant capacity of Cannabis Stock (Ca Stock), Tonka Stock (To Stock), and the 2-ingredient blend of Cannabis/Tonka (Ca/To), obtained via Folin-Ciocalteu assay. FIG. 13 shows the calculated GAEs for Ca Stock, for To Stock, and for Ca/To in mg per mL/L of solution utilized. The table under the graph shown in FIG. 13 depicts the statistical significance of Ca Stock to Ca/To versus To Stock to Ca/To, and is indicated by asterisks (as above). The 2-ingredient blend of Cannabis and Tonka had a similar antioxidant capacity as the Cannabis ingredient. Tonka provided a slight enhancement of the antioxidant capacity, despite its relatively low proportion in the blend.

FIG. 14 illustrates the total antioxidant capacity of Psilocybe Stock (Ps Stock), Pyropia Stock (Py Stock), and the 2-ingredient blend of Psilocybe/Pyropia (Ps/Py), obtained via Folin-Ciocalteu assay. FIG. 14 shows the calculated GAEs for Ps Stock, for Py Stock, and for Ps/Py in mg per mL/L of solution utilized. The table under the graph shown in FIG. 14 depicts the statistical significance of Ps Stock to Ps/Py versus Py Stock to Ps/Py, and is indicated by asterisks (as above). The 2-ingredient blend of Psilocybe and Pyropia had slightly lower antioxidant capacity than Psilocybe alone. This suggests that compounds in the Pyropia stock solution interacted in a way that reduced the antioxidant capacity, i.e., the ability to donate electrons to the chemical reaction in the assay.

FIG. 15 illustrates the total antioxidant capacity of Psilocybe Stock (Ps Stock), Tonka Stock (To Stock), and the 2-ingredient blend of Psilocybe/Tonka (Ps/To), obtained via Folin-Ciocalteu assay. FIG. 15 shows the calculated GAEs for Ps Stock, for To Stock, and for Ps/To in mg per mL/L of solution utilized. The table under the graph shown in FIG. 15 depicts the statistical significance of Ps Stock to Ps/To versus To Stock to Ps/To, and is indicated by asterisks (as above). The 2-ingredient blend of Psilocybe and Tonka had a lower antioxidant capacity than Psilocybe alone. This suggests that compounds in Tonka interacted with compounds in Psilocybe in a way that reduced the antioxidant capacity, i.e., the ability to donate electrons to the chemical reaction in the assay.

FIG. 16 illustrates the total antioxidant capacity of Pyropia Stock (Py Stock), Tonka Stock (To Stock), and the 2-ingredient blend of Pyropia/Tonka (Py/To), using Folin-Ciocalteu assay. FIG. 16 shows the calculated GAEs for Py Stock, for To Stock, and for Py/To in mg per mL/L of solution utilized. The table under the graph shown in FIG. 16 depicts the statistical significance of Py Stock to Py/To versus To Stock to Py/To, and is indicated by asterisks (as above). The 2-ingredient blend of Pyropia and Tonka had a similar antioxidant capacity as Pyropia alone. Tonka's effects alone were undetectable at the dose range tested.

FIG. 17 illustrates the total antioxidant capacity for the blend of Cannabis/Psilocybe/Pyropia/Tonka (Ca/Ps/Py/To) compared to matching doses of Cannabis Stock (Ca Stock), Psilocybe Stock (Ps Stock), Pyropia Stock (Py Stock), and Tonka Stock (To Stock), obtained via Folin-Ciocalteu assay. FIG. 17 shows the calculated GAEs for Ca/Ps/Py/To, for Ca Stock, for Ps Stock, for Py Stock, and for To Stock in mg per mL/L of solution utilized. The table under the graph shown in FIG. 17 depicts the statistical significance of Ca/Ps/Py/To to Ca Stock versus Ca/Ps/Py/To to Ps Stock versus Ca/Ps/Py/To to Py Stock versus Ca/Ps/Py/To to To Stock, and is indicated by asterisks (as above).

FIG. 18 illustrates the total antioxidant capacity for the blend of Cannabis/Psilocybe/Pyropia/Tonka (Ca/Ps/Py/To) compared to matching doses of each of the 2-ingredient blends, including Cannabis/Psilocybe (Ca/Ps), Cannabis/Pyropia (Ca/Py), Cannabis/Tonka (Ca/To), Psilocybe/Pyropia (Ps/Py), Psilocybe/Tonka (Ps/To), and Pyropia/Tonka (Py/To), obtained via Folin-Ciocalteu assay. FIG. 18 shows the calculated GAEs for Ca/Ps/Py/To, for Ca/Ps, for Ca/Py, for Ca/To, for Ps/Py, for Ps/To, and for Py/To in mg per mL/L of solution utilized. The table under the graph shown in FIG. 18 depicts the statistical significance of Ca/Ps/Py/To to Ca/Ps versus Ca/Ps/Py/To to Ca/Py versus Ca/Ps/Py/To to Ca/To versus Ca/Ps/Py/To to Ps/Py versus Ca/Ps/Py/To to Ps/To versus Ca/Ps/Py/To to Py/To, and is indicated by asterisks (as above).

FIG. 19 illustrates the total antioxidant capacity of the 4-ingredient blend of Cannabis/Psilocybe/Pyropia/Tonka (Ca/Ps/Py/To) in comparison to Applicant's “4+” Blend (i.e., Applicant's final product disclosed herein as ABS-108 and NIM-01, and as the composition of EXAMPLE 6) obtained via Folin-Ciocalteu assay.

FIG. 19 shows the calculated GAEs for Ca/Ps/Py/To and for 4+ Blend in mg per mL/L of solution utilized. FIG. 19 depicts data as the average ±std. deviation of duplicate data points for each dose. FIG. 19 describes the statistical significance of Ca/Ps/Py/To versus 4+ Blend, and is indicated by asterisks (as above). The 4+ blend showed a higher antioxidant capacity than the 4-ingredient blend of Cannabis/Psilocybe/Pyropia/Tonka. This shows the additional ingredients in the 4+ blend contribute to the antioxidant properties of the final product.

### DETAILED DESCRIPTION OF THE INVENTION

While various aspects and features of certain embodiments are summarized above, the following detailed description illustrates some exemplary embodiments in further detail to enable one having ordinary skill in the art to which the invention belongs (equivalently as shorthand, “one of skill”) to practice such embodiments and to make and use the full scope of the invention claimed.

Many modifications, substitutions, changes, and variations in the described examples, embodiments, applications, and details of the invention illustrated herein can be made by one of skill without departing from the spirit of the invention, or the scope of the invention as described in the appended claims, and the general principles defined herein may be applied to a wide range of aspects. Thus, the invention is not intended to be limited to the aspects presented, but is to be accorded the widest scope consistent with the principles and features disclosed. The description below is designed to make such embodiments apparent to one of skill, in that the embodiments shall be both readily cognizable and readily creatable without undue experimentation, solely using the teachings herein together with the general knowledge in the art.

Among the aspects of the disclosure are therapeutic combinations comprising fungi, plants, and algae, including extracts and bioactive molecules derived therefrom. Among other aspects of the disclosure are pharmaceutical compositions, formulations, and kits comprising the therapeutic combinations. Among other aspects of the disclosure are methods of using the combinations, compositions, and formulations to treat or to prevent inflammatory conditions or disorders.

### I. GENERAL DEFINITIONS AND TERMS

As used in the specification and claims, the singular forms “a,” “an,” and “the” include plural referents unless context clearly dictates otherwise. Thus, for example, reference to “an excipient” includes reference to one or more excipients, and reference to “a bioactive molecule” includes reference to one or more bioactive molecules. Further, “a bioactive molecule” includes reference to bioactive molecule(s) within another substance, such as an extract, unless the bioactive molecule(s) are indicated expressly or by context as being purified or isolated therefrom, and even then may include another substance as long as the bioactive molecule(s) are, as one example, within the degree of purity indicated.

The terms “comprising,” “including,” “such as,” and “having” are intended to be inclusive and not exclusive (i.e., there may be other elements in addition to the recited elements). Thus, the term “including” herein means, and is used interchangeably with, the phrase “including but not limited to.” The term “or” is used herein to mean, and is used interchangeably with, the term “and/or,” unless context clearly indicates otherwise, and the specific use of the term “and/or” does not signify that any uses of “or” are disjunctive only; rather, such use simply underscores the possibility that the term “and/or” may be conjunctive in particular embodiments, but otherwise may be disjunctive, like “or.” The term “and” will be understood to be conjunctive.

Compositions “consisting of” specific bioactive molecules may include only the bioactive molecules recited, and those “consisting essentially of” specific bioactive molecules may include the recited bioactive molecules, optionally together with additional compounds or other elements that do not materially affect the basic and novel characteristic(s) of the claimed combination, composition, or method. While the term “one or more” may be used, its absence (or its replacement by the singular) does not signify the singular only; rather, such use simply underscores the possibility of multiple agents or ingredients in particular embodiments.

Where ranges are given herein, the disclosure includes embodiments in which the endpoints are included, embodiments in which both endpoints are excluded, and embodiments in which one endpoint is included and the other is excluded. It should be assumed that both endpoints are included unless indicated otherwise. It is also understood that unless otherwise indicated or otherwise evident from the context and understanding of one of skill, values that are expressed as ranges can assume any specific value or subrange within the stated ranges in different embodiments, to the tenth of the unit of the lower limit of the range, unless the context clearly dictates otherwise. It is further understood that where a series of numerical values is stated herein, the disclosure includes embodiments that relate analogously to any intervening value or range defined by any two values in the series, and that the lowest value may be taken as a minimum and the greatest value may be taken as a maximum. Numerical values, as used herein, include values expressed as percentages. For any embodiment in which a numerical value is prefaced by “about” or “approximately,” the disclosure includes an embodiment in which the exact value is recited. For any embodiment in which a numerical value is not prefaced by “about” or “approximately,” the disclosure includes an embodiment in which the value is prefaced by “about” or “approximately.” Unless otherwise indicated, all numbers expressing quantities of ingredients, properties such as concentration, reaction conditions, and so forth, used to describe and claim certain embodiments, thus are to be understood as being modified in some instances by the term “about.” “Approximately” or “about” is intended to encompass numbers that fall within a range of ±10% of a number, in embodiments within ±5% of a number, in some embodiments (including in some embodiments) within ±2% of a number, in some embodiments within ±1% of a number, in some embodiments within ±0.5% of a number, in some embodiments within ±0.1% of a number unless otherwise stated or otherwise evident from the context (except where such number would impermissibly exceed 100% of a possible value).

In some embodiments, the numerical parameters set forth in the description and claims are approximations that can vary depending upon the desired properties sought to be obtained by a particular embodiment (and as would be understood to one of skill). The term “substantially,” where it is applied to modify a feature or limitation herein, will be read in the context of the disclosure and in light of the knowledge in the art to provide the appropriate certainty, e.g., by using a standard that is recognized in the art for measuring the meaning of “substantially” as a term of degree, or by ascertaining the scope as would one of skill. In some embodiments, the numerical parameters should be construed in light of the number of reported significant digits and by applying ordinary rounding techniques. Notwithstanding that the numerical ranges and parameters setting forth the broad scope of some embodiments are approximations, the numerical values set forth in the specific examples are reported as precisely as practicable. The numerical values presented in embodiments may contain certain errors necessarily resulting from the standard deviation found in their respective testing measurements.

A comprehensive list of the abbreviations utilized by organic chemists of ordinary skill appears in the first issue of each volume of the Journal of Organic Chemistry, typically presented in the table “Standard List of Abbreviations.” The current list as of the filing date is incorporated by reference as if fully set forth herein.

Unless explicitly defined otherwise, all technical and scientific terms herein have the meaning as commonly understood by one of skill. Further definitions that may assist a reader in understanding the disclosed and exemplary embodiments are below; however, it will be appreciated that such definitions are not intended to limit the scope of the disclosure, which shall be properly interpreted and understood by reference to the full specification (as well as any plain meaning known to one of skill) in view of the language used in the claims. Terminology is for the purpose of describing particular embodiments and not intended to be limiting.

Terms having a specific meaning within the regulatory law of a jurisdiction in which this application is filed or may be in force generally should be given such meaning unless context dictates otherwise. For example, “Botanical Drug Substance” may refer to the term as defined through FDA implementing rules and regulations, and as described in the Guidance for Industry Botanical Drug Products, December 2016 (Docket No. FDA-2000-D-0103), U.S. Department of Health and Human Services, Food and Drug Administration Center for Drug Evaluation and Research (FDA. Botanical drug development guidance for industry. 2016. CDER pharmaceutical quality. CMC). Other such terms, including “Botanical Drug Product” and “Botanical Raw Materials,” shall be known similarly.

For avoidance of doubt, even though “botanical” is commonly used to mean “relating to plants,” for purposes herein “botanical,” as in a “botanical” drug substance or drug product, will be understood to include compounds, substances, and products (e.g., extracts) obtained or derived from fungal material as well, or present therein, such as psilocybin-containing or other fungi, such as defined by the FDA.

A “botanical” drug substance or drug product also will be understood to include compounds, substances, and products (e.g., extracts) obtained or derived from algal material, or present therein, wherein “algal material” refers broadly to any material from the polyphyletic group of diverse photosynthetic eukaryotic organisms that is referred to or understood in the field as “algae,” and especially those parts comprising the bioactive molecules of interest, as known by those of skill.

Botanical drug substances and drug products, and compositions comprising them, may be available by prescription or over-the-counter (“OTC”), as nutritional or dietary supplements, as “nutraceuticals,” or under any other regulatory regime; they also may be unregulated (e.g., “natural products”).

Herein the term “plant material” encompasses whole plants and also parts thereof which contain the bioactive molecules sought, for example, the aerial parts of a plant or isolated leaves, stems, flowers, fruits, roots, or combinations of any of the foregoing. With Cannabis plant material, it will be understood that the parts used primarily shall be the inflorescences (“buds”) of the flowering female plant, which generally comprise the greatest concentration of bioactive molecules, such as terpenoids and cannabinoids. Other plant parts however also shall be able to be used in the disclosed compositions and methods, as will be appreciated by those of skill.

Where fungal material is used (e.g., in an extraction process), it may be from any part of fungal fruiting bodies (“mushrooms”), from fungal sclerotia (“truffles”), as well as from mycelia or other fungal material (e.g., bioreactor biomass), unless context indicates otherwise, and any or all such parts, as well as combinations thereof, may be referred to as “fungal material.”

Generally, the nomenclature used and procedures performed herein are those known in fields relating to an aspect of the disclosure, such as biology, chemistry, natural products extraction, botany, mycology, phycology, pharmacology, and medicine, and are those that will be well known and commonly employed in such fields. Standard techniques and procedures will be those generally performed according to conventional methods in the art.

### II. COMPONENTS OF THERAPEUTIC COMBINATIONS, COMPOSITIONS, AND FORMULATIONS

In some embodiments, a therapeutic combination comprises a fungal portion, a plant portion, and an algal portion. In some embodiments, a therapeutic combination comprises a fungal portion, a first plant portion, an optional second plant portion, and an algal portion. In embodiments, a therapeutic combination comprises a fungal portion, a first plant portion, a second plant portion, and an algal portion. In some aspects, provided therapeutic combinations are useful to prevent or treat an inflammatory condition or disorder.

In some embodiments, a fungal, a plant, or an algal portion comprises an extract (i.e., a fungal, plant, or algal extract, respectively). In embodiments, a therapeutic combination comprises a fungal extract, a plant portion, and an algal portion. In some embodiments, a therapeutic combination comprises a fungal extract, a first plant portion, an optional second plant portion, and an algal portion. In some embodiments, a therapeutic combination comprises a fungal extract, a first plant portion, a second plant portion, and an algal portion. In some embodiments, a therapeutic combination comprises a fungal portion, a first plant extract, an optional second plant extract, and an algal portion. In some embodiments, a therapeutic combination comprises a fungal portion, a first plant extract, a second plant extract, and an algal portion. In some embodiments, a therapeutic combination comprises a fungal portion, a first plant portion, an optional second plant portion, and an algal extract. In some embodiments, a therapeutic combination comprises a fungal portion, a first plant portion, a second plant portion, and an algal extract. In some embodiments, a therapeutic combination comprises a fungal extract, a first plant extract, an optional second plant extract, and an algal portion. In some embodiments, a therapeutic combination comprises a fungal extract, a first plant extract, a second plant extract, and an algal portion. In some embodiments, a therapeutic combination comprises a fungal extract, a first plant portion, an optional second plant portion, and an algal extract. In some embodiments, a therapeutic combination comprises a fungal extract, a first plant portion, a second plant portion, and an algal extract. In some embodiments, a therapeutic combination comprises a fungal extract, a plant extract, and an algal extract. In some embodiments, a therapeutic combination comprises a fungal extract, a first plant extract, an optional second plant extract, and an algal extract. In some embodiments, a therapeutic combination comprises a fungal extract, a first plant extract, a second plant extract, and an algal extract.

In some embodiments, the therapeutic combination comprises a bioactive molecule from a fungus, a plant, and/or an algae. In some embodiments, a therapeutic combination comprises a bioactive molecule from a fungus, and a bioactive molecule from a plant. In some embodiments, a therapeutic combination comprises a bioactive molecule from a plant, and a bioactive molecule from an algae. In some embodiments, a therapeutic combination comprises a bioactive molecule from a fungus, a bioactive molecule from a plant, and a bioactive molecule from an algae. In some embodiments, a therapeutic combination comprises a bioactive molecule from a fungus, a bioactive molecule from a plant, a bioactive molecule from an algae, and a bioactive molecule from a fungus, a plant, and/or an algae. In some embodiments, a therapeutic combination comprises a bioactive molecule from a fungus, a first bioactive molecule from a plant, a second bioactive molecule from a plant, and a bioactive molecule from an algae.

In some embodiments, a therapeutic combination comprises a bioactive molecule from Psilocybe cubensis, a bioactive molecule from Cannabis sativa, a bioactive molecule from Pyropia yezoensis, and/or a bioactive molecule from Dipteryx odorata. In some embodiments, a therapeutic combination comprises two or more of a bioactive molecule from Psilocybe cubensis, a bioactive molecule from Cannabis sativa, a bioactive molecule from Pyropia yezoensis, and a bioactive molecule from Dipteryx odorata. In some embodiments, a therapeutic combination comprises three or more of a bioactive molecule from Psilocybe cubensis, a bioactive molecule from Cannabis sativa, a bioactive molecule from Pyropia yezoensis, and a bioactive molecule from Dipteryx odorata. In some embodiments, a therapeutic combination comprises all four of a bioactive molecule from Psilocybe cubensis, a bioactive molecule from Cannabis sativa, a bioactive molecule from Pyropia yezoensis, and a bioactive molecule from Dipteryx odorata.

In some embodiments, a disclosed therapeutic combination is a botanical formulation, such as prepared in embodiments herein, comprising whole extracts of Psilocybe cubensis fungi, Cannabis sativa plant, Pyropia yezoensis algae, and Dipteryx odorata bean. In some embodiments, a disclosed therapeutic combination is a botanical formulation comprising whole extracts of two or more of Psilocybe cubensis fungi, Cannabis sativa plant, Pyropia yezoensis algae, and Dipteryx odorata bean. In some embodiments, a disclosed therapeutic combination is a botanical formulation comprising whole extracts of three or more of Psilocybe cubensis fungi, Cannabis sativa plant, Pyropia yezoensis algae, and Dipteryx odorata bean. In embodiments, a disclosed therapeutic combination is a botanical formulation comprising whole extracts of all four of Psilocybe cubensis fungi, Cannabis sativa plant, Pyropia yezoensis algae, and Dipteryx odorata bean.

As will be further appreciated in view of the disclosure, a bioactive molecule may be provided in an extract, such as in a whole extract, in a fraction or subfraction thereof, or may be provided as an isolated compound, a substantially purified compound, or a purified compound, including one produced by biosynthetic or synthetic means, and combinations thereof.

In some embodiments, a bioactive molecule from a fungus is not also a bioactive molecule from a plant, nor is it a bioactive molecule from an algae (i.e., the bioactive molecule from a fungus is only found in fungi). In some embodiments, a bioactive molecule from a plant is not also a bioactive molecule from a fungus, nor is it a bioactive molecule from an algae (i.e., the bioactive molecule from a plant is only found in plants). In embodiments, a bioactive molecule from an algae is not also a bioactive molecule from any fungus, nor is it a bioactive molecule from any plant (i.e., the bioactive molecule from an algae is only found in algae).

In some embodiments, a bioactive molecule from a fungus is also either a bioactive molecule from a plant, or a bioactive molecule from an algae (i.e., it is also found in plants or algae, but not both). In some embodiments, a bioactive molecule from a plant is also either a bioactive molecule from any fungus, or a bioactive molecule from an algae (i.e., it is also found in fungi or algae, but not both). In some embodiments, a bioactive molecule from an algae is also either a bioactive molecule from any fungus, or a bioactive molecule from a plant (i.e., it is also found in fungi or plants, but not both).

In some embodiments, a bioactive molecule from a fungus is also a bioactive molecule from a plant, or a bioactive molecule from an algae (i.e., it is also found in plants or algae). In some embodiments, a bioactive molecule from a plant is also a bioactive molecule from a fungus, or a bioactive molecule from an algae (i.e., it is also found in fungi or algae). In embodiments, a bioactive molecule from an algae is also a bioactive molecule from a fungus, or a bioactive molecule from a plant (i.e., it is also found in fungi or plants).

The term “secondary,” as in “secondary bioactive,” is used herein to mean, and may be used interchangeably with, the terms “second” or “additional,” and is not intended to necessarily signify any specific degree of priority when compared to the term “primary,” as in “primary bioactive,” and hence should not necessarily imply a lower degree of importance or significance, such as importance or significance in any specific combination, composition, kit, or method; in providing or contributing to any specific advantage, benefit, or synergy; or for any specific disclosed use.

### A. Fungi

Fungi are organisms that, unlike plants, do not contain chlorophyll and are saprophytic (they feed on dead plant and animal material), parasitic (they feed off a living host), or symbiotic (they share a mutually beneficial relationship with another organism). Fungi reproduce both sexually and asexually through spores developed in various ways. Exemplary fungi include the classes Phycomycetes, Ascomycetes, and Basidiomycetes. Fungi may include what are commonly referred to as yeasts, rusts, smuts, mildews, molds, and mushrooms. Herein, “fungi” should be appreciated to include both filamentous and non-filamentous fungi species, and the term will be understood to include all forms of the word, including “fungal” and “fungus.”

In some exemplary embodiments, the fungi are psilocybin-producing fungi. A “psilocybin-producing” fungus (or psilocybin-producing fungi) is any fungus that produces or is capable of producing psilocybin. Over 100 species in the Psilocybe genus of fungi produce psilocybin. Psilocybin-producing species also can be found in a number of other genera, including Athelia, Conocybe, Copelandia, Fibularhizoctonia, Galerina, Gymnopilus, Inocybe, Mycena, Panaeolus, Pholiotina, and Pluteus. In some embodiments, the psilocybin-producing species is from any of these genera. In embodiments, the psilocybin-producing species is from any of the genera Copelandia, Galerina, Gymnopilus, Inocybe, Panaeolus, Pholiotina, Pluteus, and Psilocybe. In embodiments, it is from Psilocybe. Different species of psilocybin mushrooms, and different strains thereof, will be readily known or readily identifiable to those in the art.

In some embodiments, the psilocybin-producing fungus is a Psilocybe spp. fungus. In some embodiments, the Psilocybe spp. fungus is any of a P. acutipilea, P. allenii, P. alutacea, P. angulospora, P. antioquiensis, P. araucariicola, P. atlantis, P. aquamarina, P. armandii (Mexicana), P. aucklandiae, P. azteconim, P. azurescens, P. baeocystis, P. banderillensis, P. bispora, P. brasiliensis, P. bnunneocystidiata, P. caenjleoannulata, P. caerulescens, P. caenulipes, P. callosa, P. carbonaria, P. caribaea, P. chuxiongensis, P. collybioides, P. columbiana, P. congolensis, P. cordispora, P. cubensis, P. cyanescens, P. cyanofibrillosa, P. dumontii, P. egonii, P. eximia, P. fagicola, P. farinacea, P. fimetaria, P. fuliginosa, P. furtadoana, P. galindoi, P. gallaeciae, P. graveolens, P. guatapensis, P. heimii, P. herrerae, P. hispanica, P. hoogshagenii, P. inconspicua, P. indica, P. isabelae, P. jacobsii, P. jaliscana, P. kumaenorum, P. laurae, P. lazoi, P. liniformans, P. mexicana, P. mairei, P. makarorae, P. mammillata, P. medullosa, P. meridensis, P. meridionalis, P. mescaleroensis, P. moseri, P. muliercula, P. naematoliformis, P. natalensis, P. natarajanii, P. neorhombispora, P. neoxalapensis, P. ovoideocystidiata, P. papuana, P. paulensis, P. pelliculosa, P. pintonii, P. pleurocystidiosa, P. plutonia, P. portoricensis, P. pseudoaztecorum, P. puberula, P. quebecensis, P. rickii, P. rostrate, P. rzedowskii, P. samuiensis, P. schultesii, P. semilanceata, P. septentrionalis, P. serbica, P. sierrae, P. sylvatica, P. singer; P strictipes, P. stuntzii, P. subacutipilea, P. subaenuginascens, P. subaenuginosa, P. subcaerulipes, P. subcubensis, P. subpsilocybioides, P. subtropicalis, P. tampanensis, P. thaicordispora, P. thaiaerugineomaculans, P. thaiduplicatocystidiata, P. uruguayensis, P. uxpanapensis, P. venenata, P. villarrealiae, P. weilii, P. weldenii, P. weraroa, P. wrightii, P. yungensis, P. zapotecoantillarum, P. zapotecocaribaea, or P. zapotecorum species, including strains thereof.

In some embodiments, the psilocybin-producing fungus is not a Psilocybe spp. fungus. Other psilocybin-producing fungi, not of the Psilocybe genus, will be readily known to those in the art. Non-limiting examples include Conocybe siligineoides, Conocybe velutipes, Copelandia tropica, Inocybe aeruginascens, Inocybe caenulata, Inocybe coelestium, Inocybe corydalina, Inocybe haemacta, Inocybe tricolor, Galerina steglichii, Gymnopilus aeruginosus, Gymnopilus braendlei, Gymnopilus cyanopalmicola, Gymnopilus dilepis, Gymnopilus dunensis, Gymnopilus intermedius, Gymnopilus lateritius, Gymnopilus luteofolius, Gymnopilus luteoviridis, Gymnopilus luteus, Gymnopilus palmicola, Gymnopilus purpuratus, Gymnopilus subpurpuratus, Gymnopilus subspectabilis, Gymnopilus validipes, Gymnopilus viridans, Panaeolus venezolanus, Panaeolus tropicalis, Panaeolus tinunelveliensis, Panaeolus nubricaulis, Panaeolus olivaceus, Panaeolus moellerianus, Panaeolus microsporus, Panaeolus lentisponus, Panaeolus fimicola, Panaeolus cyanescens, Panaeolus cinctulus, Panaeolus chlorocystis, Panaeolus cambodginiensis, Panaeolus bisporus, Panaeolus axfordii, Panaeolus africanus, Panaeolus affinis, Pholiotina cyanopus, Pholiotina smithii, Pluteus albostipitatus, Pluteus americanus, Pluteus cyanopus, Pluteus glaucus, Pluteus glaucotinctus, Pluteus nigroviridis, Pluteus phaeocyanopus, Pluteus salicinus, Pluteus saupei, Pluteus velutinomatus, and Pluteus villosus.

In embodiments, a bioactive molecule from a fungus is any of those described in Venturella et al. Int J Mol Sci. 2021; 22(2):634; Anusiya et al. Bioengineered. 2021; 12(2):11239-11268; Thu et al. Molecules. 2020; 25(8):1972; Muszynska et al. Food Chem. 2018; 243:373-381; and Mishraki-Berkowitz et al. J Forensic Sci. 2020; 65(5):1450-1457.

1. Primary and Secondary Bioactive Molecules from Fungi

In some embodiments, a therapeutic combination comprises a fungal portion. In some embodiments, the fungal portion comprises a fungal extract. In some embodiments, the fungal portion comprises a bioactive molecule from a fungus. In some embodiments, the fungal extract comprises a bioactive molecule from a fungus. In embodiments, the fungal portion comprises a primary bioactive molecule and/or a secondary bioactive molecule from a fungus. In embodiments, the fungal extract comprises a primary bioactive molecule and/or a secondary bioactive molecule from a fungus. In embodiments, a disclosed therapeutic combination comprises a primary bioactive molecule and/or a secondary bioactive molecule from a fungi.

In some embodiments (equivalently, and simply as shorthand to save space, “in embodiments”), the fungal portion is from a psilocybin-producing fungus. The fungus can be any psilocybin-producing fungus known or ascertainable by those of skill, including, as non-limiting examples, certain species from the genera Athelia, Conocybe, Copelandia, Fibularhizoctonia, Galerina, Gymnopilus, Inocybe, Mycena, Panaeolus, Pholiotina, Pluteus, and Psilocybe. Different species of psilocybin-producing fungi, and different strains thereof, are described herein and readily known or readily identifiable to those in the art.

In embodiments wherein the fungal portion is described as being “from” a fungus, it will be understood that the fungal portion comprises matter derived from the fungus, including, as non-limiting examples, fungal material such as raw (i.e., unprocessed) fungal biomass, a fungal extract (e.g., extracts described herein, such as aqueous and/or ethanolic extracts), or a molecule that is naturally occurring in the fungus (e.g., a primary or secondary bioactive molecule described in embodiments herein), whether or not said molecule is actually obtained by isolating the molecule from the fungus, or another means (e.g., by chemical synthesis).

In some embodiments, the fungal portion of a therapeutic combination is from a psilocybin-producing species. In some embodiments, the fungal portion of a therapeutic combination is from a species of the genera Athelia, Conocybe, Copelandia, Fibularhizoctonia, Galerina, Gymnopilus, Inocybe, Mycena, Panaeolus, Pholiotina, Pluteus, or Psilocybe. In some embodiments, the fungal portion comprises a fungal extract from a psilocybin-producing species. In some embodiments, the fungal portion comprises a fungal extract from a species of the genera Athelia, Conocybe, Copelandia, Fibularhizoctonia, Galerina, Gymnopilus, Inocybe, Mycena, Panaeolus, Pholiotina, Pluteus, or Psilocybe. In embodiments, the fungal portion comprises a bioactive molecule from a psilocybin-producing species. In embodiments, the fungal portion comprises a bioactive molecule from a species of the genera Athelia, Conocybe, Copelandia, Fibularhizoctonia, Galerina, Gymnopilus, Inocybe, Mycena, Panaeolus, Pholiotina, Pluteus, or Psilocybe. In some embodiments, the fungal portion is from a species of the genera Copelandia, Galerina, Gymnopilus, Inocybe, Panaeolus, Pholiotina, Pluteus, or Psilocybe. In some embodiments, the fungal portion comprises a fungal extract from a species of the genera Copelandia, Galerina, Gymnopilus, Inocybe, Panaeolus, Pholiotina, Pluteus, or Psilocybe. In embodiments, the fungal portion comprises a bioactive molecule from a species of the genera Copelandia, Galerina, Gymnopilus, Inocybe, Panaeolus, Pholiotina, Pluteus, or Psilocybe.

In some embodiments, the fungi is a Psilocybe spp. fungi. In some embodiments, the fungal portion is from a Psilocybe spp. fungi. In some embodiments, the fungal portion is from any of the species P. azurescens, P. bohemica, P. semilanceata, P. baeocystis, P. cyanescens, P. tampanensis, P. cubensis, P. weilii, P. hoogshagenii, P. stuntzii, P. cyanofibrillosa, and/or P. liniformans. In some embodiments, the fungal portion comprises a fungal extract from a Psilocybe spp. fungi. In some embodiments, the fungal portion comprises a fungal extract from any of the species P. azurescens, P. bohemica, P. semilanceata, P. baeocystis, P. cyanescens, P. tampanensis, P. cubensis, P. weilii, P. hoogshagenii, P. stuntzii, P. cyano-fibrillosa, and/or P. liniformans. In some embodiments, the fungal portion comprises a fungal extract from a Psilocybe spp. fungi. In embodiments, the fungal portion comprises a bioactive molecule from any of the species P. azurescens, P. bohemica, P. semilanceata, P. baeocystis, P. cyanescens, P. tampanensis, P. cubensis, P. weilii, P. hoogshagenii, P. stuntzii, P. cyanofibrillosa, and/or P. liniformans.

In embodiments, the fungal portion comprises any of psilocybin, psilocin, baeocystin, norbaeocystin, norpsilocin, and aeruginascin. In embodiments, the fungal portion comprises a fungal extract comprising any of psilocybin, psilocin, baeocystin, norbaeocystin, norpsilocin, and aeruginascin. In embodiments, the fungal portion comprises a primary bioactive molecule from a fungus that is selected from the group consisting of psilocybin, psilocin, baeocystin, norbaeocystin, norpsilocin, and aeruginascin. In embodiments, the fungal portion comprises psilocybin. In embodiments, the fungal portion comprises psilocin. In embodiments, the fungal portion comprises baeocystin. In embodiments, the fungal portion comprises norbaeocystin. In embodiments, the fungal portion comprises norpsilocin. In embodiments, the fungal portion comprises aeruginascin. In embodiments, the fungal portion comprises a fungal extract comprising psilocybin. In embodiments, the fungal portion comprises a fungal extract comprising psilocin. In embodiments, the fungal portion comprises a fungal extract comprising baeocystin. In embodiments, the fungal portion comprises a fungal extract comprising norbaeocystin. In embodiments, the fungal portion comprises a fungal extract comprising norpsilocin. In embodiments, the fungal portion comprises a fungal extract comprising aeruginascin. In embodiments, the fungal portion comprises a primary bioactive molecule from a fungus, wherein the molecule is psilocybin. In embodiments, the fungal portion comprises a primary bioactive molecule from a fungus, wherein the molecule is psilocin. In embodiments, the fungal portion comprises a primary bioactive molecule from a fungus, wherein the molecule is baeocystin. In embodiments, the fungal portion comprises a primary bioactive molecule from a fungus, wherein the molecule is norbaeocystin. In embodiments, the fungal portion comprises a primary bioactive molecule from a fungus, wherein the molecule is norpsilocin. In embodiments, the fungal portion comprises a primary bioactive molecule from a fungus, wherein the molecule is aeruginascin.

In some embodiments, the fungal portion comprises both psilocybin and psilocin. In some embodiments, the fungal portion comprises a fungal extract comprising both psilocybin and psilocin. In some embodiments, the fungal portion comprises, as primary bioactive molecules from a fungus, both of psilocybin and psilocin. In some embodiments, the fungal portion comprises both psilocybin and psilocin in a weight ratio of between about 100:1 and 1:100, 50:1 and 1:50, 10:1 and 1:10, 5:1 and 1:5, 2:1 and 1:2, 5:3 and 3:5, or 3:2 and 2:3, all ranges inclusive. In some embodiments, fungal portion comprises psilocybin and psilocin in a molar ratio of 100:1 and 1:100, 50:1 and 1:50, 10:1 and 1:10, 5:1 and 1:5, 2:1 and 1:2, 5:3 and 3:5, or 3:2 and 2:3, all ranges inclusive.

In some embodiments, the fungal portion comprises both psilocybin and psilocin in a weight ratio of about 100:1, 50:1, 10:1, 5:1, 2:1, 5:3, 3:2, or 1:1. In some embodiments, the fungal portion comprises both psilocybin and psilocin in a molar ratio of about 100:1, 50:1, 10:1, 5:1, 2:1, 5:3, 3:2, or 1:1. In some embodiments, the fungal portion comprises both psilocybin and psilocin in a weight ratio of about 1:100, 1:50, 1:10, 1:5, 1:2, 3:5, 2:3, or 1:1. In some embodiments, the fungal portion comprises both psilocybin and psilocin in a molar ratio of about 1:100, 1:50, 1:10, 1:5, 1:2, 3:5, 2:3, or 1:1.

In some embodiments, the fungal portion comprises both psilocybin and psilocin in a weight ratio of about 5:3, 3:2, 1:1, 2:3, or 3:5. In some embodiments, the fungal portion comprises both psilocybin and psilocin in a molar ratio of about 5:3, 3:2, 1:1, 2:3, or 3:5.

In some embodiments, a fungal portion, a fungal extract, or a mixture of bioactive molecules from a fungus comprises psilocybin and psilocin in a weight ratio of about 5:3, 3:2, or 1:1. In embodiments, a fungal portion, a fungal extract, or a mixture of bioactive molecules from a fungus comprises psilocybin and psilocin in a molar ratio of about 5:3, 3:2, or 1:1. In some embodiments, a fungal portion, a fungal extract, or a mixture of bioactive molecules from a fungus comprises psilocybin and psilocin in a weight ratio of about 5:3. In embodiments, a fungal portion, a fungal extract, or a mixture of bioactive molecules from a fungus comprises psilocybin and psilocin in a molar ratio of about 5:3.

In some embodiments, the fungal portion comprises a β-carboline (beta-carboline). In some embodiments, the fungal portion comprises a fungal extract comprising a β-carboline. In some embodiments, the fungal portion comprises a primary bioactive molecule from a fungus, wherein the primary bioactive molecule is a β-carboline. In embodiments, the β-carboline is harmane, harmine, harmol, harmalol, harmaline, tetrahydroharmine, pinoline, cordysinin C, cordysinin D, norharmane, perlolyrine, β-carboline (9H-pyrido[3,4-b]indole), or another L-tryptophan-derived β-carboline. Although “β-carboline” may refer to both the individual compound and the class of related compounds, use of the term herein will be understood as referring to the class of compounds (including when recited as “a β-carboline”) unless context demands otherwise.

In embodiments, the secondary bioactive molecule from a fungus is a polysaccharide (including a and β-glucans and polysaccharide-protein complexes), a peptide (including proteins such as lectins), a terpene or terpenoid (including mono and sesquiterpene oils, diterpenes, triterpenoids and sterols, and carotenoid pigments), a phenolic compound (including phenolic acids, hydroxycinnamic acids, hydroxybenzoic acids, ligans, tannins, flavonoids, stilbenes, and oxidized polyphenols), a mineral (including potassium, phosphorous, sodium, calcium, magnesium, copper, selenium, iron, and zinc), a vitamin (including ascorbic acid, vitamin D, riboflavin, folate, thiamine, pantothenic acid, and niacin), an amino acid (including the essential amino acids, and including histidine, isoleucine, leucine, lysine, methionine, phenylalanine, taurine, threonine, tryptophan, and valine), a lipid (including saturated, monounsaturated, and polyunsaturated fatty acids such as oleic, linoleic, and linolenic acids), a choline, or a lactone.

### B. Plants

In some embodiments, a therapeutic combination comprises a plant portion. In some embodiments, the plant portion comprises a plant extract. In some embodiments, the plant portion comprises a bioactive molecule from a plant. In some embodiments, the plant extract comprises a bioactive molecule from a plant. For example, in embodiments, the plant portion comprises a primary bioactive molecule and/or a secondary bioactive molecule from a plant. In embodiments, the plant extract comprises a primary bioactive molecule and/or a secondary bioactive molecule from a plant. In some embodiments, a disclosed therapeutic combination comprises a primary bioactive molecule and/or a secondary bioactive molecule from a plant.

In embodiments wherein the plant portion is described as being “from” a plant, it will be understood that the plant portion comprises matter derived from the plant, including, as non-limiting examples, plant material such as raw (i.e., unprocessed) plant biomass, a plant extract (e.g., extracts described herein, such as aqueous and/or ethanolic extracts), or a molecule that is naturally occurring in the plant (e.g., a primary or secondary bioactive molecule described in embodiments herein), whether or not said molecule is actually obtained by isolating the molecule from the plant, or another means (e.g., by chemical synthesis).

Plants are photosynthetic eukaryotes of the kingdom Plantae. In some embodiments, a therapeutic combination comprises a plant extract. In some embodiments, a therapeutic combination comprises a primary bioactive molecule and/or a secondary bioactive molecule from a plant. In some embodiments, a therapeutic combination comprises a Cannabis extract and/or Dipteryx extract. In some embodiments, a therapeutic combination comprises a Cannabis extract. In some embodiments, a therapeutic combination comprises a Dipteryx extract. In some embodiments, a therapeutic combination comprises both a Cannabis extract and a Dipteryx extract. In some embodiments, the therapeutic combination comprises a primary bioactive molecule and/or a secondary bioactive molecule from a plant in the genera Cannabis and/or Dipteryx. In some embodiments, the therapeutic combination comprises a primary bioactive molecule and/or a secondary bioactive molecule from a plant in the genera Cannabis.

### 1. Cannabis

Cannabis is a genus of flowering plant in the family Cannabaceae. The number of species in the genus is disputed, and some recognize three separate species, Cannabis sativa, Cannabis indica, and Cannabis ruderalis; some include C. ruderalis within C. sativa; some include C. sativa, C. indica, and C. ruderalis as subspecies of a single species, C. sativa; and some treat C. sativa L as a single undivided species. Herein, “Cannabis” refers to all such encompassed species, subspecies, cultivars, varieties, variants, strains, chemovars, and the like, comprising the genus Cannabis, independent of any such terminology. Simply for shorthand herein, and without a position on the above, the term “Cannabis” will refer to C. sativa, C. indica, and C. ruderalis, and will further include any genetic crosses, self-crosses, and hybrids thereof.

In some embodiments, the plant portion of a therapeutic combination is from Cannabis. In embodiments wherein the plant portion is described as being “from” Cannabis, it will be understood that the plant portion comprises matter derived from Cannabis, including, as non-limiting examples, Cannabis material such as raw (i.e., unprocessed) Cannabis biomass, a Cannabis extract (e.g., extracts described herein, such as aqueous and/or ethanolic extracts), or a molecule that is naturally occurring in Cannabis (e.g., a primary or secondary bioactive molecule described in embodiments herein), whether or not said molecule is actually obtained by isolating the molecule from Cannabis, or another means (e.g., by chemical synthesis).

In some embodiments, a therapeutic combination comprises a Cannabis extract. In some embodiments, a therapeutic combination comprises a primary bioactive molecule from Cannabis. In some embodiments, a combination comprises a secondary bioactive molecule from Cannabis.

a. Primary and Secondary Bioactive Molecules from Cannabis

In some embodiments, a therapeutic combination comprises a primary and/or secondary bioactive molecule from Cannabis.

In some embodiments, the primary bioactive molecule from Cannabis is a cannabinoid. Cannabinoids are a diverse class of small molecules grouped together because of their ability to act on cannabinoid receptors. Cannabinoid receptors are found in the brain and throughout the central and peripheral nervous systems of humans and other mammals. There are two major types of cannabinoid receptors, known as the cannabinoid receptor 1 (CB₁) and cannabinoid receptor 2 (CB₂). CB₁ receptors are primarily found in the central nervous system (i.e., the brain and spinal cord), as well as in the lungs, liver, and kidneys. CB₁ receptor-mediated signaling plays a critical role in neural circuitry that mediates mood, motivation, and emotional behaviors. CB₂ receptors are primarily found in the immune system and hematopoietic stem and progenitor cells, and also may be found in neurons.

Cannabinoids will be known to those in the art and some are also set forth and described in Radwan et al. Molecules. 2021 May 8; 26(9):2774 (“Radwan”), which is incorporated by reference as if fully set forth herein. Without being limited thereby, cannabinoids according to the categorization of Radwan include compounds with a characteristic C21 terpenophenolic backbone that are part of one of 11 cannabinoid sub-classes, namely: cannabichromene (CBC)-type, cannabidiol (CBD) type, cannabielsoin (CBE) type, cannabigerol (CBG) type, cannabicyclol (CBL) type, cannabinol (CBN) type, cannabinodiol (CBND) type, cannabitriol (CBT) type, (−)-Δ⁸-trans-tetrahydrocannabinol (Δ⁸-THC) type, (−)-Δ⁹-trans-tetrahydrocannabinol (Δ⁹-THC) type, and miscellaneous-type cannabinoids. Non-limiting examples of cannabinoids, which will be understood to be useful in the practice of the disclosure, are known by reference to the disclosure of Radwan and the below. In embodiments, the primary bioactive molecule from Cannabis is any such cannabinoid.

In embodiments, the primary bioactive molecule from Cannabis is any of Δ⁹-THC-type cannabinoid, a Δ⁸-THC-type cannabinoid, a CBG-type cannabinoid, a CBD-type cannabinoid, a CBND-type cannabinoid, a CBE-type cannabinoid, a CBL-type cannabinoid, a CBN-type cannabinoid, a CBC-type cannabinoid, a CBT-type cannabinoid, or a miscellaneous-type cannabinoid.

Examples of Δ⁹-THC-type cannabinoids include Δ⁹-THC-C₅, Δ⁹-THCAA-C₅, Δ⁹-THCAB-C₅, Δ⁹-THC-C₄, Δ⁹-THCAA-C₄, Δ⁹-THCV, Δ⁹-THCVAA, Δ⁹-THCO, Δ⁹-THCOAA, Δ⁹-THC-aldehyde, β-fenchyl (−)-Δ9-trans-tetrahydrocannabinolate, α-fenchyl (−)-Δ9-trans-tetrahydrocannabinolate, epi-bornyl (−)-Δ9-trans-tetrahydrocannabinolate, bornyl (−)-Δ9-trans-tetrahydrocannabinolate, α-terpenyl (−)-Δ9-trans-tetrahydrocannabinolate, 4-terpenyl (−)-Δ9-trans-tetrahydrocannabinolate, α-cadinyl (−)-Δ9-trans-tetrahydro-cannabinolate, γ-eudesmyl (−)-Δ9-trans-tetrahydrocannabinolate, 8α-hydroxy-(−)-Δ9-trans-tetrahydro-cannabinol, 8γ-hydroxy-(−)-Δ9-trans-tetrahydrocannabinol, 11-acetoxy-(−)-Δ9-trans-tetrahydro-cannabinolic acid A, 8-oxo-(−)-Δ9-trans-tetrahydrocannabinol, cannabisol, (−)-Δ9-trans-tetrahydrocannabiphorol, and (−)-Δ9-trans-tetrahydrocannabihexol.

Examples of Δ⁸-THC-type cannabinoids include Δ⁸-THC, Δ⁸-THCA, 10α-OH-Δ⁸-THC, 10β-OH-Δ⁸-THC, and 10a-α-hydroxy-10-oxo-A8-THC.

Examples of CBG-type cannabinoids include (E)CBG, (E)CBGA, (E)CBGG, (E)CBGAM, (E)CBGV, (E)CBGVA, (Z)CBGA, 5-acetyl-4-hydroxy-cannabigerol, (±)-6,7-trans-epoxycannabigerolic acid, (±)-6,7-cis-epoxycannabigerolic acid, (±)-6,7-cis-epoxycannabigerol, (±)-6,7-trans-epxoycannabigerol, camagerol, and sesquicannabigerol.

Examples of CBD-type cannabinoids include CBD-C₅, CBDA-C₅, CBDM-C₅, CBD-C₄, CBDV, CBDVA, CBD-C₁, CBDH, CBDP, and CBDD. Examples of CBND-type cannabinoids include CBND-C₃ and CBND-C₅. Examples of CBE-type cannabinoids include CBE-C₅, CBEAA-C₅, CBEAB-C₅, CBE-C₃, and CBEAB-C₃. Examples of CBL-type cannabinoids include CBL, CBLA, and CBLV. Examples of CBC-type cannabinoids include CBC, CBCA, ±CBCV, +CBCV, CBCVA, 4-acetoxy-CBC, (±)-3″-hydroxy-A4″-cannabichromene, (−)-7-hydroxy-canna-bichromene, and CBC-C₃. Examples of CBN-type cannabinoids include CBN-C₅, CBNA-C₅, CBN-C₄, CBN-C₃, CBN-C₂, CBN-C₁, CBNM-C₅, 8-OH-CBN, 8-OH-CBNA, 1'S—OH-CBN, and 4-terpenyl-cannabinolate. Examples of CBT-type cannabinoids include (−)-trans-CBT-C₅, (+)-trans-CBT-C₅, (±)-cis-CBT-C₅, (±)-trans-CBT-C₃, CBT-C₃-homologue, (−)-trans-CBT-OEt-C₅, (−)-trans-CBT-OEt-C₃, 8,9-Di-OH-CBT-C₅, CBDA-C₅, and 9-OH-CBT-C₅ ester.

Examples of miscellaneous-type cannabinoids include DCBF-C₅, CBF-C₅, OH-iso-HHCV-C₃ OTHC, cannabicitran, cis-Δ⁹-THC, CBCON-C₅, CBR, CBTT, CBCN-C₅, CBCN-C₃, cis-iso-Δ⁷-THCV, trans-iso-Δ⁷-THCV, trans-iso-Δ⁷-THC, CBCNB, CBCNC, CBCND, (−)-(7R)-cannabicoumarononic acid, 4-acetoxy-2-geranyl-5-hydroxy-3-n-pentylphenol, 2-geranyl-5-hydroxy-3-n-pentyl-1,4-benzoquinone, 5-acetoxy-6-geranyl-3-n-pentyl-1,4-benzoquinone, CBM, CBX, 10α-hydroxy-Δ^{9,11}-hexahydrocannabinol, 9β,1β-epoxy-hexahydrocannabinol, 9α-hydroxyhexahydrocannabinol, 7-oxo-9α-hydroxyhexahydrocannabinol, 10α-hydroxyhexahydrocannabinol, 10αR-hydroxyhexahydrocannabinol, and 9α-hydroxy-10-oxo-Δ^{6a,10a}-THC.

In embodiments, the primary bioactive molecule from Cannabis is any of Δ⁹-THC-C₅, Δ⁹-THCAA-C₅, Δ⁹-THCAB-C₅, Δ⁹-THC-C₄, Δ⁹-THCAA-C₄, Δ⁹-THCV, Δ⁹-THCVAA, Δ⁹-THCO, Δ⁹-THCOAA, Δ⁹-THC-aldehyde, β-fenchyl (−)-Δ9-trans-tetrahydrocannabinolate, α-fenchyl (−)-Δ9-trans-tetrahydro-cannabinolate, epi-bornyl (−)-Δ9-trans-tetrahydrocannabinolate, bornyl (−)-Δ9-trans-tetrahydrocannabinolate, α-terpenyl (−)-Δ9-trans-tetrahydrocannabinolate, 4-terpenyl (−)-Δ9-trans-tetrahydrocannabinolate, α-cadinyl (−)-Δ9-trans-tetrahydrocannabinolate, γ-eudesmyl (−)-Δ9-trans-tetrahydrocannabinolate, 8α-hydroxy-(−)-Δ9-trans-tetrahydrocannabinol, 8β-hydroxy-(−)-Δ9-trans-tetrahydrocannabinol, 11-acetoxy-(−)-Δ9-trans-tetra-hydrocannabinolic acid A, 8-oxo-(−)-Δ9-trans-tetrahydrocannabinol, cannabisol, (−)-Δ9-trans-tetrahydro-cannabiphorol, (−)-Δ9-trans-tetrahydrocannabihexol, Δ⁷-THC, Δ⁷-THCA, 10α-OH-Δ⁷-THC, 10β-OH-Δ⁸-THC, 10a-α-hydroxy-10-oxo-Δ8-THC, (E)CBG, (E)CBGA, (E)CBGG, (E)CBGAM, (E)CBGV, (E)CBGVA, (Z)CBGA, 5-acetyl-4-hydroxy-cannabigerol, (±)-6,7-trans-epoxycannabigerolic acid, (±)-6,7-cis-epoxycannabigerolic acid, (±)-6,7-cis-epoxycannabigerol, (±)-6,7-trans-epxoycannabigerol, camagerol, sesquicannabigerol, CBD-C₅, CBDA-C₅, CBDM-C₅, CBD-C₄, CBDV, CBDVA, CBD-C₁, CBDH, CBDP, CBDD, CBND-C₃, CBND-C₅, CBE-C₅, CBEAA-C₅, CBEAB-C₅, CBE-C₃, CBEAB-C₃, CBL, CBLA, CBLV, CBC, CBCA, CBCV, + CBCV, CBCVA, 4-acetoxy-CBC, (±)-3″-hydroxy-A4″-cannabichromene, (−)-7-hydroxy-cannabichromane, CBC-C₃, CBN-C₅, CBNA-C₅, CBN-C₄, CBN-C₃, CBN-C₂, CBN-C₁, CBNM-C₅, 8-OH-CBN, 8-OH-CBNA, 1'S—OH-CBN, 4-terpenyl-cannabinolate, (−)-trans-CBT-C₅, (+)-trans-CBT-C₅, (±)-cis-CBT-C₅, (±)-trans-CBT-C₃, CBT-C₃-homologue, (−)-trans-CBT-OEt-C₅, (−)-trans-CBT-OEt-C₃, 8,9-Di-OH-CBT-C₅, CBDA-C₅, 9-OH-CBT-C₅ ester, DCBF-C₅, CBF-C₅, OH-iso-HHCV—C₃ OTHC, cannabicitran, cis-Δ⁹-THC, CBCON-C₅, CBR, CBTT, CBCN-C₅, CBCN-C₃, cis-iso-Δ⁷-THCV, trans-iso-Δ⁷-THCV, trans-iso-Δ⁷-THC, CBCNB, CBCNC, CBCND, (−)-(7R)-cannabicoumarononic acid, 4-acetoxy-2-geranyl-5-hydroxy-3-n-pentylphenol, 2-geranyl-5-hydroxy-3-n-pentyl-1,4-benzo-quinone, 5-acetoxy-6-geranyl-3-n-pentyl-1,4-benzoquinone, CBM, CBX, 10α-hydroxy-Δ^{9,11}-hexahydrocannabinol, 9β,10β-epoxyhexahydrocannabinol, 9α-hydroxyhexahydrocannabinol, 7-oxo-9α-hydroxyhexahydrocannabinol, 10α-hydroxyhexahydrocannabinol, 10αR-hydroxyhexahydrocannabinol, and 9α-hydroxy-10-oxo-Δ^{6a,10}a-THC. In embodiments, a “cannabinoid” also includes cannabinoid carboxylic acids and their carboxylate salts (see U.S. Pat. No. 9,376,367).

Each cannabinoid also will be understood to include its isomers, such as structural isomers and stereoisomers (including enantiomers), the -A and -B isomers for each cannabinoid, double bond isomers, and other such isomers known to those of skill. Thus, “THC” will, in embodiments, be understood to include THC-A and THC-B. Reference to a cannabinoid will include the various alkyl chain lengths associated therewith, as illustrated by Cn, wherein “n” refers to the number of carbon atoms in each alkyl chain. Thus, “THC” also includes THC-C1, THC-C2, THC-C3, THC-C4, THC-C5, THC-C6, and THC-C7. Reference to a given cannabinoid also will include all possible isomers, such as but not limited to, its -A and -B isomers, together with all possible combinations of alkyl chain lengths, including chains comprised of 1, 2, 3, 4, 5, 6, or 7 carbon atoms. So, herein, reference to “THC” will include THC-C1 A, THC-C1 B, THC-C2 A, THC-C2 B, THC-C3 A, THC-C3 B, THC-C4 A, THC-C4 B, THC-C5 A, THC-C5 B, THC-C6 A, THC-C6 B, THC-C7 A, and THC-C7 B. As will be apparent to one of skill, such logic applies to all cannabinoids disclosed herein; THC is merely used to illustrate such logic, and should not be construed as limiting.

In embodiments, a cannabinoid may further comprise an additional chemical moiety substituted thereon, including methyl, alkyl, alkenyl, methoxy, alkoxy, acetyl, carboxyl, carbonyl, oxo, ester, hydroxyl, aryl, heteroaryl, cycloalkyl, cycloalkenyl, cycloalkylalkenyl, cycloalkenylalkyl, cycloalkenylalkenyl, heterocyclylalkenyl, heteroarylalkenyl, arylalkenyl, heterocyclyl, aralkyl, cycloalkylalkyl, heterocyclylalkyl, heteroarylalkyl, and the like. In embodiments, a cannabinoid includes a “synthetic cannabinoid.”

In some embodiments, the plant portion comprises both THC and CBD. In some embodiments, the plant portion comprises a plant extract comprising both THC and CBD. In some embodiments, the plant portion comprises, as primary bioactive molecules from a plant (e.g., Cannabis), both THC and CBD. In some embodiments, the plant portion comprises both THC and CBD in a weight ratio of between about 100:1 and 1:100, 50:1 and 1:50, 10:1 and 1:10, 5:1 and 1:5, 2:1 and 1:2, 5:3 and 3:5, or 3:2 and 2:3, all ranges inclusive. In some embodiments, the plant portion comprises both THC and CBD in a molar ratio of between about 100:1 and 1:100, 50:1 and 1:50, 10:1 and 1:10, 5:1 and 1:5, 2:1 and 1:2, 5:3 and 3:5, or 3:2 and 2:3, all ranges inclusive.

In some embodiments, the plant portion comprises both THC and CBD in a weight ratio of about 100:1, 50:1, 10:1, 5:1, 2:1, 5:3, 3:2, or 1:1. In some embodiments, the plant portion comprises both THC and CBD in a molar ratio of about 100:1, 50:1, 10:1, 5:1, 2:1, 5:3, 3:2, or 1:1. In some embodiments, the plant portion comprises THC and CBD in a weight ratio of about 1:100, 1:50, 1:10, 1:5, 1:2, 3:5, 2:3, or 1:1. In some embodiments, the plant portion comprises both THC and CBD in a molar ratio of about 1:100, 1:50, 1:10, 1:5, 1:2, 3:5, 2:3, or 1:1. In embodiments, the plant portion comprises both THC and CBD in a weight ratio of from about 1:5 to 5:1, including about 1:5, 1:4, 1:3, 1:2, 1:1, 2:1, 3:1, 4:1, and 5:1. In some embodiments, the plant portion comprises both THC and CBD in a molar ratio of from about 1:5 to 5:1, including about 1:5, 1:4, 1:3, 1:2, 1:1, 2:1, 3:1, 4:1, and 5:1. In embodiments, the plant portion comprises both THC and CBD in a weight ratio of about 1:1. In embodiments, the plant portion comprises both THC and CBD in a molar ratio of about 1:1. In embodiments, the plant portion comprises a plant extract (e.g., a Cannabis extract) comprising both THC and CBD, including in any of the above weight ratios. In embodiments, the Cannabis extract comprises both THC and CBD in any of the above molar ratios. In embodiments, the Cannabis extract comprises both THC and CBD in any of the above molar ratios.

In some embodiments, a therapeutic combination comprises a secondary bioactive molecule from Cannabis. In some embodiments, the secondary bioactive molecule from Cannabis is a flavone and/or flavonoid, a terpene and/or terpenoid, a carbohydrate, a fatty acid or a fatty acid ester (FAE), an amide, an amine, a phytosterol, or a phenolic compound.

Flavonoids are a broad class of water-soluble polyphenolic molecules (i.e., comprising a phenyl group (—C₆H₅) bonded to a hydroxy group (—OH)), of which about 20 are widely found in Cannabis. A primary function of flavonoids is to provide color pigmentation to plants, particularly flowers. In Cannabis, deep purple strains owe their coloration to the flavonoids, anthocyanins and anthoxanthins. Besides providing color, flavonoids also have been shown to provide health benefits through modulation of cell signaling pathways and through various anti-inflammatory, antioxidant, anti-fungal, anti-cancer, and other effects. For example, the Cannabis flavonoid, apigenin, has potent anti-anxiety, anti-inflammatory, and anti-cancer properties; butin has been shown to reduce oxidative stress-related cell dysfunction. Other bioactive flavonoids found in Cannabis include cannaflavins, kaempferol, orientin, luteolin, quercetin, silymarin, and vitexin.

In embodiments, flavonoids include those broadly described in Radwan 2021, including orientin, vitexin, isovitexin, apigenin, luteolin, kaempferol, and quercetin flavonoids; which, in embodiments, may be methylated, glycosylated, prenylated, or geranylated.

In embodiments, the flavonoid is any of orientin, orientin-O-glucoside, orientin-7-O-glucoside, orientin-7-O-rhamnoglucoside, vitexin, vitexin-O-glucoside, vitexin-7-O-glucoside, vitexin-7-O-rhamnoglucoside, cytisoside, cytisoside-glucoside, isovitexin, isovitexin-O-glucoside, isovitexin-7-O-glucoarbinoside, isovitexin-7-O-rhamnoglucoside, apigenin-7-O-glucoside, apigenin-7-O-glucuronoid, apigenin-7-O′P-coumaroyl-glucoside, 6-prenylapigenin, apigenin-6,8-di-gluco-pyranoside, luteolin-C-glucuronide, luteolin-7-O-glucuronide, canniflavin A, canniflavin B, canniflavin C, chrysoeriol, kaempferol-3-O-diglucoside, quercetin-3-O-glucoside, quercetin-3-O-diglucoside, kaempferol-3-O-sophoroside, quercetin-3-O-sophoroside, rutin, quercetin, naringenin, and naringin.

Terpenes are a large class of organic hydrocarbon compounds containing one or more repeating units of a five-carbon building block known as an isoprene unit (i.e., 2-methyl-1,3-butadiene, having the molecular formula CH₈). Being isoprene polymers, terpenes as a group are also often referred to as “isoprenoids.” Isoprene units may be linked together end-to-end to form linear chains, or may be arranged so as to form rings (thus having the molecular formula (C₅H₈)ₙ, where n is the number of linked isoprene units). Terpenes are classified in families according to the number of isoprene units from which they are constituted: as hemiterpenes (one unit), monoterpenes (two), sesquiterpenes (three), diterpenes (four), sesterterpenes (five), triterpenes (six), sesquarterpenes (seven), tetraterpenes (eight), and polyterpenes (nine or more).

Terpenes may be referred to as “terpenoids” when they have experienced oxidation and have an additional oxygen-containing functional group (for instance, after Cannabis is cured and dried), or when they are otherwise modified by addition or removal of a functional group (e.g., a methyl group (—CH₃)). The presence and particular combination of terpenes gives different plants (and different Cannabis strains) their distinctive smells and tastes. In Cannabis, terpenes are the largest group of phytochemicals, with at least 120 identified molecules. Terpenes generally make up between 10-20 percent of the total oil content produced by Cannabis resin glands. Terpenes constitute the majority of chemicals in the heated or vaporized smoke of Cannabis flowers, often consisting of greater than 50%, with cannabinoids normally accounting for 10-20%.

Although cannabinoids are more popularly understood to be responsible for the mental and physical effects of Cannabis, terpenes have demonstrated a wide array of such effects as well (see, e.g., Russo. Br J Pharmacol. 2011; 163(7):1344-1364). For example, the terpene β-myrcene has been shown to have a sedative effect, and is believed to be responsible for a heavy “body high.” β-myrcene has also demonstrated the ability to reduce inflammation and block hepatic carcinogenesis, and act as an analgesic and muscle relaxant. The terpenes linalool, nerolidol, and pulegone have also shown sedative effects. Others, such as limonene and terpinolene, have, by contrast, shown stimulating effects. Additional terpenes show yet different effects. For example, α-humulene acts as an appetite suppressant. Limonene exhibits anti-cancer, anxiolytic, and immunostimulating properties, and nerolidol also has anti-cancer properties. β-caryophyllene possesses anti-inflammatory and gastric cytoprotective effects. Pentacyclic triterpenes, such as β-amyrin and cycloartenol, show anti-bacterial, anti-fungal, anti-inflammatory, and anti-cancer properties.

Terpenes may cause effects when consumed because of the modulation of neurotransmitter systems in the brain, as terpenes easily cross the blood-brain barrier (BBB). Linalool, for example, is shown to modulate the glutamatergic and gabaminergic neurotransmitter systems, which may explain its analgesic, anti-anxiety, anti-inflammatory, and anticonvulsant properties. α-Pinene is an acetylcholinesteral inhibitor, and may thereby aid memory. Phytol, a diterpene, increases gabaminergic expression. Other terpenes have been shown to affect serotonergic and dopaminergic neurotransmitter systems.

Some terpenes interact directly with the ECS. For example, β-caryophyllene selectively binds to CB₂ receptors as a functional CB₂ agonist, supporting β-caryophyllene as having an anxiolytic and antidepressant effect. Terpenes also have been shown to alter the permeability of cell membranes and thereby modulate the effects of THC and other cannabinoids. Since terpenes are lipophilic, they interact with lipid membranes, ion channels, a variety of different receptors (including both G protein-coupled odorant and neurotransmitter receptors), and enzymes. Through these and other mechanisms, the terpenes in Cannabis not only cause effects individually, and in combination with other terpenes, but also may modulate (including synergistically) the effects of the different cannabinoids that are present.

In embodiments, the secondary bioactive molecule from Cannabis is any of a hemiterpene, a mono-terpene, a sesquiterpene, a diterpene, a sesterterpene, a triterpene, a sesquiterpene, a tetraterpene, a poly-terpene, a carbohydrate, a fatty acid or FAE, an amide, an amine, a phytosterol, and a phenolic compound.

Examples of monoterpenes include myrcene, cis-β-ocimene, trans-β-ocimene, p-cymene, α-terpinene, β-phellandrene, γ-terpinene, α-terpinolene, α-phellandrene, 3-phenyl-2-methyl-prop-1-ene, α-pinene, β-pinene, camphene, Δ³-carene, Δ⁴-carene, sabinene, α-thujene, linalool, citral B, nerol, geraniol, ipsienol, citronellol, 2-methyl-2-heptene-6-on, geranyl acetone, m-mentha-1,8-(9)-dien-5-ol, carvacrol, carvone, α-terpineol, terpinene-4-ol, pulegone, dihydrocarvone, β-terpineol, dihydrocarveyl acetate, p-cymene-8-ol, β-cyclocitral, safranal, cis-linalool oxide, perillene, sabinol, thujyl alcohol, linalool oxide, cis-carveol, cis-sabinene hydrate, sabinene hydrate, 8-cineol, 1,4-cineol, piperitone oxide, piperitenone oxide, fenchyl alcohol, fenchone, borneol, bornyl acetate, camphor, camphene hydrate, α-pinene oxide, pinocarveol, and pinocarvone.

Examples of sesquiterpenes include α-caryophyllene, β-caryophyllene, caryophyllene oxide, curcumene, α-trans-bergamotene, α-selinene, β-farnesene, longifolene, humulene epoxide I, humulene epoxide II, caryophyllene alcohol (caryophyllenol), β-bisabolene, allo-aromadendrene, calamenene, α-copaene, nerolidol, α-gurjunene, iso-caryophyllene, β-selinene, selina-3,7(11)-diene, selina-4(14),7(11)-diene, α-bisabolol, α-cedrene, α-cubebene, 5-cadinene, epi-β-santalene, farnesol, γ-cadinene, γ-elemene, γ-eudesmol, guaiol, ledol, trans-trans-α-farnesene, (Z)-β-farnesene, farnesyl acetone, α-cadinene, α-cis-bergamotene, α-eudesmol, α-guaiene, α-longipinene, α-ylangene, β-elemene, β-eudesmol, epi-α-bisabolol, γ-cis-bisabolene, γ-curcumene, γ-muurolene, γ-trans-bisabolene, viridiflorene, germacrene-B, and clovandiol. Examples of diterpenes include phytol and neophytadiene. Examples of triterpenes include friedeline and epifriedelanol. Examples of miscellaneous terpenes include vomifoliol, dihydrovomifoliol, β-ionone, and dihydroactinidiolide.

Examples of phenolic compounds, in addition to those disclosed as part of, e.g., terpenes and flavonoids, include lignans, spiro-indans, dihydrostilbenes, dihydrophenanthrene derivatives, stilbenoids, cannabispirans, denbinobin, catechin, chlorogenic acid, caffeic acid, epicatechin, luteolin-7-O-glucoside, p-coumaric acid, caffeoyl, tyramine, ferulic acid, quercetin-3-glucoside, kaempferols, apigenin-7-glucoside, luteolin, cannabisins, and apigenin.

In embodiments, the secondary bioactive molecule from Cannabis may be any of a flavonoid, including orientin, orientin-O-glucoside, orientin-7-O-glucoside, orientin-7-O-rhamnoglucoside, vitexin, vitexin-O-glucoside, vitexin-7-O-glucoside, vitexin-7-O-rhamnoglucoside, cytisoside, cytisoside-glucoside, isovitexin, isovitexin-O-glucoside, isovitexin-7-O-glucoarbinoside, isovitexin-7-O-rhamnoglucoside, apigenin-7-O-glucoside, apigenin-7-O-glucuronoid, apigenin-7-O′P-coumaroylglucoside, 6-prenylapigenin, apigenin-6,8-di-glucopyranoside, luteolin-C-glucuronide, Iuteolin-7-O-glucuronide, canniflavin A, canniflavin B, canniflavin C, chrysoeriol, kaempferol-3-O-diglucoside, quercetin-3-O-glucoside, quercetin-3-O-diglucoside, kaempferol-3-O-sophoroside, quercetin-3-O-sophoroside, rutin, quercetin, naringenin, and naringin; a terpene, including myrcene, cis-β-ocimene, trans-β-ocimene, p-cymene, α-terpinene, β-phellandrene, γ-terpinene, α-terpinolene, α-phellandrene, 3-phenyl-2-methyl-prop-1-ene, α-pinene, β-pinene, camphene, γ-carene, γ-carene, sabinene, α-thujene, linalool, citral B, nerol, geraniol, ipsienol, citronellol, 2-methyl-2-heptene-6-on, geranyl acetone, m-mentha-1,8-(9)-dien-5-ol, carvacrol, carvone, α-terpineol, terpinene-4-ol, pulegone, dihydrocarvone, β-terpineol, dihydrocarveyl acetate, p-cymene-8-ol, β-cyclocitral, safranal, cis-linalool oxide, perillene, sabinol, thujyl alcohol, linalool oxide, cis-carveol, cis-sabinene hydrate, sabinene hydrate, 8-cineol, 1,4-cineol, piperitone oxide, piperitenone oxide, fenchyl alcohol, fenchone, borneol, bornyl acetate, camphor, camphene hydrate, α-pinene oxide, pinocarveol, pinocarvone, α-caryophyllene, β-caryophyllene, caryophyllene oxide, curcumene, α-trans-bergamotene, α-selinene, β-farnesene, longifolene, humulene epoxide I, humulene epoxide II, caryophyllene alcohol (caryophyllenol), β-bisabolene, allo-aromadendrene, calamenene, α-copaene, nerolidol, α-gurjunene, iso-caryophyllene, β-selinene, selina-3,7(11)-diene, selina-4(14),7(11)-diene, α-bisabolol, α-cedrene, α-cubebene, 5-cadinene, epi-β-santalene, farnesol, γ-cadinene, γ-elemene, γ-eudesmol, guaiol, ledol, trans-trans-α-farnesene, (Z)-β-farnesene, farnesyl acetone, α-cadinene, α-cis-bergamotene, α-eudesmol, α-guaiene, α-longipinene, α-ylangene, β-elemene, β-eudesmol, epi-α-bisabolol, γ-cis-bisabolene, γ-curcumene, γ-muurolene, γ-trans-bisabolene, viridiflorene, germacrene-B, clovandiol, phytol, neophytadiene, friedeline, epifriedelanol, vomifoliol, dihydrovomifoliol, β-ionone, and dihydroactinidiolide; a carbohydrate; a fatty acid and its esters; an amide; an amine; a phytosterol; and a phenolic compound, including lignans, spiro-indans, dihydrostilbenes, dihydrophenanthrene derivatives, stilbenoids, cannabispirans, denbinobin, catechin, chlorogenic acid, caffeic acid, epicatechin, luteolin-7-O-glucoside, p-coumaric acid, caffeoyl, tyramine, ferulic acid, quercetin-3-glucoside, kaempferols, apigenin-7-glucoside, luteolin, cannabisins, and apigenin.

In embodiments, bioactive molecules from Cannabis additionally include those outlined in Russo. Br J Pharmacol. 2011; 163(7):1344-1364; Gertsch et al. Br J Pharmacol. 2010; 160(3):523-529; Tahir et al. J Cannabis Res. 2021 Mar. 15; 3(1):7; Thomas & EISohly. Biosynthesis and pharmacology of phytocannabinoids and related chemical constituents. The Analytical Chemistry of Cannabis; Elsevier: Amsterdam, The Netherlands, 27-41; De Backer et al. J Chromatogr B Analyt Technol Biomed Life Sci. 2009; 877(32): 4115-4124; and Hazekamp et al. J Liq Chromatogr Relat Technol. 2004; 27(15): 2421-2439).

### 2. Dipteryx

In some embodiments, the plant portion of a therapeutic combination is from Dipteryx. In embodiments wherein the plant portion is described as being “from” Dipteryx, it will be understood that the plant portion comprises matter derived from Dipteryx, including, as non-limiting examples, Dipteryx material such as raw (i.e., unprocessed) Dipteryx biomass, a Dipteryx extract (e.g., extracts described herein, such as aqueous and/or ethanolic extracts), or a molecule that is naturally occurring in Dipteryx (e.g., a primary or secondary bioactive molecule described in embodiments herein), whether or not said molecule is actually obtained by isolating the molecule from Dipteryx, or another means (e.g., by chemical synthesis).

In some embodiments, a therapeutic combination comprises a Dipteryx extract. In some embodiments, the therapeutic combination comprises a primary bioactive molecule and/or a secondary bioactive molecule from a species in the genus Dipteryx.

Although in some embodiments, the primary bioactive molecule and/or secondary bioactive molecule are from Dipteryx, in other embodiments, the primary bioactive molecule and/or secondary bioactive molecule are from another genus from the tribe Dipterygeae, which further includes the genera Monopteryx, Pterodon, and Taralea.

In embodiments, the primary and/or secondary bioactive molecules are from Dipteryx, including from D. odorata, also herein referred to as the “Cumaru tree” or “Cumaru.” Dipteryx is a genus of large trees belonging to the family Fabaceae, native to South and Central America and the Caribbean, and previously known as the genus Coumarouna. Although in some embodiments, a bioactive molecule, such as a coumarin, is from a plant of the genus Dipteryx, such as Dipteryx odorata, i.e., Cumaru, in other embodiments, a bioactive molecule, such as a coumarin, is from a plant of another genus, including any genera in the tribe Amburaneae, such as Amburana, Cordyla, Dupuya, Dussia, Mildbraediodendron, Myrocarpus, Myrospermum, Myroxylon, and Petaladenium. Accordingly, in some embodiments, species from such other genera, and extracts and bioactive molecules therefrom, will be considered as equivalents of like extracts and bioactive molecules from Dipteryx.

In some embodiments, the therapeutic combinations comprise primary and/or secondary bioactive molecules from the species Dipteryx odorata.

Dipteryx odorata is known by many names, including Coumarouna odorata, Cumaru tree (Brazil), Tonka bean tree, Brazilian teak, Tonquin bean, rumara, Kumaru (Guyana), cumaruzeiro (Portuguese), charapilla (Peru), charapilla del murcielago (Peru), shihuahuaco (Peru), and sarapia (Venezuela, Columbia), all of which may be used interchangeably or viewed equivalently according to the disclosure herein. Dipteryx odorata is a large rainforest canopy tree that grows up to 30 meters in height in the Amazon. It can be found in Brazil, Venezuela, Guyana, French Guiana, Suriname, Nigeria, Peru and Colombia. Historically, the seed and bark of Dipteryx odorata have been employed by local Amazonians, the seeds fermented in rum and used for snakebites, cuts, contusions, coughs, rheumatism, and as shampoo; the seed oil used to prevent earaches and ear infections, and bark to create a bath for fever patients.

The seeds (also referred to as the beans) produced by the Cumaru tree (also, “tonka beans”), containing the bioactive molecule, coumarin, have a pleasant vanilla-like odor and have been used in perfumery, soaps, and as a flavoring agent in foods and tobacco products. For their preparation, the beans are usually fermented (such as in a local rum) and then dried (generally, air-dried). This results in the formation of coumarin crystals dusting the outside of the seeds, making them appear frosted.

In embodiments, a disclosed combination (e.g., a “therapeutic combination,” the terms also including by implication a disclosed composition) comprising a bioactive molecule from Dipteryx, such as a coumarin, can exhibit antispasmodic, emmenagogue, cardiotonic, antiasthmatic, and anti-inflammatory effects.

a. Primary and Secondary Bioactive Molecules from Dipteryx

In some embodiments, a therapeutic combination comprises a primary and/or secondary bioactive molecule from Dipteryx.

In embodiments, the primary bioactive molecule from Dipteryx is coumarin (2H-chromen-2-one; 2H-1-benzopyran-2-one), a colorless crystalline solid that gives tonka beans a characteristic vanilla-like aroma and flavor, and which may also act as a chemical defense against predators.

Coumarin may be obtained from Dipteryx and from myriad other plant species, including vanilla grass (Anthoxanthum odoratum), sweet woodruff (Galium odoratum), sweet grass (Hierochloe odorata), sweet-clover (genus Melilotus), cinnamon, including ceylon cinnamon or “true cinnamon” (Cinnamomum verum), Chinese cinnamon or Chinese cassia (C. cassia), Indonesian cinnamon or Padang cassia (C. burmannii), Saigon cinnamon or Vietnamese cassia (C. loureiroi), deertongue (Carphephorus odoratissimus), Tilo (Justicia pectoralis), Mullein (genus Verbascum), many cherry blossom tree varieties of the genus Prunus, and in trace amounts in strawberries, black currants, apricots, and cherries (Ananthakrishnan et al. Int J Food Prop. 2018; 21:50-57; Wang et al. J Agric Food Chem. 2013; 61(18):4470-476; Khan & Ehab. Deertongue. In: Leung's Encyclopedia of Common Natural Ingredients Used in Food, Drugs, and Cosmetics. John Wiley & Sons. 2010. 240-242; leri et al. Food Chem. 2012; 135(4):2157-2162; and National Center for Biotechnology Information. PubChem Compound Summary for CID 323, Coumarin). In some alternative embodiments, a coumarin is extracted, isolated, or otherwise obtained from any of the above or other such species, instead of from Dipteryx.

In some embodiments, a bioactive molecule is a compound derived from coumarin (e.g., a phenylpropanoid, a coumarin, or a coumarinoid) and which may be used in disclosed combinations, compositions, and methods; such compounds include, e.g., the bioactive molecules umbelliferone, aesculetin, herniarin, psoralen, dicoumarol, imperatorin, brodifacoum, bromadiolone, difenacoum, auraptene, ensaculin, phenprocoumon, PSB-SB-487, PSB-SB-1202, scopoletin, and warfarin (see, e.g., Laposata et al. N Engl J Med. 2007; 356(2):174-182; Syah et al. Nat Prod Res. 2009; 23(7):591-594).

In embodiments, the secondary bioactive molecule from Dipteryx is any of cumaru, a coumarin derivative, an isoflavone, a lupeol derivative, a fatty acid ester, (±)-balanophonin, (−)-lariciresinol, 3′-hydroxyretusin-8-methyl-ether, 5-methoxyxanthocercin A, 6,4′-dihydroxy-3′-methoxyaurone, 7-hydroxy-chromone, 7,3′-dihydroxy-8,4′-dimethoxy-isoflavone, betulin, butin, coumaric-acid-beta-glucoside, dipteryxin, dipteryxic acid, eriodictyol, ferulic-acid, isoliquiritigenin, Iupeol, melilotoside, melilotoside-1-p-coumaryl-beta-d-glucose, methyl-linolenate, methyl-oleate, O-coumaricacid, O-hydroxycoumaric-acid, odoratin, P-hydroxy-benzoic-acid, retusin, retusin-8-methyl-ether, sulfuretin, salicylic-acid, afrormisin, castinin, linoleic acid, oleic acid, 3′,4′,7′-trihydroxyflavone, luteolin, and umbelliferone.

In embodiments, bioactive molecules from Dipteryx additionally include those in Trincone, A. (Ed.). (2019). Enzymatic Technologies for Marine Polysaccharides (1st ed.). CRC Press; Jofre et al. Mar Drugs. 2020; 18(2):75; or Gomez-Zavaglia et al. Antioxidants (Base/). 2019; 8(9):406.

### C. Algae

In some embodiments, a therapeutic combination comprises an algal portion. In some embodiments, the algal portion comprises an algal extract. In some embodiments, the algal portion comprises a bioactive molecule from an algae. In some embodiments, the algal extract comprises a bioactive molecule from an algae. For example, in some embodiments, the algal portion comprises a primary bioactive molecule and/or a secondary bioactive molecule from an algae. In embodiments, the algal extract comprises a primary bioactive molecule and/or a secondary bioactive molecule from an algae. In embodiments, a disclosed therapeutic combination comprises a primary bioactive molecule and/or a secondary bioactive molecule from an algae.

In embodiments wherein the algae portion is described as being “from” an algae, it will be understood that the algae portion comprises matter derived from the algae, including, as non-limiting examples, algae material such as raw (i.e., unprocessed) algae biomass, an algae extract (e.g., extracts described herein, such as aqueous and/or ethanolic extracts), or a molecule that is naturally occurring in the algae (e.g., a primary or secondary bioactive molecule described in embodiments herein), whether or not said molecule is actually obtained by isolating the molecule from the algae, or another means (e.g., by chemical synthesis).

Algae broadly refers to a large, polyphyletic group of photosynthetic eukaryotic organisms. Generally, no definition of algae is universally accepted; however, one means of describing the multiple clades encompassing “algae” is that they have chlorophyll as their primary photosynthetic pigment and lack a sterile covering of cells around their reproductive cells (Lee. Phycology. Cambridge University Press. 2008).

Algae includes varieties capable of living in freshwater, and/or salt water, and generally includes Euglenophyta (Euglenoids), Chrysophyta (golden-brown algae and diatoms), Pyrrophyta (fire algae), Chlorophyta (green algae), Rhodophyta (red algae), Phaeophyta (brown algae), and Xanthophyta (yellow-green algae) (Baweja & Sahoo. (2015). Classification of algae. The algae world, 31-55).

In embodiments, a disclosed combination comprises a marine algae. In embodiments, the marine algae is any of a brown algae (Phaeophyta), a green algae (Chlorophyta), and a red algae (Rhodophyta). In embodiments, the marine algae is a marine red algae. In embodiments, the marine red algae is from the family Bangiacea. In embodiments, the marine red algae is from the genus Pyropia or Porphyra.

Culturally, Porphyra has been cultivated in East Asia and utilized in the making of “nori” and other forms of edible seaweed such as that used in sushi to bind and secure rice to other proteins, such as fish. Some species of Porphyra also are harvested in Western Europe to produce laverbread, a traditional food consumed primarily in Wales as part of local traditional cuisine (Harford. Laver. https://www.eatweeds.co.uk/laver-porphyra. n.d. Accessed Jun. 6, 2022).

In embodiments, the red algae is Porphyra umbilicalis. Broadly, umbilicalis is a membranous, single-layered algae with centrally attached irregularly lobed, olive to brown fronds (up to 200 mm long) that split from a central holdfast and generally have a lettuce-like appearance. Like other Porphyra species, umbilicalis may be found growing on rocks and sometimes mussels in midtidal to splash zones and are distributed in abundance in spring and summer. In embodiments, the red algae is Pyropia perforata. Perforata is generally purple/greenish in color, has lobed blades, and can reach 30 cm in width and length. Perforata may be found growing on rocks, or as epiphytes on other species of algae. In embodiments, the red algae is Pyropia yezoensis. Yezoensis has a discoid holdfast and short stipe, and folded blades which are membranous and monostromatic, coming in red, brown, and dark green colorations.

1. Primary and Secondary Bioactive Molecules from Algae

In some embodiments, a therapeutic combination comprises a primary and/or secondary bioactive molecule from algae.

In embodiments, the primary bioactive molecule from an algae is a primary bioactive molecule from a genus in the family Bangiaceae. In embodiments, the primary bioactive molecule from an algae is a primary bioactive molecule from a species in the genus Pyropia or Porphyra.

In embodiments, the primary bioactive molecule from an algae is a primary bioactive molecule from Porphyra umbilicalis. In embodiments, the primary bioactive molecule from an algae is a primary bioactive molecule from Pyropia perforata. In embodiments, the primary bioactive molecule from an algae is a primary bioactive molecule from Pyropia yezoensis.

In embodiments, the primary bioactive molecule from Pyropia or Porphyra is any of a porphyran or oligo-porphyran, a polysaccharide, a peptide (e.g., monopeptides, dipeptides, tripeptides, proteins); a phycobiliprotein (e.g., phycoerythrin, phycoerythrobilin, phycocyanin, allophycocyanin); a mycosporine amino acid (e.g., porphyra-334, shinorine); an essential amino acid (e.g., isoleucine, leucine, threonine, methionine, phenylalanine, lysine, histidine, valine, arginine, cysteine); a nonessential amino acid (e.g., aspartic acid, glutamic acid, glycine, tyrosine, serine, alanine, proline); a carotene (e.g., lutein, zeoxanthin, α-carotene, β-carotene, astaxanthin); an intermediate carotenoid (e.g., α-cryptoxanthin, zeinoxanthin, β-cryptoxanthin); a glycoprotein; and an amino sulfonic acid (e.g., taurine).

In embodiments, the primary bioactive molecule from Pyropia or Porphyra is porphyran or oligo-porphyran. In embodiments, the primary bioactive molecule from Pyropia or Porphyra is a polysaccharide. In embodiments, the polysaccharide is any of glucose, fructose, galactose, and mannose. In embodiments, the primary bioactive molecule from Pyropia or Porphyra is a peptide. In embodiments, the peptide is any of a monopeptide, a dipeptide, a tripeptide, and a protein. In embodiments, the primary bioactive molecule from Pyropia or Porphyra is a phycobiliprotein. In embodiments, the phycobiliprotein is any of phycoerythrin, phycoerythrobilin, phycocyanin, and allophycocyanin. In embodiments, the primary bioactive molecule from Pyropia or Porphyra is a mycosporine amino acid. In embodiments, the mycosporine amino acid is any of porphyra-334 and shinorine. In embodiments, the primary bioactive molecule from Pyropia or Porphyra is an essential amino acid. In embodiments, the essential amino acid is any of isoleucine, leucine, threonine, methionine, phenylalanine, lysine, histidine, valine, arginine, and cysteine. In embodiments, the primary bioactive molecule from Pyropia or Porphyra is a nonessential amino acid. In embodiments, the nonessential amino acid is any of aspartic acid, glutamic acid, glycine, tyrosine, serine, alanine, and proline. In embodiments, the primary bioactive molecule from Pyropia or Porphyra is a carotene. In embodiments, the carotene is any of lutein, zeoxanthin, α-carotene, β-carotene, and astaxanthin. In embodiments, the primary bioactive molecule from Pyropia or Porphyra is an intermediate carotenoid. In embodiments, the intermediate carotenoid is any of α-cryptoxanthin, zeinoxanthin, and β-cryptoxanthin. In embodiments, the primary bioactive molecule from Pyropia or Porphyra is a glycoprotein. In embodiments, the primary bioactive molecule from Pyropia or Porphyra is an amino sulfonic acid. In embodiments, the amino sulfonic acid is taurine.

In embodiments, a therapeutic combination comprises a bioactive molecule from an algae, wherein the bioactive molecule, in embodiments, is a secondary bioactive molecule from an algae. In embodiments, the secondary bioactive molecule from an algae is a secondary bioactive molecule from a genus in the family Bangiaceae. In embodiments, the secondary bioactive molecule from an algae is a secondary bioactive molecule from a species in the genus Pyropia or Porphyra. In embodiments, the secondary bioactive molecule from an algae is a secondary bioactive molecule from Porphyra umbilicalis. In embodiments, the secondary bioactive molecule from an algae is a secondary bioactive molecule from Pyropia Yezoensis.

In embodiments, the secondary bioactive molecule from Pyropia or Porphyra is any of a mineral, a vitamin, a lipid, a phenolic compound, and a phlorotannin.

In embodiments, the secondary bioactive molecule from Pyropia or Porphyra is a mineral. In embodiments, the mineral is any of potassium, phosphorus, magnesium, sodium, calcium, manganese, iron, copper, and zinc. In embodiments, the secondary bioactive molecule from Pyropia or Porphyra is a vitamin. In embodiments, the vitamin is any of vitamin K, ascorbic acid, folate, and cobalamin. In embodiments, the secondary bioactive molecule from Pyropia or Porphyra is a lipid. In embodiments, the lipid is a fatty acid, such as eicosapentaenoic acid or palmitic acid. In embodiments, the secondary bioactive molecule from Pyropia or Porphyra is a phenolic compound. In embodiments, the phenolic compound is any of a flavonoid, a phenolic acid, a polyphenolic amide, and an other phenolic compound as would be appreciated by one skilled in the art. In embodiments, the secondary bioactive molecule from Pyropia or Porphyra is a phlorotannin. In embodiments, the phlorotannin is any of a fucol, a phloroethol, a fucophloroethol, an eckol, a fuhalol, and a carmalol.

### III. OBTAINING BIOACTIVE MOLECULES

In embodiments, a bioactive molecule of a therapeutic combination is commercially available, and can be commercially sourced (e.g., Cayman Chemical Co., Ann Arbor, MI; Sigma-Aldrich, Burlington, MA).

In embodiments, the disclosed primary and secondary bioactive molecules may be obtained via any of, as non-limiting examples: extraction, synthesis, biosynthesis methods, as a whole plant, as Cannabis flower, Cannabis biomass, a fruiting fungal body, fungal mycelium mass, bioreactor-produced fungal biomass, a truffle (a fungal sclerotia), a cumaru seed (tonka bean), or whole Porphyra or Pyropia algae, isolated, and/or gathered as fractions. Exemplary means of obtaining bioactive molecules are disclosed.

### A. Extraction

In some embodiments, extracts used in a disclosed therapeutic combination (e.g., a fungal extract, plant extract, or algal extract) is obtained through the use of extraction techniques known to those of skill in the art. In some embodiments, a disclosed primary and secondary bioactive molecule is provided in the form of an extract produced according to extraction techniques known to those of skill in the art.

Broadly, but without being bound by theory, an extraction system works by introducing a material, such as a fungal material, a plant material, and/or an algal material (e.g., containing a desired primary and/or secondary bioactive molecule), to a solvent capable of separating desired primary and/or secondary bioactive molecules from the material to form a solution containing the solvent and the primary and/or secondary bioactive molecules (an extract).

An extract may be used directly in a disclosed combination, composition, or method, or may be first further processed, such as by further extraction, filtration, distillation, fractionation, subfractionation, isolation, and/or purification, and other such methods known in the art, and including combinations thereof.

Various types of extraction systems exist, utilizing different methods, and having different parameters (e.g., temperature, pressure, solvent) which may be tailored to the desired end product, for example, the desired primary and/or secondary bioactive molecules. Many such properties will be known to those in the art. For example, and without being bound by theory, extraction systems generally follow the rule “like dissolves like.” Thus, when extracting a polar bioactive molecule, a polar solvent may be utilized; and when extracting a nonpolar bioactive molecule, a nonpolar solvent may be utilized (Lowery & Richardson. Mechanism and Theory in Organic Chemistry. 3rd ed. Harper Collins Publishers; 1987).

Methods of extracting primary and/or secondary bioactive molecules, of creating plant, algal, and fungal extracts thereof, and of generally obtaining purified products containing desired compounds free from undesired plant, algal, or fungal matter, chemicals, and other impurities are known in the art, see e.g., U.S. Pat. Nos. 6,403,126, 8,846,409, 8,895,078, 10,059,684, 10,239,808, 10,246,431, 10,300,494, 10,307,447, 10,406,453, 10,413,845, and 10,414,709; and U.S. App. Nos. 2003/0017216A1 and 2016/0038437A1, and references cited, all of which are incorporated by reference as if fully set forth herein.

Exemplary extraction methods and systems disclosed in such references and otherwise herein should not be construed as being limiting, and many variations will be appreciated by those of skill. While exemplary extraction methods, including those described herein, are disclosed as a series of steps, other extraction methods useful in the practice of the disclosure and in obtaining bioactive molecules therefore may deviate from such steps, including by the modification to, removal of, addition of, or rearrangement of any such steps, as will be appreciated by those of skill, in view of the disclosure and general knowledge in the art.

Herein, “extract” may refer to a botanical extract (e.g., a fungal, Psilocybe, plant, Cannabis, Dipteryx, algal, or other extract containing a primary and/or secondary bioactive molecule disclosed herein) prepared, for example, from a botanical source (as “botanical” is defined herein). In some embodiments, an extract undergoes further extraction, filtration, fractionation, subfractionation, partial purification, substantial purification, or complete purification, or other processing to obtain a specific bioactive molecule(s) separate from other constituents, wherein such bioactive molecule(s) are found in the filtrate, fraction, subfraction, partially purified product, substantially purified product, completely purified product, isolate, or the like, and in some cases may be obtained separated from all other components as a single component or as single components. In some embodiments, an extract does not undergo fractionation, subfractionation, partial purification, substantial purification, or complete purification, or other processing to obtain a specific bioactive molecule(s) separate from other constituents, and is obtained as a whole plant, whole fungal, or whole algal extract, which may be referred to herein as a “whole extract.” In alternative embodiments of any exemplary disclosed embodiment herein, a whole extract is substituted with a fraction, subfraction, partially purified product, substantially purified product, completely purified product, isolate, or the like, as well as by a single bioactive molecule or one or more molecule(s) from an extract (including in an extract), or by molecule(s) produced synthetically, such as by partial or complete chemical synthesis, or by biosynthesis.

Extracts thus may include purified extracts. “Purified extract” may refer to a botanical extract that has undergone further processing after preparation, as will be understood by those in the art. In some exemplary embodiments, purified extracts may be the product of, e.g., soaking or heating the preparation in water and/or alcohol (e.g., depending on whether and to what degree the bioactive molecules sought are water soluble), agitating, cooling the resulting liquid, straining, filtering, and removing unwanted products (repeating if necessary), and then evaporating sufficient liquid solvent to obtain a desired concentration (or entirely, e.g., to obtain an amorphous or crystalline precipitate), or using a spray dryer to create a purified dried powder. Other purification techniques will be known to those of skill, and in general, extraction and purification techniques for obtaining high purity bioactive compounds will be known in the art.

A starch or other carrier can be added to a purified extract to maintain the purified dried powder as a free-flowing powder that is easy to work with during formulation, for instance if the dried powder will be added to capsules; however, when referring to the weight of a “purified extract,” any such carrier, diluent, or excipient is excluded from the total amount.

In embodiments, extracts may be “standardized,” which refers to extracts that include a primary and/or secondary bioactive molecule in a specific concentration. Methods to produce standardized extracts, such as by quantifying the concentration of an extract, and then adding a carrier, diluent, or excipient to dilute the extract to a standardized concentration, or further concentrating an extract to increase its concentration, will be known to those of skill.

“Natural” may refer to a substance which was isolated, extracted, or otherwise obtained from a natural source, such as a fungus, plant, or algae. A natural primary or secondary bioactive molecule, such as a natural cannabinoid, therefore may be isolated or extracted from a Cannabis plant. Thus, in various disclosed embodiments, the compositions shall be derived from botanical sources including fungi, plants, and algae, as extracts or by other means, and shall comprise a botanical drug substance or a Cannabis-derived drug substance. In embodiments, such compositions shall comprise a botanical drug product or a Cannabis-derived drug product. In other embodiments, such compositions shall comprise a botanical drug product or a Cannabis-derived drug product, which is substantially free from impurities.

In some embodiments, wherein a disclosed combination or composition comprises multiple extracts (e.g., fungal extracts, plant extracts, algal extracts), the concentration of bioactive molecules contained therein is quantified (e.g., according to methods disclosed herein and otherwise known to one of skill). The concentrations of bioactive molecules in the extracts may vary, based on the concentration of bioactive molecules in the corresponding source material (e.g., raw fungal, plant, or algal biomass). Accordingly, the concentrations of bioactive molecules in disclosed compositions may vary from batch to batch, based on variability between batches of extracts. In conforming with FDA rules (FDA. Botanical drug development guidance for industry. 2016. CDER pharmaceutical quality. CMC), bioactive molecules (e.g., a primary and/or secondary bioactive molecule as disclosed herein) may be added to an extract disclosed herein (e.g., a fungal, plant, or algal extract); or to a disclosed combination or composition comprising said extracts. This may be conducted, e.g., to standardize the disclosed combination or composition and ensure a fixed concentration of a bioactive molecule between batches, even when the source materials or extracts thereof may vary in their concentrations of bioactive molecules.

1. Exemplary Extraction of Molecules from Fungi

In embodiments, a therapeutic combination comprises a primary and/or secondary bioactive molecule from a fungus. In embodiments, the primary and/or secondary bioactive molecule may be obtained via extraction of fungal material.

Without being bound by theory, fungal extraction methods can be generally described as follows. First, the fungal material comprising the desired primary and/or secondary bioactive molecules is obtained, optionally dried (using, e.g., means such as a low-temperature oven, food dehydrator, and/or commercial drier), and optionally ground, pulverized, macerated, and/or milled to form a substantially fine powder, which is optionally then sieved, e.g., for consistency.

The fungal material is then combined with a solvent capable of extracting the desired primary and/or secondary bioactive molecules (e.g., methanol, ethanol, water, or a mixture thereof) to form a slurry, and the slurry is agitated to facilitate extraction for a specified duration of time, for example about 24 hours. The solvent is then filtered and collected. In embodiments, filters may include a cheesecloth, filter paper, and/or a filtration system. Optionally, the extract may be evaporated, e.g., using ambient evaporation, rotary evaporation, vacuum evaporation, and evaporation methods where heat is applied, such as utilizing an oven, as well as combinations thereof, to create a more concentrated extract. In embodiments, the fungal material may be re-saturated with the same or different solvent after the initial filtration to complete a secondary, tertiary, or further extraction. Extraction methods and variations thereto may be chosen using ordinary skill.

In embodiments, heat extraction is utilized. In embodiments, heat extraction follows a process as generally described above, except that, for example, the solvent is heated, the agitation is completed in a heated environment, or the extraction otherwise takes place at a raised temperature relative to ambient temperature. In embodiments, extraction is completed at temperatures at which psilocybin is substantially dephosphorylated to psilocin. In embodiments, heat extraction is completed in temperatures exceeding about 70° C. In embodiments, extraction occurs at temperatures below which there is substantial dephosphorylation of psilocybin. In embodiments, extraction occurs at a temperature or temperature range chosen to produce a desired ratio of psilocybin to psilocin, e.g., based on the proportions in the starting material.

In embodiments, ultrasonic extraction is utilized. In some embodiments, mushrooms or other fungal matter is dried and cut and/or pulverized prior to ultrasonic extraction. In embodiments, an ultrasonic extraction machine is utilized to expose fungal material to ultrasonic vibrations via an ultrasonic probe. In embodiments, the ultrasonic probe vibrates at 20 kHz or more. Without being bound by theory, as ultrasonic waves travel through a liquid, they create alternating high-pressure (compression) and low-pressure (rarefaction or expansion) cycles (Hielscher Ultrasonics. Fast and Simple Ultrasonic Cannabis Extraction. https://www.hielscher.com/fast-simple-ultrasonic-Cannabis-extraction.htm. Accessed Feb. 19, 2024). During the low-pressure vacuum cycle, minute vacuum bubbles or cavities occur in the liquid, which grow over several pressure cycles. During the compression phase of the liquid and bubbles, the pressure is positive, while the rarefaction phase produces a vacuum (negative pressure) (id.). During the compression-expansion cycles, the cavities in the liquid grow until they reach a size at which they cannot absorb more energy, at which point they implode. The implosion of those cavities results in various highly energetic sonomechanical effects, known as coustic/ultrasonic cavitation (id.), during which bubbles are created in the sonicated liquid that disrupt cell walls of plant tissues and release the intracellular compounds, including the primary and/or secondary bioactive molecules (Suslick. Sonoluminescence and Sonochemistry. In: Meyers, Ed. Encyclopedia of Physical Science and Technology. 3rd ed. Academic Press. 2001).

In some embodiments, extraction with a Soxhlet extractor is utilized. In some embodiments, Soxhlet extraction is conducted using an ethanol and water solvent (e.g., 80% ethanol and 20% water). In some embodiments, the solvent is heated to reflux, and the solvent vapor travels up a distillation arm and floods into the chamber housing the thimble of fungal material. The condenser at the top of the device ensures that any solvent vapor cools, and drips back down into the chamber housing the fungal material. As a result, the chamber containing the fungal material slowly fills with warm solvent. When the Soxhlet chamber is almost full, the chamber is emptied by the siphon, and the solvent is returned to the distillation flask. In some embodiments, using standard equipment known to those of skill, Soxhlet extraction is conducted for between about 6 and about 10 hours, including about 6 hours, about 7 hours, about 8 hours, about 9 hours, or about 10 hours. In embodiments, Soxhlet extraction is conducted for about 7 hours. In other embodiments, Soxhlet extraction is conducted for less than 6 or greater than 10 hours.

Extracts of fungal material (comprising a primary and/or secondary bioactive molecule) produced according to different methods described herein or otherwise known to one of skill may be combined to provide various advantages. For example, fungal extracts produced according to different techniques may contain different concentrations of primary and/or secondary bioactive molecules, and combining such extracts in a disclosed therapeutic combination may confer advantageous or synergistic properties.

In some embodiments, the fungal portion of a disclosed therapeutic combination comprises a mixture of fungal extracts. In some embodiments, the fungal portion of a disclosed therapeutic combination comprises a mixture of an ultrasonic extract and a Soxhlet extract. In some embodiments, the mixture comprises the ultrasonic extract and the Soxhlet extract in an ultrasonic:Soxhlet ratio (e.g., a volume ratio (v/v) or weight ratio (w/w) of the extract solutions, or a molar ratio, such as a molar ratio of the constituent bioactive molecule(s) in the extracts) of between about 0.5:1 to 10:1, including 0.5:1, 1:1, 1.25:1, 1.5:1, 1.75:1, 2:1, 2.5:1, 3:1, 3.5:1, 4:1, 5:1, 6:1, 7:1, 8:1, 9:1, and 10:1, including ranges in between these values.

In some embodiments, the mixture of fungal extracts comprises the ultrasonic extract and the Soxhlet extract in an ultrasonic:Soxhlet volume ratio of between about 0.5:1 to 10:1, including 0.5:1, 1:1, 1.25:1, 1.5:1, 1.75:1, 2:1, 2.5:1, 3:1, 3.5:1, 4:1, 5:1, 6:1, 7:1, 8:1, 9:1, and 10:1, including ranges in between these values. In some embodiments, the mixture comprises the ultrasonic extract and the Soxhlet extract in a weight ratio from about 0.5:1 to about 5:1, including 5:1, 1:1, 1.25:1, 1.5:1, 1.75:1, 2:1, 2.5:1, 3:1, 3.5:1, 4:1, and 5:1. In some embodiments, the mixture comprises the ultrasonic extract and the Soxhlet extract in an ultrasonic:Soxhlet molar ratio (e.g., the molar ratio of the constituent bioactive molecule(s) in the extracts) of between about 0.5:1 to 10:1, including 0.5:1, 1:1, 1.25:1, 1.5:1, 1.75:1, 2:1, 2.5:1, 3:1, 3.5:1, 4:1, 5:1, 6:1, 7:1, 8:1, 9:1, and 10:1, including ranges in between these values.

In some embodiments, the mixture comprises the ultrasonic extract and the Soxhlet extract in an ultrasonic:Soxhlet weight ratio of about 2:1.

Advantages of combining different fungal extracts in disclosed combinations can include, for example, improved solution stability during extraction of a bioactive molecule from a fungus as described herein. For example, certain compositions disclosed herein comprise, as a component of a fungal portion, psilocin and/or psilocybin. The solution instability of psilocybin, and especially psilocin, has long been recognized. However, the exact chemistry of the mechanisms and products of decomposition remains a matter of ongoing research. Studies of Psilocybe mushrooms that have been damaged to expose its alkaloids to environmental oxygen have revealed that psilocybin is dephosphorylated to psilocin, whose hydroxy group is subsequently oxidized to produce 4-oxo degradation products (Lenz et al. Angew ChemInt Ed Engl. 2020; 59(4):1450-1454) (“Lenz et al., 2020”). Research into possible solution decomposition mechanisms has revealed that oxidative dimerization may also contribute to solution instability of psilocin (Lenz et al. Chemistry. 2021; 27(47):12166-12171) (“Lenz et al., 2021”). In some embodiments, combining fungal extracts (e.g., as in disclosed combinations wherein the fungal portion comprises both an ultrasonic extract and a Soxhlet extract) protects the resulting combination against oxidative decomposition. Without being bound by theory, the differing levels of primary and/or secondary bioactive molecules in the different fungal extracts, which may include naturally occurring stabilizers and antioxidants, may in combination produce additive or synergistic effects that stabilize the resulting formulations against decomposition pathways, such as oxidative dimerization. In embodiments, oxidative decomposition of a composition can be monitored quantitatively (e.g., by chemical analysis methods, such as those disclosed in Lenz et al., 2020 and Lenz et al., 2021; or otherwise known to one of skill) or qualitatively (e.g., by visual monitoring of the characteristic “blueing” reaction of psilocybin- and/or psilocin-containing extracts and materials).

In embodiments, disclosed combinations wherein a fungal portion comprises multiple fungal extracts may also possess other advantageous properties when extracting a bioactive molecule from a fungus as disclosed herein, such as increased mixability with the other portions of the combination (e.g., plant and/or algal portions). By way of example, some disclosed compositions comprise components with high water solubility (e.g., a fungal extract comprising psilocybin and/or psilocin, or a fungal extract comprising these compounds) and low water solubility (e.g., a plant portion comprising a cannabinoid or Cannabis extract). During the preparation and storage of such compositions, precipitation may occur due to mutual incompatibility between primary and/or secondary bioactive molecules and the solvent system. Without being bound by theory, differing levels of primary and/or secondary bioactive molecules in different fungal extracts may produce additive or synergistic effects that stabilize the resulting formulations against precipitation.

In some embodiments, disclosed combinations wherein the fungal portion comprises multiple different fungal extracts possess improved stability (e.g., against oxidation and/or precipitation) such that additional stabilizing excipients (e.g., antioxidants, stabilizers) are not necessary for the combination to have sufficient stability under ambient conditions. Reducing or eliminating the need for such additional stabilizing excipients may result in further advantages, such as improved bioavailability, as these ingredients may interfere with absorption of a primary and/or secondary bioactive molecule.

2. Exemplary Extraction of Molecules from Cannabis

In embodiments, a therapeutic combination comprises a primary and/or secondary bioactive molecule from a Cannabis species. In embodiments, the primary and/or secondary bioactive molecule may be obtained via extraction of Cannabis plant material.

In embodiments, an exemplary extraction system is as follows: First, one may obtain Cannabis plant material which, in embodiments, has been substantially dried. In embodiments, the dried Cannabis plant material may then be ground, and optionally pulverized to yield a fine powder, which may optionally be sieved. As noted, grinding and pulverization may increase the surface area available for a solvent to interact with a material and, thus, may increase the yield of desired primary and/or secondary bioactive molecules within the material. In embodiments, the dried, and optionally ground and/or pulverized plant material is then placed into an extraction system generally containing a loop, wherein chilled ethanol (kept at between −30° C. and −40° C.) is circulated. Evaporation is used to remove solvent, followed by distillation and filtration to further concentrate the extract and remove impurities.

In such an exemplary extraction system, the high temperature and pressure would yield decarboxylation (occurring at about 110° C. at standard pressure). If, for example, one wants a product free of the intoxicating effects of THC, one may choose an extraction process with temperatures and pressures below the threshold. One also may utilize, e.g., column chromatography to remove THC after extraction.

It will be appreciated that, while an exemplary extraction process or system is disclosed as a series of steps that are performed, or are performed on a system, deviation from the steps is within the scope of the disclosure, as is modification to, removal of, and addition of various steps at any point during the extraction.

In some exemplary embodiments, ethanol extraction is used; however, it will be appreciated to be just one extraction process capable of producing an extract useful in a therapeutic combination. In other embodiments, other extraction methods and solvents may be utilized, non-limiting examples of which include subcritical and supercritical CO₂ extraction, hydrocarbon extraction such as with butane, propane, other hydrocarbon liquids and gasses as well as mixtures thereof in any proportions, alcohol extraction such as with methanol or isopropyl alcohol, and other such extraction methods known to those of skill, including variations thereof, e.g., to temperature, pressure, and equipment. While any such extraction methods may be utilized, it will be appreciated that certain extraction methods may provide specific benefits, depending on the desired composition of the extract; such will be known in view of the disclosure and ordinary skill in the art.

As non-limiting examples, supercritical extraction, such as supercritical CO₂ extraction, is useful for the extraction of cannabinoids, and preservation of terpenoids; warm alcohol extraction is useful in extracting and decarboxylating cannabinoids, but also extracts various pigments and impurities; cold alcohol extraction is more successful at isolating terpenes and cannabinoids from impurities than warm ethanol extraction, but is less efficient; hydrocarbon extraction, including butane and propane extraction, is effective in reducing impurities and pigments in the extract, and may be more efficient than cold alcohol extraction; and the like.

Primary and/or secondary bioactive molecules from Cannabis may be obtained via extraction of Cannabis plant material, and may be used to prepare Cannabis-derived drug substances and Cannabis-derived drug products.

“Cannabis-derived drug substance” may refer to a botanical drug substance which is derived from Cannabis plants (including plant parts, plant part biomass, and plant exudates), as non-limiting, purely illustrative examples, primary extracts prepared by processes including maceration, percolation, extraction with solvents such as C1-C5 alcohols (e.g., ethanol), hydrocarbons (e.g., propane, butane), and subcritical or supercritical carbon dioxide.

“Cannabis-derived drug product” may refer to a primary Cannabis extract that is further purified, for example by distillation or chromatography. It will be known to those of skill that when certain solvents are used to prepare primary extracts, the resultant extract may contain non-specific lipid-soluble material. Those of skill will know that such impurities can be removed by a variety of processes including winterization (e.g., chilling to −20° C. followed by filtration to remove waxy ballast), further extraction or filtration, and distillation.

3. Exemplary Extraction of Molecules from Dipteryx

In embodiments, a therapeutic combination comprises a primary and/or secondary bioactive molecule from Dipteryx. In embodiments, the primary and/or secondary bioactive molecule may be obtained via extraction of cumaru seeds (tonka beans).

A primary bioactive molecule in Dipteryx is coumarin, which may, in embodiments, be extracted from cumaru seeds via extraction with a polar solvent, including water, alcohols, including ethanol and methanol; and various ethers. Below, an exemplary extraction system utilizing ethanol is described.

In embodiments, ethanol extraction may be utilized to extract coumarin from cumaru seeds. In embodiments, the ethanol extraction may be ethanol percolation which, in embodiments, uses anhydrous ethanol. In embodiments, the ethanol percolation extraction system functions by first crushing the cumaru seeds so that the ethanol solvent is capable of entering and exiting the beans. Preferably, the seeds are sufficiently ground to increase surface area for extraction, forming a collection of plant matter. The seed matter is placed in a filter sitting atop a collection device. Ethanol is then poured over the seed matter, which then passes through the filter, and is collected in the collection device. In embodiments, this is completed until the color of the ethanol indicates substantially no additional bioactive molecules are being collected.

Optionally, the excess ethanol is then evaporated. This may be completed by applying heat to the resultant solution, or utilizing ambient evaporation. Other extraction methods also may be used, as known to those of skill. Additionally, while coumarin is directly mentioned, it should be readily appreciated that, e.g., secondary bioactive molecules from cumaru may additionally be extracted in the same or a similar manner, and may be present in the extract when the exemplary ethanol extraction method is utilized. Moreover, the concentration of a primary and/or secondary bioactive molecule may be determined as disclosed herein.

### 4. Determining the Concentration of a Primary or Secondary Bioactive Molecule

It will be appreciated that determining the concentration of a primary and/or secondary bioactive molecule within an extract or within fungi, plant, and/or algae material is within the ability of one of skill. Several such means are disclosed below, and may be used in embodiments.

As a non-limiting example of such means, the concentration of primary bioactive molecules from various Psilocybe species are presented in TABLE 1 below (obtained from Mahmood. Bioactive Alkaloids from Fungi: Psilocybin. In: Ramawat, K., Merillon, J M. (eds) Natural Products. Springer, 2013, and organized by psilocybin content; see also Stamets. Psilocybin Mushrooms of the World: An Identification Guide. Ten Speed Press. 1996) showing the w/w % of psilocybin, psilocin, and baeocystin in dried mushrooms (containing negligible water weight, or sometimes referred to as “cracker dry”), as is additionally discussed further herein:

TABLE 1
Psilocybin, Psilocin, & Baeocystin Content
of Exemplary Psilocybe Species by %

| Psilocybe Species | Psilocybin % | Psilocin % | Baeocystin % |
|---|---|---|---|
| P. azurescens | 1.70 | 0.38 | 0.35 |
| P. bohemica | 1.34 | 0.11 | 0.02 |
| P. semilanceata | 0.98 | 0.02 | 0.36 |
| P. baeocystis | 0.85 | 0.59 | 0.10 |
| P. cyanescens | 0.85 | 0.36 | 0.03 |
| P. tampanensis | 0.68 | 0.32 | n/a |
| P. cubensis | 0.63 | 0.60 | 0.025 |
| P. weilii | 0.61 | 0.27 | 0.05 |
| P. hoogshagenii | 0.60 | 0.10 | n/a |
| P. stuntzii | 0.36 | 0.12 | 0.02 |
| P. cyanofibrillosa | 0.21 | 0.04 | n/a |
| P. liniformans | 0.16 | n/a | 0.005 |

Thus, an extract of 100 mg P. cubensis may, in some embodiments, contain approximately 0.63 mg of psilocybin. An extract of 275 mg P. azurescens may, in embodiments, contain approximately 4.895 mg.

One will appreciate that growing conditions of organisms such as fungi (as well as plants and algae) may influence the concentration of bioactive molecules found therein. The concentration of bioactive molecules may differ depending on the organism part from which they are obtained (e.g, cap vs. stipe or mycelium; flower vs. leaves or stalk; etc.), as well as other variables known in the art. One will appreciate how to select such growing conditions, organism parts, and the like, and understand how to determine the concentration of bioactive molecules with disclosed methods or those generally known in the art.

In embodiments, the concentration of a primary and/or secondary bioactive molecule in fungi, plants, and/or algae is determined utilizing liquid chromatography, such as high-performance liquid chromatography (HPLC). Broadly, HPLC works by using pumps to pass a pressurized liquid solvent containing the sample mixture (in this case, a fungal extract) through a column filled with a solid adsorbent material. As each individual component in the sample interacts differently with the adsorbent material, it causes different flow rates for different components leading to the separation of the components as they flow out of the column.

In embodiments, the concentration of a primary and/or secondary bioactive molecule in fungi, plants, and/or algae is determined utilizing reversed-phase HPLC and single-wavelength detection, as disclosed in Samuelsson et al. Physiol Plant. 1977; 40:315-319, which is incorporated herein by reference. Broadly, reverse-phase HPLC generally proceeds in the same manner as the exemplary HPLC method disclosed above, but has a hydrophobic rather than a hydrophilic stationary phase. Meaning, hydrophobic molecules will absorb to the column packing, while hydrophilic molecules will be eluted and detected first.

In embodiments, the concentration of a primary and/or secondary bioactive molecule in fungi, plants, and/or algae is determined utilizing liquid chromatography and mass spectrometry (LC/MS), as illustrated by Goff et al. Anal Chim Acta. 2024; 1288:342161, which is incorporated herein by reference.

In embodiments, the concentration of a primary and/or secondary bioactive molecule in fungi, plants, and/or algae is determined utilizing hydrophilic interaction liquid chromatography (HILIC), described in Veress et al. The Applications Book. 2019; 32(7):387-388, and incorporated herein by reference. Broadly, HILIC is a normal phase HPLC technique utilizing reversed-phase type eluents. The column has a hydrophilic stationary phase, and the eluent contains water, a buffer, and a high concentration of water-miscible organic solvent.

In embodiments, the concentration of a primary and/or secondary bioactive molecule in fungi, plants, and/or algae is determined utilizing a rapid personal quantification means usable for determination of concentration, one example of which for fungi is the PSILO-QTest (Miraculix; Jena, Germany), useful for determining the concentration of psilocybin. The PSILO-QTest uses chemical color reaction to detect concentration, with color intensity proportional to the concentration of the bioactive molecule. In some embodiments, characterization of the fungal, plant, and/or algal portion of a disclosed combination (e.g., characterization of the primary and/or secondary bioactive molecules) comprises characterizing free and/or bound amino acids using known chromatographic techniques (e.g., LC-MS, HPLC). In embodiments, the characterization comprises determining the concentration of taurine in the fungal, plant, and/or algal portion.

### B. Synthesis

In embodiments, the primary and secondary bioactive molecules disclosed herein may be synthetic, where “synthetic” herein may refer to a substance which is manufactured in a laboratory, by means of chemical synthesis (e.g., by a series of chemical processes or reactions using chemical substrates, reagents, and optionally a catalyst) or biosynthesis, discussed herein, (e.g., from a bioengineered organism, and thus including those compounds also referred to as “biosynthetic” or as involving “synthetic biology” or “synbio”).

Generally, methods for chemical synthesis are well known in the art, including for the bioactive molecules disclosed herein. Methods for synthesis of the bioactive molecules and/or starting materials therefore will be readily apparent to the skilled artisan in view of general references known in the art (see, e.g., Greene & Wuts. Protective Groups in Organic Chemistry. 2nd ed. Wiley. 1991; Harrison et al. Compendium of Synthetic Organic Methods. Vol. 1-8. John Wiley and Sons, 1971-1996; Beilstein. Beilstein Handbook of Organic Chemistry. 1990; Feiser et al. Reagents for Organic Synthesis. Vol. 1-17. Wiley Interscience. 1967-1994. Trost & Fleming. Comprehensive Organic Synthesis. Pergamon Press. 1991; Theilheimer's Synthetic Methods of Organic Chemistry. Volumes 1-45. Karger. 1991; March. Advanced Organic Chemistry. Wiley Interscience. 1991; Larock. Comprehensive Organic Transformations. VCH Publishers. 1989; Paquette. Encyclopedia of Reagents for Organic Synthesis. John Wiley & Sons. 1995) and may be used to synthesize the bioactive molecules, where not otherwise obtained, such as by extraction. In general, the approaches used for similar compounds (Shulgin & Shulgin, PiHKAL, A chemical love story, Transform Press, Berkeley CA, 1992; Glennon et al., J Med Chem. 1986; 29(2):194-199; Nichols et al. J Med Chem. 1991; 34(1):276-281; Kedrowski et al., Organic Letters. 2007; 9(17):3205-3207; Heravi & Zadsirjan. Current Organic Synthesis. 2016; 13(6):780-833; Keri et al. Eur J Med Chem. 2017; 138:1002-1033; Perez Silanes et al. J Heterocycl Chem. 2001; 38(5):1025-1030; and references therein), such adaptation being that known and understood to those of ordinary skill.

In embodiments, the disclosed primary and secondary bioactive molecules may be obtained via biosynthesis. Biosynthesis refers to the production of molecules, such as the primary and/or secondary bioactive molecules utilized in the therapeutic combinations, within a cell or a cell-free system. In embodiments, the primary and/or secondary bioactive molecules useful in the therapeutic combinations may be produced via biosynthesis. Methods for biosynthesis of the bioactive molecules will be readily apparent to the skilled artisan in view of general references known in the art (see, e.g., PCT Pub. Nos. WO2021/052989, WO2019/173797, WO2021/086513, and WO2019/180309, all incorporated by reference herein). Non-limiting examples of known biosynthetic pathways in the art include those for cannabinoids, flavonoids, carotenoids, psilocybin, psilocin, and other indole alkaloids (such as the primary and secondary bioactive molecules found in fungi), amino acids, and peptides (including monopeptides, dipeptides, tripeptides, and proteins).

In embodiments, the primary bioactive molecule and/or secondary bioactive molecule from a fungus are produced by biosynthesis. In embodiments, the primary bioactive molecule and/or secondary bioactive molecule from a plant are produced by biosynthesis. In embodiments, the primary bioactive molecule and/or secondary bioactive molecule from algae are produced by biosynthesis.

### C. Isolation, Fractionation, and Purification

In embodiments, the primary and/or secondary bioactive molecules may be obtained via isolation, fractionation, or purification. Fractionation refers to a separation process wherein a certain quantity of a mixture is divided during a phase transition into a number of smaller quantities termed fractions, the composition of which varying according to a gradient.

In embodiments, fractions containing target primary and/or secondary bioactive molecules may be obtained via fractional distillation, column chromatography, fractional crystallization, fractional freezing, and bioassay-guided fractionation.

In embodiments, isolation of primary and/or secondary bioactive molecules from extracts containing the primary and/or secondary bioactive molecules may be completed. Numerous means of isolating compounds from extracts are known to those of skill in the art. However, exemplary means include thin layer chromatography, column chromatography, flash chromatography, sephadex chromatography, and high performance liquid chromatography (Sasidharan et al. AJTCAM. 2011; 8(1):1-10).

In embodiments, extracts may be further purified to isolate a particular primary and/or secondary bioactive molecule. In embodiments, such purification techniques will be those known to those of skill, but may include HPLC and centrifugal partition chromatography (CPC).

### D. Compounds

In embodiments, the disclosed primary and/or secondary bioactive molecules are pure or substantially pure. The terms “pure” or “substantially pure,” as used herein, refer to material that is substantially or essentially free from components that normally accompany the material when the material is synthesized, manufactured, or otherwise produced. A “pure” or “substantially pure” preparation of a primary and/or secondary bioactive molecule is accordingly defined as a preparation having a chromatographic purity (of the desired bioactive molecule) of greater than 90%, more preferably greater than 95%, more preferably greater than 96%, more preferably greater than 97%, more preferably greater than 98%, more preferably greater than 99%, more preferably greater than 99.5%, and most preferably greater than 99.9%, as determined by area normalization of an HPLC profile or other similar detection method. Preferably the pure or substantially pure primary and/or secondary bioactive molecules are substantially free of any other active compounds which are not intended to be administered to a subject. In this context, “substantially free” refers to the fact that no active compound(s), other than the primary and/or secondary bioactive molecules intended to be administered to a subject, are detectable by HPLC or another similar detection method, or are below a desired threshold of detection such as defined above.

The disclosed primary and/or secondary bioactive molecules may contain one or more asymmetric centers and give rise to enantiomers, diastereomers, and other stereoisomeric forms. The disclosure includes all such possible isomers, as well as mixtures thereof, including racemic and optically pure forms. Optically active isomers may be prepared using chiral synthons or chiral reagents, or resolved using conventional techniques. Various methods are known in the art for preparing optically active forms and determining activity.

The disclosure also includes primary and/or secondary bioactive molecules with at least one desired isotopic substitution of an atom, at an amount above the natural abundance of the isotope, i.e., isotopically enriched. Isotopes are atoms having the same atomic number but different mass numbers, i.e., the same number of protons but a different number of neutrons. By way of example, isotopes of hydrogen including deuterium (²H) and tritium (³H) may be used anywhere in described structures that achieves the desired result. Alternatively or in addition, isotopes of carbon, e.g., ¹¹C and ¹⁴C, may be used. Isotopic substitutions, for example deuterium substitutions, can be partial or complete. Partial deuterium substitution means that at least one hydrogen is substituted with deuterium. In certain embodiments, the isotope is at least 60%, 70%, 80%, 90%, 95%, or 99% or more enriched in an isotope at any location of interest. In one embodiment, deuterium is 90%, 95%, or 99% enriched at a desired location.

The primary and/or secondary bioactive molecules used in the disclosed methods will be understood to also encompass pharmaceutically acceptable salts of such molecules. The term “pharmaceutically acceptable salt” refers to salts prepared from pharmaceutically acceptable non-toxic acids or bases, which may be synthesized by conventional chemical methods. Generally, such salts are prepared by reacting the free acid or base forms of these agents with a stoichiometric amount of the appropriate base or acid in water or in an organic solvent, or in a mixture of the two; generally, nonaqueous media (e.g., ether, ethyl acetate, ethanol, isopropanol, or acetonitrile) are preferred. For therapeutic use, salts of the compounds are those wherein the counter-ion is pharmaceutically acceptable. Exemplary salts include 2-hydroxyethanesulfonate, 2-naphthalenesulfonate, 2-napsylate, 3-hydroxy-2-naphthoate, 3-phenylpropionate, 4-acetamidobenzoate, acefyllinate, acetate, aceturate, adipate, alginate, aminosalicylate, ammonium, amsonate, ascorbate, aspartate, benzenesulfonate, benzoate, besylate, bicarbonate, bisulfate, bitartrate, borate, butyrate, calcium edetate, calcium, camphocarbonate, camphorate, camphorsulfonate, camsylate, carbonate, cholate, citrate, clavulariate, cyclopentanepropionate, cypionate, d-aspartate, d-camsylate, d-lactate, decanoate, dichloroacetate, digluconate, dodecylsulfate, edentate, edetate, edisylate, estolate, esylate, ethanesulfonate, ethyl sulfate, fumarate, furate, fusidate, galactarate (mucate), galacturonate, gallate, gentisate, gluceptate, glucoheptanoate, gluconate, glucuronate, glutamate, glutarate, glycerophosphate, glycolate, glycollylarsanilate, hemisulfate, heptanoate (enanthate), heptanoate, hexafluorophosphate, hexanoate, hexylresorcinate, hippurate, hybenzate, hydrabamine, hydrobromide, hydrobromide/bromide, hydrochloride, hydroiodide, hydroxide, hydroxybenzoate, hydroxynaphthoate, iodide, isethionate, isothionate, I-aspartate, I-camsylate, I-lactate, lactate, lactobionate, laurate, laurylsulphonate, lithium, magnesium, malate, maleate, malonate, mandelate, meso-tartrate, mesylate, methanesulfonate, methylbromide, methylnitrate, methylsulfate, mucate, myristate, N-methylglucamine ammonium salt, napadisilate, naphthylate, napsylate, nicotinate, nitrate, octanoate, oleate, orotate, oxalate, p-toluenesulfonate, palmitate, pamoate, pantothenate, pectinate, persulfate, phenylpropionate, phosphate, phosphateldiphosphate, picrate, pivalate, polygalacturonate, potassium, propionate, pyrophosphate, saccharate, salicylate, salicylsulfate, sodium, stearate, subacetate, succinate, sulfate, sulfosaliculate, sulfosalicylate, suramate, tannate, tartrate, teoclate, terephthalate, thiocyanate, thiosalicylate, tosylate, tribrophenate, triethiodide, undecanoate, undecylenate, valerate, valproate, xinafoate, zinc and the like (see Berge et al. J Pharm Sci. 1977; 66(1):1-19).

Prodrugs of the primary and/or secondary bioactive molecules also will be appreciated to be within the scope of the disclosure. The term “prodrug” refers to a precursor of a biologically active pharmaceutical agent. Prodrugs undergo a chemical or a metabolic conversion to become a biologically active pharmaceutical agent, such as the conversion of the prodrug psilocybin to its active metabolite psilocin.

In the case of solid compositions, it is understood that the disclosed primary and/or secondary bioactive molecules may exist in different forms. For example, the primary and/or secondary bioactive molecules may exist in stable and metastable crystalline forms, isotropic and amorphous forms, milled forms and nano-particulate forms, all of which are intended to be within the scope of the disclosure. In addition, the primary and/or secondary bioactive molecules include crystalline forms, also known as polymorphs. Polymorphs include the different crystal packing arrangements of the same elemental composition of a bioactive molecule. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

For any of the disclosed primary and/or secondary bioactive molecules, substitution of the primary and/or secondary bioactive molecule by its ion, free base, salt form, polymorph, hydrate or solvate form, co-crystal, or an isomer or enantiomerically enriched mixture, shall be understood to provide merely an alternative embodiment still within the scope of the disclosure (with modifications to the formulation and dosage amounts made according to the teachings herein and ordinary skill, if necessary or desired). Further, compositions within the scope of the disclosure should be understood to be open-ended and may include additional primary and/or secondary bioactive molecules, active or inactive agents, and ingredients.

In some embodiments, disclosed primary and/or secondary bioactive molecules, or pharmaceutically acceptable salts, hydrates, solvates, or prodrugs thereof, are produced and tested in compliance with Good Laboratory Practice (GLP) or Good Manufacturing Practice (GMP) requirements, and/or their equivalents and any related practices, e.g., in Canada, when referring to Cannabis plants, the Good Production Practices (GPP) of the Cannabis Regulations.

### IV. THERAPEUTIC COMBINATIONS AND COMPOSITIONS

In some embodiments, the therapeutic combination comprises:
a. a fungal portion;
b. a first plant portion;
c. a second plant portion; and
d. an algal portion.

In some embodiments, the therapeutic combination comprises:
a. a fungal extract;
b. a first plant portion;
c. a second plant portion; and
d. an algal portion.

In some embodiments, the therapeutic combination comprises:
a. a fungal extract from a psilocybin-producing fungus;
b. a first plant portion;
c. a second plant portion; and
d. an algal portion.

In some embodiments, the therapeutic combination comprises:
a. a fungal extract from a species in the Psilocybe genus;
b. a first plant portion;
c. a second plant portion; and
d. an algal portion.

In some embodiments, the therapeutic combination comprises:
a. a Psilocybe cubensis extract;
b. a first plant portion;
c. a second plant portion; and
d. an algal portion.

In some embodiments, the therapeutic combination comprises:
a. a bioactive molecule from a fungus;
b. a first plant portion;
c. a second plant portion; and
d. an algal portion.

In some embodiments, the therapeutic combination comprises:
a. psilocybin;
b. a first plant portion;
c. a second plant portion; and
d. an algal portion.

In some embodiments, the therapeutic combination comprises:
a. psilocybin;
b. psilocin;
c. a first plant portion;
d. a second plant portion; and
e. an algal portion.

In some embodiments, the therapeutic combination comprises:
a. a fungal portion;
b. a plant extract;
c. a second plant portion; and
d. an algal portion.

In some embodiments, the therapeutic combination comprises:
a. a fungal portion;
b. a plant extract from the genus Cannabis;
c. a second plant portion; and
d. an algal portion.

In some embodiments, the therapeutic combination comprises:
a. a fungal portion;
b. a bioactive molecule from a plant;
c. a second plant portion; and
d. an algal portion.

In some embodiments, the therapeutic combination comprises:
a. a fungal portion;
b. a Cannabis sativa extract;
c. a second plant portion; and
d. an algal portion.

In some embodiments, the therapeutic combination comprises:
a. a fungal portion;
b. a bioactive molecule from Cannabis;
c. a second plant portion; and
d. an algal portion.

In some embodiments, the therapeutic combination comprises:
a. a fungal portion;
b. a cannabinoid;
c. a second plant portion; and
d. an algal portion.

In some embodiments, the therapeutic combination comprises:
a. a fungal portion;
b. THC;
c. a second plant portion; and
d. an algal portion.

In some embodiments, the therapeutic combination comprises:
a. a fungal portion;
b. CBD;
c. a second plant portion; and
d. an algal portion.

In some embodiments, the therapeutic combination comprises:
a. a fungal portion;
b. a Dipteryx extract;
c. a second plant portion; and
d. an algal portion.

In some embodiments, the therapeutic combination comprises:
a. a fungal portion;
b. a Dipteryx odorata extract;
c. a second plant portion; and
d. an algal portion.

In some embodiments, the therapeutic combination comprises:
a. a fungal portion;
b. a bioactive molecule from Dipteryx;
c. a second plant portion; and
d. an algal portion.

In some embodiments, the therapeutic combination comprises:
a. a fungal portion;
b. a bioactive molecule from Dipteryx odorata;
c. a second plant portion; and
d. an algal portion.

In some embodiments, the therapeutic combination comprises:
a. a fungal portion;
b. a bioactive molecule from tonka beans;
c. a second plant portion; and
d. an algal portion.

In some embodiments, the therapeutic combination comprises:
a. a fungal portion;
b. coumarin;
c. a second plant portion; and
d. an algal portion.

In some embodiments, the therapeutic combination comprises:
a. a fungal portion;
b. a first plant portion;
c. a second plant portion; and
d. an algal extract.

In some embodiments, the therapeutic combination comprises:
a. a fungal portion;
b. a first plant portion;
c. a second plant portion; and
d. a bioactive molecule from an algae.

In some embodiments, the therapeutic combination comprises:
a. a fungal portion;
b. a first plant portion;
c. a second plant portion; and
d. a bioactive molecule from a red algae (Rhodophyta).

In some embodiments, the therapeutic combination comprises:
a. a fungal portion;
b. a first plant portion;
c. a second plant portion; and
d. a bioactive molecule from a genus in the family Bangiaceae.

In some embodiments, the therapeutic combination comprises:
a. a fungal portion;
b. a first plant portion;
c. a second plant portion; and
d. a Pyropia extract.

In some embodiments, the therapeutic combination comprises:
a. a fungal portion;
b. a first plant portion;
c. a second plant portion; and
d. a Pyropia yezoensis extract.

In some embodiments, the therapeutic combination comprises:
a. a fungal portion;
b. a first plant portion;
c. a second plant portion; and
d. a bioactive molecule from the genus Pyropia.

In some embodiments, the therapeutic combination comprises:
a. a fungal portion;
b. a first plant portion;
c. a second plant portion; and
d. a bioactive molecule from Pyropia yezoensis.

In some embodiments, the therapeutic combination comprises:
a. a fungal portion;
b. a first plant portion;
c. a second plant portion; and
d. a bioactive molecule from Pyropia perforata.

In some embodiments, the therapeutic combination comprises:
a. a fungal portion;
b. a first plant portion;
c. a second plant portion; and
d. a bioactive molecule from the genus Porphyra.

In some embodiments, the therapeutic combination comprises:
a. a fungal portion;
b. a first plant portion;
c. a second plant portion; and
d. a bioactive molecule from Porphyra umbilicalis.

In some embodiments, the therapeutic combination comprises:
a. a fungal portion;
b. a first plant portion;
c. a second plant portion; and
d. porphyran.

In some embodiments, the therapeutic combination comprises:
a. a fungal portion;
b. a first plant portion;
c. a second plant portion; and
d. taurine.

In some embodiments, the therapeutic combination comprises:
a. a fungal extract;
b. a first plant extract;
c. a second plant extract; and
d. an algal extract.

In some embodiments, the therapeutic combination comprises:
a. a Psilocybe extract;
b. a Cannabis extract;
c. a Dipteryx extract; and
d. a Pyropia extract.

In some embodiments, the therapeutic combination comprises:
a. a Psilocybe extract;
b. a Cannabis extract;
c. a Dipteryx extract; and
d. a Porphyra extract.

In some embodiments, the therapeutic combination comprises:
a. a Psilocybe cubensis extract;
b. a Cannabis sativa extract;
c. a Dipteryx odorata extract; and
d. a Pyropia yezoensis extract.

In some embodiments, the therapeutic combination comprises:
a. a bioactive molecule from a fungus;
b. a bioactive molecule from a first plant;
c. a bioactive molecule from a second plant; and
d. a bioactive molecule from an algae.

In some embodiments, the therapeutic combination comprises:
a. a bioactive molecule from Psilocybe cubensis;
b. a bioactive molecule from Cannabis sativa;
c. a bioactive molecule from Dipteryx odorata; and
d. a bioactive molecule from Pyropia yezoensis.

In some embodiments, the therapeutic combination comprises:
a. a Psilocybe extract;
b. THC;
c. CBD;
d. coumarin; and
e. a Pyropia extract.

In some embodiments, the therapeutic combination comprises:
a. psilocybin;
b. THC;
c. coumarin; and
d. a Pyropia extract.

In some embodiments, the therapeutic combination comprises:
a. psilocin;
b. THC;
c. coumarin; and
d. taurine.

In some embodiments, the therapeutic combination comprises:
a. psilocybin;
b. CBD;
c. coumarin; and
d. a Pyropia extract.

In some embodiments, the therapeutic combination comprises:
a. psilocin;
b. CBD;
c. coumarin; and
d. a Pyropia extract.

In some embodiments, the therapeutic combination comprises:
a. psilocybin;
b. THC;
c. coumarin; and
d. taurine.

In some embodiments, the therapeutic combination comprises:
a. psilocybin;
b. THC;
c. coumarin; and
d. porphyran.

In embodiments, the therapeutic combination comprises a fungal portion, a fungal extract (e.g., a Psilocybe cubensis extract), or a bioactive molecule from a fungus (e.g. from Psilocybe cubensis). In embodiments, the fungal portion, the fungal extract, or the bioactive molecule from a fungus constitutes at least 99%, 99%, 98%, 95%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, or less than 1%, including ranges in between these values, by volume of the therapeutic combination. In embodiments, the fungal portion, the fungal extract, or the bioactive molecule from a fungus constitutes from about 10% to about 70%, about 20% to about 60%, about 30% to about 50%, or about 40% to about 50% by volume of the therapeutic combination. In embodiments, the fungal portion, the fungal extract, or the bioactive molecule from a fungus constitutes about 45% by volume of the therapeutic combination.

In embodiments, the therapeutic combination comprises a first plant portion, a first plant extract (e.g., a Cannabis sativa extract), or a bioactive molecule from a first plant (e.g., from Cannabis sativa). In embodiments, the first plant portion, the first plant extract, or the bioactive molecule from a first plant constitutes at least 99%, 99%, 98%, 95%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, or less than 1%, including ranges in between these values, by volume of the therapeutic combination. In embodiments, the first plant portion, the first plant extract, or the bioactive molecule from a first plant constitutes from about 1% to about 50%, about 5% to about 30%, or about 10% to about 20% by volume of the therapeutic combination. In embodiments, the first plant portion, the first plant extract, or the bioactive molecule from a first plant constitutes about 15% by volume of the therapeutic combination.

In embodiments, the therapeutic combination comprises a second plant portion, a second plant extract (e.g., a Dipteryx odorata extract), or a bioactive molecule from a second plant (e.g., from Dipteryx odorata). In embodiments, the second plant portion, the second plant extract, or the bioactive molecule from a second plant constitutes at least 99%, 99%, 98%, 95%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, or less than 1%, including ranges in between these values, by volume of the therapeutic combination. In embodiments, the second plant portion, the second plant extract, or the bioactive molecule from a second plant constitutes from about 1% to about 20%, about 1% to about 10%, or about 1% to about 3% by volume of the therapeutic combination. In embodiments, the second plant portion, the second plant extract, or the bioactive molecule from a second plant constitutes about 2% by volume of the therapeutic combination.

In embodiments, the therapeutic combination comprises an algal portion, an algal extract (e.g., a Pyropia yezoensis extract), or a bioactive molecule from an algae (e.g., from Pyropia yezoensis). In embodiments, the algal portion, the algal extract, or the bioactive molecule from an algae constitutes at least 99%, 99%, 98%, 95%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, or less than 1%, including ranges in between these values, by volume of the therapeutic combination. In embodiments, the algal portion, the algal extract, or the bioactive molecule from an algae constitutes from about 1% to about 50%, about 5% to about 30%, or about 10% to about 20% by volume of the therapeutic combination. In embodiments, the algal portion, the algal extract, or the bioactive molecule from an algae constitutes about 15% by volume of the therapeutic combination.

In embodiments, the first plant portion and the second plant portion are from different plant genera. In embodiments, the first plant portion and the second plant portion are from different plant species. In embodiments, the first plant portion and the second plant portion are from different plant extracts. In embodiments, the first plant portion and the second plant portion comprise different primary bioactive molecules. In embodiments, the first plant portion and the second plant portion comprise different secondary bioactive molecules. In embodiments, the first plant portion and the second plant portion are from the same plant genus. In embodiments, the first plant portion and the second plant portion are from the same plant species. In embodiments, the first plant portion and the second plant portion are from the same plant extract. In embodiments, the first plant portion and the second plant portion comprise the same primary bioactive molecules. In embodiments, the first plant portion and the second plant portion comprise the same secondary bioactive molecules.

In some embodiments, the single composition further comprises any of a flavorant, colorant, and diluent. For example, in embodiments, the flavorant or colorant comprises ginger or bay laurel. In embodiments, the flavorant or colorant is ethanol infused with ginger and bay leaf. In embodiments, the flavorant or colorant constitutes from about 1% to about 50%, about 5% to about 50%, about 5% to about 40%, about 5% to about 30%, about 5% to about 20%, about 10% to about 30%, or about 10% to about 20% by volume of the single composition. In embodiments, the flavorant or colorant constitutes about 15% by volume of the single composition.

In embodiments, the diluent is water. In embodiments, the diluent (e.g., water) constitutes from about 1% to about 30%, about 5% to about 30%, about 5% to about 20%, or about 5% to about 15% by volume of the single composition. In embodiments, the water constitutes about 8% by volume of the single composition.

In embodiments, a therapeutic combination comprising a primary and/or secondary bioactive molecule from a fungus and a primary and/or secondary bioactive molecule from a plant will be synergistic or have synergistic effects. In embodiments, a therapeutic combination comprising a primary and/or secondary bioactive molecule from a fungus, and a primary and/or secondary bioactive molecule from a plant will be synergistic or have synergistic effects. In embodiments, a therapeutic combination comprising a primary and/or secondary bioactive molecule from a fungus and a primary and/or secondary bioactive molecule from an algae will be synergistic or have synergistic effects. In embodiments, a therapeutic combination comprising a primary and/or secondary bioactive molecule from a plant and a primary and/or secondary bioactive molecule from an algae will be synergistic or have synergistic effects. In embodiments, a therapeutic combination comprising a primary and/or secondary bioactive molecule from a fungus, a primary and/or secondary bioactive molecule from a plant, and a primary and/or secondary bioactive molecule from an algae will be synergistic or have synergistic effects.

### A. Synergistic Effects

In embodiments, “synergistic” may refer to a combination that is more effective than the additive effects of any two or more single primary and/or secondary bioactive molecules. A synergistic effect, for example, permits the effective treatment of a disease using lower amounts (doses) of individual therapy. This includes lower doses of a first primary and/or secondary bioactive molecule or a second primary and/or secondary bioactive molecule (“apparent one-way synergy”), or lower doses of both primary and/or secondary bioactive molecules (“two-way synergy”), than would normally be required when either primary and/or secondary bioactive molecule is used alone. Lower doses, for example, may result in increased safety and/or lower toxicity without reduced efficacy. A synergistic effect may additionally result in improved efficacy, including an improved avoidance or reduction of disease as compared to any single therapy.

“Synergistic effects” also will be understood to include increases in potency, bioactivity, bioaccessibility, bioavailability, or therapeutic effect (including one or more additional therapeutic effects), greater than the additive contributions of the components acting alone, and/or are greater than the contribution of the isolated primary and/or secondary bioactive molecules on their own.

Numerous methods known to those of skill exist to determine whether there is synergy as to a particular effect, i.e., whether, when two or more components are mixed together, the effect is greater than the sum of the effects of the individual components applied alone, thereby producing “1+1>2.” Suitable methods include isobologram (or contour) analysis (Huang et al. Front Pharmacol. 2019; 10:1222), or the equation of Loewe additivity (Loewe & Muischnek. Archiv f experiment Pathol u Pharmakol. 1926; 114:313-326). A synergistic effect also may be calculated using methods such as the Sigmoid-Emax equation (Holford & Scheiner. Clin Pharmacokinet. 1981; 6(6):429-453) and the median-effect equation (Chou & Talalay. Adv Enzyme Regul. 1984; 22:27-55). The corresponding graphs associated with the equations above are the concentration-effect curve and combination index curve, respectively. The equations referred to above may be applied to experimental data to generate a corresponding graph to aid in assessing effects of a combination.

In embodiments, a combination comprises a primary and/or secondary bioactive molecule from a psilocybin-producing fungus and a primary and/or secondary bioactive molecule from Cannabis. In some embodiments, the combination will be synergistic or have synergistic effects.

In embodiments, a combination comprises a primary and/or secondary bioactive molecule from a psilocybin-producing fungus and a primary and/or secondary bioactive molecule from Dipteryx. In some embodiments, the combination will be synergistic or have synergistic effects.

In embodiments, a combination comprises a primary and/or secondary bioactive molecule from a psilocybin-producing fungus and a primary and/or secondary bioactive molecule from Pyropia or Porphyra. In some embodiments, the combination will be synergistic or have synergistic effects.

In embodiments, a combination comprises a primary and/or secondary bioactive molecule from Cannabis and a primary and/or secondary bioactive molecule from Dipteryx. In some embodiments, the combination will be synergistic or have synergistic effects.

In embodiments, a combination comprises a primary and/or secondary bioactive molecule from Cannabis and a primary and/or secondary bioactive molecule from Pyropia or Porphyra. In embodiments, the combination will be synergistic or have synergistic effects.

In embodiments, a combination comprising a primary and/or secondary bioactive molecule from Dipteryx and a primary and/or secondary bioactive molecule from Pyropia or Porphyra. In some embodiments, the combination will be synergistic or have synergistic effects.

In embodiments, a combination comprises a primary and/or secondary bioactive molecule from a psilocybin-producing fungus, a primary and/or secondary bioactive molecule from Cannabis, and a primary and/or secondary bioactive molecule from Dipteryx. In some embodiments, the combination will be synergistic or have synergistic effects.

In embodiments, a combination comprises a primary and/or secondary bioactive molecule from a psilocybin-producing fungus, a primary and/or secondary bioactive molecule from Cannabis, and a primary and/or secondary bioactive molecule from Pyropia or Porphyra. In some embodiments, the combination will be synergistic or have synergistic effects.

In embodiments, a combination comprises a primary and/or secondary bioactive molecule from a psilocybin-producing fungus, a primary and/or secondary bioactive molecule from Dipteryx, and a primary and/or secondary bioactive molecule from Pyropia or Porphyra. In some embodiments, the combination will be synergistic or have synergistic effects.

In embodiments, a combination comprises a primary and/or secondary bioactive molecule from Cannabis, a primary and/or secondary bioactive molecule from Pyropia or Porphyra, and a primary and/or secondary bioactive molecule from Dipteryx. In some embodiments, the combination will be synergistic or have synergistic effects.

In embodiments, a combination comprises a primary and/or secondary bioactive molecule from a psilocybin-producing fungus, a primary and/or secondary bioactive molecule from Cannabis, a primary and/or secondary bioactive molecule from Dipteryx, and a primary and/or secondary bioactive molecule from Pyropia or Porphyra. In some embodiments, the combination will be synergistic or have synergistic effects.

In embodiments, a therapeutic combination comprises a primary and/or secondary bioactive molecule from one or more species of Copelandia, Gymnopilus, Inocybe, Panaeolus, Pholiotina, Pluteus, and Psilocybe, and a primary and/or secondary bioactive molecule from one or more species of Cannabis. In some embodiments, the therapeutic combination will be synergistic or have synergistic effects.

In embodiments, a therapeutic combination comprises a primary and/or secondary bioactive molecule from one or more species of Copelandia, Gymnopilus, Inocybe, Panaeolus, Pholiotina, Pluteus, and Psilocybe, and a primary and/or secondary bioactive molecule from one or more species of cumaru. In some embodiments, the combination will be synergistic or have synergistic effects.

In embodiments, a therapeutic combination comprises a primary and/or secondary bioactive molecule from one or more species of Copelandia, Gymnopilus, Inocybe, Panaeolus, Pholiotina, Pluteus, and Psilocybe, and a primary and/or secondary bioactive molecule from one or more species of Pyropia or Porphyra. In some embodiments, the therapeutic combination will be synergistic or have synergistic effects.

In some embodiments, a disclosed therapeutic combination is a botanical formulation comprising minimal or low doses of whole extracts of C. sativa plant, P. cubensis fungi, P. yesoensis algae, and D. odorata bean, relative to those that would otherwise be understood or expected in the art, and a synergy is an ability to provide an effect at the given dose amounts. Because the disclosed combinations, compositions, and methods synergistically permit the effective use of surprisingly low doses of one or more of the bioactive molecules, in some embodiments these low doses will avoid receptor exhaustion, avoid the activation of self-regulating compensatory measures, or avoid induced compensatory physiological effects.

### B. Additional Active Agents

A disclosed therapeutic combination may also be used together with an additional active agent, which may, for example, contribute to or provide an additional therapeutic effect, or contribute to or provide a synergistic effect. “Agent” refers to an agent that affects or modulates the activity of a target, such as a receptor. In embodiments, an agent is a ligand for a receptor and modulates the activity of the receptor. In embodiments, an agent binds to, blocks, activates, inhibits, or otherwise influences (e.g., via an allosteric reaction) activity at a given receptor system.

In embodiments, the additional active agent is an anti-inflammatory agent. “Anti-inflammatory agent” refers to agents that can reduce inflammation or swelling. In embodiments, the anti-inflammatory agent is selected from the group consisting of a salicylate, a steroid, a phosphodiesterase inhibitor, an interleukin inhibitor, a non-steroidal anti-inflammatory drug (NSAID), a disease-modifying antirheumatic drug (DMARD), and an other agent useful in treating an inflammatory disease.

In embodiments, the anti-inflammatory agent is a salicylate. In embodiments, the salicylate is selected from the group consisting of magnesium salicylate, aspirin, diflunisal, aspirin/citric acid/sodium bicarbonate, salsalate, and choline salicylate. In embodiments, the salicylate is magnesium salicylate. In embodiments, the salicylate is aspirin. In embodiments, the salicylate is diflunisal. In embodiments, the salicylate is aspirin/citric acid/sodium bicarbonate. In embodiments, the salicylate is salsalate. In embodiments, the salicylate is choline salicylate.

In embodiments, the anti-inflammatory agent is a steroid. In embodiments, the steroid is a corticosteroid or a glucocorticoid. In embodiments, the steroid is a corticosteroid. In embodiments, the corticosteroid is selected from the group consisting of betamethasone, budesonide, cortisone, dexamethasone, hydrocortisone, methylprednisolone, prednisolone, prednisone, and triamcinolone. In embodiments, the corticosteroid is betamethasone. In embodiments, the corticosteroid is budesonide. In embodiments, the corticosteroid is cortisone. In embodiments, the corticosteroid is dexamethasone. In embodiments, the corticosteroid is hydrocortisone. In embodiments, the corticosteroid is methylprednisolone. In embodiments, the corticosteroid is prednisolone. In embodiments, the corticosteroid is prednisone. In embodiments, the corticosteroid is triamcinolone.

In embodiments, the steroid is a glucocorticoid. In embodiments, the glucocorticoid is selected from the group consisting of triamcinolone, methylprednisolone, budesonide, dexamethasone, prednisone, hydrocortisone, betamethasone, prednisolone, and deflazacort. In embodiments, the glucocorticoid is triamcinolone. In embodiments, the glucocorticoid is methylprednisolone. In embodiments, the glucocorticoid is budesonide. In embodiments, the glucocorticoid is dexamethasone. In embodiments, the glucocorticoid is prednisone. In embodiments, the glucocorticoid is hydrocortisone. In embodiments, the glucocorticoid is betamethasone. In embodiments, the glucocorticoid is prednisolone. In embodiments, the glucocorticoid is deflazacort.

In embodiments, the anti-inflammatory agent is a phosphodiesterase inhibitor. In embodiments, the phosphodiesterase inhibitor is selected from the group consisting of a phosphodiesterase type-1 inhibitor, a phosphodiesterase type-2 inhibitor, a phosphodiesterase type-3 inhibitor, a phosphodiesterase type-4 inhibitor, or a phosphodiesterase type-5 inhibitor. In embodiments, the phosphodiesterase inhibitor is a phosphodiesterase type-4 inhibitor. In embodiments, the phosphodiesterase type-4 inhibitor is selected from the group consisting of apremilast, crisaborole, and roflumilast. In embodiments, the phosphodiesterase type-4 inhibitor is apremilast. In embodiments, the phosphodiesterase type-4 inhibitor is crisaborole. In embodiments, the phosphodiesterase type-4 inhibitor is roflumilast.

In embodiments, the anti-inflammatory agent is an interleukin inhibitor. In embodiments, the interleukin inhibitor is selected from the group consisting of dupilumab, ustekinumab, secukinumab, ixekizumab, mepolizumab, guselkumab, benralizumab, tocilizumab, risankizumab, sarilumab, brodalumab, anakinra, reslizumab, tildrakizumab, canakinumab, rilonacept, tralokinumab, daclizumab, siltuximab, spesolimab, basiliximab, and satralizumab. In embodiments, the interleukin inhibitor is dupilumbab. In embodiments, the interleukin inhibitor is ustekinumab. In embodiments, the interleukin inhibitor is secukinumab. In embodiments, the interleukin inhibitor is ixekizumab. In embodiments, the interleukin inhibitor is mepolizumab. In embodiments, the interleukin inhibitor is guselkumab. In embodiments, the interleukin inhibitor is benralizumab. In embodiments, the interleukin inhibitor is tocilizumab. In embodiments, the interleukin inhibitor is risankizumab. In embodiments, the interleukin inhibitor is sarilumab. In embodiments, the interleukin inhibitor is brodalumab. In embodiments, the interleukin inhibitor is anakinra. In embodiments, the interleukin inhibitor is reslizumab. In embodiments, the interleukin inhibitor is tildrakizumab. In embodiments, the interleukin inhibitor is canakinumab. In embodiments, the interleukin inhibitor is rilonacept. In embodiments, the interleukin inhibitor is tralokinumab. In embodiments, the interleukin inhibitor is daclizumab. In embodiments, the interleukin inhibitor is siltuximab. In embodiments, the interleukin inhibitor is spesolimab. In embodiments, the interleukin inhibitor is basiliximab. In embodiments, the interleukin inhibitor is satralizumab.

In embodiments, the anti-inflammatory agent is a non-steroidal anti-inflammatory drug (NSAID). In embodiments, the NSAID is selected from the group consisting of diclofenac, etodolac, fenoprofen, flurbiprofen, ibuprofen, indomethacin, meclofenamate, mefenamic acid, nabumetone, naproxen, tolmetin, piroxicam, meloxicam, ketorolac, ketoprofen, sulindac, esomeprazole/naproxen, diclofenac/misoprostol, famotidine/ibuprofen, diflunisal, oxaprozin, lansoprazole/naproxen, and a selective COX-2 inhibitor.

In embodiments, the NSAID is diclofenac. In embodiments, the NSAID is etodolac. In embodiments, the NSAID is fenoprofen. In embodiments, the NSAID is flurbiprofen. In embodiments, the NSAID is ibuprofen. In embodiments, the NSAID is indomethacin. In embodiments, the NSAID is meclofenamate. In embodiments, the NSAID is mefenamic acid. In embodiments, the NSAID is nabumetone. In embodiments, the NSAID is naproxen. In embodiments, the NSAID is tolmetin. In embodiments, the NSAID is piroxicam. In embodiments, the NSAID is meloxicam. In embodiments, the NSAID is ketorolac. In embodiments, the NSAID is ketoprofen. In embodiments, the NSAID is sulindac. In embodiments, the NSAID is esomeprazole/naproxen. In embodiments, the NSAID is diclofenac/misoprostol. In embodiments, the NSAID is famotidine/ibuprofen. In embodiments, the NSAID is diflunisal. In embodiments, the NSAID is oxaprozin. In embodiments, the NSAID is Iansoprazole/naproxen.

In embodiments, the NSAID is a selective COX-2 inhibitor. In embodiments, the selective COX-2 inhibitor is selected from the group consisting of celecoxib, etoricoxib, lumiracoxib, rofecoxib, valdecoxib, and amlodipine/celecoxib. In embodiments, the selective COX-2 inhibitor is celecoxib. In embodiments, the selective COX-2 inhibitor is etoricoxib. In embodiments, the selective COX-2 inhibitor is lumiracoxib. In embodiments, the selective COX-2 inhibitor is rofecoxib. In embodiments, the selective COX-2 inhibitor is valdecoxib. In embodiments, the selective COX-2 inhibitor is amlodipine/celecoxib.

In embodiments, the anti-inflammatory agent is a disease-modifying antirheumatic drug (DMARD). In embodiments, the DMARD is a synthetic DMARD. In embodiments, the synthetic DMARD is selected from the group consisting of methotrexate, leflunomide, hydroxychloroquine, azathioprine, and sulfasalazine. In embodiments, the synthetic DMARD is methotrexate. In embodiments, the synthetic DMARD is leflunomide. In embodiments, the synthetic DMARD is hydroxychloroquine. In embodiments, the synthetic DMARD is azathioprine. In embodiments, the synthetic DMARD is sulfasalazine.

In embodiments, the DMARD is a biological DMARD. In embodiments, the biological DMARD is selected from the group consisting of a TNF-inhibitor, an interleukin-1 inhibitor, an interleukin-6 inhibitor, a T-cell inhibitor, a B-cell inhibitor, and a Janus kinase inhibitor. In embodiments, the biological DMARD is a TNF-inhibitor. In embodiments, the biological DMARD is an interleukin-1 inhibitor. In embodiments, the biological DMARD is an interleukin-6 inhibitor. In embodiments, the biological DMARD is a T-cell inhibitor. In embodiments, the biological DMARD is a B-cell inhibitor. In embodiments, the biological DMARD is a Janus kinase inhibitor.

In embodiments, the TNF-inhibitor is selected from the group consisting of adalimumab, etanercept, infliximab, golimumab, and certolizumab. In embodiments, the TNF-inhibitor is adalimumab. In embodiments, the TNF-inhibitor is etanercept. In embodiments, the TNF-inhibitor is infliximab. In embodiments, the TNF-inhibitor is golimumab. In embodiments, the TNF-inhibitor is certolizumab. In embodiments, the interleukin-1 inhibitor is anakinra.

In embodiments, the interleukin-6 inhibitor is tocilizumab or sarulimab. In embodiments, the interleukin-6 inhibitor is tocilizumab. In embodiments, the interleukin-6 inhibitor is sarulimab. In embodiments, the T-cell inhibitor is abatacept. In embodiments, the B-cell inhibitor is rituximab. In embodiments, the Janus kinase inhibitor is selected from the group consisting of tofacitinib, baricitinib, and upadacitinib. In embodiments, the Janus kinase inhibitor is tofacitinib. In embodiments, the Janus kinase inhibitor is baricitinib. In embodiments, the Janus kinase inhibitor is upadacitinib.

In embodiments, the anti-inflammatory agent is another agent useful in treating an inflammatory disease. The term “other agent useful in treating an inflammatory disease” may be used interchangeably with the term “other agent” herein, unless context expressly dictates otherwise. In embodiments, the other agent is selected from the group consisting of an analgesic, an anesthetic, a peptide, an enzyme, a coenzyme, a calcium channel blocker, an immunomodulator, an immunosuppressant, a proton-pump inhibitor, an antidepressant (e.g., citalopram, escitalopram, fluoxetine, fluvoxamine, paroxetine, lithium, bupropion, sertraline, amitriptyline, clomipramine, desipramine, doxepin, imipramine, nortriptyline, or venlafaxine), an opioid (e.g., fentanyl, methadone, morphine, or buprenorphine), a biguanide (e.g., metformin), a central nervous system agent (e.g., sodium phenylbutyrate/taurursodiol), an antiviral (e.g., an uncoating inhibitor, an entry inhibitor, a protease inhibitor, such as a Mpro inhibitor, ritonavir, nirmatrelvir, amprenavir, atazanavir, darunavir, fosamprenavir, indinavir, lopinavir, nelfinavir, saquinavir, tipranavir, asunaprevir, boceprevir, grazoprevir, glecaprevir, paritaprevir, simeprevir, telaprevir, GC376, nelfinavir, ensitrelvir (Xocova), or simnotrelvir, an immunostimulatory agent, a latency reversal agent, a reverse transcriptase, a reverse transcriptase inhibitor, such as azvudine, an integrate inhibitor, a nucleoside analogue, such as remdesivir, ganciclovir, ribavarin, valganciclovir, cidofovir, deuremidevir (VV116), obeldesivir (GS-5245), or acyclovir, an ACE2 decoy receptor drug, a replication inhibitor, a polymerase inhibitor, such as a RdRp inhibitor, molnupiravir, favilavir, bemnifosbuvir (AT-527), or sofosbuvir, an agent that acts on viral uncapping, a chemokine receptor antagonist, such as maraviroc, a neuraminidase inhibitor, such as zanamivir or oseltamivir, a fusion inhibitor, an interferon, such as interferon alpha, interferon beta, or interferon gamma, an integrase strand transfer inhibitor, such as dolutegravir, a nucleoside reverse transcriptase inhibitor (NRTI), such as abacavir, stavudine, entecavir, lamivudine, or zidovudine, or a non-nucleoside reverse transcriptase inhibitor), and an anticonvulsant (e.g., carbamazepine, oxcarbazepine, lamotrigine, valproic acid, topiramate, levetiracetam, brivaracetam, or seletracetam).

In embodiments, the other agent is an analgesic. In embodiments, the other agent is an anesthetic. In embodiments, the other agent is a peptide. In embodiments, the other agent is an enzyme. In embodiments, the other agent is a coenzyme. In embodiments, the other agent is a calcium channel blocker. In embodiments, the other agent is an immunomodulator. In embodiments, the other agent is an immunosuppressant, such as a macrolide, including rapamycin. In embodiments, the other agent is a proton-pump inhibitor. In embodiments, the other agent is an antidepressant. In embodiments, the other agent is an opioid. In embodiments, the other agent is a biguanide. In embodiments, the other agent is a central nervous system agent. In embodiments, the other agent is an antiviral. In embodiments, the other agent is an anticonvulsant.

In embodiments, the additional active agent is a serotonergic agent. In embodiments, a “serotonergic agent” refers to any compound that binds to, blocks, or otherwise influences (e.g., via an allosteric reaction) activity at a serotonin receptor, including any serotonin receptor subtypes. In embodiments, the serotonergic agent binds to a serotonin receptor. In embodiments, the serotonergic agent indirectly affects a serotonin receptor, e.g., via interactions affecting the reactivity of other molecules at the serotonin receptor. In embodiments, the serotonergic agent is an agonist, e.g., a compound activating a serotonin receptor. In embodiments, the serotonergic agent is an antagonist, e.g., a compound binding but not activating a serotonin receptor, e.g., blocking a receptor. In embodiments, the serotonergic agent is an effector molecule, e.g., a compound binding to an enzyme for allosteric regulation. In embodiments, the serotonergic agent acts (either directly or indirectly) at more than one type of receptor, including receptors other than serotonergic or other monoaminergic receptors. In embodiments, the serotonergic agent blocks the serotonin transporter (SERT) and results in an elevation of the synaptic concentration of serotonin, and an increase of neurotransmission. In embodiments, the serotonergic agent is a serotonin uptake or reuptake inhibitor. In embodiments, the serotonergic agent acts as a reuptake modulator and inhibits the plasmalemmal transporter-mediated reuptake of serotonin from the synapse into the presynaptic neuron, leading to an increase in extracellular concentrations of serotonin and an increase in neurotransmission. In embodiments, the serotonergic agent inhibits the activity of one or both monoamine oxidase enzymes, resulting in an increase in concentrations of serotonin and an increase in neurotransmission. In embodiments, the serotonergic agent is an antidepressant or anxiolytic, such as an SSRI, serotonin-norepinephrine reuptake inhibitor (SNRI), tricyclic antidepressant (TCA), monoamine oxidase inhibitor (MAOI), or atypical antidepressant. In other embodiments, the serotonergic agent is selected from the group consisting of: (1) serotonin transport inhibitors; (2) serotonin receptor modulators; (3) serotonin reuptake inhibitors; (4) serotonin and norepinephrine reuptake inhibitors; (5) serotonin dopamine antagonists; (6) monoamine reuptake inhibitors; (7) pyridazinone aldose reductase inhibitors; (8) stimulants of serotonin receptors; (9) stimulants of serotonin synthesis; (10) serotonin receptor agonists; (11) serotonin receptor antagonists; and (12) serotonin metabolites.

In embodiments, the additional active agent is any of a phenolic compound, a terpene, a polysaccharide, a polyphenol, a lipid, an organic acid, a polyunsaturated fatty acid (PUFA), an imminosugar, and a tocopherol extracted from fungi, algae, and/or plants. In embodiments, the additional active agent is extracted from genera including any of Cheirolophus, Rhaponticoides, Volutaria, Zingiber; Rosmarinus, Salvia, Thymus, Origanum, Ocimum, Melissa, Mentha, Origanum, Satureja, Hyssopus, Laurus, Bacopa, Bupleunum, Camellia, Berberis, and Lathyrus. In embodiments, the additional active agent is extracted from the genus Zingiber, such as from Zingiber officinale. In embodiments, the additional active agent is extracted from Zingiber officinale rhizomes. In embodiments, the additional active agent is extracted from the genus Laurus, e.g., from Laurus nobilis. In embodiments, the additional active agent is extracted from Laurus nobilis leaves. In embodiments, a disclosed composition comprises an additional active agent from Zingiber, e.g., from Zingiber officinale rhizomes, and an additional active agent from Laurus, e.g., from L. nobilis leaves.

In embodiments, the additional active agent may be any of an amino acid, an antioxidant, an anti-inflammatory agent, an analgesic, an antineuropathic agent, an antinociceptive agent, an antimigraine agent, an anxiolytic, an antidepressant, an antipsychotic, an anti-PTSD agent, a cannabinoid, a NMDA antagonist, a dissociative, an immunostimulant, an anti-cancer agent, an antiemetic, an orexigenic, an antiulcer agent, an antihistamine, an antihypertensive, an anticonvulsant, an antiepileptic, a bronchodilator, a neuroprotectant, a nootropic, an entheogen, an entactogen, an empathogen, a psychedelic, a monoamine oxidase inhibitor (e.g., a RIMA), a tryptamine, a terpene, a phenethylamine, a sedative, a stimulant, a metabolic or glucose modulator (e.g., metformin), a serotonergic agent, and a vitamin. These agents may be in ion, freebase, or salt form, and may be isomers, prodrugs, derivatives (preferably physiologically functional derivatives), or analogs.

In embodiments, the one or more additional active agents may be administered in separate dosage forms or as a single dosage form comprising one or more primary and/or secondary bioactive molecules according to the disclosure in combination with one or more additional active agents.

### V. PHARMACEUTICAL COMPOSITIONS

In some aspects are disclosed pharmaceutical compositions comprising a therapeutic combination. “Pharmaceutical compositions” (also as shorthand, unless context indicates otherwise, “compositions”) comprise a therapeutic combination together in an amount (for example, in a unit dosage form) with a pharmaceutically acceptable carrier, diluent, or excipient. It will be understood that some embodiments do not have a single carrier, diluent, or excipient alone, but have multiple carriers, diluents, and/or excipients. It will also be understood that the term “formulation” used in embodiments herein is equivalent to the terms “composition” and “pharmaceutical composition” unless context clearly indicates otherwise.

In some embodiments, a disclosed combination or composition will comprise a non-naturally occurring carrier, diluent, or excipient, examples of which will be known to those in the art. In some embodiments, “non-naturally occurring” refers to a carrier, diluent, or excipient used in a disclosed combination or composition that does not naturally occur in a fungus, a plant, and/or an algae, or in any of the same fungi, plants, and/or algae from which the extracts and/or the bioactive molecules of the combination or composition are or can be derived.

Compositions can be prepared by standard pharmaceutical formulation techniques such as disclosed in Remington: The Science and Practice of Pharmacy (2005) 21th ed., Mack Publishing Co., Easton, Pa.; The Merck Index (1996) 12th ed., Merck Publishing Group, Whitehouse, N.J.; Pharm. Principles of Solid Dosage Forms (1993), Technomic Publishing Co., Inc., Lancaster, Pa.; and Ansel and Stoklosa, Pharm. Calculations (2001) 11th ed., Lippincott Williams & Wilkins, Baltimore, Md.; and Poznansky et al. Drug Delivery Systems (1980), R. L. Juliano, ed., Oxford, N.Y., pp. 253-315. “Pharmaceutically acceptable,” used in connection with an agent, means the agents are generally safe and, within the scope of sound medical judgment, suitable for use in contact with the cells of humans and other animals without undue toxicity, irritation, allergic response, or complication, and commensurate with a reasonable risk/benefit ratio.

Compositions comprising a therapeutic combination can be formulated into any suitable dosage form, such as aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilised formulations, tablets, capsules, pills, powders, patches, inhalers, nebulizers, pulsatile release formulations, multi-particulate formulations, immediate release, controlled release, sustained release, extended release, modified release formulations, and mixed immediate release and controlled release formulations.

In embodiments, a composition is formulated in a unit dosage form. “Unit dosage form” refers to a physically discrete unit suited as unitary dosages for the subject to be treated, each unit containing a predetermined quantity of bioactive molecules calculated to produce the desired therapeutic effect(s), in association with a suitable pharmaceutical carrier, diluent, or excipient. Unit dosage forms may be used for ease of administration and uniformity of dosage. Unit dosage forms can contain a single or individual dose or unit, a sub-dose, or an appropriate fraction thereof (e.g., one half a “full” dose), of the composition administered. Unit dosage forms include capsules, troches, cachets, lozenges, tablets, ampules and vials, which may include a composition in a freeze-dried or lyophilised state; a sterile liquid carrier, for example, can be added prior to administration or delivery in vivo. Unit dosage forms include ampules and vials with liquid compositions disposed therein. Unit dosage forms include compounds for transdermal administration, such as patches that contact the epidermis for an extended or brief period of time.

In embodiments, a pharmaceutical composition is formulated in a pharmaceutically acceptable oral dosage form, including oral solid dosage forms and oral liquid dosage forms.

In embodiments, the compositions are formulated as a pharmaceutically acceptable oral solid dosage form, including lozenges, troches, tablets, capsules, caplets, powders, pellets, multiparticulates, beads, spheres, capsules, pills, and/or any combinations thereof. Oral solid dosage forms may be formulated as immediate release, controlled release, sustained release, extended release, or modified release formulations. In embodiments, solid dosage forms may comprise pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof. In embodiments, solid dosage forms may comprise pharmaceutically acceptable additives, such as a compatible carrier, complexing agent, ionic dispersion modulator, disintegrating agent, surfactant, lubricant, colorant, moistening agent, plasticizer, stabilizer, wetting agent, anti-foaming agent, alone or in combination, as well as supplementary active agent(s), including preservatives, antioxidants, antimicrobial agents including biocides, and biostats, such as antibacterial, antiviral and antifungal agents. Preservatives can be used to inhibit microbial growth or increase stability of the active ingredient thereby prolonging the shelf life of a pharmaceutical composition, and include EDTA, EGTA, benzalkonium chloride, benzoic acid, or benzoates, such as sodium benzoate. Antioxidants include vitamin A, vitamin C (ascorbic acid), vitamin E, tocopherols, other vitamins or provitamins, and compounds, such as alpha lipoic acid (ALA).

In embodiments, the compositions are formulated as a pharmaceutically acceptable oral liquid dosage form. Non-limiting examples of oral liquid dosage forms include tinctures, drops, emulsions, syrups, elixirs, suspensions, and solutions, and the like. In embodiments, oral liquid dosage forms may be formulated with any pharmaceutically acceptable excipient known to those of skill for the preparation of liquid dosage forms, and with solvents, diluents, carriers, excipients, and the like, chosen as appropriate to the solubility and other properties of the primary and/or secondary bioactive molecules disclosed herein and other ingredients. Non-limiting examples of solvents include, e.g., water, glycerin, simple syrup, alcohol, medium chain triglycerides (MCT), and combinations thereof.

In embodiments, oral liquid dosage forms may be monophasic or biphasic, the former being a substantially homogenous solution dissolved in water or non-aqueous solvent, while the latter refers to oral liquid dosage forms in which the bioactive molecules do not fully dissolve in common solvents. In embodiments, over time, the solid particles (i.e., the bioactive molecules) within the oral liquid dosage form may form a precipitate at the bottom of the container-requiring vigorous shaking to redisperse the bioactive molecules. Non-limiting examples of monophasic liquid forms include syrups, linctuses, spirits/essences, elixirs, and fluid extracts. Non-limiting examples of biphasic liquid forms include oral suspensions, oral emulsions, and mixtures.

Liquid dosage forms for oral administration may be prepared as liquid suspensions or solutions using a sterile liquid, such as, but not limited to, an oil, water, an alcohol, combinations of pharmaceutically suitable surfactants, suspending agents, and emulsifying agents. In embodiments, liquid formulations also may be prepared as single dose or multi-dose beverages. In embodiments, suspensions may include oils. Such oils include but are not limited to peanut oil, sesame oil, cottonseed oil, corn oil, and olive oil. Suitable oils also include carrier oils, such as MCT and long chain triglyceride (LCT) oils. In embodiments, a suspension preparation may also contain esters of fatty acids, such as ethyl oleate, isopropyl myristate, fatty acid glycerides, and acetylated fatty acid glycerides. In embodiments, suspension formulations may include alcohols, such as ethanol, isopropyl alcohol, hexadecyl alcohol; glycerol, and propylene glycol. In embodiments, ethers, such as polyethylene glycol; petroleum hydrocarbons, such as mineral oil and petrolatum; water also may be used in suspension formulations. In embodiments, a suspension can include an aqueous liquid or a non-aqueous liquid, an oil-in-water liquid emulsion, or a water-in-oil emulsion.

In addition to the primary and/or secondary bioactive molecules, the liquid dosage forms may comprise additives, such as (a) disintegrating agents, (b) dispersing agents, (c) wetting agents, (d) preservatives, (e) viscosity enhancing agents, (f) sweetening agents, and/or (g) flavoring agents. In addition to the additives listed above, the liquid formulations of the disclosure, in embodiments, may comprise inert diluents commonly used in the art, such as water or other solvents, solubilizing agents, emulsifiers, flavoring agents, and/or sweeteners. In embodiments, co-solvents and adjuvants may be added to a formulation.

In embodiments of modified release formulations, the plasma half-life compared to the plasma half-life of an immediate release formulation is greater by at least 5%, at least 10%, at least 15%, at least 20%, at least 25%, at least 30%, at least 35%, at least 50%, at least 75%, at least 100%, or values in between. In embodiments of modified release formulations, the formulations are designed to result in a comparable area under the curve, or AUCO-24, and a similar safety and efficacy profile, but having a delayed time to peak concentration (tmax) of at least 5%, at least 10%, at least 15%, at least 20%, at least 25%, at least 30%, at least 35%, at least 50%, at least 75%, at least 100%, or values in between. In embodiments, a formulation is designed to be a product with a specific time course based on an optimum therapeutic window, such as less than about 30 minutes, about 30 minutes, about 45 minutes, about 60 minutes, about 90 minutes, about 2 hours, about 3 hours, about 4 hours, and greater than 4 hours, including times in between.

In embodiments, a formulation is chosen based on its area of absorption. In embodiments, one may choose a tincture, such as the tincture described in the formulation examples below, based on its pre-digestion absorption by epithelial tissue.

In some embodiments, multiple formulations are combined, and the therapeutic combination comprises two or more pharmaceutical compositions together, which may be provided as a single pharmaceutical kit. In some embodiments, one or more portions of a therapeutic combination are provided as an inhalable formulation, such as for a soft mist inhaler, and the remaining portions of the combination are provided as a tincture. In embodiments, one or more portions of a therapeutic combination are provided as an inhalable formulation, and the remaining portions of the combination are provided as an oral dissolving strip. In embodiments, one or more portions of a therapeutic combination are provided as a tincture, and the remaining portions of the combination are provided as an oral dissolving strip. In embodiments, one or more portions of a therapeutic combination are provided as an inhalable formulation, and the remaining portions of the combination are provided as an oral spray, such as an oral mucosal spray. In embodiments, one or more portions of a therapeutic combination are provided as a tincture, and the remaining portions of the combination are provided as an oral spray.

In embodiments, more than two separate formulations are combined, and the therapeutic combination therefore comprises more than two pharmaceutical compositions together, which may be provided as a single pharmaceutical kit. In embodiments, more than three, more than four, more than five, or more than six separate formulations are combined, and the therapeutic combination therefore comprises more than two, more than three, more than four, more than five, or more than six pharmaceutical compositions together, which may be provided as a single pharmaceutical kit.

In embodiments, a pharmaceutical composition is formulated as an inhaled formulation. Non-limiting examples of inhaled formulations include soft mist inhaler formulations, dry powder aerosol formulations, and vaporizer formulations. In embodiments, the inhaled formulation is a soft mist inhaler formulation (“soft mist” formulation). Soft mist formulations may be produced by combining liquids containing bioactive molecules in a cartridge or syringe. The cartridge or syringe is then placed within a soft mist inhaler device, such as those described in WO 2020/167893 and U.S. Pat. No. 9,108,011, both incorporated by reference herein. One may also choose to include a solubilizer to assist in homogenizing the formulation (e.g., polysorbate 80, also known as TWEEN-80). In embodiments, the liquids containing the bioactive molecules may be liquid extracts obtained via the methods disclosed herein, with or without a solubilizing agent. In embodiments, the inhaled formulation may be a dry powder aerosol formulation, where bioactive molecules are obtained as a powder and are aerosolized by an inhaler device, such as a metered dose inhaler (MDI).

In embodiments, the inhaled formulation is a vaporizer formulation. A vaporizer formulation may be any of a vape juice, an e-liquid, an e-juice, and the like. In embodiments, vaporizer formulations may be a liquid (including, e.g., an oil) and/or a solid (including, e.g., a wax or a dry powder). In embodiments, vaporizer formulations may be produced by combining a bioactive molecule in a suitable liquid (a “base liquid”), the resultant formulation capable of being vaporized by a heating element (such as those found on vape pens, e-cigs, e-pipes, and other such e-smoking devices). Examples of suitable liquids include propylene glycol (PG), vegetable glycerin (VG), polyethylene glycol (PEG), and mixtures thereof, in any proportions. In embodiments, the base liquid may contain a liquid of lower viscosity to act as a thinning agent, including water and/or ethanol (including where ethanol is an alcoholic spirit, e.g., vodka). In embodiments, the vaporizer formulation contains a flavorant, which may be any flavor or flavor concentrate.

In embodiments, a pharmaceutical composition is formulated as an effervescent powder.

In embodiments, a pharmaceutical composition is formulated in a pharmaceutically acceptable transdermal application, capable of being administered transdermally. Non-limiting examples of transdermal formulations include ointments, creams, suspensions, lotions, pastes, gels, sprays, foams, oils, and the like, and any combination thereof.

In embodiments, a pharmaceutical composition is formulated for subcutaneous, intravenous, intra-arterial, intraperitoneal, intraosseous, intramuscular, intrathecal, or intracerebroventricular injection (“injectable formulations”). In embodiments, injectable formulations may be prepared by dissolving, suspending, or emulsifying the primary and/or secondary bioactive molecules in an aqueous or nonaqueous solvent, non-limiting examples of which include oils, such as vegetable oil, synthetic aliphatic acid glycerides, and esters of higher aliphatic acids or propylene glycol; and may also comprise additives such as solubilizers, stabilizers, and suspending, preserving, wetting, emulsifying, dispensing, and isotonic agents.

In some embodiments, a composition is formulated for a specific tissue or for a specific route of administration, other than for oral gastrointestinal administration, such as mucosal (e.g., sublingual, buccal, rectal, vaginal, nasal), IV, intramuscular (IM), subcutaneous (SC), cutaneous, intranasal, inhaled, and the like. Such administration, in some embodiments, may result in a reduction of side effects, reduced toxicity, increased efficacy, improved selectivity, enhanced bioavailability, and minimized drug-drug interactions.

Injectable formulations may comprise additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like, and may comprise isotonic agents, such as sugars, sodium chloride, and the like. Prolonged drug absorption of the injectable pharmaceutical form can be brought about by the use of agents delaying absorption, such as aluminum monostearate and gelatin. Injectable formulations designed for extended-release via SC or IM injection can avoid first-pass metabolism and lower dosages of the primary and/or secondary bioactive molecules will be necessary to maintain desired plasma levels. In such formulations, the particle size and the range of the particle sizes of the primary and/or secondary bioactive molecules can be used to control the release of the primary and/or secondary bioactive molecules by controlling the rate of dissolution in fat or muscle.

In embodiments, a composition is formulated in a pharmaceutically acceptable nanostructured formulation, such as a nanoemulsion, a nanocapsule, a nanoparticle conjugate, or a nano-encapsulated oral, sublingual, buccal, or nasal spray. In embodiments, nanostructured formulations are prepared by reference to general knowledge of the art (see, e.g., Jaiswal et al. 3 Biotech. 2015; 5(2):123-127).

In some embodiments, where a therapeutic combination, pharmaceutical composition, or other formulation comprises natural compounds or compounds found in nature, at least one of the carrier(s), diluent(s), and/or excipient(s) used in the combination, composition, or formulation are non-natural or non-naturally-occurring, or other agent(s), such as additional active agent(s), in the disclosed combination, composition, or formulation are non-natural or non-naturally-occurring, i.e., the natural compound(s) are combined with non-natural ingredient(s), so that the combination, composition, or formulation comprises at least one non-natural ingredient, and additionally, in embodiments, provides surprising synergistic effects. Where each of the compounds in a combination, composition, or formulation are natural or are found in nature, the combination, composition, or formulation, and/or the compound(s) therein, in embodiments, will demonstrate markedly different characteristics than than the natural products themselves.

By way of non-limiting and merely suggestive examples, the following formulations may be prepared, and which also may be used in the methods of the disclosure.

In all disclosed exemplary formulation embodiments below, the formulation may solely comprise a bioactive molecule from a fungus and a bioactive molecule from a plant (where “solely comprise” refers to no additional disclosed bioactive molecules, although a formulation may further comprise one or more carriers, diluents, or excipients, other inactive ingredients, and/or in some embodiments, additional active agents as disclosed herein). In embodiments, the formulation may solely comprise a bioactive molecule from a single genus of fungus and a bioactive molecule from a single genus of plant (that is, where the molecule(s) are extracted, isolated, derived, or otherwise obtained from a single genus, and not necessarily that they are only found in one genus). In embodiments, the formulation may solely comprise a bioactive molecule from a single species of fungus and a bioactive molecule from a single species of plant (that is, where the molecule(s) are extracted, isolated, derived, or otherwise obtained from a single species, and not necessarily that they are only found in one species). In embodiments, the formulation may solely comprise one bioactive molecule from a fungus and one bioactive molecule from a plant.

In some embodiments, the formulation may solely comprise a bioactive molecule from a psilocybin-producing species, and a bioactive molecule from a Cannabis plant. In embodiments, the formulation may solely comprise a bioactive molecule from a Psilocybe azurescens, Psilocybe bohemica, Psilocybe semilanceata, Psilocybe baeocystis, Psilocybe cyanescens, Psilocybe tampanensis, Psilocybe cubensis, Psilocybe weilii, Psilocybe hoogshagenii, Psilocybe stuntzii, Psilocybe cyanofibrillosa, or Psilocybe liniformans species, and a bioactive molecule from a Cannabis plant. In embodiments, the formulation may solely comprise one bioactive molecule from a psilocybin-producing species, and one bioactive molecule from a Cannabis plant. In embodiments, the formulation may solely comprise one bioactive molecule from a Psilocybe azurescens, Psilocybe bohemica, Psilocybe semilanceata, Psilocybe baeocystis, Psilocybe cyanescens, Psilocybe tampanensis, Psilocybe cubensis, Psilocybe weilii, Psilocybe hoogshagenii, Psilocybe stuntzii, Psilocybe cyanofibrillosa, or Psilocybe liniformans species, and one bioactive molecule from a Cannabis plant. In embodiments, a formulation may solely comprise a bioactive molecule from a Psilocybe fungus, and a bioactive molecule from a Cannabis plant. A “Psilocybe fungus” refers to a Psilocybe spp. fungus. In embodiments, a formulation may solely comprise one bioactive molecule from a Psilocybe fungus, and one bioactive molecule from a Cannabis plant.

For example, each of the below embodiments may be made with psilocybin (or psilocin) only, or both; with CBD (or THC) only, or both; and with each of coumarin, whole Pyropia extract, and ethanol and ginger (and any other disclosed active or inactive ingredients) optional, i.e., in some embodiments any one or more of coumarin, whole Pyropia extract, and ethanol and a flavorant and/or colorant such as ginger (and any other disclosed active or inactive ingredients) may not be included. Accordingly, in some embodiments, the exemplary formulation solely comprises psilocybin and CBD. In embodiments, the exemplary formulation solely comprises psilocin and CBD. In embodiments, the exemplary formulation solely comprises psilocybin, psilocin, and CBD. In embodiments, the exemplary formulation solely comprises psilocybin, THC, and CBD. In embodiments, the exemplary formulation solely comprises psilocin, THC, and CBD. In embodiments, the exemplary formulation solely comprises psilocybin, psilocin, and THC. In embodiments, the exemplary formulation solely comprises psilocybin and THC. In embodiments, the exemplary formulation solely comprises psilocin and THC. As above, “solely comprises” refers to no additional disclosed bioactive molecules, although a formulation may further comprise one or more carriers, diluents, or excipients, other inactive ingredients, and/or in some embodiments, additional active agents as disclosed herein, or as otherwise appropriate to the formulation, such as known in the art.

In some embodiments, any one or more of psilocybin, psilocin, THC, and CBD may be replaced with another bioactive molecule, such as another bioactive molecule from the same fungus or the same plant. In some embodiments, a flavorant and/or colorant (equivalently and as shorthand “flavorant/colorant”) is optional. Accordingly, any of the below examples without a flavorant/colorant is an additional embodiment. In some embodiments, the “flavorant/colorant” in an example is ethanol and ginger. In some embodiments, a formulation comprises an additional active agent. Where an active and/or inactive ingredient from a disclosed exemplary formulation is not present, the formulation will be prepared as described, together with general skill and knowledge in the art, and with such modifications as will be appreciated by ordinary artisans. Where an ingredient may be provided as a solid or as a liquid, one of skill will appreciate how to convert between mass amounts and volume amounts, including for ingredients, whether as a solid or a liquid, of an alternate or variable concentration.

### Example 1: Tincture formulation

A tincture is prepared with the proportions as follows:

| Ingredient | Quantity (units) | Extract % |
|---|---|---|
| Psilocybin | 250 μg | 45 |
| Psilocin | 150 μg | (30% from |
| | | sonified extraction |
| | | and 15% from |
| | | Soxhlet extraction) |
| CBD | 1 mg | 15 |
| THC | 1 mg | |
| Coumarin | 1 mg | 2 |
| Whole Pyropia extract | 8 mg | 15 |
| Flavorant/colorant | 8 mg | 15 |
| Water | | 8 |

The solution is prepared by combining the bioactive molecules from a fungus, a plant, and an algae, and/or extracts comprising them (“bioactive molecules and/or extracts comprising them”) with a flavoring agent and a solvent. In embodiments, an amount or a proportion of an ingredient, such as when expressed as a mass or as a percentage, includes the term “about.” In some embodiments, “about” a mass or “about” a percentage, including where the term “about” is implied by the context, refers to a range of ±2%. Thus for example, in embodiments where psilocybin (or a psilocybin-containing fungal extract) is 45% of a formulation, it will be understood that the amount in embodiments refers to psilocybin or a psilocybin-containing extract of “about” 45%, and in some such embodiments, an amount of “about” 45% refers to an amount of 45%±2%, i.e., between 43% and 47% of psilocybin or a psilocybin-containing extract, inclusive. In some other embodiments, an amount, such as expressed as a mass or a percentage, will also refer to “about” that amount, with “about” having the meaning as elsewhere described.

In embodiments, a fungal extract comprises about 45% of each dose, and contains about 400 μg of combined psilocybin and psilocin per dose; a Cannabis extract comprises about 15% of each dose, and contains about 1 mg CBD and about 1 mg THC per dose; a cumaru extract comprises about 2% of each dose, and contains about 1 mg of coumarin per dose; and an algal extract, which in embodiments is a Pyropia extract, comprises about 15% of each dose; the flavorant and/or colorant, which in some preferred embodiments is ethanol infused with ginger and bay leaf, and in some embodiments is another flavoring and/or coloring agent, which may be in ethanol, water, or another diluent, comprises about 15% of each dose; and added water constitutes about 8% of each dose. In some embodiments, a tincture or other liquid formulation herein further comprises a solubilizing agent, such as a cyclodextrin, lecithin, propylene glycol, xanthan gum, or a combination thereof.

Tinctures may be prepared by exposing fungal, plant, and/or algal matter to a solvent capable of extracting the desired primary and/or secondary bioactive molecules, combining the extracts, and then optionally adding a flavorant and/or coloring agent.

In some embodiments, the solvent is alcohol. In embodiments, the alcohol may be, e.g., 40% to 60% alcohol, or may be 80% to 90% alcohol, and then diluted to between 40% and 60% alcohol. In some embodiments, the solvent is water. In some embodiments, the solvent may be an acid, for example, acetic acid.

As an example, food coloring may be added to the tincture in order to provide a certain appearance to the solution and may contain additional compounds sufficient to improve the taste of the tincture. In embodiments, the food coloring may comprise a compound which improves the biological activity of the primary and/or secondary bioactive molecules. Examples of suitable food coloring for use with disclosed extracts, compositions and tinctures include turmeric extracts, cinnamon extracts, beetroot extracts, carrot extracts, caramel, blueberry extracts, blackberry extracts, and ginger extracts.

In some embodiments, a subject will be administered, on a daily basis, one dose of a tincture having ingredients in the amounts as formulated above, two doses of a tincture in the amounts as formulated above, three such doses, four such doses, five such doses, or greater than five such doses daily. In some preferred embodiments, a daily dosage amount is four such doses.

### Example 2: Oral Spray and Oral Mucosal Spray Formulation

An oral spray may be prepared comprising the same ingredients, quantities (units) of ingredients, and overall proportions as in EXAMPLE 1. Where an oral spray is formulated for oral mucosal (e.g., sublingual or buccal) administration, it may further comprise a penetration enhancer and/or a mucoadhesive polymer.

### Example 3: Soft Mist Inhaler Formulation

A soft mist inhaler formulation is prepared as follows, comprising the same ingredients, quantities (units) of ingredients, and overall proportions as in EXAMPLE 1 above: The solution is prepared by combining the bioactive molecules from a fungus, a plant, and an algae, and/or extracts comprising them with the flavorant/colorant and the solvent.

The bioactive molecules and/or extracts comprising them are mixed and combined. The combined bioactive molecules and/or extracts comprising them may then be combined with ethanol and/or water, and an optional added preservative, for example, benzalkonium chloride, to a volume of, for example, 15 mL. Reference may be made to Anderson. Int J Chron Obstruct Pulmon Dis. 2006; 1(3):251-259 and Dalby et al. Med Devices (Auckl). 2011; 4:145-155, both of which are incorporated by reference herein in their entirety.

### Example 4: Vaporizer Formulation

A vaporizer formulation, comprising 500 μg psilocybin, 300 μg psilocin, 2 mg CBD, 2 mg THC, 2 mg coumarin, 16 mg whole Pyropia extract, 16 mg flavorant/colorant, and 10 mL 50:50 base liquid (50% PG, 50% VG), is prepared as follows: The bioactive molecules and/or extracts comprising them are mixed and combined (in solid form, and/or as part of one or more liquid extracts) with the 50:50 base liquid prepared for use with any liquid vaporization device or appliance, such as e-liquid vaporizers, e-cigs, mods, vape pens, and the like. A flavorant/colorant (e.g., comprising ginger and/or bay laurel) optionally may be added. The formulation also can be prepared for any oil, thin oil, e-juice, or e-liquid vaporizer, according to ordinary skill.

### Example 5: Tablet and Scorable Double-Strength Tablet Formulation

Tablets comprising 500 μg psilocybin, 300 μg psilocin, 1 mg CBD, 1 mg THC, 1 mg coumarin, 16 mg whole Pyropia extract, 170 mg microcrystalline cellulose, 10 mg colloidal silicon dioxide, and 7.5 mg stearic acid are prepared by blending the bioactive molecules and/or extracts comprising them together with the other ingredients, and compressing it to form tablets.

Scorable double strength tablets comprising 1000 μg psilocybin, 600 μg psilocin, 2 mg CBD, 2 mg THC, 2 mg coumarin, 32 mg whole Pyropia extract, 35 mg microcrystalline cellulose, 45 mg starch, 4.5 mg sodium carboxymethyl starch, 0.5 mg magnesium stearate, 1 mg talc, and 4 mg polyvinylpyrrolidone (PVP) (as 10% solution in water), are prepared as follows: The bioactive molecules and/or extracts comprising them, starch, and cellulose are passed through a No. 20 mesh U.S. sieve and mixed thoroughly. The solution of PVP is mixed with the resultant powders, which are then passed through a 16 mesh U.S. sieve. The granules so produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets. Tablets are scored to provide the ability to create equal half doses.

### Example 6: Capsule Formulation

Capsules, each comprising 500 μg psilocybin, 300 μg psilocin, 2 mg CBD, 2 mg THC, 2 mg coumarin, 16 mg whole Pyropia extract, 119 mg cellulose and/or starch, and 1 mg magnesium stearate are made by blending the bioactive molecules and/or extracts comprising them, together with the cellulose and/or starch and magnesium stearate, passing them through a No. 20 mesh U.S. sieve, and filling the blended mixture into hard or soft gelatin capsules.

### Example 7: Alternate Capsule Formulation, Optionally with Additional Active Agent(s)

Capsules, each comprising 1000 μg psilocybin, 600 μg psilocin, 2 mg CBD, 2 mg THC, 2 mg coumarin, 16 mg whole Pyropia extract, 50 mg serotonergic agent, 100 mg cellulose and/or starch, and 1 mg magnesium stearate are made as follows: The bioactive molecules and/or extracts comprising them, cellulose and/or starch, and magnesium stearate are blended, passed through a No. 20 mesh U.S. sieve, and filled into hard or soft gelatin capsules. Optionally, an additional active agent may be added during blending. In some embodiments, the additional active agent is a serotonergic agent, such as an antidepressant or an anxiolytic. In some embodiments, the additional active agent is an anti-inflammatory agent, as described herein or generally known in the art.

### Example 8: Suspension Formulation

A suspension, comprising 250 μg psilocybin, 150 μg psilocin, 1 mg CBD, 1 mg THC, 1 mg coumarin, and 8 mg whole Pyropia extract is made as follows: The bioactive molecules and/or extracts comprising them are blended together with 1.75 g sucrose and 4 mg xanthan gum, passed through a No. 10 mesh U.S. sieve, and then mixed with a previously made solution of 50 mg sodium carboxymethyl cellulose (11%) and 50 mg microcrystalline cellulose (89%) in water. 10 mg sodium benzoate and optional flavorant/colorant to taste (qv), are diluted with some of the water and added with stirring. Sufficient water is then added to make 5 mL.

### Example 9: Intravenous Solution Formulation

An intravenous formulation, comprising 250 μg psilocybin, 150 μg psilocin, 1 mg CBD, 1 mg THC, 1 mg coumarin, 8 mg whole Pyropia extract, and 1,000 mL isotonic saline, may be prepared as follows: The bioactive molecules and/or extracts comprising them are dissolved in appropriate solvent, such as isotonic saline or another suitable solvent; additional active or inactive ingredients such as solubilizers and preservatives may be added, as otherwise described above, and within the general knowledge of the art. It will be understood that the amount of each bioactive molecule and/or extract can be adjusted accordingly to reach the desired mg/mL.

### Example 10: Injectable Solution Formulation

An injectable formulation, comprising 250 μg psilocybin, 150 μg psilocin, 1 mg CBD, 1 mg THC, 1 mg coumarin, 8 mg whole Pyropia extract, and 5 mL isotonic saline, may be prepared as follows: The bioactive molecules and/or extracts comprising them are dissolved in appropriate solvent, such as isotonic saline or another suitable solvent; additional active or inactive ingredients such as preservatives may be added, as otherwise described above, and within the knowledge in the art. It will be understood that the amount of each bioactive molecule and/or extract can be adjusted accordingly to reach the desired mg/mL.

### Example 11: Topical Formulation for Transdermal Administration

A topical formulation, comprising 400 μg psilocybin, 250 μg psilocin, 2 mg CBD, 2 mg THC, 2 mg coumarin, 8 mg whole Pyropia extract, 30 mg emulsifying wax, 20 mg liquid paraffin, in an amount of white soft paraffin to equal 100 g total, may be prepared as follows: White soft paraffin is heated until molten. Liquid paraffin and emulsifying wax are incorporated and stirred until dissolved. Bioactive molecules and/or extracts comprising them are added and stirring is continued until dispersed. The mixture is then cooled until solid.

### Example 12: Cut Matrix Sublingual or Buccal Tablet Formulation

Sublingual or buccal tablets, comprising 500 μg psilocybin, 300 μg psilocin, 2 mg CBD, 2 mg THC, 2 mg coumarin, 8 mg whole Pyropia extract, 210.5 mg glycerol, 143 mg water, 4.5 mg sodium citrate, 26.5 mg polyvinyl alcohol, and 15.5 mg polyvinylpyrrolidone (PVP), are made as follows: The glycerol, water, sodium citrate, polyvinyl alcohol, and PVP are admixed by continuous stirring while maintaining a temperature of about 90° C. When the polymers have gone into solution, the solution is cooled to about 50-55° C. and the bioactive molecules and/or extracts comprising them are slowly admixed. The homogenous mixture is poured into forms made of an inert material to produce a drug-containing diffusion matrix having a thickness of about 2-4 mm. The diffusion matrix is then cut to form individual tablets of the appropriate size.

### Example 13: Individually Formed Sublingual or Buccal Lozenge Formulation

Sublingual or buccal lozenges, comprising 500 μg psilocybin, 300 μg psilocin, 2 mg CBD, 2 mg THC, 2 mg coumarin, 8 mg whole Pyropia extract, 350 mg silica gel powder, 400 mg citric acid powder, 600 mg acacia powder, 1 g polyethylene glycol (PEG), and optionally 100 mg flavorant/colorant, are made as follows: The silica gel powder, citric acid powder, acacia powder, optional flavorant/colorant, and PEG are mixed by continuous stirring at a temperature of about 90° C. When the PEG has melted and the other ingredients have gone into solution, the solution is cooled to about 50-55° C. and the bioactive molecules and/or extracts comprising them are slowly admixed. The homogenous mixture is poured into separate molds and allowed to cool. Reference may also be made to the incorporated U.S. Pat. No. 10,034,832 and the Examples therein.

### Example 14: Intranasal Delivery Formulation

A nasal spray formulation for intranasal delivery, comprising 250 μg psilocybin, 150 μg psilocin, 1 mg CBD, 1 mg THC, 1 mg coumarin, 8 mg whole Pyropia extract, 50 μL DMSO, 5 mL MCT, in saline (1% cremophor) to a total formulation of 10 mL, may be prepared as follows: The solution at approximately 1 mg/mL of bioactive molecules and/or extracts comprising them in 49.5% MCT, 49.5% saline, 0.5% DMSO, and 0.5% cremophor is prepared for use in nasal spray device. In other embodiments, a nasal formulation can be prepared as a dry powder for inhalation, e.g., by combining the bioactive molecules and/or extracts comprising them with lactose and mixing for use with a dry powder inhaling appliance, or as in U.S. Pub. No. US2015/0367091A1 and references cited therein.

In some exemplary embodiments, a disclosed therapeutic combination or composition comprises the primary bioactive molecules in the table below, from each listed component of the combination or composition, and such combination or composition may be formulated according to any of the disclosed Examples, or as another exemplary formulation according to the disclosure and general knowledge in the art:

| Component | Cannabis sativa | | |
|---|---|---|---|
| Primary bioactive(s) | D9-THC | Cannabidiol | |
| Chemical structure | 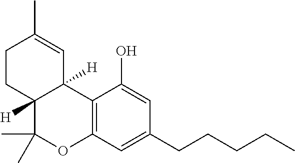 | 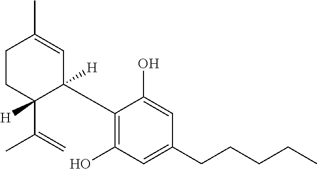 [SMILES: C=C(C)[C@@H]1CCC(C)=C[C@H]1c1c(O)cc(CCCCC)cc1O] | |
| Molecular Formula | C₂₁H₃₀O₂ | C₂₁H₃₀O₂ | |
| Molecular weight (g/mol) | 314.469 | 314.464 | |
| Exemplary Source | National Institute on Drug Abuse, National Institute of Health. | | |
| Component | Psilocybe cubensis | | |
| Primary bioactive(s) | Psilocin | Psilocybin | |
| Chemical structure | 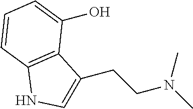 | 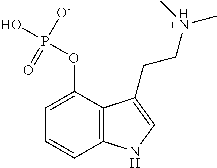 | |
| Molecular Formula | C₁₂H₁₆N₂O | C₁₂H₁₇N₂O₄P | |
| Molecular weight (g/mol) | 204.27 | 284.25 | |
| Exemplary Source | Numinus Wellness (Vancouver, BC) | | |
| Component | Dipteryx odorata | Pyropia yezoensis | |
| Primary bioactive(s) | Coumarin | Porphyran | Taurine |
| Chemical structure | 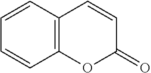 | 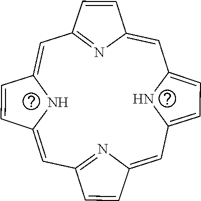 [SMILES: C1=Cc2cc3ccc(cc4nc(cc5ccc(cc1n2)[nH]5)C=C4)[nH]3] | 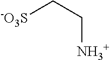 |
| Molecular Formula | C₉H₆O₂ | C₂₀H₁₄N₄ | C₂H₇NO₃S |
| Mol. weight (g/mol) | 146.14 | 310.4 | 125.14 g/mol |

| | |
|---|---|
| Exemplary Source | Mountain Rose Herbs (Eugene, OR) |

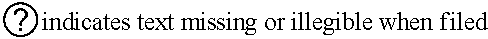

Note, while certain primary and/or secondary bioactive molecules, as well as certain extracts comprising them, are disclosed above, this should not be construed as limiting the disclosure, nor should it be construed as limiting the formulation of EXAMPLE 1 to the primary and/or secondary bioactive molecules disclosed above. Thus, in embodiments, the formulation of EXAMPLE 1 (or any other Example herein) contains additional, or fewer, primary and/or secondary bioactive molecules than are disclosed; including any of the primary and/or secondary bioactive molecules disclosed herein for each of the fungal, Cannabis, cumaru, and algal extracts.

In some alternative embodiments of this Example, the formulation comprises at least one bioactive molecule from a fungus, and at least one bioactive molecule from a plant, the bioactive molecule(s) from fungi comprising those from a psilocybin-producing species, such as from the genera Copelandia, Galerina, Gymnopilus, Inocybe, Panaeolus, Pholiotina, and Pluteus, and the bioactive molecule(s) from plant(s) comprising those from a cannabinoid-producing species, such as from the genus Cannabis. In some such alternative embodiments, the formulation comprises no further bioactive molecule(s) from fungi and bioactive molecule(s) from plant(s) except for those from a psilocybin-comprising species and from a cannabinoid-producing species. In some such alternative embodiments, the formulation comprises no further bioactive molecule(s) from fungi and bioactive molecule(s) from plant(s) except for those from the genera Copelandia, Galerina, Gymnopilus, Inocybe, Panaeolus, Pholiotina, and Pluteus, and from the genus Cannabis. In some such alternative embodiments, the formulation comprises no bioactive molecules from the genus Dipteryx. In some such alternative embodiments, the formulation comprises no bioactive molecules from algae, a marine algae, the family Bangiaceae, or the genera Pyropia and Porphyra. In some such alternative embodiments, the only bioactive molecules are psilocybin, psilocin, CBD, and THC. In some such alternative embodiments, the only bioactive molecules are psilocybin, CBD, and THC. In some such alternative embodiments, the only bioactive molecules are psilocybin and CBD. In some such alternative embodiments, the only bioactive molecules are psilocybin and THC. In some such alternative embodiments, the only bioactive molecules are psilocin, CBD, and THC. In some such alternative embodiments, the only bioactive molecules are psilocin and CBD. In some such alternative embodiments, the only bioactive molecules are psilocin and THC.

It will be appreciated that the above formulation examples are illustrative only. Any of the disclosed primary and/or secondary bioactive molecules may be utilized in the formulation examples, in a dosage range applicable for said primary and/or secondary bioactive molecules. It will be understood that reference to a particular primary and/or secondary bioactive molecule is merely illustrative, and bioactive molecules in any Example may be substituted by other primary and/or secondary bioactive molecules disclosed herein.

Moreover, it will be readily appreciated that the disclosed compositions are not limited to combinations of a single primary and/or secondary bioactive molecule, or (when formulated as a pharmaceutical composition) limited to a single carrier, diluent, and/or excipient alone, but may also include combinations of multiple primary and/or secondary bioactive molecules (including additional bioactive molecules), and/or multiple carriers, diluents, and excipients. Pharmaceutical compositions of this disclosure thus may comprise any of the primary and/or secondary bioactive molecules disclosed above. Meaning, in embodiments, the disclosed composition comprises a primary and/or secondary bioactive molecule from a fungus, a plant, and an algae, optionally together with one or more other bioactive molecules (or their derivatives and analogs) in combination, together with one or more pharmaceutically-acceptable carriers, diluents, and/or excipients, and additionally with one or more other active agents.

In embodiments, a disclosed composition will be prepared so as to increase an existing therapeutic effect, provide an additional therapeutic effect, increase adherence or ease-of-use, increase a desired property, such as stability or shelf-life, decrease an unwanted effect or property, alter a property in a desirable way (such as pharmacokinetics or pharmacodynamics), modulate a desired system or pathway (e.g., a neurotransmitter system), or provide synergistic effects.

“Therapeutic effects” that may be increased or added in embodiments of the disclosure include, but are not limited to, antioxidant, anti-inflammatory, analgesic, antineuropathic, antinociceptive, antimigraine, anxiolytic, antidepressant, antipsychotic, anti-PTSD, dissociative, immunostimulant, anti-cancer, antiemetic, orexigenic, antiulcer, antihistamine, antihypertensive, anticonvulsant, antiepileptic, bronchodilator, neuroprotective, nootropic, empathogenic, psychedelic, sedative, or stimulant effects, as well as such effects as are known to ameliorate symptoms caused by a inflammatory conditions or disorder.

The goal of increasing an existing therapeutic effect, providing an additional therapeutic effect, increasing adherence or ease-of-use, increasing a desired property, such as stability or shelf-life, decreasing an unwanted effect or property, altering a property in a desirable way (such as pharmacokinetics or pharmacodynamics), modulating a desired system or pathway (e.g, a neurotransmitter system), or otherwise inducing synergy, in embodiments, is achieved by the inclusion of an additional active agent.

The type of formulation employed for the administration of the disclosed primary and/or secondary bioactive molecules employed in the methods generally may be dictated by the primary and/or secondary bioactive molecule(s) employed, the type of pharmacokinetic profile desired from the route of administration and the primary and/or secondary bioactive molecule(s), and the state of the subject. It will be appreciated that any of the above embodiments also be combined to form additional embodiments.

### A. Routes of Administration

The disclosed pharmaceutical compositions are suitable for administration by a variety of routes. Non-limiting examples of routes of administration include enteral administration, such as oral, sublingual, buccal, and rectal administration, parenteral administration, including bolus injection or continuous infusion, intravenous, intra-arterial, intraperitoneal, intraosseous, intramuscular, intrathecal, intracerebroventricular, vaginal, ocular, nasal, cutaneous, topical, otic, ocular, transdermal, and subcutaneous administration.

In embodiments, a pharmaceutical composition is administered as oral solid and oral liquid dosage forms; sublingually or buccally; as injections, including intravenous, intra-arterial, intraperitoneal, intraosseous, intramuscular, intrathecal, and intracerebroventricular; rectally, vaginally, ocularly, nasally, cutaneously, topically, oticly, transdermally, and subcutaneously.

In embodiments, in which administration is enteral, parenteral, or both, an effective amount of a primary and/or secondary bioactive molecule is systemically administered to a subject. In embodiments, an effective amount of a primary and/or secondary bioactive molecule is administered orally to a subject. In embodiments, an effective amount of a primary and/or secondary bioactive molecule is intravenously administered to a subject. In embodiments, an effective amount of a primary and/or secondary bioactive molecule is administered by inhalation to a subject. In embodiments, an effective amount of a primary and/or secondary bioactive molecule is administered by nasal administration to a subject. In embodiments, an effective amount of a primary and/or secondary bioactive molecule is administered by injection to a subject. In embodiments, an effective amount of a primary and/or secondary bioactive molecule is administered topically (dermally) to a subject. In embodiments, an effective amount of a primary and/or secondary bioactive molecule is administered by ophthalmic administration to a subject. In embodiments, an effective amount of a primary and/or secondary bioactive molecule is administered rectally to a subject. In embodiments, the primary and/or secondary bioactive molecules disclosed herein and employed in the methods described herein are effectively administered to a subject via other means, and prepared as any acceptable composition known to those of skill. In embodiments, such compositions may be prepared in any manner known in the pharmaceutical arts that comprise at least one bioactive molecule (Sheth et al. Compressed tablets. In: Pharmaceutical Dosage Forms: Tablets Eds. H. A. Lieberman and L. Lachman. Vol. 1. 1980. 109. Marcel Dekker).

In embodiments, the primary and/or secondary bioactive molecules disclosed herein are administered by multiple routes, which may differ between subjects, such as a patient, according to subject preferences, comorbidities, side effect profiles, pharmacokinetic and pharmacodynamic considerations, and other factors. In embodiments are the presence of other substances with the primary and/or secondary bioactive molecules, known to those skilled in the art, such as modifications in the preparation to facilitate absorption through various routes (e.g., gastrointestinal, transdermal, etc.), to extend the effect of the drugs, and/or attain higher or more stable serum levels or enhance the therapeutic effect of the primary and/or secondary bioactive molecules disclosed herein.

In embodiments, the pharmaceutical compositions are suitable as oral solid or oral liquid dosage forms, administered sublingually, buccally, rectally, vaginally, ocularly, oticly, nasally, cutaneously, topically, and transdermally; or as intravenous, intra-arterial, intraperitoneal, intraosseous, intramuscular, intrathecal, intracerebroventricular, and subcutaneous injection, wherein such injections comprise physiologically acceptable sterile aqueous or non-aqueous solutions, dispersions, suspensions or emulsions, liposomes, and sterile powders for reconstitution into sterile injectable solutions or dispersions.

In embodiments, a composition may be administered via enteral or parenteral means, wherein enteral means includes, but is not limited to, oral solid and oral liquid dosage forms, sublingual and buccal administration, and rectal administration; and parenteral administration means includes, but is not limited to, bolus injection or continuous infusion, intravenous, intra-arterial, intraperitoneal, intraosseous, intramuscular, intrathecal, intracerebroventricular, vaginal, ocular, nasal, cutaneous, topical, otic, transdermal, and subcutaneous administration; in addition to other equivalent means known to those of skill.

Enteral administration includes administration involving any part of the gastrointestinal tract. Non-limiting examples include those by mouth (orally), including oral solid and oral liquid dosage forms, and rectal, and in some embodiments may be preferably formulated as tinctures. Parenteral administration refers to administration from any means not involving the gastrointestinal tract, including intravenous (into a vein), intra-arterial (into an artery), intraosseous infusion (into bone marrow), intramuscular (into a muscle), intracerebral (into a brain parenchyma), intracerebroventricular (into a cerebral ventricular system), intrathecal (an injection into a spinal canal), otic (through an ear), ocular (through an eye), vaginal (into a vagina), and subcutaneous (under skin). In embodiments, parenteral administration may include sublingual and/or buccal administration. In embodiments, a pharmaceutical composition may be administered to a subject via injection. In embodiments, a pharmaceutical composition may be administered to a subject via nasal systems or the mouth through, for example, an oral solid and/or oral liquid dosage forms; inhalation, via a nasal spray or oral inhaler; nebulization, from a nebulizer, such as a machine that turns liquid medicine into a mist; or buccally/sublingually. In embodiments, a pharmaceutical composition may be administered to a subject via a combination of administration means. In embodiments, the pharmaceutical composition may be administered to a subject via an enteral administration means. In embodiments, the pharmaceutical composition may be administered to a subject via a parenteral administration means. In embodiments, the composition may be administered to a subject via at least one enteral administration means, and at least one parenteral administration means. In embodiments, an equivalent route of administration known to one of skill is utilized.

In some embodiments, a therapeutic combination may be administered via multiple routes of administration. For example, in embodiments, the fungal portion of a disclosed combination is administered via one route of administration, and the plant portion(s) and algal portion are administered via a different route of administration. In embodiments, the plant portion(s) of a disclosed combination is/are administered via one route of administration, and the fungal and algal portions are administered via a different route of administration. In embodiments, the algal portion of a disclosed combination is administered via one route of administration, and the plant portion(s) and fungal portion are administered via a different route of administration.

Administration of different (fungal, plant, or algal) portions of a disclosed combination to different tissues may maximize the therapeutic effects of the combination, result in reduction of side effects, reduction of patient discomfort, and/or increase of bioavailability. In one illustrative example, a disclosed combination comprises a plant portion comprising a cannabinoid (e.g., THC) or a Cannabis extract, which is administered to the oral mucosa via spray; while remaining ingredients (e.g., fungal, plant, and/or algal portions) are delivered to epithelial cells in the lung via a soft-mist inhaler. In some embodiments, administration according to this exemplary procedure results in reduced throat irritation (e.g., in individuals sensitive to inhaling Cannabis or cannabinoids), maximizes bioavailability for the remaining ingredients (e.g., fungal, plant, and/or algal portions), and improves the pharmacokinetic profiles of the components of the therapeutic combination; for example, by expediting the uptake of the longer-lasting plant portion (e.g., the THC or Cannabis extract) while moderating the uptake of shorter-lasting portions.

In another example, a disclosed combination comprises an algal portion comprising Pyropia or a Pyropia extract, which is administered to the oral mucosa via oral spray; while remaining ingredients (e.g., fungal, plant, and/or algal portions) are delivered to epithelial cells in the lungs via soft-mist inhaler. In some embodiments, administration according to this exemplary procedure results in reduced throat irritation, maximizes bioavailability of ingredients (e.g., fungal, plant, and/or algal portions), and improves the pharmacokinetic profiles of the components of the therapeutic combination; for example, by expediting the uptake of the long-lasting portions while moderating the uptake of the shorter-lasting algal portion (e.g., the Pyropia or Pyropia extract).

### B. Methods of Administration

In some aspects, provided are methods of administration or methods of administering a primary and/or secondary bioactive molecule disclosed herein. As used herein, the terms “subject,” “user,” “patient,” and “individual” are used interchangeably, and refer to any mammal, preferably a human. Such terms will be understood to include one who has an indication for which the combinations, compositions, or methods described herein may be efficacious, or who otherwise may benefit by the invention. In general, all of the combinations, compositions, and methods of the disclosure will be appreciated to work for all individuals, although individual variation is to be expected, and will be understood.

Provides are methods for using therapeutically effective amounts of the pharmaceutical compositions of the disclosure containing the primary and/or secondary bioactive molecules disclosed herein in a mammal, and preferably a human. Such methods include those for treating inflammatory conditions and disorders, including in a healthy individual.

Administration of pharmaceutical compositions in an “effective amount,” a “therapeutically effective amount,” a “therapeutically effective dose,” or a “pharmacologically effective amount,” refers to an amount of a primary and/or secondary bioactive molecule that is sufficient to provide the desired therapeutic effect, for example, relieving to some extent one or more of the symptoms of the disease or condition being treated. The result can be reduction and/or alleviation of the signs, symptoms, or causes of a disease or disorder, or any other desired alteration of a biological system. “Therapeutically effective amount” includes, for example, a prophylactically effective amount.

An “effective amount” of a primary and/or secondary bioactive molecule disclosed herein is an amount effective to achieve a desired pharmacologic effect or meaningful therapeutic improvement. It is understood that “an effective amount” or “a therapeutically effective amount” can vary from subject to subject due to variation in metabolism of a compound, such as the primary and/or secondary bioactive molecules disclosed herein, of age, weight, general condition of the subject, the condition being treated, the severity of the condition being treated, and the judgment of the prescribing physician. The effective amount will vary depending upon the subject and the disease condition being treated or health benefit sought, the weight and age of the subject, the severity of the disease condition or degree of health benefit sought, the manner of administration, and the like, all of which can readily be determined by one of skill. Additionally, pharmacogenomic (the effect of genotype on the pharmacokinetic, pharmacodynamic or efficacy profile of a therapeutic) information about a particular patient may affect dosage used.

As used herein, “therapeutic effect” or “therapeutic efficacy” means the responses(s) in a mammal, and preferably a human, after treatment that is judged to be desirable and beneficial. Hence, depending on the disorder to be treated, or improvement in physiological or psychological functioning sought, and depending on the particular constituent(s) in the compositions of the disclosure under consideration, those responses shall differ, but would be readily understood by those of skill. For example, in embodiments, “therapeutic effect” may refer to an effect caused by the disclosed composition, or its use in a method of the disclosure, such as the treatment of inflammatory conditions and disorders, as disclosed herein.

“Therapeutically effective dose” refers to the dose necessary to elicit a desired result within a patient undergoing treatment. A therapeutically effective dose therefore may, in embodiments, refer to a dose of the pharmaceutical composition or therapeutic combination necessary to deliver measurable patient-specific biologic effects in the treatment or prevention of a condition or disorder. A “therapeutically effective dose” may be used interchangeably with a “therapeutically effective amount” or an “effective amount.”

### 1. Dosing

It will be readily appreciated that dosages may vary depending upon whether the treatment is therapeutic or prophylactic, the onset, progression, severity, frequency, duration, probability of or susceptibility of the symptom to which treatment is directed, clinical endpoint desired, previous, simultaneous or subsequent treatments, general health, age, gender, and race of the subject, bioavailability, potential adverse systemic, regional or local side effects, the presence of other disorders or diseases in the subject, and other factors that will be appreciated by the skilled artisan (e.g., medical or familial history).

In all embodiments herein that include dose amounts of less than about 1 mg, such dose amounts will be understood to include further specific dose amounts of about 0.5 mg or less, about 0.25 mg or less, about 0.1 mg or less, about 0.05 mg or less, about 0.005 mg or less, about 0.001 mg or less, and about 0.0005 mg or less.

In all embodiments herein that include dose amounts of at least about 1 mg or more, up to and including about 75 mg, such dose amounts will be understood to include further specific dose amounts of (with all such milligram dose amounts to be understood also to be preceded by the modifier “about”) 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, as well as amounts within these ranges.

In all embodiments herein that include dose amounts of 75 mg, as well as greater than 75 mg, including 100 mg, 150 mg, 200 mg, or greater than 200 mg, such dose amounts will be understood to include further specific dose amounts of 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, and 200 mg. Such dose amounts additionally will be understood to include amounts within these ranges, and in all such embodiments, a single dose moreover may be greater than 200 mg, including 225 mg, 250 mg, or greater than 250 mg.

In embodiments, wherein the combination and/or composition comprises a primary and/or secondary bioactive molecule from a fungus, the primary and/or secondary bioactive molecule may be present in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg.

In embodiments, wherein the combination and/or composition comprises a primary and/or secondary bioactive molecule from a fungus, the primary and/or secondary bioactive molecule may be present in an amount so that a single dose is (whether or not such dose is present in a unit dosage form), about 100 μg or less, (including 95 μg, 90 μg, 85 μg, 80 μg, 75 μg, 70 μg, 65 μg, 60 μg, 55 μg, 50 μg, 45 μg, 40 μg, 35 μg, 30 μg, 25 μg, 20 μg, 15 μg, 10 μg, 5 μg, and 5 μg or less), or at least about 100 μg, or more, and greater than 1,000 μg, including 1,500 μg, 2,000 μg, up to and including 5,000 μg, and in some embodiments, greater than 5,000 μg.

In some embodiments, wherein the primary bioactive molecule from a fungus is any of psilocybin, psilocin, baeocystin, norbaeocystin, norpsilocin, aeruginascin, and a β-carboline, the primary bioactive molecule may be present in an amount so that a single dose is (whether or not such dose is present in a unit dosage form), about 100 μg or less, at least about 100 μg, or more, and greater than 1,000 μg, including 1,500 μg, 2,000 μg, up to and including 5,000 μg.

In embodiments, the primary bioactive molecule from a fungus is psilocybin. In embodiments, the psilocybin is present in an amount so that a single dose is between about 10 and 1000 μg. In embodiments, the psilocybin is present in an amount so that a single dose is between about 50 and 500 μg. In embodiments, the psilocybin is present in an amount so that a single dose is between about 100 and 400 μg. In embodiments, the psilocybin is present in an amount so that a single dose is between about 200 and 300 μg. In embodiments, the psilocybin is present in an amount so that a single dose is about 250 μg.

In embodiments, the primary bioactive molecule from a fungus is psilocin. In embodiments, the psilocin is present in an amount so that a single dose is between about 10 and 500 μg. In embodiments, the psilocin is present in an amount so that a single dose is between about 20 and 300 μg. In embodiments, the psilocin is present in an amount so that a single dose is between about 20 and 200 μg. In embodiments, the psilocin is present in an amount so that a single dose is between about 100 and 200 μg. In embodiments, the psilocin is present in an amount so that a single dose is about 150 μg.

In embodiments, wherein the secondary bioactive molecule from a fungus is any of a polysaccharide, a peptide, a terpene or terpenoid, a phenolic compound, a mineral, a vitamin, an amino acid, a lipid, choline, and a lactone, the secondary bioactive molecule may be present in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg.

In all embodiments herein that include dose amounts of about 100 μg or less, such embodiments will be understood to include further specific dose amounts including about 95 μg, 90 μg, 85 μg, 80 μg, 75 μg, 70 μg, 65 μg, 60 μg, 55 μg, 50 μg, 45 μg, 40 μg, 35 μg, 30 μg, 25 μg, 20 μg, 15 μg, 10 μg, 5 μg, and about 5 μg or less.

In all embodiments herein that include dose amounts of at least about 100 μg, or more, and greater than 1,000 μg, including 1,500 μg, 2,000 μg, up to and including 5,000 μg, such embodiments will be understood to include further specific dose amounts including 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 μg, 300 μg, 310 μg, 320 μg, 330 μg, 340 μg, 350 μg, 360 μg, 370 μg, 380 μg, 390 μg, 400 μg, 410 μg, 420 μg, 430 μg, 440 μg, 450 μg, 460 μg, 470 μg, 480 μg, 490 μg, 500 μg, 510 μg, 520 μg, 530 μg, 540 μg, 550 μg, 560 μg, 570 μg, 580 μg, 590 μg, 600 μg, 610 μg, 620 μg, 630 μg, 640 μg, 650 μg, 660 μg, 670 μg, 680 μg, 680 μg, 700 μg, 710 μg, 720 μg, 730 μg, 740 μg, 750 μg, 760 μg, 770 μg, 780 μg, 790 μg, 800 μg, 810 μg, 820 μg, 830 μg, 840 μg, 850 μg, 860 μg, 870 μg, 880 μg, 890 μg, 900 μg, 910 μg, 920 μg, 930 μg, 940 μg, 950 μg, 960 μg, 970 μg, 980 μg, 990 μg, and 1000 μg, as well as in some embodiments, greater than 5,000 μg.

In embodiments, wherein the combination and/or composition comprises a primary and/or secondary bioactive molecule from a plant, the primary and/or secondary bioactive molecule may be present in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg.

In embodiments, wherein the primary bioactive molecule from a plant is any of a cannabinoid (e.g., THC, CBD), coumarin, a compound derived from coumarin, i.e., a coumarin derivative (e.g., phenylpropanoids, coumarins, or coumarinoids), the primary bioactive molecule may be present in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg.

In embodiments, the primary bioactive molecule from a plant is a cannabinoid. In embodiments, the cannabinoid is THC. In embodiments, the THC is present in an amount so that a single dose is (whether or not such dose is present in a unit dosage form) between about 0.1 and 5 mg. In embodiments, the THC is present in an amount so that a single dose is between about 0.5 and 2 mg. In embodiments, the THC is present in an amount so that a single dose is between about 0.5 and 1.5 mg. In embodiments, the THC is present in an amount so that a single dose is about 1 mg. In embodiments, the cannabinoid is CBD. In embodiments, the CBD is present in an amount so that a single dose is (whether or not such dose is present in a unit dosage form) between about 0.1 and 5 mg. In embodiments, the CBD is present in an amount so that a single dose is between about 0.5 and 2 mg. In embodiments, the CBD is present in an amount so that a single dose is between about 0.5 and 1.5 mg. In embodiments, the CBD is present in an amount so that a single dose is about 1 mg.

In embodiments, the primary bioactive molecule from a plant is coumarin. In embodiments, the coumarin is present in an amount so that a single dose is (whether or not such dose is present in a unit dosage form) between about 1 and 10 mg. In embodiments, the coumarin is present in an amount so that a single dose is between about 0.1 and 10 mg. In embodiments, the coumarin is present in an amount so that a single dose is between about 0.1 and 5 mg. In embodiments, the coumarin is present in an amount so that a single dose is between about 0.5 and 2 mg. In embodiments, the coumarin is present in an amount so that a single dose is between about 0.5 and 1.5 mg. In embodiments, the coumarin is present in an amount so that a single dose is about 1 mg.

In embodiments, wherein the secondary bioactive molecule from a plant is any of a flavone or a flavonoid, a terpene or a terpenoid, a carbohydrate, a fatty acid or fatty acid ester (FAE), an amide, an amine, a phytosterol, a phenolic compound, cumaru, an isoflavone, a lupeol derivative, (±)-balanophonin, (−)-lariciresinol, 3-hydroxy-retusin-8-methyl-ether, 5-methoxyxanthocercin A, 6,4′-dihydroxy-3′-methoxyaurone, 7-hydroxychromone, 7,3′-dihydroxy-8,4′-dimethoxyisoflavone, betulin, butin, coumaric-acid-beta-glucoside, dipteryxin, dipteryxic acid, eriodictyol, ferulic-acid, isoliquiritigenin, lupeol, melilotoside, melilotoside-1-p-coumaryl-beta-d-glucose, methyl-linolenate, methyl-oleate, 0-coumaricacid, O-hydroxycoumaric-acid, odoratin, P-hydroxy-benzoic-acid, retusin, retusin-8-methyl-ether, sulfuretin, salicylic-acid, afrormisin, castinin, linoleic acid, oleic acid, 3′,4′,7′-trihydroxyflavone, luteolin, and umbelliferone, the secondary bioactive molecule may be present in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg.

In embodiments, wherein the combination and/or composition comprises a primary and/or secondary bioactive molecule from an algae, the primary and/or secondary bioactive molecule may be present in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg.

In embodiments, the primary bioactive molecule from an algae is a primary bioactive molecule from a species in the genus Pyropia or Porphyra. In embodiments, the primary bioactive molecule from a species in the genus Pyropia or Porphyra is any of porphyran or oligo-porphyran, a polysaccharide, an oligo-polysaccharide, a monosaccharide, a peptide, a phycobiliprotein, a mycosporine-like amino acid, an essential amino acid, a nonessential amino acid, a carotene or an intermediate carotenoid, a glycoprotein, an amino sulfonic acid (such as taurine), it may be present in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg.

In embodiments, the primary bioactive molecule from Pyropia or Porphyra may be present in an amount so that a single dose is (whether or not such dose is present in a unit dosage form) between about 1 and 20 mg. In embodiments, the primary bioactive molecule from Pyropia or Porphyra may be present in an amount so that a single dose is (whether or not such dose is present in a unit dosage form) about 8 mg.

In embodiments wherein a combination or composition comprises Pyropia yezoensis extract, the extract may be present in an amount so that a single dose is (whether or not such dose is present in a unit dosage form) between about 1 and 20 mg. In embodiments, the Pyropia yezoensis extract is present in an amount so that a single dose is between about 1 and 10 mg. In embodiments, the Pyropia yezoensis extract is present in an amount so that a single dose is between about 5 and 10 mg. In embodiments, the Pyropia yezoensis extract is present in an amount so that a single dose is between about 8 mg.

In embodiments, wherein the secondary bioactive molecule from an algae is any of a mineral, a vitamin, a lipid, a phenolic compound, or a phlorotannin, it may be present in an amount so that a single dose is (whether or not such dose is present in a unit dosage form), less than about 1 mg, about 1 mg, or more than about 1 mg, up to and including about 75 mg. In embodiments, a single dose may be greater than 75 mg, including 100 mg, 150 mg, 200 mg, or greater than 200 mg.

In some embodiments, a disclosed therapeutic combination (i.e., a disclosed therapeutic combination comprising, inter alia, a bioactive molecule from a fungus and a bioactive molecule from a plant) is administered a total daily dose of between about 50 mg and 1 g. In embodiments, the total daily dose is between about 50 mg and 1 g, 100 mg and 500 mg, and preferably 100 mg and 400 mg. In embodiments, each individual dose of a disclosed therapeutic combination is between 5 mg and 500 mg, or 10 mg and 400 mg, or between 10 mg and 100 mg.

In embodiments, for example in embodiments wherein a disclosed combination is administered in a single composition, a single dose is between about 10 mg and 1000 mg, about 10 mg and 200 mg, about 20 mg and 200 mg, about 10 mg and 100 mg, about 20 mg and 100 mg, about 20 mg and 80 mg, or about 30 mg and 60 mg. In embodiments, a single dose is about 50 mg.

In embodiments, the single composition is administered between 1 and 8 doses daily. In embodiments, the single composition is administered once per day. In embodiments, the single composition is administered twice per day. In embodiments, the single composition is administered three times per day. In embodiments, the single composition is administered four times per day. In embodiments, the single composition is administered five times per day. In embodiments, the single composition is administered six times per day. In embodiments, the single composition is administered seven times per day. In embodiments, the single composition is administered eight times per day. In embodiments, the single dose is administered between 1 and 8 doses daily, for at least one week, two weeks, three weeks, 1 month, 2 months, or 3 months.

In embodiments, a patient will be administered a therapeutically effective dose of a primary and/or secondary bioactive molecule on a regular or chronic basis, wherein the patient is administered the primary and/or secondary bioactive molecule daily, several times per day (at least one, at least two, at least three, at least four, or greater than four times per day); on a set, repeating schedule, wherein the patient is administered a therapeutically effective dose of the primary and/or secondary bioactive molecule every other day, every three days, every four days, every five days, every six days, every seven days, or more than every seven days; or, in some embodiments, a varying schedule comprised of a plurality of days “on” (wherein the therapeutically effective dose of the primary and/or secondary bioactive molecule is administered), and a plurality of days “off” (wherein no administration occurs), such as one day on two days off, two days on three days off, three days on four days off, or other such schedules as would be apparent to those of skill (see, e.g., what are known as the Paul Stamets or James Fadiman microdosing protocols).

It will be understood that, in some embodiments, the dose actually administered may be determined by a physician, or adjusted by a physician, in light of the relevant circumstances, the method of delivery, the age of the patient, the weight of the patient, whether the patient has any comorbidities, other medications the patient is taking (routinely or presently), and any patient-specific aspects that could affect the way in which the primary and/or secondary bioactive molecules interact with the patient, such as variations in metabolism, variations in patient response, etc., all taken together with the teachings of this disclosure, and therefore any disclosed dosage ranges are not intended to be limiting. In some instances, dosage levels below the lower limit of a disclosed range may be more than adequate, while in other cases doses above a range may be employed without causing any harmful side effects, provided for instance that such larger doses also may be divided into several smaller doses for administration, either taken together or separately.

In some embodiments, the primary and/or secondary bioactive molecules may be administered and dosed in accordance with good medical practice, taking into account the method and scheduling of administration, prior and concomitant medications and medical supplements, the clinical condition of the individual patient and the severity of the underlying disease, the patient's age, sex, body weight, tolerance, and other such factors relevant to medical practitioners, and knowledge of the particular compound(s) used, all taken together with the teachings of this disclosure. Dosage levels may differ from patient to patient, for individual patients across time, and for different combinations and formulations, but shall be able to be determined with ordinary skill. Determination of appropriate dosing shall include not only the determination of single dosage amounts, but also the routes of dose administration, determination of the number and timing of doses, and the time(s) of day or time(s) during a psychotherapeutic session preferable for their administration.

In embodiments, a patient may be on a dosing schedule as described above, but may administer the dose to themselves. In embodiments, the combination and/or disclosed composition may be prescribed to a patient, wherein the patient obtains a therapeutically effective dose from a pharmacy or healthcare provider.

Dose amount, frequency, or duration may be increased or reduced, as indicated by the clinical outcome desired, status of the pathology or symptom, any adverse side effects of the treatment or therapy, or concomitant medications. Concentrations or ratios of concentrations of components of disclosed combinations and compositions also may be altered as indicated by the clinical outcome desired, status of the pathology or symptom, any adverse side effects of the treatment or therapy, or concomitant medications. For example, in some embodiments, a disclosed combination comprises a tryptamine (such as, psilocin and/or psilocybin, e.g., as a component of a fungal portion, such as a fungal extract from a psilocin- and/or psilocybin-containing fungus). In some such embodiments, a patient taking a monoamine oxidase inhibitor (MAOI) medication may be administered a composition with a reduced concentration of the tryptamine (e.g., the psilocin and/or psilocybin), as the MAOI would be expected to reduce the metabolic excretion of the tryptamine, thereby affecting the ratios and synergistic effects of bioactive molecules upon administration of the disclosed composition. In another example, the percentage of a component of a disclosed composition may be increased or decreased according to individual tolerance. For example, individuals who frequently consume Cannabis products often develop a tolerance to the effects of cannabinoids. In some embodiments, an individual with appreciable tolerance for Cannabis or a cannabinoid (e.g., THC) may be administered a composition with an increased concentration of THC to account for such tolerance. In yet another example, individuals with certain medical conditions may be particularly sensitive or tolerant to the effects of a disclosed composition. By way of example only, in some embodiments, an individual with Lewy Body Dementia (LBD) may be administered a disclosed composition comprising a reduced concentration of a cannabinoid (e.g., THC) or a Cannabis extract, for example, if it is reasonably suspected (e.g., per the discretion of a physician or another medical practitioner) that the cannabinoid or Cannabis extract could aggravate cognitive or psychiatric symptoms of LBD, and/or increase the patient's sensitivity to the composition or any of its constituents. In another merely illustrative example, an individual with clinical depression may be administered a disclosed composition with an increased concentration of a tryptamine (such as, psilocin and/or psilocybin, e.g., as a component of a fungal portion, such as a fungal extract from a psilocin- and/or psilocybin-containing fungus), which may provide clinical advantages, such as promoting serotonin regulation, neuroplasticity, and connectivity.

The skilled artisan with the benefit of this disclosure will appreciate the factors that may influence the dosage, frequency, and timing required to provide an amount sufficient or effective for providing a therapeutic effect or benefit, and to do so depending on the type of therapeutic effect desired, as well as to avoid or minimize adverse effects.

In other embodiments, appropriate dosages to achieve a therapeutic effect, including the upper and lower bounds of any dose ranges, can be determined by an individual, including an individual who is not a clinician, by reference to available public information and knowledge, and reference to subjective considerations regarding desired outcomes and effects.

In embodiments, the bioactive molecules in a therapeutic combination may be administered to a patient as a single composition. In embodiments, the bioactive molecules in a therapeutic combination may be administered to a patient separately, sequentially, or simultaneously. In embodiments, the bioactive molecules in a therapeutic combination can be individually formulated as pharmaceutical compositions that are then administered to a patient separately, sequentially, or simultaneously.

In embodiments, sequential administration refers to administration of one combination drug immediately following administration of another combination drug (e.g., within about 5 minutes of the administration of the first combination drug), simultaneous administration refers to administration of each combination drug at substantially the same time, while separate administration refers to administration with time elapsing between the administrations. In embodiments wherein the combination drugs are administered separately, such administration may include elapsed time between the each administration of between about 5 minutes to about 30 minutes, about 10 minutes to about 60 minutes, about 30 minutes to about 180 minutes, about 180 minutes to about 360 minutes, or more than 360 minutes, such as 8 hours, 12 hours, 16 hours, 20 hours, 24 hours, 36 hours, 48 hours, 72 hours, 5 days, 7 days, 10 days, 14 days, 21 days, 30 days, and any such durations in between, as would be readily understood by one of skill.

The time elapsed between doses will not be confused with the frequency at which dosing occurs. As will be appreciated by one of skill, the frequency of dosing may depend on the disease and symptoms being treated, and by the administration of the primary and/or secondary bioactive molecule disclosed herein. As a non-limiting example, it may be desirable to administer combination drugs between about one and about eight times per day, including once per day, twice per day, 3 times per day, 4 times per day, 5 times per day, 6 times per day, 7 times per day, and 8 times per day; in some embodiments, as an example, one of the combination drugs (i.e., one of the bioactive compounds in a combination) may be administered twice per day or 3 times per day, while another combination drug is administered once per day or twice per day, and any further combination drug(s) are administered on other appropriate administration schedules as applicable thereto.

Further, the dosing schedule may continue in the same pattern or a modified one for a specific duration of time, including 1-7 days, or greater than 7 days, including 14, 21, 28, 29, 30, 31, 35, 42, 49, 60, 75, 90, 100, 125, 150, 175, 200, 225, 250, 275, 300, 325, 350, 375, 400, 425, 450, 475, 500, 525, 550, 575, 600, 625, 650, 675, and 700 days; and the manner in which administration is completed (i.e., via separate, sequential, or simultaneous administration) may be adjusted as needed, such that the entire dosing schedule may only include sequential, simultaneous, or separate dosing; or may include a combination of two of sequential, simultaneous, or separate dosing; or may include all of sequential, simultaneous, or separate dosing. That said, in embodiments, the frequency of dosing and duration of elapsed time between administrations will depend on the specific primary and/or secondary bioactive molecules utilized and the condition sought to be treated.

In embodiments, administration may comprise separate, sequential, or simultaneous oral administrations, sublingual administrations, buccal administrations, intravenous injections, intra-arterial injections, intraperitoneal injections, intraosseous injections, intramuscular injections, intrathecal injections, intracerebroventricular injections, rectal administrations, vaginal administrations, ocular administrations, nasal administrations, cutaneous administrations, topical administrations, otic administrations, transdermal administrations, or a combination thereof, as would be apparent to one of skill depending on the desired therapeutic effect. Further, as mentioned, in embodiments, a patient is administered the combination drugs through more than one administration, wherein each administration is not the same means of administration. As disclosed herein, such may be advantageous in situations wherein the primary and/or secondary bioactive molecules have varying durations of action and maximum plasma concentrations.

In embodiments, the disclosed primary and/or secondary bioactive molecules are administered in a single composition. In embodiments, the disclosed primary and/or secondary bioactive molecules are administered separately. In embodiments, the disclosed primary and/or secondary bioactive molecules are administered sequentially. In embodiments, the disclosed primary and/or secondary bioactive molecules are administered simultaneously. In embodiments, the timing of the administration of the disclosed primary and/or secondary bioactive molecules (including whether or not more than one administration of the primary and/or secondary bioactive molecules is necessary) will depend on the specific primary and/or secondary bioactive molecules administered to the individual and the indication sought to be treated.

### VI. Pharmaceutical Kits

In embodiments, especially where a formulation is prepared in single unit dosage form, suggested dosage amounts shall be known by reference to the format of the preparation itself. In other embodiments, suggested dosage amounts may be known by reference to the means of administration or by reference to the packaging and labeling, package insert(s), marketing materials, training materials, or other information and knowledge available to those of skill or the public. Another aspect of this disclosure therefore provides pharmaceutical kits containing a pharmaceutical composition or formulation of the disclosure, suggested administration guidelines or prescribing information therefore, and a suitable container. Individual unit dosage forms can be included in multi-dose kits or containers. Pharmaceutical compositions also can be packaged in single or multiple unit dosage forms for uniformity of dosage and ease of administration. Accordingly, another aspect of this disclosure provides pharmaceutical kits containing a pharmaceutical composition or formulation of the disclosure, suggested administration guidelines or prescribing information therefore, and a suitable container. Individual unit dosage forms can be included in multi-dose kits or containers. Pharmaceutical compositions also can be packaged in single or multiple unit dosage forms for uniformity of dosage and ease of administration.

In an exemplary pharmaceutical kit, capsules, tablets, caplets, or other unit dosage forms are packaged in blister packs. “Blister pack” refers to any of several types of pre-formed container, especially plastic packaging, that contains separate receptacles (e.g., cavities or pockets) for single unit doses, where such separate receptacles are individually sealed and can be opened individually.

Blister packs include such pharmaceutical packs known to those of skill, including Aclar® Rx160, Rx20e, SupRx, and UltRx 2000, 3000, 4000, and 6000 (Honeywell). Within the definition of multi-dose containers, and also often referred to as blister packs, are blister trays, blister cards, strip packs, push-through packs, and the like. Preferably, information pertaining to dosing and proper administration (if needed) is printed onto a multi-dose kit directly (e.g., on a blister pack or other interior packaging holding the disclosed combinations or compositions); however, kits of the disclosure can further contain package inserts and other printed instructions (e.g., on exterior packaging) for administering the disclosed combinations or compositions and for their appropriate therapeutic use.

In embodiments, a patient will have the option of using online software such as a website, or downloadable software such as a mobile application, to assist with compliance or to provide data relating to treatment. Such software can be used to, e.g., keep track of last dose taken and total doses taken, provide reminders and alerts for upcoming doses, provide feedback to discourage taking doses outside of set schedules, and allow for recording of specific subjective effects, or provide means for unstructured journaling. Such data collection can assist with individual patient compliance, can be used to improve or tailor individual patient care plans, and can be anonymized, aggregated, and analyzed (including by Al or natural language processing means) to allow research into the effects of various methods of treatment.

### VII. METHODS OF TREATMENT

In some aspects, provided are methods of treating a subject with an inflammatory condition or disorder. In some aspects, disclosed methods of treatment include the administration of a therapeutic combination or pharmaceutical composition as described herein.

“Treatment” covers any treatment of a disorder in a mammal, and particularly in a human, and in embodiments includes one or more of: (a) preventing a disorder from occurring in a subject who may be predisposed to the disorder but has not yet been diagnosed with it: (b) inhibiting a disorder, i.e., arresting its development (including, e.g., prophylaxis); (c) relieving a disorder, i.e., causing regression of the disorder or its clinical symptoms; (d) protection from or relief of a symptom or pathology caused by or related to a disorder; (e) reduction, decrease, inhibition, amelioration, or prevention of onset, severity, duration, progression, frequency or probability of one or more symptoms or pathologies associated with a disorder; and (f) prevention or inhibition of a worsening or progression of symptoms or pathologies associated with a disorder. One will understand that a therapeutic amount necessary to effect treatment for purposes of this disclosure will, for example, be an amount that provides for objective indicia of improvement in patients having clinically-diagnosable symptoms. The effect may be prophylactic in terms of completely or partially preventing a disorder or symptom thereof and/or may be therapeutic in terms of a partial or complete cure for a disorder and/or adverse effect attributable to the disorder.

The terms “inflammation”, “inflammatory condition”, and “inflammatory disorder” may be used interchangeably herein, such as when speaking generally about the methods, and unless context suggests otherwise; however, it also will be understood that the terms may have distinct meanings in certain contexts.

The term “proinflammatory” is used in some embodiments to describe a factor which increases, upregulates, or otherwise leads to inflammation. The term “anti-inflammatory” is used in some embodiments to describe a factor which downregulates, decreases, or otherwise lessens inflammation.

Inflammation is an immune response to tissue insults such as microbial infection, acute injury, oxidative stress, chemical irritants or other such dysregulation of normal tissue functioning. Inflammation responses are coordinated by the innate immune system, which can sense molecular patterns of pathogens and tissue damage and initiate various inflammation pathways which act to remove harmful stimuli and return damaged tissue to a state of homeostasis. Although these responses are self-terminating, termination may fail for multiple reasons, extending the inflammation response into a chronic stage (Ahmed. Front Biol. 2011:6(4):274-281).

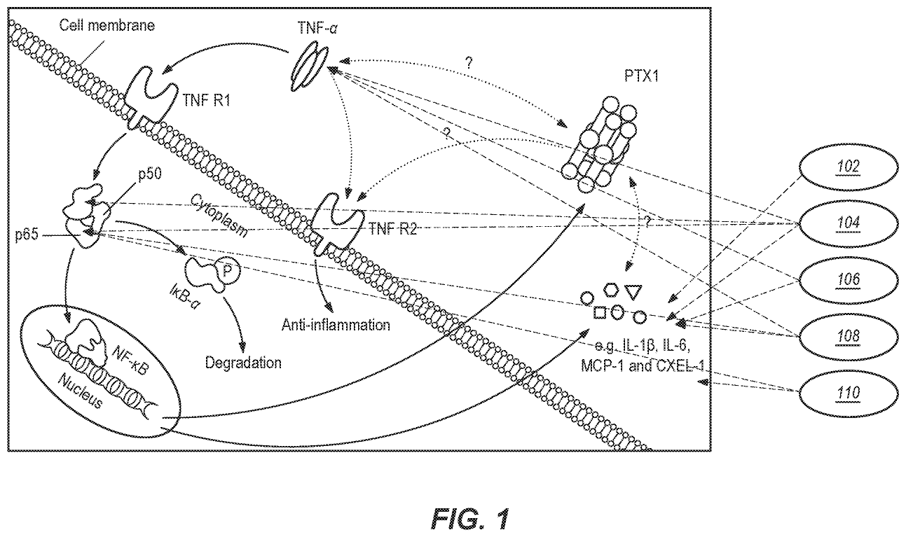

Inflammation is often associated with or underlies a variety of pathological conditions, including major cardiovascular and neuropsychiatric disorders (Nichols. Cardiovasc Psychiatry Neurol. 2009:475108). In the brain, for instance, microglia are responsible for sensing pathogens and cellular damage, and initiate adequate immune responses (Nayak et al. Annu Rev Immunol. 2014; 32:367-402). Microglia consequently induce neuroinflammation through phagocytosis and the release of cytokines to “call” for the right type of back-up cellular response, coordinated via the NF-κB inflammatory amplification and TNF pathways (Kettenmann et al. Physiol Rev. 2011; 91(2):461-553). This microglial inflammatory response, and its reduction by bioactive molecules in disclosed compositions, is illustrated in FIG. 1, in which 102 represents an exemplary bioactive molecule from a first plant portion (e.g., THC), 104 represents an additional exemplary biomolecule from a first plant portion (e.g., CBD), 106 represents an exemplary biomolecule from a fungal portion (e.g., psilocybin, psilocin), 108 represents an exemplary biomolecule from a second plant portion (e.g., coumarin), and 110 represents an exemplary biomolecule from an algal portion (e.g., taurine).

Inflammation can be classified as either acute or chronic. Acute inflammation refers to the inflammatory response initiated by the innate immune system in response to its initial sensing of a detrimental insult such as pathogen invasion, tissue damage and other noxious conditions. The innate immune system encompasses a variety of cell types, proteins, receptors and signaling pathways. Immune cells including macrophages, dendritic cells, mast cells, neutrophils and lymphocytes and non-immune cells including epithelial cells, endothelial cells and fibroblasts host a diverse set of pattern recognition receptors (PRRs). PRRs recognize structures conserved in microbes called pathogen-associated molecular patterns (PAMPs) and endogenous molecules associated with cell damage called damage-associated molecular patterns (DAMPs) (Tang et al. Immunol Rev. 2012; 249(1):158-75). Activation of PRRs results in the production of inflammatory mediators such as cytokines (e.g. tumor necrosis factor (TNF), interleukin-1 (IL-1) and interleukin-6 (IL-6)) and chemokines which mediate the inflammatory processes at the site of pathogen and/or damage (Ahmed. Front Biol. 2011:6(4): 274-281). These pathways initiate processes to eliminate pathogens and/or return a site of damage to homeostasis, including but not limited to increasing blood flow, vasodilation, plasma delivery (compromising antibodies and complement factors), immune cell recruitment, and increased pain sensitivity (Sreedhar et al. “General Mechanisms of Immunity and Inflammation.” Significance of Japanese Kampo Medicine in Supportive Care of Heart Failure/Function. 2017:23). These processes also underlie the classic symptoms of acute inflammation; heat, redness, swelling, pain, and loss of function at the site of inflammation (Antonelli et al. FASEB J. 2017; 31(5):1781-1791).

The innate immune system includes negative feedback systems to resolve the pro-inflammatory response once the initial trigger or cellular damage is resolved. Often pro-inflammatory pathways also trigger the expression of anti-inflammatory mediators such as IL-10, TGF-β, and glucocorticoids (Lawrence et al. Int J Exp Pathol. 2007; 88(2):85-94, Ahmed. Front Biol. 2011:6(4): 274-281). A failure in the resolution of any aspect of acute inflammation can lead to chronic inflammation. Chronic inflammation thus refers to inflammation which persists beyond its physiological function to return homeostasis, and instead contributes to the continuation or occurrence of pathological conditions. Chronic inflammation is the primary cause of inflammatory conditions, and is the major driver of their pathogenesis (Nasef et al. Arch Toxicol. 2017; 91(3):1131-1141, Ahmed. Front Biol. 2011:6(4): 274-281).

In embodiments, a disclosed therapeutic combination is useful in the treatment of inflammatory conditions and disorders associated with oxidative stress. Without being bound by theory, oxidative stress results from an imbalance between cellular oxidants and antioxidants in favor of the oxidants, leading to a disruption of redox signaling and control and/or molecular damage (Sies, H. and Jones, D. Oxidative stress. In: Encyclopaedia of Stress, G. Fink, ed. Elsevier. 2007. 45-48). Reactive oxidative species (ROS), such as free radicals, contain one or more unpaired electrons in their molecular orbitals, which then stabilize by removing electrons from surrounding biomolecules (Pacher et al. Physiol Rev. 2007; 87(1):315-424). The consequences of oxidative damage can range from altered signal transduction to biomolecular damage and cell death (Lugrin et al. Biol Chem. 2014; 395(2):203-230). Inflammation is an innate immune response that is coordinated by signal transduction pathways that originate from the sensing of either pathogen-associated molecular patterns (PAMPs) released from invading pathogens, or damage-associated molecular patterns (DAMPs) released from sites of tissue injury (Matzinger, Science. 2002; 296:301-305). Oxidative species induce modifications to proteins and lipids which produce particularly potent DAMPs such as oxidized 1-palmytoyl-2-arachidonyl-sn-glycero-3-phosphocholine (Imai et al. Cell. 2008; 133(2):235-249; Kampfrath et al. Circ Res. 2011; 108(6):716-26), oxidized cholesteryl esters (Choi et al. Circ Res. 2009; 104(12):1355-63), and oxidized low-density lipoproteins (Lahoute et al. Nat Rev Cardiol. 2011; 8(6):348-358). Further, DAMPs are sensed by immune cells, which contain various toll-like receptors (TLRs) in their membranes which aid in coordinating the proper immune responses to the damage sensed. Oxidative stress increases the amount of TLRs present in the immune cell membranes, thereby priming these cells to sense damage and increasing the resulting inflammatory response (Lugrin et al. Biol Chem. 2014; 395(2):203-230).

Cellular damage and immune signal disruptions caused by oxidative stress can cause or prolong inflammatory disorders. Without being bound by theory, the effects of oxidative stress inflammatory disorders can be ameliorated by bringing the cellular redox balance closer to homeostasis by increasing the antioxidant level of cells. In embodiments, a disclosed combination increases the cellular antioxidant level in cells, tissues, and/or organs involved in an inflammatory condition or disorder. In embodiments, a disclosed combination reduces the cellular oxidant level in cells, tissues, and/or organs involved in an inflammatory condition or disorder. In embodiments, an effect of a disclosed combination is the achievement of cellular redox homeostasis in cells, tissues, and/or organs involved in an inflammatory condition or disorder. In embodiments, an effect of a disclosed combination is the preservation of cellular redox homeostasis in cells, tissues, and/or organs involved in an inflammatory condition or disorder. In embodiments, a disclosed combi-nation exerts antioxidant effects that are useful in treating or preventing inflammatory conditions or disorders.

In some embodiments, antioxidant properties of disclosed therapeutic combinations result from synergistic and overlapping effects of the fungal, plant, and algal portions, said synergy being defined in embodiments as an antioxidant effect that is greater than the additive antioxidant effects or antioxidant properties of the portions when administered alone. In embodiments, the synergy is a synergistic effect in, e.g., antioxidant capacity, potency, bioactivity, bioaccessibility, bioavailability, therapeutic effect, or a combination thereof.

There has been limited success in attempts to treat inflammatory conditions and disorders, especially those which cause chronic inflammation. This is partly due to the fact that some inflammatory mediators produced by the immune system induce persistent or recurrent inflammation, such as is the case in auto-inflammatory disorders. In addition, many current anti-inflammatory drugs not only block the production of pro-inflammatory mediators, but also inhibit the control loop that causes release of endogenous anti-inflammatory mediators.

The primary challenges to treating inflammatory conditions include redundancy in the inflammatory response, the ability for proinflammatory pathways to compensate for the inhibition of others, and the essentiality of the inflammatory response to the individual's health and survival (Tabas et al. Science. 2013; 339(6116):166-172). To overcome these challenges, many existing anti-inflammatory drugs block both pro- and anti-inflammatory mediators. For example, non-steroidal anti-inflammatory drugs (NSAIDs) reduce inflammation by blocking the enzymatic activity of cyclooxygenase, an important enzyme that catalyzes the conversion of arachidonic acid to prostaglandins and leukotrienes. NSAIDs thus prevent the synthesis of all prostaglandins, including anti-inflammatory prostaglandins. While beneficial for treating acute inflammation, NSAID use may also lead to chronic inflammation, and therefore appear to be unsuccessful in treating many inflammatory conditions and disorders.

In embodiments, the disclosed combinations, compositions, and methods are useful to treat inflammatory conditions and disorders due to their redundant, promiscuous effects on cellular signaling systems involved in the inflammatory response. In embodiments, the disclosed combinations, compositions, and methods act on pro- and anti-inflammatory pathways to reduce the pathophysiology of inflammatory conditions and disorders. In embodiments, each component of the disclosed methods may affect cellular signaling in multiple ways, for example, with differing pharmacodynamics.

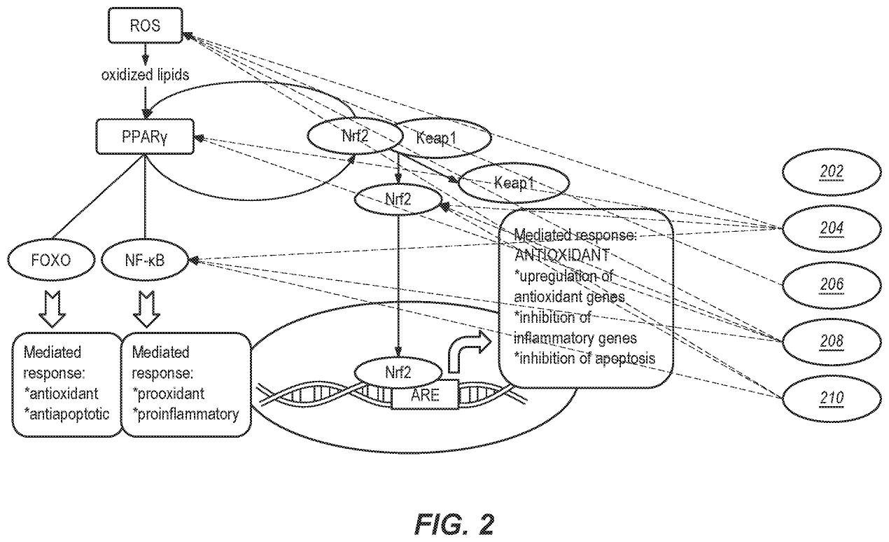

In embodiments, the redundant, promiscuous regulation of the inflammation response of the disclosed combinations, compositions, and methods is due to the inflammation regulating capabilities of the components as well as synergies achieved by administering them in the disclosed combinations. In embodiments, the components of disclosed combinations may, for example, reduce cytokines TNF-α and IL-1R through direct and indirect (amplification) interference with NF-κB and AP-1 transduction mechanisms, reduce reactive oxidative species (ROS), NO, and MPO, and activate anti-inflammatory networks PPAR-γ and NRf2. These effects are illustrated in FIG. 2, in which 202 represents an exemplary bioactive molecule from a first plant portion (e.g., THC), 204 represents an additional exemplary biomolecule from a first plant portion (e.g., CBD), 206 represents an exemplary biomolecule from a fungal portion (e.g., psilocybin, psilocin), 208 represents an exemplary biomolecule from a second plant portion (e.g., coumarin), and 210 represents an exemplary biomolecule from an algal portion (e.g., taurine).

Cannabis. In some embodiments, administration of a disclosed combination comprising Cannabis (e.g., one or more bioactive molecules from Cannabis, such as THC, CBD; and/or a Cannabis extract) decreases the concentrations of any of the proinflammatory cytokines IL-1R, IL-6, and IL-1R. In some embodiments, administration of a disclosed combination inhibits the NF-κB signaling pathway. Administration of a disclosed combination may also increase NRF2 expression, leading to decreased ROS, which in turn may suppress NLRP3 inflammasome activation by reducing NF-κB levels (e.g., by way of promoters p65 and p52). Administration of a disclosed combination may also activate PPAR-γ networks and directly repress NF-kB transduction through CB₁ and CB₂ receptors. In some embodiments, administration of a disclosed combination triggers apoptosis in T-cells and dendritic cells, resulting in immunosuppression. Further, in some embodiments, administration of a disclosed combination may downregulate proinflammatory cytokine and chemokine production and upregulate the production of T-regulatory cells (TREGs), which can suppress the inflammatory response.

Dipteryx. In some embodiments, administration of a disclosed combination comprising one or more bioactive molecules from Dipteryx (e.g., one or more bioactive molecules from Dipteryx, such as coumarin; and/or a Dipteryx extract) decreases the production of nitric oxide (NO), tumor necrosis factor alpha (TNF-α), and/or interleukin-1 beta (IL-13). In some embodiments, administration of a disclosed combination inhibits LPS-induced protein and mRNA expression levels of nitric oxide synthase (iNOS) and/or cyclooxygenase-2 (COX-2), e.g., in RAW264.7 cells. In embodiments, administration of a disclosed combination reduces the LPS-stimulated phosphorylation of any of IKKα, IKKβ, p-IκBα, and IκBα, and/or the nuclear translocation of the p65 subunit of pro-inflammatory transcription factor NF-κB. In embodiments, administration of a disclosed combination activates PPAR-γ, which may counter-regulate inflammatory activity in a tissue-specific way; and/or transcription factor NRf2, further modulating NF-kB activity and exerting useful cytoprotective mechanisms.

Algae. In some embodiments, administration of a disclosed combination comprising one or more bioactive molecules from algae (e.g., one or more bioactive molecules from algae, such as coumarin; and/or an algae extract) reduces neuropathy through down-regulation of NF-κB and/or activation of Nrf2 signaling cascades. Neuropathy can be a feature of certain inflammatory conditions, such as long COVID. In some embodiments, administration of a disclosed combination may also reduce serum levels of IL-6. In some embodiments, administration of a disclosed combination may also stabilize membranes, encourage redox homeostasis, and scavenge oxidative factors. In some embodiments, administration of a disclosed combination may inhibit the production of any of the pro-inflammatory cytokines TNF-α, IL-1β, and IL-6.

Fungi. In some embodiments, administration of a disclosed combination comprising one or more bioactive molecules from fungi (e.g., one or more bioactive molecules from fungi, such as psilocybin and/or psilocin; and/or a fungal extract) interacts with signaling pathways in the nervous and immune systems. In embodiments, administration of a disclosed combination activates dopamine and serotonin receptors, both of which are implicated in modulating inflammation pathways underlying certain inflammatory disorders. In embodiments, administration of a disclosed combination regulates both proinflammatory and anti-inflammatory pathways to reduce overall inflammation, such as by lowering IL-6 and/or COX-2 concentrations, without lowering the concentration of the anti-inflammatory cytokine IL-10. In embodiments, administration of a disclosed combination reduces proinflammatory cytokines TNF-α and IL-1β, as well as ROS.

Whether a patient has an inflammatory condition or disorder can be determined according to ordinary skill. Without being bound by theory, inflammatory conditions or disorders are generally diagnosed after symptoms present. Examples of methods used to diagnose inflammatory conditions or disorders include genetic testing to detect known abnormalities that may be indicative of an inflammatory condition or disorder; testing of blood, urine, and cerebrospinal fluid; a trial of condition-specific medication useful in alleviating a symptom of a condition; nuclear imaging; an X-ray; magnetic resonance imaging (MRI); computerized tomography (CT); observance of present symptoms; patient history information; and additional testing to exclude one or more similar conditions. Because a variety of diseases and conditions have symptoms characteristic of one or more inflammatory conditions or disorders, diagnosis may include a combination of the aforementioned methods, and may include additional methods known to those of skill.

In embodiments, the inflammatory conditions or disorders is any of long COVID, including post-acute sequelae of SARS-CoV-2 infection (PASC), rheumatoid arthritis, psoriatic arthritis, reactive arthritis, tendonitis, gout, scleroderma, systemic scleroderma, localized scleroderma, CREST syndrome, sciatica, post-treatment Lyme disease (PTLD), chronic Lyme disease (CLD) syndrome, a neuropathy, peripheral neuropathy, hereditary neuropathy, acquired neuropathy, motor neuropathy, sensory neuropathy, autonomic neuropathy, combination neuropathies, sciatic neuritis, myalgic encephalomyelitis, chronic fatigue and immune dysfunction syndrome (CFIDS)/chronic fatigue syndrome (CFS), an inflammatory skin condition, fatty liver disease, endometriosis, type 1 diabetes mellitus, type 2 diabetes mellitus, inflammatory bowel disease, asthma, obesity, Alzheimer's disease, Parkinson's disease, cancer, Kawasaki's disease, vasculitis, uveitis, Crohn's disease, ulcerative colitis, meningitis, allergies, psoriasis, Hashimoto's disease, Guillain Barre syndrome, hepatitis, celiac disease, multiple sclerosis, migraine, fibromyalgia, lupus, and Sjogren's syndrome.

In embodiments, the inflammatory condition or disorder is long COVID. COVID (i.e., COVID-19) is a contagious viral disease caused by infection of the SARS-CoV-2 virus. Long COVID is a continuation or emergence of COVID-19 symptoms weeks or longer after the initial SARS-CoV-2 infection is experienced. These symptoms may be fever, chills, cough, shortness of breath, difficulty breathing, fatigue, muscle ache, body ache, headache, loss of taste, loss of smell, sore throat, congestion, runny nose, nausea, vomiting, diarrhea, difficulty thinking or concentrating (i.e., brain fog), sleep problems (e.g., insomnia), dizziness, neuropathic pain, nerve sensitivity, change in taste, change in smell, depression, anxiety, heart palpitations, joint pain, chest pain, or stomach pain. The mechanism of action of long COVID is not well understood, however, chronic inflammation is believed to be responsible for a range of symptoms associated with the disorder. For example, recent research has determined that certain inflammatory markers (such as TNF-α) are remain persistently elevated in long COVID patients (Raveendran et al. Diabetes Metab Syndr. 2021; 15(3):869-875; Peluso et al. Cell Rep. 2021; 36(6):109518; Frere et al. Sci Trans/Med. 2022; 14(664):eabq3059). It is known that serotonin (5-HT) affects immune regulation via 5-HT receptors expressed in immune cells (Wan et al. Front Immunol. 2020; 11:186). Moreover, drugs that target 5-HT signaling have been shown to be beneficial in mouse models of various inflammatory diseases, including multiple sclerosis (MS) and inflammatory bowel disease (IBD) (Id.). Without being bound by theory, it is proposed that therapeutic combinations of the disclosure may, by one mechanism of action, improve inflammatory diseases in which an immune response is implicated by a similar mechanism of interacting with 5-HT receptors on immune cells to change the course of the disease.

In embodiments, administering a therapeutic combination or composition according to the disclosed methods is effective in treating a patient with long COVID (or PASC). In embodiments, administering a therapeutic combination according to the disclosed methods relieves long COVID in a patient by causing regression of the long COVID and its symptoms. In embodiments, administering a therapeutic combination according to the disclosed methods provides protection from or relief of a symptom caused by or related to long COVID. In embodiments, administering a therapeutic combination according to the disclosed methods reduces, decreases, inhibits, and/or ameliorates the severity, frequency, duration, and/or progression of one or more symptoms of long COVID. For example, administering a therapeutic combination according to the disclosed methods improves sleep, improves mental clarity, reduces brain fog, reduces fatigue, improves mood, improves the ability to participate in regular activities negatively affected by the long COVID (e.g., work, school), improves job functioning, reduces headaches, and improves the ability to navigate technology (e.g., computer, email) in patients with long COVID. In embodiments, administering a therapeutic combination according to the disclosed methods prevents or inhibits the worsening or progression of symptoms of long COVID.

In embodiments, the inflammatory condition or disorder is psoriatic arthritis (PA). PA is a type of arthritis linked with psoriasis, a chronic skin and nail disease. In embodiments, administering a therapeutic combination or composition to a subject with psoriatic arthritis according to the disclosed methods treats one or more symptoms of the psoriatic arthritis, such as joint swelling, joint inflammation, itching, pitted fingernails, and red scaly rashes.

In embodiments, administering a therapeutic combination or composition according to the disclosed methods is effective in treating a patient with PA. In embodiments, administering a therapeutic combination according to the disclosed methods relieves PA in a patient by causing regression of the PA and its symptoms. In embodiments, administering a therapeutic combination according to the disclosed methods provides protection from or relief of a symptom caused by or related to PA. In embodiments, administering a therapeutic combination according to the disclosed methods reduces, decreases, inhibits, and/or ameliorates the severity, frequency, duration, and/or progression of one or more symptoms of PA. For example, administering a therapeutic combination according to the disclosed methods reduces amplitude and frequency of flare-ups, reduces plaques, improves energy, reduces joint pain, reduces itching, and reduces or eliminates calcification in patients with PA. In embodiments, administering a therapeutic combination according to the disclosed methods prevents or inhibits the worsening or progression of symptoms of PA.

In embodiments, the inflammatory condition or disorder is sciatica or peripheral neuritis. In these conditions, peripheral nerves become damaged and inflamed for a variety of reasons. The inflammation becomes self-amplifying and very difficult to reduce for any length of time with known treatments. In embodiments, administering a therapeutic combination to a subject with sciatica or peripheral neuritis according to the disclosed methods treats one or more symptoms of the sciatica or peripheral neuritis, such as inflammation, nerve damage, pain, difficulty walking, muscle weakness, and numbness.

In embodiments, administering a therapeutic combination or composition according to the disclosed methods is effective in treating a patient with sciatica or peripheral neuritis. In embodiments, administering a therapeutic combination according to the disclosed methods relieves sciatica or peripheral neuritis in a patient by causing regression of the sciatica or peripheral neuritis and its symptoms. In embodiments, administering a therapeutic combination according to the disclosed methods provides protection from or relief of a symptom caused by or related to sciatica or peripheral neuritis. In embodiments, administering a therapeutic combination according to the disclosed methods reduces, decreases, inhibits, and/or ameliorates the severity, frequency, duration, and/or progression of one or more symptoms of sciatica or peripheral neuritis. For example, administering a therapeutic combination according to the disclosed methods reduces amplitude and frequency of flare-ups, improves peripheral sensation, and reduces pain in patients with sciatica or peripheral neuritis. In embodiments, administering a therapeutic combination according to the disclosed methods prevents or inhibits the worsening or progression of symptoms of sciatica or peripheral neuritis.

In embodiments, the inflammatory condition or disorder is Hashimoto's disease, also known as lymphocytic thyroiditis. Hashimoto's disease is an inflammatory disorder, wherein the immune system makes antibodies that attack and damage the thyroid tissue. Initially, attacks to the thyroid may cause inflammation resulting in the production of excess thyroid hormone, a phenomenon known as hyperthyroidism. Eventually, as a disclosed combination may result in a decrease of a biomarker of oxidation (e.g., a biomarker of oxidative stress) of damage, the thyroid gland becomes inflamed and the ability to make thyroid hormone is degraded, eventually leading to hypothyroidism. In embodiments, administering a therapeutic combination to a subject with Hashimoto's disease according to the disclosed methods treats one or more symptoms of the Hashimoto's disease, such as stiffness, reduced motor function, fatigue, reduced endurance, weight gain, muscle weakness, inflammation, goiter, and pain.

In embodiments, administering a therapeutic combination or composition according to the disclosed methods is effective in treating a patient with Hashimoto's disease. In embodiments, administering a therapeutic combination according to the disclosed methods relieves Hashimoto's disease in a patient by causing regression of Hashimoto's disease and its symptoms. In embodiments, administering a therapeutic combination according to the disclosed methods provides protection from or relief of a symptom caused by or related to Hashimoto's disease. In embodiments, administering a therapeutic combination according to the disclosed methods reduces, decreases, inhibits, and/or ameliorates the severity, frequency, duration, and/or progression of one or more symptoms of Hashimoto's disease. For example, administering a therapeutic combination according to the disclosed methods reduces stiffness, improves motor function, reduces amplitude and frequency of or eliminates flare-ups, improves sleep, improves mood, reduces fatigue/exhaustion, and improves endurance in patients with Hashimoto's disease. In embodiments, administering a therapeutic combination according to the disclosed methods prevents or inhibits the worsening or progression of symptoms of Hashimoto's disease.

In embodiments, the inflammatory condition or disorder is scleroderma. Scleroderma is a disorder in which a person's immune system destroys otherwise healthy tissues. Scleroderma is often characterized either as “systemic” or “localized.” Localized scleroderma only impacts skin tissue, whereas systemic scleroderma may impact both skin tissue and tissues under the skin, including blood vessels and major organs. In embodiments, the inflammatory condition or disorder is a type of systemic scleroderma, such as CREST syndrome. CREST is a mnemonic that describes common symptoms of CREST syndrome. The ‘C’ stands for calcinosis, which is the deposition of calcium in the skin and tissues. The ‘R’ stands for Raynaud's phenomenon or Raynaud's spasms, which is a decrease in blood flow in response to stressors that causes a pins and needles sensation and discoloration in the fingers or toes. The ‘E’ stands for esophageal dysmotility, which is muscle dysfunction in the esophagus that can impair the ability to swallow. The ‘S’ stands for sclerodactyly, which is the hardening of skin in the hand that causes fingers to curl inward and form a claw-like shape. The ‘T’ stands for telangiectasias, which are enlarged blood vessels that can appear as red spots on the skin. Patients with CREST may experience extreme systemic inflammation. In embodiments, administering a therapeutic combination to a subject with scleroderma according to the disclosed methods treats one or more symptoms of the scleroderma, such as joint stiffness, pain, and fatigue.

In embodiments, administering a therapeutic combination or composition according to the disclosed methods is effective in treating a patient with CREST syndrome. In embodiments, administering a therapeutic combination according to the disclosed methods relieves CREST syndrome in a patient by causing regression of the CREST syndrome and its symptoms. In embodiments, administering a therapeutic combination according to the disclosed methods provides protection from or relief of a symptom caused by or related to CREST syndrome. In embodiments, administering a therapeutic combination according to the disclosed methods reduces, decreases, inhibits, and/or ameliorates the severity, frequency, duration, and/or progression of one or more symptoms of CREST syndrome. For example, administering a therapeutic combination according to the disclosed methods reduces joint stiffness, improves mood, improves motor function, reduces amplitude and frequency of or eliminates episodes/flare-ups, reduces or eliminates calcification, improves Raynaud's spasm, reduces fatigue, and improves sleep in patients with CREST syndrome. In embodiments, administering a therapeutic combination according to the disclosed methods prevents or inhibits the worsening or progression of symptoms of CREST syndrome.

In embodiments, the inflammatory condition or disorder is multiple sclerosis (MS). MS is a potentially disabling disease of the brain and spinal cord (central nervous system), leading to extremely debilitating inflammatory cycles. In MS, the immune system attacks the protective sheath (myelin) that covers nerve fibers and causes communication problems between the brain and the rest of the body. Eventually, the disease can cause permanent damage or deterioration of the nerves. In embodiments, administering a therapeutic combination to a subject with MS according to the disclosed methods treats one or more symptoms of the MS, such as numbness or weakness in one or more limbs, tingling, electric-shock sensations that occur with certain neck movements, lack of coordination, unsteady gait or inability to walk, partial or complete loss of vision, prolonged double vision, blurry vision, vertigo, fatigue, slurred speech, cognitive problems, mood disturbances, and problems with sexual, bowel, or bladder function.

In embodiments, administering a therapeutic combination or composition according to the disclosed methods is effective in treating a patient with MS. In embodiments, administering a therapeutic combination according to the disclosed methods relieves MS in a patient by causing regression of the MS and its symptoms. In embodiments, administering a therapeutic combination according to the disclosed methods provides protection from or relief of a symptom caused by or related to MS. In embodiments, administering a therapeutic combination according to the disclosed methods reduces, decreases, inhibits, and/or ameliorates the severity, frequency, duration, and/or progression of one or more symptoms of MS. For example, administering a therapeutic combination according to the disclosed methods improves energy, improves sleep, improves mood, and reduces amplitude and frequency of flare-ups in patients with MS. In embodiments, administering a therapeutic combination according to the disclosed methods prevents or inhibits the worsening or progression of symptoms of MS.

In embodiments, the inflammatory condition or disorder is post-treatment Lyme disease (PTLD), also known as chronic Lyme disease (CLD) syndrome. PLTD or CLD is a condition in which patients experience symptoms of Lyme disease past the initial antibiotic treatment period, lasting over six months post-treatment (see Maksimyan et al. Cureus. 2021; 13(10): e18703). Living with persistent symptoms of Lyme Disease following treatment can significantly impact mobility, cognitive skills, lifestyle, and emotional well-being. In embodiments, administering a therapeutic combination to a subject with PLTD or CLD according to the disclosed methods treats one or more symptoms of the PLTD or CLD, such as fatigue, pain, and cognitive difficulties.

In embodiments, administering a therapeutic combination or composition according to the disclosed methods is effective in treating a patient with PLTD or CLD. In embodiments, administering a therapeutic combination according to the disclosed methods relieves PLTD or CLD in a patient by causing regression of the PLTD or CLD and its symptoms. In embodiments, administering a therapeutic combination according to the disclosed methods provides protection from or relief of a symptom caused by or related to PLTD or CLD. In embodiments, administering a therapeutic combination according to the disclosed methods reduces, decreases, inhibits, and/or ameliorates the severity, frequency, duration, and/or progression of one or more symptoms of PLTD or CLD. For example, administering a therapeutic combination according to the disclosed methods reduces brain fog, reduces exhaustion, and reduces achiness in patients with PLTD or CLD. In embodiments, administering a therapeutic combination according to the disclosed methods prevents or inhibits the worsening or progression of symptoms of PLTD or CLD.

In embodiments, the inflammatory condition or disorder is chronic fatigue and immune dysfunction syndrome (CFIDS), also known as chronic fatigue syndrome (CFS). CFS is characterized by extreme fatigue that lasts at least six months. Symptoms worsen over time with physical or mental activity, but do not fully improve with rest. In embodiments, administering a therapeutic combination to a subject with CFIDS or CFS according to the disclosed methods treats one or more symptoms of the CFIDS or CFS, such as extreme exhaustion after physical or mental exercise, problems with memory or thinking skills, dizziness that worsens with moving from lying down or sitting to standing, muscle or joint pain, and unrefreshing sleep.

In embodiments, administering a therapeutic combination or composition according to the disclosed methods is effective in treating a patient with CFIDS or CFS. In embodiments, administering a therapeutic combination according to the disclosed methods relieves CFIDS or CFS in a patient by causing regression of the CFIDS or CFS and its symptoms. In embodiments, administering a therapeutic combination according to the disclosed methods provides protection from or relief of a symptom caused by or related to CFIDS or CFS. In embodiments, administering a therapeutic combination according to the disclosed methods reduces, decreases, inhibits, and/or ameliorates the severity, frequency, duration, and/or progression of one or more symptoms of CFIDS or CFS. For example, administering a therapeutic combination according to the disclosed methods reduces brain fog, improves post-exertion energy, improves quality of life, and improves mood in patients with CFIDS or CFS. In embodiments, administering a therapeutic combination according to the disclosed methods prevents or inhibits the worsening or progression of symptoms of CFIDS or CFS.

In embodiments, the inflammatory condition or disorder is an inflammatory skin condition. An inflammatory skin condition is characterized by the activation of the innate and adaptive immune system via the production of pro-inflammatory cytokines. Inflammatory skin conditions are classified as autoimmune diseases and autoinflammatory syndromes or diseases. In embodiments, administering a therapeutic combination to a subject with an inflammatory skin condition according to the disclosed methods treats one or more symptoms of the inflammatory skin condition, such as redness, rash, hives, plaques, blisters, pus, heat, swelling, pain, and itching.

In embodiments, administering a therapeutic combination or composition according to the disclosed methods is effective in treating a patient with an inflammatory skin disorder. In embodiments, administering a therapeutic combination according to the disclosed methods relieves an inflammatory skin disorder in a patient by causing regression of the inflammatory skin disorder and its symptoms. In embodiments, administering a therapeutic combination according to the disclosed methods provides protection from or relief of a symptom caused by or related to an inflammatory skin disorder. In embodiments, administering a therapeutic combination according to the disclosed methods reduces, decreases, inhibits, and/or ameliorates the severity, frequency, duration, and/or progression of one or more symptoms of an inflammatory skin disorder. For example, administering a therapeutic combination according to the disclosed methods improves skin, reduces or eliminates sensitivity to sunlight, and improves tolerance to allergens in subjects with an inflammatory skin disorder. In embodiments, administering a therapeutic combination according to the disclosed methods prevents or inhibits the worsening or progression of symptoms of an inflammatory skin disorder.

In embodiments, the inflammatory condition or disorder is any of acne vulgaris, acid reflux/heartburn, age-related macular degeneration (AMD), allergies, allergic rhinitis, Alzheimer's disease, amyotrophic lateral sclerosis, anemia, anti-NMDAR encephalitis, appendicitis, arteritis, arthritis (e.g., osteoarthritis, rheumatoid arthritis, juvenile idiopathic arthritis, spondyloarthropathy such as ankylosing spondylitis, and reactive arthritis (Reiter syndrome)), psoriatic arthritis, enteroarthritis associated with inflammatory bowel disease or Whipple and Behcet's disease, septic arthritis, gout (e.g., gouty arthritis, crystalline synovitis, metabolic arthritis, pseudogout (calcium pyrophosphate deposition disease), and Still's disease), skin inflammation, muscle inflammation, tendon inflammation, ligament inflammation, bone inflammation, cartilage inflammation, lung inflammation, heart inflammation, liver inflammation, pancreatic inflammation, kidney inflammation, bladder inflammation, gastric inflammation, intestinal inflammation, neuroinflammation, ocular inflammation, brain inflammation, long COVID, a food allergy, post-treatment lyme disease syndrome, an ulcer, asthma, atherosclerosis, autoimmune disorder, balanitis, blepharitis, bronchiolitis, bronchitis, bullous pemphigoid, a burn, bursitis, cancer (e.g., NF-κB-induced inflammatory cancer), cardiovascular disease (e.g., endocarditis, myocarditis, heart valve dysfunction, congestive heart failure, myocardial infarction, diabetic heart abnormalities, vascular inflammation such as arteritis, phlebitis, and vasculitis, arterial occlusive disease such as arteriosclerosis and stenosis, inflammatory cardiac hypertrophy, peripheral arterial disease, aneurysm, embolism, incision, pseudoaneurysm, vascular malformation, vascular nevus, thrombosis, varicose veins, stroke, cardiac arrest, and carditis), celiac disease, cellulitis, cervicitis, cholangitis, cholecystitis, chorioamnionitis, chronic obstructive pulmonary disease (COPD), cirrhosis, congestive heart failure, conjunctivitis, colitis, cyclophosphamide-induced cystitis, cystic fibrosis, cystitis, lacrimal inflammation, and dementia.

In embodiments, the inflammatory condition is acne vulgaris. In embodiments, the inflammatory condition is acid reflux/heartburn. In embodiments, the inflammatory condition is age-related macular degeneration (AMD), In embodiments, the inflammatory condition is allergies. In embodiments, the inflammatory condition is allergic rhinitis. In embodiments, the inflammatory condition is Alzheimer's disease. In embodiments, the inflammatory condition is amyotrophic lateral sclerosis. In embodiments, the inflammatory condition is anemia. In embodiments, the inflammatory condition is anti-NMDAR encephalitis. In embodiments, the inflammatory condition is appendicitis. In embodiments, the inflammatory condition is arteritis. In embodiments, the inflammatory condition is arthritis. In embodiments, the inflammatory condition is skin inflammation. In embodiments, the inflammatory condition is muscle inflammation. In embodiments, the inflammatory condition is tendon inflammation. In embodiments, the inflammatory condition is ligament inflammation. In embodiments, the inflammatory condition is bone inflammation. In embodiments, the inflammatory condition is cartilage inflammation. In embodiments, the inflammatory condition is lung inflammation. In embodiments, the inflammatory condition is heart inflammation. In embodiments, the inflammatory condition is liver inflammation. In embodiments, the inflammatory condition is pancreatic inflammation. In embodiments, the inflammatory condition is kidney inflammation. In embodiments, the inflammatory condition is bladder inflammation. In embodiments, the inflammatory condition is gastric inflammation. In embodiments, the inflammatory condition is intestinal inflammation. In embodiments, the inflammatory condition is neuroinflammation. In embodiments, the inflammatory condition is ocular inflammation. In embodiments, the inflammatory condition is brain inflammation. In embodiments, the inflammatory condition is long COVID. In embodiments, the inflammatory condition is a food allergy. In embodiments, the inflammatory condition is post-treatment Lyme disease syndrome. In embodiments, the inflammatory condition is an ulcer. In embodiments, the inflammatory condition is asthma. In embodiments, the inflammatory condition is atherosclerosis. In embodiments, the inflammatory condition is autoimmune disorder. In embodiments, the inflammatory condition is balanitis. In embodiments, the inflammatory condition is blepharitis. In embodiments, the inflammatory condition is bronchiolitis. In embodiments, the inflammatory condition is bronchitis. In embodiments, the inflammatory condition is bullous pemphigoid. In embodiments, the inflammatory condition is a burn. In embodiments, the inflammatory condition is bursitis. In embodiments, the inflammatory condition is cancer. In embodiments, the inflammatory condition is cardiovascular disease. In embodiments, the inflammatory condition is celiac disease. In embodiments, the inflammatory condition is cellulitis. In embodiments, the inflammatory condition is cervicitis. In embodiments, the inflammatory condition is cholangitis. In embodiments, the inflammatory condition is cholecystitis. In embodiments, the inflammatory condition is chorioamnionitis. In embodiments, the inflammatory condition is chronic obstructive pulmonary disease (COPD). In embodiments, the inflammatory condition is cirrhosis. In embodiments, the inflammatory condition is congestive heart failure. In embodiments, the inflammatory condition is conjunctivitis. In embodiments, the inflammatory condition is colitis. In embodiments, the inflammatory condition is cyclophosphamide-induced cystitis. In embodiments, the inflammatory condition is cystic fibrosis. In embodiments, the inflammatory condition is cystitis. In embodiments, the inflammatory condition is lacrimal inflammation. In embodiments, the inflammatory condition is dementia.

In some embodiments, the disclosed therapeutic combinations are useful for preventing or treating an inflammatory disorder or condition that causes acute inflammation, or that exhibits acute inflammation as a symptom.

In some embodiments, the disclosed therapeutic combinations are useful for preventing or treating chronic inflammation.

In some embodiments, chronic inflammation includes tissue inflammation. Tissue inflammation may be limited to a particular tissue or organ. In some embodiments, issue inflammation is skin inflammation, muscle inflammation, tendon inflammation, ligament inflammation, bone inflammation, cartilage inflammation, lung inflammation, heart inflammation, liver inflammation, pancreatic inflammation, kidney inflammation, bladder inflammation, gastric inflammation intestinal inflammation, neuroinflammation, or brain inflammation.

In some embodiments, chronic inflammation includes systemic inflammation.

In some embodiments, chronic inflammation includes arthritis. Arthritis includes a group of conditions involving damage to the joints of the body due to inflammation of the synovium, including osteoarthritis, rheumatoid arthritis, juvenile idiopathic arthritis, spondyloarthropathy such as ankylosing spondylitis, reactive arthritis (Reiter syndrome), psoriatic arthritis, enteroarthritis associated with inflammatory bowel disease, Whipple and Behcet's disease, septic arthritis, gout (also known as gouty arthritis, crystalline synovitis, metabolic arthritis), pseudogout (calcium pyrophosphate deposition disease), and Still's disease. Arthritis can affect a single joint (monoarthritis), two to four joints (oligoarthritis), or five or more joints (polyarthritis), either autoimmune or non-autoimmune disease.

In some embodiments, chronic inflammation includes myopathy. Myopathy is caused when the immune system unduly attacks muscle components, causing inflammation in the muscles. Myopathy includes inflammatory and autoimmune myopathy. Myopathy includes, but is not limited to, dermatomyositis, inclusion body myositis, and polymyositis.

In some embodiments, chronic inflammation includes vasculitis. Vasculitis is a diverse disorder characterized by inflammation of the vessel walls, including lymphatic vessels and blood vessels (such as veins (phlebitis), arteries (arteritis), and capillaries) due to leukocyte migration and the resulting damage. A group. This inflammation can affect any size of blood vessel anywhere in the body. It can affect arteries and/or veins. This inflammation can be localized (meaning that only a single site of the blood vessel is affected), or the area of inflammation can be spread over a particular organ or tissue, or even multiple It can also be extensive, including the affected organ system. Vasculitis includes Buerger's disease (obstructive thrombovasculitis), cerebral vasculitis (central nervous system vasculitis), Churg-Strauss arteritis, cryoglobulinemia, essential cryoglobulin vasculitis, giant cells (temporal) Arteritis, golfer vasculitis, Henoch-Schonlein purpura, hypersensitivity vasculitis (allergic vasculitis), Kawasaki disease, microscopic polyarteritis/polyvasculitis, nodular polyarteritis, rheumatoid polymuscular muscle Pain (PMR), rheumatic vasculitis, Takayasu arteritis, Wegener's granulomatosis, and connective tissues such as systemic lupus erythematosus (SLE), rheumatoid arthritis (RA), relapsing polychondritis, Behcet's disease Examples include, but are not limited to, vasculitis that occurs secondary to a disorder or other connective tissue disorder, or vasculitis that occurs secondary to a viral infection.

In some embodiments, chronic inflammation includes a skin disorder. Skin disorders include pressure ulcers (including acne vulgaris), bullous pemphigoid, dermatitis (including atopic dermatitis and chronic photosensitivity dermatitis) such as atopic eczema, contact eczema, dryness Eczema such as eczema, seborrheic eczema, sweating disorders, discoid eczema, venous eczema, herpetic dermatitis, neurodermatitis, and autosensitizing dermatitis, and stasis dermatitis, purulent sweat gland inflammation, lichen planus, psoriasis (including psoriasis vulgaris, nail psoriasis, prickly psoriasis, scalp psoriasis, inverse psoriasis, pustular psoriasis, erythrodermic psoriasis, and psoriatic arthritis), rosacea, and scleroderma (Including, but not limited to, morphea).

In some embodiments, chronic inflammation comprises a gastrointestinal disorder. Gastrointestinal disorders include irritable bowel disease, inflammatory bowel disease (including Crohn's disease), and ulcerative colitis such as ulcerative proctitis, left side colitis, total colitis, and fulminant colitis.

In some embodiments, chronic inflammation includes cardiovascular disease. LDL cholesterol can elicit an immune response when implanted in the arterial wall. Over time, chronic inflammation can damage arteries and cause their rupture. A cardiovascular disease is any of a number of specific diseases that affect the heart itself and/or the vascular system (particularly the veins and arteries connected to the heart). There are over 60 cardiovascular disorders, including hypertension, endocarditis, myocarditis, heart valve dysfunction, congestive heart failure, myocardial infarction, diabetic heart abnormalities, vascular inflammation (eg arteritis, Phlebitis, vasculitis), arterial occlusive disease (eg arteriosclerosis and stenosis), inflammatory cardiac hypertrophy, peripheral arterial disease, aneurysm, embolism, incision, pseudoaneurysm, vascular malformation, vascular nevus, Includes but is not limited to thrombosis, thrombophlebitis, varicose veins, stroke. Symptoms of cardiovascular disorders affecting the heart include chest pain, chest discomfort (angina), pain in one or both arms, left shoulder, neck, chin, or back, shortness of breath, dizziness, accelerated heart rate, nausea, Examples include, but are not limited to, abnormal heartbeat and fatigue. Symptoms of cardiovascular disorders affecting the brain include sudden numbness or weakness of the face, arms, or legs (especially on one side of the body), sudden confusion or difficulty in speaking or understanding, sudden difficulty seeing with both eyes, sudden dizziness, difficulty walking or loss of balance or coordination, sudden unexplained severe headache, but not limited to these. Symptoms of cardiovascular disorders that affect the legs, pelvis, and/or arms include lameness (which is pain, soreness, or convulsions in the muscles), and a feeling of coldness or paralysis of the feet or toes, especially at night.

In some embodiments, chronic inflammation includes an inflammatory cancer, such as an NF-κB-induced inflammatory cancer.

In some embodiments, chronic inflammation includes a vitamin, mineral, or nutrient deficiency. For example, vitamin A deficiency causes an increased inflammatory response.

In some embodiments, chronic inflammation includes pharmacologically induced inflammation. Certain drugs or exogenous chemicals are known to affect inflammation. For example, certain illegal drugs, such as cocaine and MDMA, may activate transcription factors that are involved in inflammation (e.g., NF-κB).

In some embodiments, chronic inflammation includes an infection. Infectious organisms can escape from areas of directly infected tissue via the circulatory system or lymphatic system and can spread to other parts of the body. If the organism is not stopped by the action of acute inflammation, it can enter the lymphatic system through nearby lymphatic vessels. A lymphatic infection is known as lymphangitis, and a lymph node infection is known as lymphadenitis. Pathogens can enter the bloodstream through lymphatic drainage to the circulatory system. Infectious diseases include, but are not limited to, bacterial cystitis, bacterial encephalitis, pandemic influenza, viral encephalitis, and viral hepatitis (types A, B, and C).

In some embodiments, chronic inflammation includes graft rejection. Graft rejection occurs when the transplant recipient's immune system attacks the organ or tissue to be transplanted so that the transplanted organ or tissue is not accepted by the recipient's body. Graft rejection is an adaptive immune response mediated by both T cell mediated and humoral immune (antibody) mechanisms. Graft rejection can be classified as hyperacute rejection, acute rejection, or chronic rejection. Chronic rejection of a transplanted organ or tissue is a rejection caused by a poorly understood chronic inflammatory immune response that occurs against the transplanted tissue. The term “graft rejection” also includes graft versus host disease (GVHD). GVHD is a common complication in allogeneic bone marrow transplantation, in which functional immune cells in the transplanted bone marrow recognize the recipient as a “foreign body” and launch an immune attack. It can also occur in blood transfusions under certain circumstances. GVHD is divided into acute and chronic forms. Acute and chronic GVHD appear to involve different subsets of immune cells, different cytokine profiles, somewhat different host targets, and have different responses to treatment.

In some embodiments, chronic inflammation comprises a Th1-mediated inflammatory disease. In a properly functioning immune system, the immune response should result in a balanced pro-inflammatory and anti-inflammatory Th2 response that is appropriate in dealing with its immune burden. Generally speaking, once a pro-inflammatory Th1 response is initiated, the body counteracts this Th1 response by an anti-inflammatory response caused by a Th2 response. This reactive response is e.g. Th2 such as IL-4, IL-5, and IL-13, which are associated with enhanced IgE and eosinophilic responses in atopy, and IL-10 with an anti-inflammatory response Including the release of types of cytokines. Th1-mediated inflammatory diseases involve an excessive pro-inflammatory response produced by Th1 cells, which leads to chronic inflammation. Th1-mediated diseases can be induced by viruses, bacteria, or chemically (eg, by the environment). For example, a virus that causes a Th1-mediated disease can cause a chronic or acute infection, which can cause respiratory problems or influenza.

In some embodiments, chronic inflammation includes chronic neurogenic inflammation. Chronic neurogenic inflammation is the release of inflammatory molecules such as SP or CGRP released from peripheral sensory nerve endings (ie, the efferent nature that contrasts with normal afferent signaling to the spinal cord in these nerves) Represents an inflammatory response initiated and/or maintained via Chronic neurogenic inflammation includes both primary and secondary neural inflammation. As used herein, the term “primary” neurogenic inflammation refers to tissue inflammation (inflammatory, initiated or caused by the release of substances from primary sensory nerve endings (eg, C and A delta fibers). Symptom). As used herein, the term “secondary” neural inflammation refers to non-neural sources of inflammatory mediators such as peptides or cytokines (eg, extravasation from the vascular bed or between tissues Refers to tissue inflammation that is initiated by quality-derived ones, such as those from mast cells or immune cells, and stimulates sensory nerve endings to cause the release of inflammatory mediators from the nerves. In both forms (primary and secondary) of chronic neurogenic inflammation, the ultimate effect is to produce an inflammatory condition that is maintained by sensitization of peripheral sensory nerve fibers. The physiological consequences of the resulting chronic neurogenic inflammation depend on the target tissue, for example skin pain (allodynia, hyperalgesia), arthralgia and/or arthritis, visceral pain and visceral dysfunction, lung dysfunction (Asthma, COPD), and bladder dysfunction (pain, overactive bladder) occur.

In some embodiments, the inflammatory condition or disorder is an immune disease, disorder, or condition that causes either acute or chronic inflammation (Ahmed A U. Front Biol. 2011:6(4): 274-281).

In some embodiments, the inflammatory condition or disorder is any of pressure ulcers, including acne vulgaris; oxalic acid/heartburn, age-related macular degeneration (AMD), allergies, allergic rhinitis, Alzheimer's disease, amyotrophic lateral sclerosis, anemia, appendicitis, arteritis, arthritis, including osteoarthritis, rheumatoid arthritis, juvenile idiopathic arthritis, spondyloarthropathy such as ankylosing spondylitis, reactive arthritis (Reiter syndrome), psoriatic arthritis, enteroarthritis associated with inflammatory bowel disease, Whipple and Behcet's disease, septic arthritis, gout (also known as gouty arthritis, crystalline synovitis, metabolic arthritis), pseudogout (calcium pyrophosphate deposition disease), and Still's disease. Arthritis can affect a single joint (monoarthritis), two to four joints (oligoarthritis), or five or more joints (polyarthritis); asthma, atherosclerosis, autoimmune disorder, balanitis, blepharitis, bronchiolitis, bronchitis, bullous pemphigoid, burns, bursitis, cancer, incluiding NF-κB-induced inflammatory cancer; cardiovascular disease, including hypertension, endocarditis, myocarditis, heart valve dysfunction, congestive heart failure, myocardial infarction, diabetic heart abnormalities, vascular inflammation, including arteritis, phlebitis, and vasculitis; arterial occlusive disease, including arteriosclerosis and stenosis; inflammatory cardiac hypertrophy, peripheral arterial disease, aneurysm, embolism, incision, pseudoaneurysm, vascular malformation, vascular nevus, thrombosis, thrombophlebitis, varicose veins, stroke, cardiac arrest, and carditis; celiac disease, cellulitis, cervicitis, cholangitis, cholecystitis, chorioamnionitis, chronic obstructive pulmonary disease (COPD), cirrhosis, congestive heart failure, conjunctivitis, colitus, cyclophosphamide-induced cystitis, cystic fibrosis, cystitis, cold, lacrimal inflammation, dementia, dermatitis, including atopic dermatitis, chronic photosensitivity dermatitis, eczema, atopic eczema, contact eczema, dryness eczema, seborrheic eczema, sweating disorders, discoid eczema, venous eczema, herpetic dermatitis, neurodermatitis, and autosensitizing dermatitis, stasis dermatitis, purulent sweat gland inflammation, lichen planus, psoriasis, including psoriasis vulgaris, nail psoriasis, prickly psoriasis, scalp psoriasis, inverse psoriasis, pustular psoriasis, erythrodermic psoriasis, and psoriatic arthritis; rosacea, and scleroderma, including morphea; pharmacologically induced inflammation, including from legal or illegal drugs, and chemicals; chronic neurogenic inflammation, including primary and secondary neural inflammation; dermatomyositis, diabetes, diabetic neuropathy, diabetic retinopathy, diabetic nephropathy, diabetic ulcer, digestive system disease, emphysema, encephalitis, endocarditis, endometritis, enterocolitis, epicondylitis, epididymis, fasciitis, fibromyalgia, fibrosis, connectitis, gastritis, gastroenteritis, gingivitis, glomerulonephritis, glossitis, heart disease, heart Valvular dysfunction, hepatitis, purulent spondylitis, Huntington's disease, hyperlipidemic pancreatitis, hypertension, ileitis, infection, including lymphangitis, lymphadenitis, bacterial cystitis, bacterial encephalitis, pandemic influenza, viral encephalitis, and viral hepatitis (types A, B, and C); inflammatory bowel disease, including Chron's disease; inflammatory heart enlargement, inflammatory neuropathy, insulin resistance, between Interstitial cystitis, interstitial nephritis, iritis, ischemia, ischemic heart disease, keratitis, keratoconjunctivitis, laryngitis, lupus nephritis, mastitis, mastoiditis, meningitis, metabolic syndrome (syndrome X), migraine, multiple sclerosis, myelitis, myocarditis, myositis, nephritis, non-alcoholic steatohepatitis, obesity, umbilitis, ovitis, testitis, osteochondritis, osteopenia, osteomyelitis, osteoporosis, osteomyelitis, otitis, pancreatitis, Parkinson's disease, parotitis, pelvic inflammatory disease, pemphigus vulgaris, pericarditis, Peritonitis, pharyngitis, phlebitis, pleurisy, interstitial pneumonia, polycystic nephritis, polymyositis, proctitis, prostatitis, psoriasis, pulpitis, pyelonephritis, portal vein, renal failure, reperfusion injury, retinitis, rheumatic fever Rhinitis, fallopianitis, sarcoidosis, salivary glanditis, sepsis, including bacteremia and viremia; sinusitis, spastic colon, stenosis, stomatitis, stroke, surgical complications, synovitis, tendonitis, tendonitis, tendonitis, thrombophlebitis, tonsillitis, trauma, traumatic brain injury, graft rejection, including graft versus host disease (GVHD); a Th1-mediated inflammatory disease, trigonitis, tuberculosis, tumor, urethritis, bursitis, uveitis, vaginitis, vasculitis, including Buerger's disease, cerebral vasculitis, Churg-Strauss arteritis, cryoglobulinemia, essential cryoglobulin vasculitis, giant cells arteritis, golfer vasculitis, Henoch-Schonlein purpura, hypersensitivity vasculitis, Kawasaki disease, microscopic polyarteritis/polyvasculitis, nodular polyarteritis, rheumatoid polymuscular muscle pain (PMR), rheumatic vasculitis, Takayasu arteritis, Wegener's granulomatosis, systemic lupus erythematosus (SLE), relapsing polychondritis, Behcet's disease; ulcerative colitis such as ulcerative proctitis, left side colitis, total colitis, and fulminant colitis; and vulvitis.

In some embodiments, the disclosed therapeutic combinations are used to reduce inflammation. In some embodiments, the disclosed therapeutic combinations are used in the manufacture of a medicament to reduce inflammation. In some embodiments, the disclosed therapeutic combinations, e.g., in a therapeutically effective amount, are administered to a subject to reduce inflammation.

A reduction in inflammation, such as a reduction in acute inflammation, a reduction in localized chronic inflammation, or a reduction in systemic chronic inflammation, may be measured according to various methods known to one of skill. Inflammatory biomarkers may be detected from biological specimens, for example, a subject's blood, such as plasma or serum, or saliva. In an example, inflammation is detected by measuring high-sensitivity C-reactive protein (CRP) and white blood cell count from a blood test. CRP may also be detected in a saliva sample. Salivary CRP is not synthesized locally in the mouth and may reflect more systemic levels of inflammation compared to other inflammatory biomarkers, such as cytokines (Szabo & Slavish, Psychoneuroendocrinology. 202; 124:105069). Clinical pathology data, e.g., hematology data on erythrocyte parameters, platelet count, total number of leukocytes, and leukocyte differentials and morphology, coagulation data on clotting times and fibrinogen, and clinical chemistry data on total protein, albumin and globulin, liver enzymes, renal parameters, electrolytes, and bilirubin can provide an indication of the presence and potentially the location of inflammation, in the absence of specific data on immune tissues. See, e.g., Germolec et al., Methods Mol Biol. 2018; 1803:57-79 and Luo et al., Clin Lab. 2019 1; 65(3).

### A. Outcome Measures

In embodiments, measures of therapeutic effect include outcome measures (primary or secondary), endpoints, effect measures, and measures of effect within clinical or medical practice or research which can be used to assess an effect (positive and/or negative) of an intervention or treatment, whether patient-reported (e.g., questionnaires); based on other patient data (e.g., patient monitoring); gathered through laboratory tests such as from blood or urine; through medical examination by a doctor or other medical professional, or by digital means, such as by using electronic tools such as online tools, smartphones, wireless devices, biosensors, or health apps.

The term “outcome measure” may refer to data collected that is related to the treatment of a subject. The outcome measure may include one or more physiological measures, one or more patient-centered outcome measures (PCOMs), or one or more behavioral factor measures.

A measure may be a physiological measure. A physiological measure monitors the physical reaction of a subject (e.g., BMI, biomarker concentration in blood). A measure may be a PCOM. A PCOM is an assessment of the patient's beliefs, opinions and needs in relation to their treatment (e.g., patient-reported negative or positive effects relating to the inflammatory condition or disorder). A measure may be a behavioral factor measure. A behavioral factor measure is a measure of specific behaviors of the subject.

In embodiments, improvements (e.g., in an inflammatory condition or disorder) may be measured by monitoring or self-monitoring of a subject. In some embodiments, the result of a disclosed method is measured by monitoring the number or content of paper diaries submitted by a subject. In some embodiments, the result of a disclosed method is measured by monitoring the number or content of Internet-based diaries submitted by a subject. In some embodiments, the result of a disclosed method is measured by monitoring the scores on a survey taken by a subject. In some embodiments, the result of a disclosed method is measured by monitoring an available database wherein a subject tracks any psychological factors experienced. In some embodiments, the result of a disclosed method is measured by monitoring use of current technology, such as smartphones with applications which track any psychological factors experienced.

In embodiments, a measure of therapeutic effect, which may be an outcome measure, will include an assessment. “Assessment” may refer to any method used with a patient, whether before, during, after, or unrelated in time to a specific treatment protocol, to measure, estimate, or evaluate a nature, ability, symptom, disorder, or other characteristic of the patient, whether qualitatively or quantitatively, and whether performed by a clinician (e.g., an interview), by a patient his or herself (e.g., a self-reported questionnaire), by a third-party or by a computer, including a medical device (e.g., as such as defined by the FDA or other regulatory body) or other device (e.g., a medical sensor or biosensor, a watch or fitness tracker, or a “wearable”), and whether graded by a human decision-maker or an artificial intelligence, machine learning, or computer algorithm, and whether or not regulated as software as a medical device (SaMD).

An assessment may be computer-assisted, and other computer-assisted assessments may be performed besides the assessments above. The term “computer-assisted” in “computer-assisted assessment” means an assessment comprising the use of electronic tools such as online tools, smartphones, wireless devices, or health apps (in some such examples, also known as “digital phenotyping”). Computer-assisted assessment will include the use of an electronic psychiatric notes system, where relevant clinical information will be recorded for the duration of the therapy by a therapist interacting face-to-face with a patient, and will also include the use of computer systems where the therapist and patient interact virtually (either synchronously or asynchronously), as well as where a patient only interacts with a computer (“computer” broadly meaning any electronic tool suitable for such purposes, including desktop, laptop, and notebook computers; tablets, smartphones, and other mobile devices; watches, fitness trackers, and personal electronic devices; and the like).

In embodiments, improvements related to an inflammatory disorder may be an improvement in the severity of symptoms of the inflammatory condition or disorder. In embodiments, the improvement in severity of symptoms of the inflammatory condition or disorder is a reduction in at least one symptom of the inflammatory condition or disorder. In embodiments, the reduction in at least one symptom of the inflammatory condition or disorder is a reduction in any of flushed skin at the site of the injury, pain or tenderness, swelling, heat, abdominal pain, chest pain, fatigue, insomnia, fever, joint pain or stiffness, mouth sores, skin rash, depression, anxiety, or other mood disorders, gastrointestinal issues, such as diarrhea, constipation, or acid reflux, weight gain or weight loss, and frequent infections. In embodiments, the reduction in at least one symptom of the inflammatory condition or disorder is compared to a baseline determination made before administration to the subject of a disclosed therapeutic combination.

In embodiments, improvements related to an inflammatory disorder may be an improvement in one or more symptoms of the inflammatory condition or disorder. In embodiments, the improvement of at least one symptom of the inflammatory condition or disorder is an improvement in any of flushed skin at the site of the injury, pain or tenderness, swelling, heat, abdominal pain, chest pain, fatigue, insomnia, fever, joint pain or stiffness, mouth sores, skin rash, depression, anxiety, or other mood disorders, gastrointestinal issues, such as diarrhea, constipation, or acid reflux, weight gain or weight loss, and frequent infections.

In embodiments, the improvement is an improvement in a symptom of long COVID, which includes an improvement in any of mental clarity, fatigue, brain fog, mood, depression, activity level, ability to perform routine tasks, and headache. In embodiments, the improvement is an improvement in a symptom of CREST syndrome, which includes an improvement in any of stiffness, motor function, amplitude and frequency of flare-ups, calcification, fatigue, Raynaud spasms, and sleep quality.

In embodiments, the improvement is an improvement in a symptom of psoriatic arthritis, which includes a reduction in any of calcification, amplitude and frequency of flare-ups, joint pain, itching, fatigue, and plaque formation. In embodiments, the improvement is an improvement of a symptom of Hashimoto's Disease, which includes a reduction in any of stiffness, amplitude and frequency of flare-ups, body mass, and fatigue. In embodiments, the improvement of a symptom of Hashimoto's Disease is an improvement in any of mood, motor function and endurance.

In embodiments, the improvement is an improvement in a symptom of Multiple Sclerosis, which includes a reduction in amplitude and frequency of flare-ups. In embodiments, the improvement of a symptom of Multiple Sclerosis is an improvement in any of the ability to communicate, energy level, mood, and sleep quality. In embodiments, the improvement is an improvement in the ability to avoid a flare-up in a situation where a flare-up would have previously been unavoidable.

In embodiments, the improvement is an improvement in a symptom of Sciatica, which includes a reduction in any of the amplitude and frequency of flare-ups, pain, nerve pain, swelling, and inflammation. In embodiments, the improvement in a symptom of Sciatica is an improvement in peripheral sensation.

In embodiments, the improvement is an improvement in peripheral neuritis, which includes a reduction in any of the amplitude and frequency of flare-ups, pain, nerve pain, swelling, and inflammation. In embodiments, the improvement in a symptom of peripheral neuritis is an improvement in peripheral sensation.

In embodiments, the improvement is an improvement in post-treatment Lyme Disease (PTLD)/Chronic Lyme Disease (CLD) Syndrome, which includes an improvement in any of fatigue, joint or muscle aches, cognitive dysfunction, and brain fog.

In embodiments, the improvement is an improvement in Chronic Fatigue and Immune Dysfunction Syndrome (CFIDS)/Chronic Fatigue Syndrome (CFS), which includes an improvement in any of brain fog; post-exertion energy; quality of life; dizziness; muscle and joint pain; sleep patterns; headache; sore throat; tender lymph nodes; heightened sensitivity to light, sound, smell, food, and medicine; and mood.

In embodiments, the improvement is an improvement in an inflammatory skin condition, which includes an improvement in any of clear skin, sensitivity to sunlight, tolerance to allergens, redness, heat, swelling, pain, and itching.

In embodiments, improvements related to an inflammatory disorder may be increased antioxidant effects. In embodiments, the antioxidant effects of disclosed methods can be measured by a total antioxidant assay, as disclosed in (Apak et al. Molecules. 2007; 12(7):1496-1547). In embodiments, the total antioxidant assay is a Folin-Ciocalteu assay. In embodiments, the total antioxidant assay is a 2,2-diphenyl-1-picryl (DPPH) assay. In embodiments, the total antioxidant assay is a Trolox Equivalent Antioxidant Capacity (TEAC) assay. In embodiments, the total antioxidant assay is a Ferric Reducing Antioxidant Power (FRAP) assay. In embodiments, the total antioxidant assay is a Total Antioxidant Capacity (TAC) assay. In embodiments, the total antioxidant assay is a Superoxide Dismutase (SOD) assay. In embodiments, the total antioxidant assay is a Lipid Peroxidation assay. In embodiments, the total antioxidant assay is a Hydroxyl Radical Scavenging assay. In embodiments, the total antioxidant assay is a cupric ion reducing antioxidant capacity (CUORAC) assay.

Furthermore, in some embodiments, the antioxidant effects of disclosed methods may be measured by a change in a biomarker of an inflammatory condition or disorder, for example, a change (e.g., reduction) in iron levels, and/or a decrease in the concentration of enzymes and substrates associated with oxidative stress. Several biomarkers of oxidative stress have been linked to inflammatory disease and disorders (Lugrin et al., 2014). In embodiments, the biomarker is nigral cell death, free iron, glutathione (GSH), glutathione disulfide (GSSG), nuclear factor erythroid 2-related factor 2 (NRF2), advanced glycation end products (AGE), aldehyde 4-hydroxynonenal (HNE), malonaldehyde (MDA), F2-isoprostanes, isolevuglandins (isoLG), nitrotyrosine (NO2-Tyr), or a combination thereof. In embodiments, the biomarker is nigral cell death. In embodiments, the biomarker is free iron. In embodiments, the biomarker is glutathione (GSH). In embodiments, the biomarker is glutathione disulfide (GSSG). In embodiments, the biomarker is nuclear factor erythroid 2-related factor 2 (NRF2). In embodiments, the biomarker is advanced glycation end products (AGE). In embodiments, the biomarker is aldehyde 4-hydroxynonenal (HNE). In embodiments, the biomarker is malonaldehyde (MDA). In embodiments, the biomarker is F2-isoprostanes. In embodiments, the biomarker is isolevuglandins (isoLG). In embodiments, the biomarker is nitrotyrosine (NO2-Tyr). In embodiments, the inflammation biomarker is the gene TNFα, IL-4, IL-5, IL-6, IL-8, IL-9, IL-1β, II-IA, IL-12, IFNα, IFNb, IFNg, TGF-β, IL-15, IL-17, IL-20, IL-22, LTA, IL-23, IL-18, VCAM1, ICAM1, MCP1, MMP-9, Muc5ac, Gm-csf, CCL2, CCL5, CCL3, CCL4, CCL11, CD11a, CD3, CD4, CD8, or CRP

In embodiments, improvements related to an inflammatory disorder may be improvements in an inflammatory response. One of skill can monitor improvements in an inflammatory response by using one or more types of laboratory analyses to detect inflammatory biomarkers in a subject. In embodiments, improvements in an inflammatory response include assessing changes in the levels of inflammation biomarkers. In embodiments, inflammation biomarkers include a gene that encodes an inflammatory protein, an mRNA that encodes an inflammatory protein, and/or an inflammatory protein. In embodiments, the inflammation biomarker is the gene TNFα, IL-4, IL-5, IL-6, IL-8, IL-9, IL-1β, Il-IA, IL-12, IFNα, IFNb, IFNg, TGF-β, IL-15, IL-17, IL-20, IL-22, LTA, IL-23, IL-18, VCAM1, ICAM1, MCP1, MMP-9, Muc5ac, Gm-csf, CCL2, CCL5, CCL3, CCL4, CCL11, CD11a, CD3, CD4, CD8, or CRP.

In embodiments, the inflammation biomarker is an mRNA or protein produced from an inflammation biomarker gene.

In embodiments, the concentration of the biomarker is decreased by about at least 99%, 99%, 98%, 95%, 90%, 85%, 80%, 75%, 70%, 65%, 60%, 55%, 50%, 45%, 40%, 35%, 30%, 25%, 20%, 15%, 10%, 5%, 4%, 3%, 2%, 1%, or less than 1%, including ranges in between these values. In embodiments, the concentration of the biomarker is decreased by at least 99%. In embodiments, the concentration of the biomarker is decreased by about 99%. In embodiments, the concentration of the biomarker is decreased by about 98%. In embodiments, the concentration of the biomarker is decreased by about 95%. In embodiments, the concentration of the biomarker is decreased by about 90%. In embodiments, the concentration of the biomarker is decreased by about 85%. In embodiments, the concentration of the biomarker is decreased by about 80%. In embodiments, the concentration of the biomarker is decreased by about 75%. In embodiments, the concentration of the biomarker is decreased by about 70%. In embodiments, the concentration of the biomarker is decreased by about 65%. In embodiments, the concentration of the biomarker is decreased by about 60%. In embodiments, the concentration of the biomarker is decreased by about 55%. In embodiments, the concentration of the biomarker is decreased by about 50%. In embodiments, the concentration of the biomarker is decreased by about 45%. In embodiments, the concentration of the biomarker is decreased by about 40%. In embodiments, the concentration of the biomarker is decreased by about 35%. In embodiments, the concentration of the biomarker is decreased by about 30%. In embodiments, the concentration of the biomarker is decreased by about 25%. In embodiments, the concentration of the biomarker is decreased by about 20%. In embodiments, the concentration of the biomarker is decreased by about 15%. In embodiments, the concentration of the biomarker is decreased by about 10%. In embodiments, the concentration of the biomarker is decreased by about 5%. In embodiments, the concentration of the biomarker is decreased by about 4%. In embodiments, the concentration of the biomarker is decreased by about 3%. In embodiments, the concentration of the biomarker is decreased by about 2%. In embodiments, the concentration of the biomarker is decreased by about 1%. In embodiments, the concentration of the biomarker is decreased by less than 1%.

In embodiments, improvements in an inflammatory response may be monitored using a C-reactive protein test. “C-reactive protein” or “CRP” may refer to an annular (ring-shaped) pentameric protein found in blood plasma. A CRP test measures the level of CRP in a subject's blood. The liver releases CRP into the bloodstream in response to inflammation. Traditional CRP tests only detect CRP in amounts ranging from between about 10 to 1,000 mg/L, whereas high sensitivity CRP tests (hs-CRPs) detect CRP in amounts ranging from between about 0.5 to 10 mg/L.

In embodiments, improvements in an inflammatory response may be monitored by determining the erythrocyte sedimentation rate (ESR) in a subject. “Erythrocyte sedimentation rate” (ESR) is the rate at which red blood cells in anticoagulated whole blood descend in a standardized tube over a period of one hour. An ESR test is common in hematology, and is a non-specific measure of inflammation. To perform an ESR test, anticoagulated blood is placed in an upright tube, known as a Westergren tube, and the distance at which the red blood cells fall is measured and reported in millimeters at the end of one hour. In inflammatory disorders, fibrinogen, other clotting proteins, and alpha globulin are positively charged, thus increasing the ESR. For example, the ESR typically begins to rise at 24-48 hours after the onset of acute self-limited inflammation, decreases slowly as inflammation resolves, and can take weeks to months to return to normal levels.

In embodiments, improvements in an inflammatory response may be monitored using a plasma viscosity test. Plasma viscosity can serve as a marker of inflammation in a subject. Plasma viscosity (PV) measures the viscosity, or the ‘thickness’ of a subject's plasma, and is affected by the amount of proteins in the blood. Protein levels in the blood can increase as part of a normal response to infection or inflammation. Normal levels of plasma viscosity in a subject are between about 1.3-1.7 mPas (millipascal second). The higher the plasma viscosity in a subject, the more viscous the blood, which in turn signals the presence of a more severe inflammatory response.

In embodiments, improvements in an inflammatory response may be monitored by analyzing procalcitonin levels in a subject. Procalcitonin (PCT) is a peptide precursor of the hormone calcitonin, the latter being involved with calcium homeostasis. PCT arises once pre-procalcitonin is cleaved by endopeptidase. The level of procalcitonin in the blood stream of healthy subjects is below the limit of detection (0.01 μg/L) of clinical assays (Schuetz et al. BMC Medicine. 2011; 9:107). However, levels of procalcitonin in the blood may be elevated in a number of different inflammatory disorders.

In embodiments, improvements in an inflammatory response may be monitored by analyzing ferritin levels in a subject. Ferritin is a universal intracellular protein that stores iron and releases it in a controlled fashion. Ferritin serves as a biomarker of inflammation in subjects. For men, a normal range for ferritin levels in the blood is between about 24 to 336 micrograms per liter. For women, a normal range for ferritin levels in the blood is between about 11 to 307 micrograms per liter. Higher levels of ferritin in the blood indicate the presence of one or more inflammatory disorders in a subject (Khan et al. J. Obes. 2016; 2016:1937320).

In embodiments, improvements in an inflammatory response may be monitored by analyzing fibrinogen levels in a subject. Fibrinogen (factor I) is a glycoprotein complex, produced in the liver, that circulates in the blood of all vertebrates. Fibrinogen is a “positive” acute-phase protein, meaning its blood levels rise in response to systemic inflammation, tissue injury, and certain other events. Elevated levels of fibrinogen in the blood due to inflammation as well as cancer or other conditions have been suggested to be the cause of thrombosis and vascular injury. Normal levels of fibrinogen in a subject are between about 1.5-3 g/L (Alexander et al. Thromb Haemost. 2011; 105(4):605-9).

In embodiments, improvement in an inflammatory response may be monitored by analyzing concentrations of metabolites associated with an inflammatory condition or disorder.

In embodiments, the improvement is an improvement in a mood symptom. In embodiments, the mood symptom is any of depression, anxiety, apathy, irritability, mood swings, impaired judgment, loss of empathy, aggression, impulsivity, delusions, and paranoia. In embodiments, the reduction in symptoms is a reduction in mood symptoms. In embodiments, the improvement in mood symptoms may be determined via clinician outcome assessments, such as those disclosed herein, as well as those known to be useful for mood assessment, including the Beck Depression Inventory (BDI), the Hospital Anxiety and Depression Scale (HADS), the Hamilton Anxiety Scale (HAM-A or HAS), the Hamilton Depression Scale (HAM-D), the Montgomery-Asberg Depression Rating Scale (MADRS), the Patient Health Questionnaire 9 (PHQ-9), the Generalized Anxiety Disorder 7 (GAD-7), the Obsessive-Compulsive Inventory (OCI), the Personality Disorders Questionnaire (PDQ-IV), Dissociative Experiences Scale (DES), the Mood Disorder Questionnaire (MDQ), and other similar questionnaires. In embodiments, assessment of mood may be completed by assessment prior to, during, and after treatment with the therapeutic combinations.

Other methods for evaluating improvements related to inflammatory disorders, useful in the practice of the disclosure, will be known to those of skill in view of these teachings and the knowledge in the art.

In embodiments, the time elapsed between administering to a subject a disclosed therapeutic combination and the reduction in the severity of symptoms is less than about 75 days, including less than 75 days, less than 70 days, less than 65 days, less than 60 days, less than 55 days, less than 50 days, less than 45 days, less than 40 days, less than 35 days, less than 30 days, less than 25 days, less than 20 days, less than 15 days, less than 10 days, less than 5 days, 1 day or less, including 18 hours or less, 12 hours or less, 6 hours or less, 3 hours or less, 2 hours or less, 1 hour or less, 30 minutes or less, 15 minutes or less, and 1 minute or less, and values in between.

In embodiments, the time elapsed between administering to a subject a therapeutic combination and the reduction in the severity of symptoms is greater than about 75 days, but less than about 100 days. In embodiments, the time elapsed between administering to a subject a therapeutic combination and the reduction in the severity of symptoms is greater than about 100 days, but less than about 125 days. In embodiments, the time elapsed between administering to a subject a therapeutic combination and the reduction in the severity of symptoms is greater than about 125 days, but less than about 150 days. In embodiments, the time elapsed between administering to a subject a therapeutic combination and the reduction in the severity of symptoms is greater than about 150 days, but less than about 175 days. In embodiments, the time elapsed between administering to a subject a therapeutic combination and the reduction in the severity of symptoms is greater than about 175 days, but less than about 200 days. In embodiments, the time elapsed between administering to a subject a therapeutic combination and the reduction in the severity of symptoms is greater than about 200 days, but less than about 225 days. In embodiments, the time elapsed between administering to a subject a therapeutic combination and the reduction in the severity of symptoms is greater than about 225 days, but less than about 250 days. In embodiments, the time elapsed between administering to a subject a therapeutic combination and the reduction in the severity of symptoms is greater than about 250 days, but less than about 275 days. In embodiments, the time elapsed between administering to a subject a therapeutic combination and the reduction in the severity of symptoms is greater than about 275 days, but less than about 300 days. In embodiments, the time elapsed between administering to a subject a therapeutic combination and the reduction in the severity of symptoms is greater than about 300 days, but less than about 325 days. In embodiments, the time elapsed between administering to a subject a therapeutic combination and the reduction in the severity of symptoms is greater than about 325 days, but less than about 350 days. In embodiments, the time elapsed between administering to a subject a therapeutic combination and the reduction in the severity of symptoms is greater than about 350 days, but less than about 1 year. In embodiments, the time elapsed between administering to a subject a therapeutic combination and the reduction in the severity of symptoms is greater than about 1 year days, but less than about 2 years. In embodiments, the time elapsed between administering to a subject a therapeutic combination and the reduction in the severity of symptoms is greater than about 2 years, but less than about 3 years. In embodiments, the time elapsed between administering to a subject a therapeutic combination and the reduction in the severity of symptoms is greater than about 3 years, but less than about 5 years. In embodiments, the time elapsed between administering to a subject a therapeutic combination and the reduction in the severity of symptoms is greater than about 5 years, but less than about 10 years. In embodiments, the time elapsed between administering to a subject a therapeutic combination and the reduction in the severity of symptoms is greater than about 10 years, but less than about 25 years.

In embodiments, the time elapsed between administering to a subject a therapeutic combination and the reduction in the severity of symptoms is less than about 1 year, less than about 2 years, less than about 3 years, less than about 4 years, less than about 5 years, less than about 6 years, less than about 7 years, less than about 8 years, less than about 9 years, less than about 10 years, less than about 15 years, less than about 20 years, or less than about 25 years.

In embodiments, the reduction in the severity of at least one symptom is a durable reduction, wherein the reduction lasts for at least about 1 week, including about 2 weeks, about 3 weeks, about 1 month, about 2 months, about 3 months, about 4 months, about 5 months, about 6 months, about 7 months, about 8 months, about 9 months, about 10 months, about 11 months, about 12 months, about 18 months, about 24 months, about 36 months, about 48 months, about 60 months, and more than about 60 months, when measured from baseline (as other such reductions or determinations of durability herein may be measured from). In embodiments, the reduction in the severity of at least one symptom is durable for at least one week, two weeks, three weeks, 1 month, 2 months, 3 months, 6 months, 9 months, or at least 12 months, when measured from baseline.

In embodiments, the time elapsed between administering to a subject a therapeutic combination and the improvement in a symptom of an inflammatory condition or disorder is less than about 75 days, including less than 75 days, less than 70 days, less than 65 days, less than 60 days, less than 55 days, less than 50 days, less than 45 days, less than 40 days, less than 35 days, less than 30 days, less than 25 days, less than 20 days, less than 15 days, less than 10 days, less than 5 days, 1 day or less, including 18 hours or less, 12 hours or less, 6 hours or less, 3 hours or less, 2 hours or less, 1 hour or less, 30 minutes or less, 15 minutes or less, and 1 minute or less, and values in between.

In embodiments, the improvement in a symptom of an inflammatory condition or disorder is a durable improvement, wherein the improvement lasts for at least about 1 week, including about 2 weeks, about 3 weeks, about 1 month, about 2 months, about 3 months, about 4 months, about 5 months, about 6 months, about 7 months, about 8 months, about 9 months, about 10 months, about 11 months, about 12 months, about 18 months, about 24 months, about 36 months, about 48 months, about 60 months, and more than about 60 months.

In embodiments, the improvement is an improvement in the severity of at least one symptom of the inflammatory condition or disorder. In embodiments, the improvement is a reduction in at least one symptom of the inflammatory condition or disorder. In embodiments, the improvement is an elimination of at least one symptom of the inflammatory condition or disorder. In embodiments, the reduction is compared to a baseline determination made before such administration. In embodiments, the improvement is durable, e.g., lasts for at least 3 months, 6 months, 9 months, 1 year, or greater than 1 year.

In embodiments, the time elapsed between administering to a subject a therapeutic combination and the reduction in the severity of symptoms is less than about 75 days, including less than 75 days, less than 70 days, less than 65 days, less than 60 days, less than 55 days, less than 50 days, less than 45 days, less than 40 days, less than 35 days, less than 30 days, less than 25 days, less than 20 days, less than 15 days, less than 10 days, less than 5 days, 1 day or less, including 18 hours or less, 12 hours or less, 6 hours or less, 3 hours or less, 2 hours or less, 1 hour or less, 30 mins or less, 15 mins or less, and 1 minute or less, and values in between.

In embodiments, the time elapsed between administering to a subject a therapeutic combination and the reduction in the severity of symptoms is greater than about 75 days, but less than about 100 days. In embodiments, the time elapsed between administering to a subject a therapeutic combination and the reduction in the severity of symptoms is greater than about 100 days, but less than about 125 days. In embodiments, the time elapsed between administering to the patient in need thereof the therapeutic combination and the reduction in the severity of symptoms is greater than about 125 days, but less than about 150 days. In embodiments, the time elapsed between administering to a subject a therapeutic combination and the reduction in the severity of symptoms is greater than about 150 days, but less than about 175 days. In embodiments, the time elapsed between administering to a subject a therapeutic combination and the reduction in the severity of symptoms is greater than about 175 days, but less than about 200 days. In embodiments, the time elapsed between administering to a subject a therapeutic combination and the reduction in the severity of symptoms is greater than about 200 days, but less than about 225 days. In embodiments, the time elapsed between administering to a subject a therapeutic combination and the reduction in the severity of symptoms is greater than about 225 days, but less than about 250 days. In embodiments, the time elapsed between administering to a subject a therapeutic combination and the reduction in the severity of symptoms is greater than about 250 days, but less than about 275 days. In embodiments, the time elapsed between administering to a subject a therapeutic combination and the reduction in the severity of symptoms is greater than about 275 days, but less than about 300 days. In embodiments, the time elapsed between administering to a subject a therapeutic combination and the reduction in the severity of symptoms is greater than about 300 days, but less than about 325 days. In embodiments, the time elapsed between administering to a subject a therapeutic combination and the reduction in the severity of symptoms is greater than about 325 days, but less than about 350 days. In embodiments, the time elapsed between administering to a subject a therapeutic combination and the reduction in the severity of symptoms is greater than about 350 days, but less than about 1 year. In embodiments, the time elapsed between administering to a subject a therapeutic combination and the reduction in the severity of symptoms is greater than about 1 year days, but less than about 2 years. In embodiments, the time elapsed between administering to a subject a therapeutic combination and the reduction in the severity of symptoms is greater than about 2 years, but less than about 3 years. In embodiments, the time elapsed between administering to a subject a therapeutic combination and the reduction in the severity of symptoms is greater than about 3 years, but less than about 5 years. In embodiments, the time elapsed between administering to a subject a therapeutic combination and the reduction in the severity of symptoms is greater than about 5 years, but less than about 10 years. In embodiments, the time elapsed between administering to a subject a therapeutic combination and the reduction in the severity of symptoms is greater than about 10 years, but less than about 20 years.

In embodiments, the time elapsed between administering to a subject a therapeutic combination and the reduction in the severity of symptoms is less than about 1 year, less than about 2 years, less than about 3 years, less than about 4 years, less than about 5 years, less than about 6 years, less than about 7 years, less than about 8 years, less than about 9 years, less than about 10 years, less than about 15 years, or less than about 20 years.

In embodiments, the reduction in the severity of at least one symptom is a durable reduction, wherein the reduction lasts for at least about 1 week, including about 2 weeks, about 3 weeks, about 1 month, about 2 months, about 3 months, about 4 months, about 5 months, about 6 months, about 7 months, about 8 months, about 9 months, about 10 months, about 11 months, about 12 months, about 18 months, about 24 months, about 36 months, about 48 months, about 60 months, and more than about 60 months.

In embodiments, the time elapsed between administering to the patient the therapeutic combinations and the improvement in inflammatory condition or disorder is less than about 75 days, including less than 75 days, less than 70 days, less than 65 days, less than 60 days, less than 55 days, less than 50 days, less than 45 days, less than 40 days, less than 35 days, less than 30 days, less than 25 days, less than 20 days, less than 15 days, less than 10 days, less than 5 days, 1 day or less, including 18 hours or less, 12 hours or less, 6 hours or less, 3 hours or less, 2 hours or less, 1 hour or less, 30 minutes or less, 15 minutes or less, and 1 minute or less, and values in between.

In embodiments, the improvement in inflammatory condition or disorder is a durable improvement, wherein the improvement lasts for at least about 1 week, including about 2 weeks, about 3 weeks, about 1 month, about 2 months, about 3 months, about 4 months, about 5 months, about 6 months, about 7 months, about 8 months, about 9 months, about 10 months, about 11 months, about 12 months, about 18 months, about 24 months, about 36 months, about 48 months, about 60 months, and more than about 60 months.

In embodiments, at least one measurable improvement will be seen within 120 days of initiating treatment with a disclosed combination. In embodiments, the time elapsed between administering to a patient a therapeutic combination and the improvement is less than about 120 days, including less than 120 days, less than 115 days, less than 110 days, less than 105 days, less than 100 days, less than 95 days, less than 90 days, less than 85 days, less than 80 days, less than 75 days, less than 70 days, less than 65 days, less than 60 days, less than 55 days, less than 50 days, less than 45 days, less than 40 days, less than 35 days, less than 30 days, less than 25 days, less than 20 days, less than 15 days, less than 10 days, less than 5 days, 1 day or less, including 18 hours or less, 12 hours or less, 6 hours or less, 3 hours or less, 2 hours or less, 1 hour or less, 30 minutes or less, 15 minutes or less, and 1 minute or less, and values in between.

In embodiments, a patient is prescribed a therapeutic combination or may obtain it over-the-counter (OTC). In embodiments, the therapeutic combinations may be obtained from a health-care provider or pharmacy as an OTC medication, and/or may be obtained via purchase at a vendor who is not a health-care provider or pharmacy.

In some embodiments, an advantageous effect of a disclosed therapeutic combination, as compared to the current standard of care in the respective indication may include any of: (1) a larger percentage of patients experiencing a clinical response; (2) a larger average clinical response; (3) an earlier onset of the clinical response; (4) a more durable clinical response; (5) a similar or better clinical response with fewer or different side effects and therefore improved compliance; and (6) a similar or better clinical response with a more convenient therapeutic regimen with fewer drug administrations and therefore improved compliance. In some embodiments, a comparison to the current standard of care consists of, or comprises, a comparison to any of the anti-inflammatory agents known or disclosed herein.

### VIII. EXAMPLES

### Example 15: Clinical Evaluation of Disclosed Combinations for Treating Inflammatory Conditions

Disclosed therapeutic combinations were initially tested by subjects with inflammatory diseases, including long COVID, CREST syndrome, psoriatic arthritis, Hashimoto's disease, multiple sclerosis, sciatica, peripheral neuritis, post-treatment Lyme disease (PTLD)/chronic Lyme disease (CLD) syndrome, chronic fatigue and immune dysfunction syndrome (CFIDS)/chronic fatigue syndrome (CFS), and inflammatory skin conditions.

The evaluation and diagnosis of each of the subjects was performed by their physician. Each dose of the therapeutic combination (“therapeutic”) was 50 mg total, and contained 250 μg of psilocybin, 150 μg of psilocin, 1 mg of THC, 1 mg of CBD, 1 mg of Coumarin, and 8 mg of whole Pyropia extract. Subjects reported evidence of a reduction of symptoms following treatment; in some cases, the reduction was durable.

### Case 1—Long-COVID:

Five participants suffering from diagnosed and confirmed persistent long COVID, meaning the participant suffered from persistent symptoms and/or signs, developing during or after an acute COVID-19 illness and lasting for at least 2 months and persisting beyond 12 weeks from the acute disease, that cannot be explained by an alternative diagnosis. Participants were evaluated for long COVID based on factors associated with fatigue, headache, confusion, depression, and sleep in accordance with tables 3 and 4 of meta-analysis as discussed in Yelin et al. Clin Microbiol Infect. 2022; 28(7):955-972, which is hereby incorporated by reference as if fully set forth herein.

Four participants were male, and one was female, ranging in age from 21-80 years old. Each participant was given 200 mg daily of the therapeutic (four 50-mg doses) for a period of 100 days, and evaluated before, during, and after treatment. In every case, scores meaningfully improved across each category. Volunteers were evaluated using a number of different methodologies, but we largely relied on patient self-reporting and caretaker/parent/friend reports. Each participant reported more restful sleep; reduced fatigue; reduced brain-fog/improved clarity of thought; fewer headaches; improved mood; a return to regular activities, including activities such as work or school that they had previously been unable to perform; and improved job functioning. Three participants reported an improved ability to navigate technology, computer, and email.

### Case 2—CREST Syndrome:

Two participants were treated for CREST syndrome using the therapeutic. Both saw significant improvement in Raynaud's spasms, and an overall decrease in the frequency and severity of episodes/flare ups. Both also reported improved mood, greatly reduced joint stiffness, improved motor ability, and better sleep. Participant 1, a 64 year old female, was diagnosed with CREST syndrome 20 years prior, took 200 mg/daily (in four 50-mg doses) of the therapeutic for 10 months, experiencing a durable recovery at some point along the way. Her joint, motor, and fatigue issues are in complete remission. Participant 2, a 46 year old female, is taking 200 mg/daily of the therapeutic and experiencing considerably reduced inflammation.

### Case 3—Psoriatic Arthritis:

One participant, a 75 year old male with psoriatic arthritis, was treated with 200 mg daily (in four 50-mg doses) of the therapeutic. Over a 90-day period, he saw reduced plaques, more energy, and greatly reduced joint pain and itching.

### Case 4—Hashimoto's Disease:

One participant with Hashimoto's disease, a 52 year old woman, was treated with the 200 mg daily (in four 50-mg doses) of the therapeutic. In a 90-day period, she saw greatly improved endurance, reduced fatigue/exhaustion, and improved sleep. Swelling subsided, her mood improved, and she was able to lose weight that had been previously unlosable.

### Case 4—Multiple Sclerosis:

One female participant with relapsing-remitting (RRMS) multiple sclerosis is currently being treated with an escalating dose from 50-200 mg/daily (in four 50-mg doses) of the therapeutic. She is experiencing a reduction in flare-ups, improved energy, sleep, mood, and resilience (in this case, resilience refers to the capability to avoid a flare-up in a situation where a flare-up would have previously been unavoidable).

### Case 5—Sciatica and Peripheral Neuritis:

One male participant with sciatica neuritis, and 1 male with peripheral neuritis was treated using the therapeutic. Both saw durable improvement or disappearance of swelling nerve pain within 120 days of treatment. The male with peripheral neuritis regained sensation in both feet once inflammation was reduced.

### Case 6—Post-Treatment Lyme Disease (PTLD)/Chronic Lyme Disease (CLD) Syndrome:

One participant, a 69 year old man, was treated for PTLD/CLD Syndrome using the therapeutic. He saw significant improvement in brain fog, exhaustion, achiness, and sensitivity. He also reported improved mood, physical resilience, and sleep.

### Case 7—Chronic Fatigue and Immune Dysfunction Syndrome (CFIDS)/Chronic Fatigue Syndrome (CFS):

Four participants, 3 male and one female, aged 42-72, were treated for CFIDS using the therapeutic. All saw significant improvements in brain fog, post-exertion energy levels, and general quality of life.

### Case 8—Inflammatory Skin Conditions:

One participant, a 72 year old male, being treated with the therapeutic for CFIDS and various other inflammatory conditions, experienced a remarkable improvement to a variety of debilitating and chronic skin conditions (most have been constant since his youth). In particular, a dermatology exam concluded a complete remission of both contact dermatitis and polymorphous light eruption.

Additional clinical observations regarding these participants are as follows:

| Indication | Participants | Key Changes |
|---|---|---|
| Long COVID | 5 | Improved mental clarity |
| | | Reduced fatigue |
| | | Improved mood |
| | | Reduced headaches |
| CREST Syndrome | 2 | Reduced stiffness |
| | | Improved motor function |
| | | Reduced amplitude and frequency |
| | | of flare-ups (in one case, to 0) |
| | | Reduced or eliminated calcification |
| | | Reduced fatigue |
| | | Improved sleep |
| Psoriatic Arthritis | 1 | Reduced amplitude and frequency of flare-ups |
| | | Reduced or eliminated calcification |
| Hashimoto's | 1 | Reduced stiffness |
| Disease | | Improved motor function |
| | | Reduced amplitude and frequency |
| | | of flare-ups (in one case, to 0) |
| | | Reduced fatigue |
| | | Improved endurance |
| Multiple Sclerosis | 1 | Reduced amplitude and frequency of flare-ups |
| Sciatica and | 2 | Reduced amplitude and frequency of flare-ups |
| Peripheral Neuritis | | Reduced pain |
| | | Improved peripheral sensation |
| Chronic Lyme | 1 | Reduced “Brain Fog” |
| Disease (CLD) | | Reduced Exhaustion |
| | | Reduced Achiness |
| Chronic Fatigue | 4 | Reduced “Brain Fog” |
| Syndrome | | Improved post-exertion energy |
| | | Improved quality of life |
| | | Improved mood |
| Other Inflammatory | 1 | Clear Skin |
| Skin Conditions | | Reduced or eliminated sensitivity to sunlight |
| | | Improved tolerance to allergens |

### Example 16: Exemplary Production of a Disclosed Composition

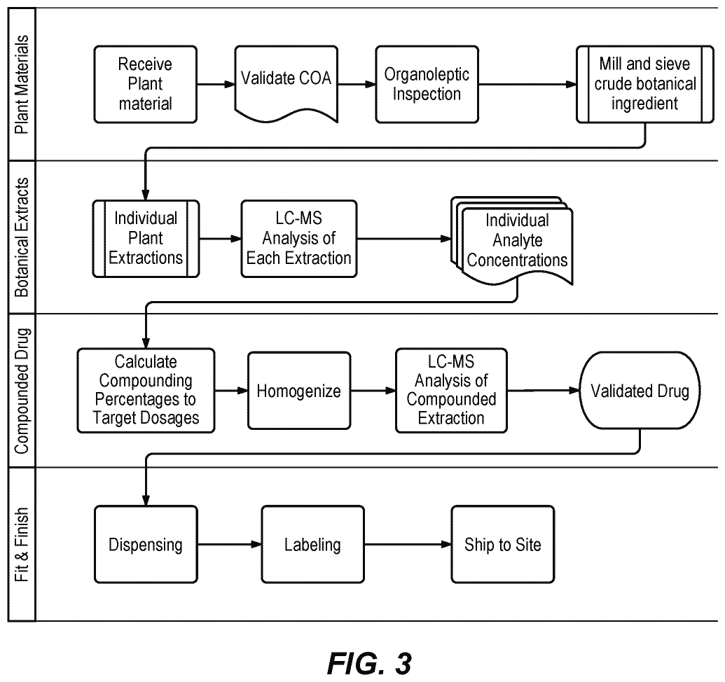

An exemplary process of producing a composition according to the methods of certain described embodiments is depicted in FIG. 3. In some embodiments, when a certain form of plant material is received for purposes of producing a composition, a Certificate of Analysis (COA) is obtained and validated for the plant, fungi, or algae material, and an organoleptic inspection is performed. A COA is a document that communicates the results of certain analyses done on a substance, including the presence of any chemicals used in the substance's manufacturing and testing, and can be created to ensure any applicable regulations are complied with. Organoleptic inspections are performed to detect disease or contamination within a substance. In some embodiments, the plant, fungi, or algae material is thereafter milled and sieved into a crude botanical ingredient.

In some exemplary embodiments, milled and sieved plant, fungi, and/or algae raw material can be prepared, extracted, and filtered as follows:

Psilocybe cubensis: 30 grams of milled and sieved P. cubensis powder is first prepared by soaking for 48 hours in a 200 mL solution of 75% ethanol and 25% water. Then, it is extracted ultrasonically for 25 minutes at ¾ power and pulse duration. Next, it is filtered by hot filtration to 0.8 microns. In another embodiment, 40 grams of milled and sieved P. cubensis powder is first prepared for extraction in a Soxhlet apparatus by adding to a solution of 400 mL ethanol and 100 mL water. Then, it is extracted using the Soxhlet apparatus at 84° C. for 7 hours. Next, it is filtered by hot filtration to 0.8 microns (μm). In some embodiments, both such methods of preparation, extraction, and filtration are used, with the results of each combined together. In some such embodiments, where both such methods of preparation, extraction, and filtration are used, and where the results of each are combined together, the resulting combination comprises 80% of the ultrasound extraction, and 20% of the Soxhlet extraction, such as by w/w %. In some other embodiments, the combination is 2:1 ultrasonic to Soxhlet solutions.

Cannabis sativa: 40 grams of milled and sieved C. sativa powder is first prepared for extraction in a Soxhlet apparatus by adding to a solution of 500 mL ethanol. Then, it is extracted using the Soxhlet apparatus at 84° C. for 7 hours. Next, it is filtered by hot filtration to 0.8 μm.

Pyropia yezoensis: 40 grams of milled and sieved P. yezoensis powder is first prepared by soaking for 48 hours in a solution of 200 mL ethanol and 40 mL water. Then, it is extracted ultra-sonically for 1 hour at ¾ power and pulse duration. Next it is filtered by hot filtration to 0.8 μm.

Dipteryx odorata: 50 grams of milled and sieved D. odorata powder is prepared, extracted, and filtered by percolation with 100 mL ethanol through a 0.4 μm filter until complete.

In some embodiments, after each of the milled and sieved plant, fungi, and/or algae raw material is prepared, extracted, and filtered, an LC-MS fingerprint is obtained for each.

In some embodiments, each of the prepared, extracted, and filtered plant, fungi, and/or algae material is blended and adjusted to the dose target, such as by the proportions of each. In some embodiments, a blend includes all of the filtered extracts of P. cubensis, C. sativa, P. yezoensis, and D. odorata, as described above. In some embodiments, a blend includes all of the filtered extracts of P cubensis, C. sativa, P. yezoensis, and D. odorata, as described above, wherein the filtered extract of P cubensis comprises a mixture of ultrasound and Soxhlet extraction methods, such as described above. In some embodiments, wherein the filtered extract of P. cubensis comprises a mixture of ultrasound and Soxhlet extraction methods, it comprises a mixture of 80% ultrasound and 20% Soxhlet extraction methods, such as described above. In some embodiments, a blend includes two or more of the filtered extracts of P. cubensis, C. sativa, P. yezoensis, and D. odorata, including a blend of two such extracts, three such extracts, four such extracts, five such extracts, and greater than five such extracts, including multiple extracts of any of P cubensis, C. sativa, P. yezoensis, and D. odorata, as above or otherwise.

In some embodiments, the blend includes an excipient. In some embodiments, the blend includes water as the excipient. In some embodiments, the blend includes water and a flavorant/colorant as the excipients. In some embodiments, the blend includes ginger and/or bay laurel as the flavorant/colorant. In some embodiments, the blend includes additional excipients. In some embodiments, the blend includes no additional excipients. In some embodiments, the blend includes additional active agents. In some embodiments, the blend includes no additional active agents. In some embodiments, the excipients are added to the extracts before the extracts are blended. In some embodiments, the excipients are added to the extracts while the extracts are blended. In embodiments, the excipients are added to the extracts after the extracts are blended.

In some embodiments, after blending, the blend is homogenized. In some embodiments, homogenization is performed by ultrasonic homogenization for 15 minutes at 25% power.

In some embodiments, the homogenized blend is hot filtered to 0.4 microns. In some embodiments, the hot filtrate is next cooled or let cool, and then filtered to 0.22 microns. In some embodiments, an LC-MS fingerprint is obtained of the homogenized filtrate. In some embodiments, including if the fingerprint demonstrates that the product meets the characterization sought, the product may be administered to patients, including as the pharmaceutical composition ABS-108 or NIM-01.

Through this exemplary embodiment, it will be appreciated that the preparation may be viewed broadly as comprising the steps of (1) milling and sieving the raw material to obtain a crude botanical ingredient; (2) preparation of the crude ingredient to obtain a prepared ingredient; (3) extraction of the prepared ingredient to obtain an extract; (4) filtration of the extract to obtain a filtrate; (5) blending and adjustment of the filtrates to obtain a blended botanical mixture; (6) homogenization and filtration of the blended mixture to obtain a final botanical product.

It will be readily appreciated that steps may be performed in a different order, and steps may be combined, removed, or modified, and further steps may be added. It will be further understood that “botanical” may refer to any plant, fungi, and/or algae ingredients or products.

In embodiments, individual extractions are performed on each resulting formulation of a crude botanical ingredient, according to the U.S. Pharmacopeia (USP) guidelines, and liquid chromatography-mass spectrometry (LC-MS) testing is conducted to establish the primary and/or secondary bioactive molecule concentration for each extraction, for example the concentration of Δ9-THC, CBD, psilocin, psilocybin, coumarin, and porphyran.

In some embodiments, relative ratios of each extraction are calculated according to the desired dosages of the primary and/or secondary bioactive molecules in the formulation, which may be any of the formulations disclosed in EXAMPLES 1-14. The primary and/or secondary bioactive molecules are then compounded according to the calculated ratios and homogenized. A final LC-MS test is performed to confirm the concentration of primary and/or secondary bioactive molecules are within the target identified dose range. Excess raw material is then destroyed. The final homogenized compound is thereafter validated as a pharmaceutical to be dispensed, labeled, and shipped to the appropriate site for distribution.

### Example 17: Exemplary Analysis of a Disclosed Composition

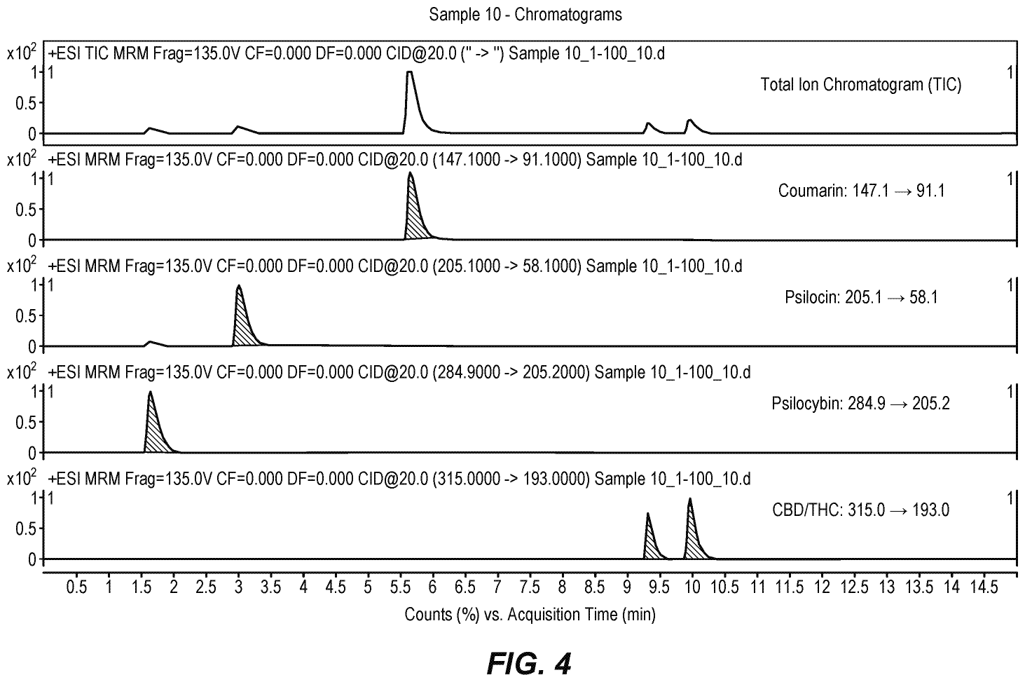

An exemplary composition comprising a therapeutic combination was prepared according to methods disclosed herein, such as in EXAMPLE 16. This composition was subjected to LC-MS and NMR analysis. Exemplary LC-MS data for the composition (which is labeled “Sample 10”) are provided in FIG. 4, which depicts chromatograms showing the concentrations of constituent bioactive molecules contained in the composition, including the total ion concentration of the formulation, the concentration of coumarin contained in the formulation, the concentration of psilocin contained in the formulation, the concentration of psilocybin contained in the formulation, and the concentration of CBD/THC contained in the formulation.

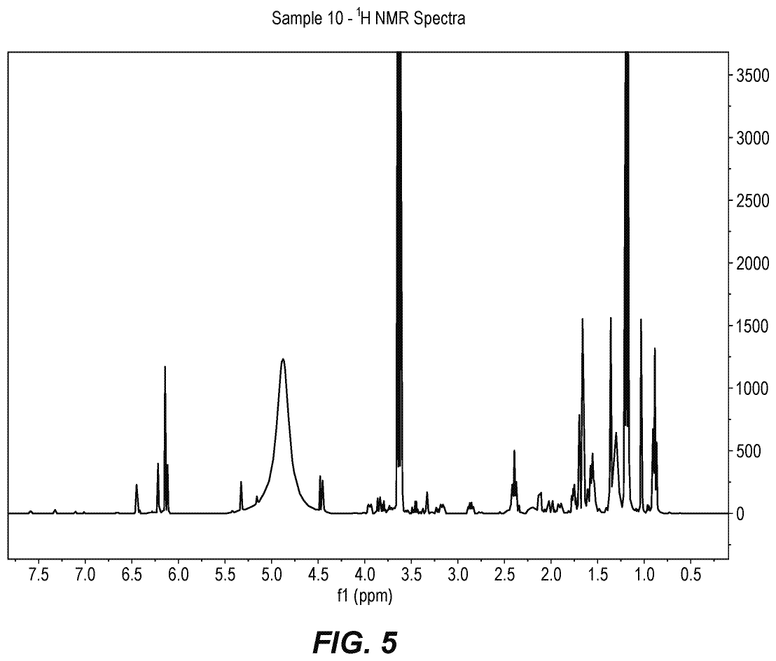

Exemplary proton (¹H) nuclear magnetic resonance (NMR) spectroscopy data is provided in FIG. 5, which depicts an ¹H NMR spectrum for the composition (which is labeled “Sample 10”). The magnitude of the signal is shown on the ordinate axis (γ-axis), and frequency (i.e., chemical shift) is shown on the horizontal axis (x-axis) in parts per million (ppm).

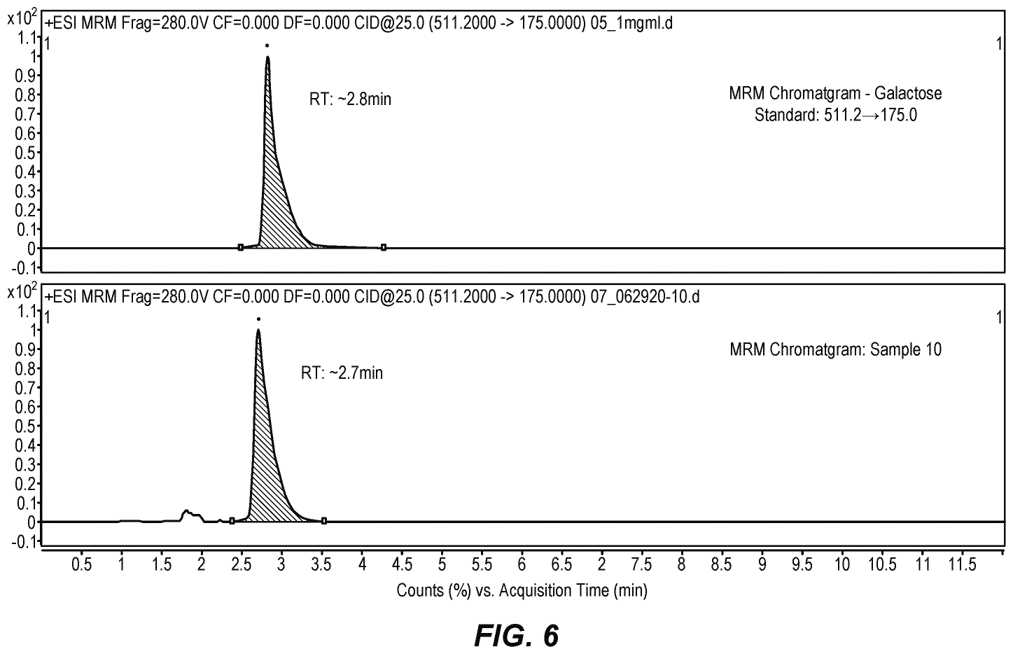

Exemplary multiple reaction monitoring (MRM) LC-MS data are provided in FIG. 6, which shows two MRM chromatograms, including a MRM chromatogram for the composition (which is labeled “Sample 10”) and a MRM chromatogram for a standard sample containing galactose. MRM is a mass spectrometry method that allows for the identification and quantification of fragments of a selected precursor ion. This technique is particularly useful for the analysis of large and complex biomolecules, such as polypeptides and other biopolymers. In some embodiments, MRM is used for quantifying the Pyropia-derived polysaccharide concentration in a disclosed composition. Pyropia contains porphyran, a highly substituted agarose with a linear backbone consisting of 3 linked β-D-galactosyl units alternating with either 4-linked α-L-galactosyl 6-sulfate or 3-6-anhydro-α-L-galactosyl units. The precise composition of porphyran shows seasonal and environmental variations (Jofre et al., 2020). In some embodiments, MRM techniques are used to quantify the amount of galactose (a porphyran fragmentation product) in a sample, thereby providing insight into the composition with regard to porphyran and Pyropia concentration. FIG. 6 shows a retention time (RT) of ˜2.8 minutes for galactose and a RT of ˜2.7 minutes for Sample 10.

Concentrations of the bioactive molecules in the composition, determined according to methods of this Example, are shown below in TABLE 6.

TABLE 6
LC-MS formulation parameters

| | | Concentration | Psilocin | Psilocybin | Coumarin | CBD | THC |
|---|---|---|---|---|---|---|---|
| Sample # | Description | (mg/mL)* | (mg/mL) | (mg/mL) | (mg/mL) | (mg/mL) | (mg/mL) |
| 062920-10 | Full | 7.5 Psilocybin | 1.63 | 12.15 | 39.19 | 56.20 | 52.82 |
| | formulation | 50 THC/CBD | | | | | |

*Estimates based on Applicant's calculations.
BLD = below limit of detection (~100 mg/mL).

### Example 18: Placebo Controlled Study

A double-blind placebo controlled study will be completed where subjects with an inflammatory condition or disorder are separated into two or more groups. The inflammatory condition or disorder may be, for example, long COVID. Half of the participants will receive one or more doses of the combinations disclosed herein (e.g., the tincture of EXAMPLE 1), while the other half will receive one or more doses of a placebo. The study will last for between 1 to 10 weeks.

Inflammatory condition or disorder symptoms, such as those disclosed herein, will be assessed prior to and after the duration of the study. At the conclusion of the study, it will be determined that the participants receiving one or more doses of the combinations disclosed herein will have equal or greater improvements in one or more symptoms of an inflammatory condition or disorder compared to those receiving one or more doses of placebo, as determined by the measured symptomatology.

### Example 19: Clinical Mechanical Models

In some further examples, data are collected at baseline (e.g., at the start of treatment, or a selected point before treatment initiation) and at one or more points during and/or following treatment, such as about 1 week, 2 weeks, 3 weeks, 4 weeks, 6 weeks, 2 months, 3 months, 4 months, 5 months, 6 months, 7 months, 8 months, 9 months, 10 months, 11 months, 1 year, 15 months, 18 months, 21 months, 2 years, 2.5 years, 3 years, and more than 3 years following treatment. The data at baseline are compared with the one or more points during and/or following treatment (for a single subject, or between subjects, using statistical methods as will be commonly known) and analyzed to determine the treatment effect. Treatment effect (e.g., post-treatment delta from baseline) will be determined by self-reporting or other methods disclosed herein or otherwise known to those of skill in the art.

### Example 20: Synergistic Effects

Purpose: A study will be conducted to determine if the disclosed therapeutic combinations have synergistic anti-inflammatory effects.

Methods: Subjects having an inflammatory condition or disorder will be selected for treatment during a treatment period and their symptoms will be measured prior to the study to obtain a baseline and in regular intervals throughout the treatment period to monitor their responsiveness to treatment.

During the treatment treatment period, subjects suffering from an inflammatory condition or disorder will be divided into 5 treatment groups, which will receive either a daily oral dose of a placebo; 250 μg of psilocybin, and 150 μg of psilocin; 1 mg of THC and 1 mg of CBD; 250 μg of psilocybin, 150 μg of psilocin, 1 mg of THC and 1 mg of CBD; or 250 μg of psilocybin, 150 μg of psilocin, 1 mg of THC, 1 mg of CBD, 1 mg of Coumarin, and 8 mg of whole Pyropia extract.

Results & Significance: At the conclusion of the study, it will be determined that those given the therapeutic combinations will exhibit a reduction in at least one symptom of an inflammatory condition or disorder. The reduction in inflammatory condition or disorder symptoms will be observed to be greatest in the treatment group receiving 250 μg of psilocybin, 150 μg of psilocin, 1 mg of THC, 1 mg of CBD, 1 mg of Coumarin, and 8 mg of whole Pyropia extract due to the synergistic effects of the ingredients in this therapeutic combination.

### Example 21: Additional In Vitro and In Vivo Studies

Additional in vitro and in vivo studies may be performed and are expected to confirm the results of the other studies herein as described in the Examples above and/or to provide additional support for the utility and benefits of the disclosed invention.

Non-limiting examples of contemplated in vitro studies include: (1) Inflammatory immune cell activation and profiling of pro- and anti-inflammatory cytokines, restorative growth factors, and neuroendocrine panels; (2) Stem cell effects in vitro lab testing under inflamed culture conditions: screening assays; (3) Neuronal cell models: anti-inflammatory and protective properties; and (4) Stem cell mobilization. Non-limiting examples of contemplated in vitro studies include: (1) Serum pro- and anti-inflammatory cytokines, restorative growth factors; (2) Serum neuroendocrine communication markers; and (3) Mitochondrial resilience testing.

### Example 22: Total Antioxidant Capacity (TPH) Assay

Purpose: A study was conducted to determine the antioxidant capacity of Cannabis stock, Psilocybe stock, Pyropia stock, tonka stock, as well as multi-ingredient combinations.

Methods: The products were tested in the Folin-Ciocalteu assay (also known as the total phenolics assay), according to the methods of Huang et al. J Agric Food Chem. 2005; 53(6):1841-56 and Kupina et al. J AOAC Int. 2018; 101(5):1466-14722018. The assay was performed by adding the Folin-Ciocalteu's phenol reagent to serial dilutions of extract, thoroughly mixing, and incubating for 5 minutes. Sodium carbonate was added, starting a chemical reaction producing a color. The reaction was allowed to continue for 30 minutes at 37° C. Optical absorbance was measured at 765 nm in a colorimetric plate reader. Gallic acid was used as a reference standard, and data was reported in Gallic Acid Equivalents (GAE) per gram product. The details of test products utilized are provided in TABLE 7.

TABLE 7
Test products

| Name | Abbreviation | Percentage | Description | Handling |
|---|---|---|---|---|
| Cannabinoid extract | Ca | 15% | CBD, THC | Not water-soluble* |
| Psilocybe extract | Ps | 45% | Complex extract** | water-soluble |
| Pyropia extract | Py | 15% | Complex extract | water-soluble |
| Tonka bean extract | To | 2.0% | 50% w/v coumarin | water-soluble |

*In the test for total antioxidant capacity, Cannabis was diluted in glycerin, followed by serial dilutions in distilled water. In subsequent tests, Cannabis was emulsified using soy lecithin, followed by serial dilutions in physiological saline. Cannabis blended well with Psilocybe and no emulsifier was needed when blending these two ingredients.
**20 mg/mL, with a 50:50 split between psilocybin and psilocin. Also contains polysaccharides, beta-carbolines, MBAPs, and other complex compounds.

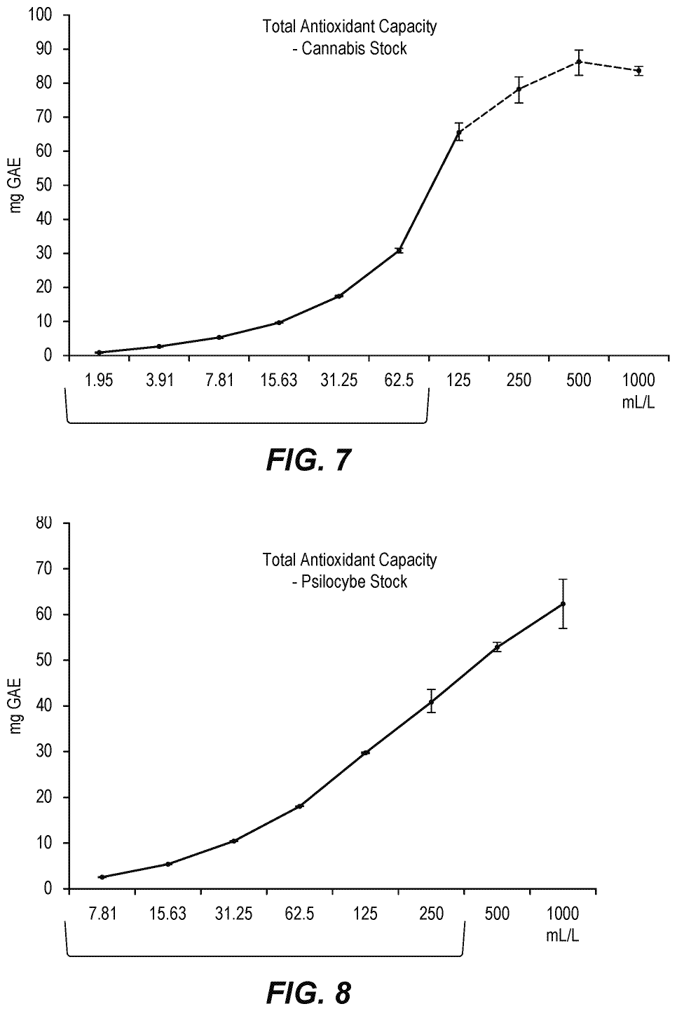

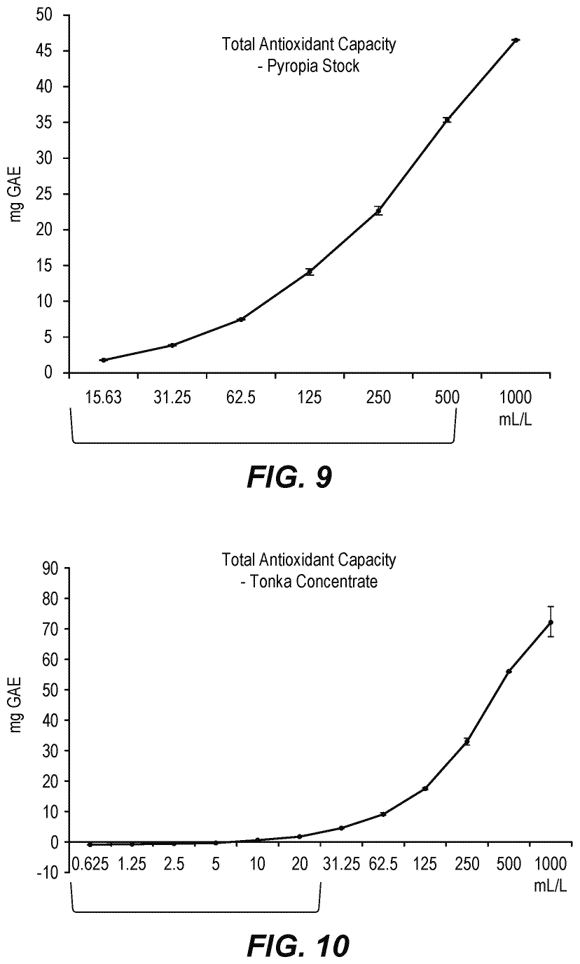

Results & Significance: The antioxidant capacities of disclosed compounds and combinations are provided in TABLE 8 below. The individual total antioxidant capacities obtained via the Folin-Ciocalteu assay for Cannabis Stock, for Psilocybe Stock, for Pyropia Stock, and for Tonka Stock are depicted in FIGS. 7-10, respectively. FIGS. 7-10 show the calculated GAEs for Cannabis Stock, for Psilocybe Stock, for Pyropia Stock, and for Tonka Stock, respectively, in milligrams (mg) per milliliter/liter (mL/L) of solution utilized. The data shown in FIGS. 7-10 includes the average±standard deviation of duplicate data points for each dose, and the bracket located under the abscissa of the graph indicates the dose range used for subsequent assays. The dashed line shown in FIG. 7 indicates that precipitates formed as part of the chemical reaction, and that the light absorbance readings are not reliable. Individually, all ingredients had antioxidant capacity, with Cannabis and Psilocybe having the most robust antioxidant capacity. Tonka also provided an enhancement of the antioxidant capacity, despite its very low proportion in the blend. Pyropia showed approximately half of the antioxidant capacity seen for Psilocybe. Further details and conclusions are in the Brief Description of the Figures.

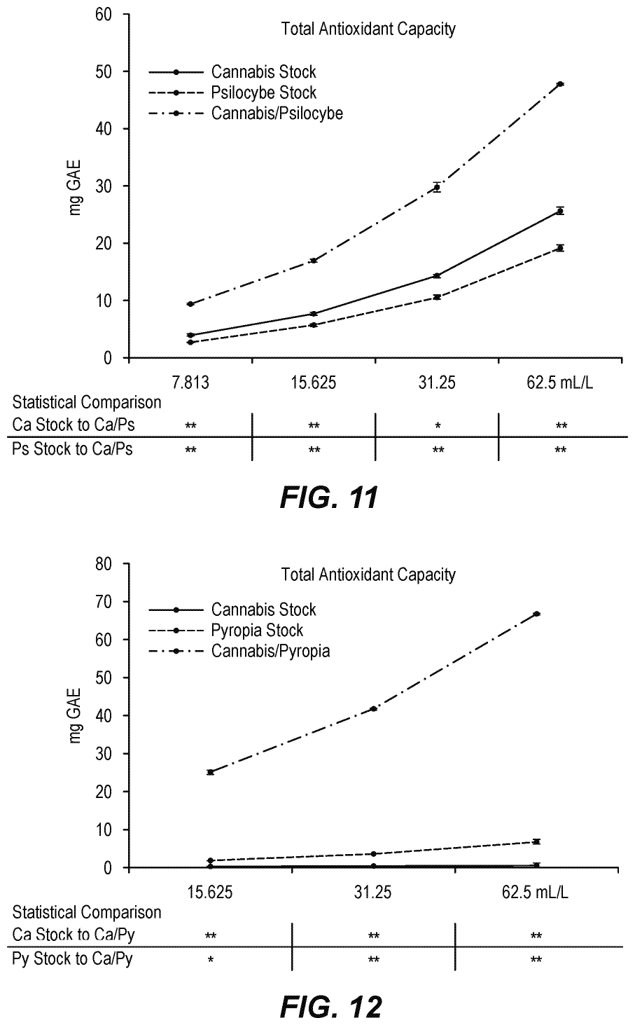

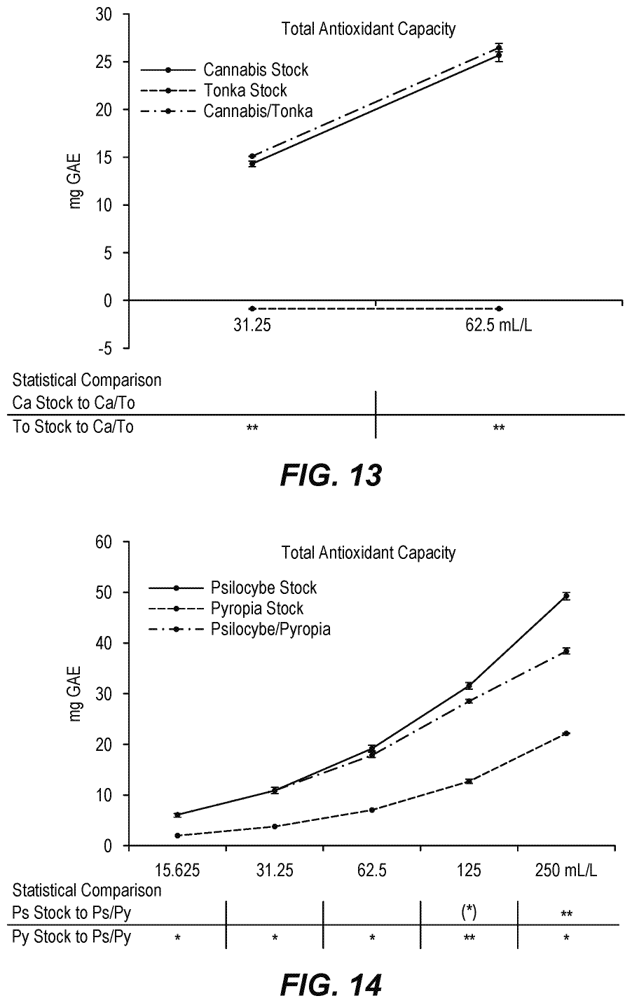

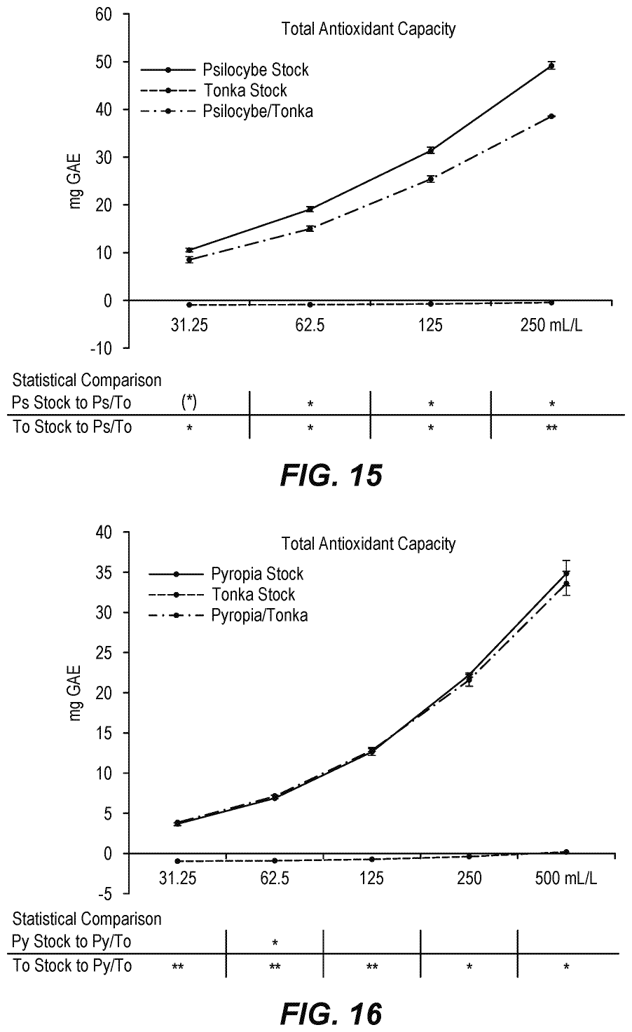

All 2-ingredient blends had antioxidant capacity. The total antioxidant capacities obtained via Folin-Ciocalteu assay for 2-ingredient blends are described in FIGS. 11-16. In FIGS. 11-16, the data is shown as the average±standard deviation of duplicate data points for each dose.

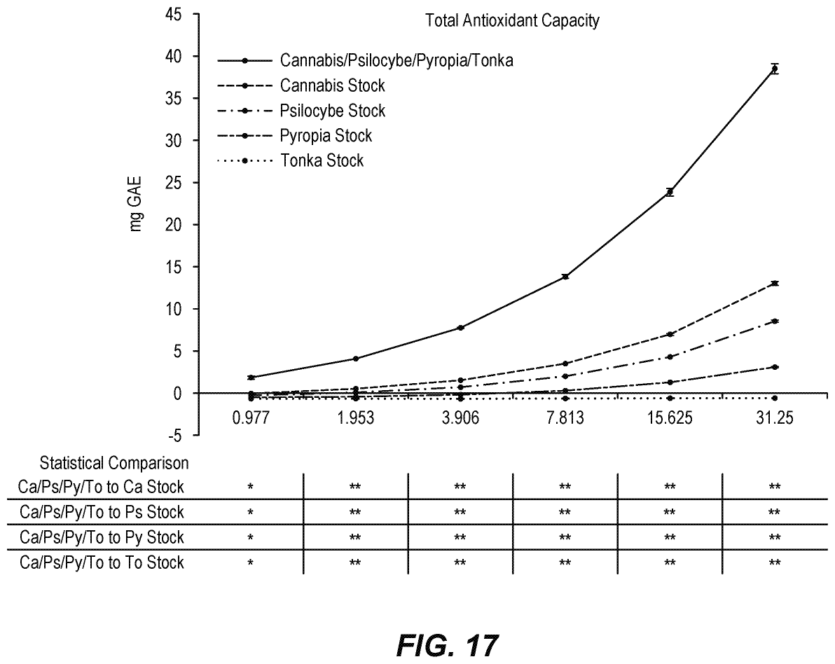

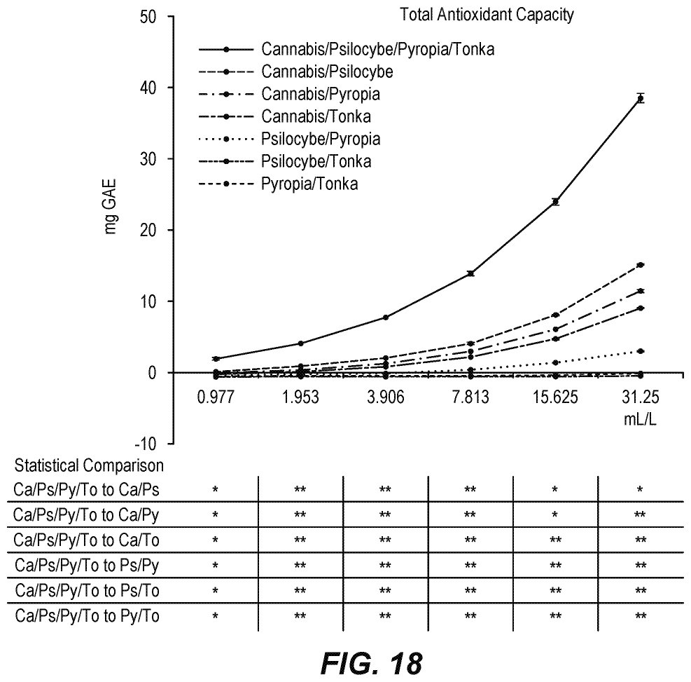

The total antioxidant capacities obtained via the Folin-Ciocalteu assay for the 4-ingredient blends are described in FIGS. 17-18. In FIGS. 17-18, the data is shown as the average±standard deviation of duplicate data points for each dose. The 4-ingredient blend showed a higher antioxidant capacity than what would be expected by adding the contribution of each ingredient together. These data suggest a synergistic effect between the ingredients. The 4-ingredient blend showed a higher antioxidant capacity than what would be expected by adding the contributions of each 2-ingredient blend together. These data suggest a synergistic effect between the ingredients.

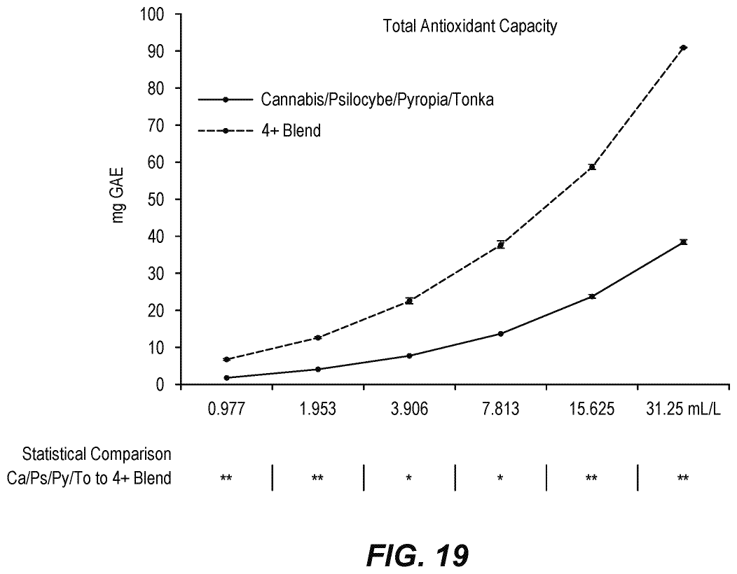

The total antioxidant capacity obtained via the Folin-Ciocalteau assay for the 4+ blend, e.g. Applicant's final product disclosed herein as ABS-108 and NIM-01, and as the formulation of EXAMPLE 1, is described in FIG. 19. FIG. 19 depicts data as the average±standard deviation of duplicate data points for each dose. The 4+ blend additionally comprises a flavorant/colorant, which is not included in the composition of the 4-ingredient blend. The flavorant/colorant is ethanol infused with ginger and bay leaf. The 4+ blend showed advantages above any of the individual ingredients and the 2-ingredient blends. Moreover, as depicted in FIG. 19, the 4+ blend surprisingly showed a higher antioxidant capacity than the 4-ingredient blend. This demonstrates that the additional ingredients in the 4+ blend contribute to the antioxidant properties of the final product. Furthermore, the 4+ blend showed a higher antioxidant capacity than what would be expected by adding the contribution of each ingredient together, which demonstrates an unexpected synergistic effect between the ingredients of the 4+ blend.

TABLE 8
Total antioxidant capacity

| | mg GAE per |
|---|---|
| Product | mL of product |
| Cannabis Stock | 0.4 |
| Psilocybe Stock | 0.3 |
| Pyropia Stock | 0.1 |
| Tonka Stock | 0.0 |
| Tonka Concentrate | 0.1 |
| Cannabis/Psilocybe | 0.3 |
| Cannabis/Pyropia | 1.1 |
| Cannabis/Tonka | 0.4 |
| Psilocybe/Pyropia | 0.2 |
| Psilocybe/Tonka | 0.2 |
| Pyropia/Tonka | 0.1 |
| Cannabis/Psilocybe/ | 1.2 |
| Pyropia/Tonka | |
| 4+ Blend | 2.9 |

### Example 23: Strategic Research Plan for Additional Experimental Work

Applicant has developed therapeutic combinations and methods for treating inflammatory disorders and providing whole system biological support. A structured Strategic Research Plan will be initiated, including analytical chemistry, animal safety studies, in vitro bioassays, and human clinical trials for immediate and long-term efficacy. This work will include culturing immune cells under unstressed versus inflamed conditions and monitoring immune activation and production of a broad panel of cytokines and growth factors following administration of a disclosed combination.

Test articles are prepared for testing according to EXAMPLE 22. Each ingredient will be tested alone. In addition, each pair of ingredients will be tested as 2-ingredient blends, and the blend of all 4 ingredients will also be tested. An additional comparison involves the 4-ingredient blend to the final product that contains additional ingredients (e.g., flavorants, colorants, diluents; referred to herein as the “4+ blend”). Tests will include (1) documentation of immune cell activation by flow cytometry: cell surface expression of the growth factor receptor CD25 and the Very Early Activation antigen CD69 on Natural Killer cells, NKT cells, T lymphocytes, and non-NK non-T cells; and (2) Expression of a broad panel of 27 cytokines, anti-viral peptides, anti-inflammatory cytokines, and regenerative growth factors.

Methods: Cell cultures: Human peripheral blood mononuclear cell (PBMC) cultures are used for this testing. A set of cultures are left untreated as negative control cultures for immune activation. Triplicate sets of cultures will be treated with serial dilutions of the test product. The highly inflammatory bacterial lipopolysaccharide LPS from E. coli is used as a positive control for activation and is also used in one of the three cultures to induce inflammation after treating the immune cells with a natural product. The testing will be performed such that all treatments, including each dose of test product and each positive and negative control, are tested in triplicate. The cultures are incubated for 24 hours, after which the cells and the culture supernatants are harvested and used to monitor the reactions in each culture. The testing will be performed on cells from a healthy blood donor.

Methods: Documentation of immune cell activation by flow cytometry: Cells are stained with a combination of monoclonal antibodies to monitor activation, and analyzed by multi-parameter flow cytometry, using an acoustic dual laser Attune flow cytometer. The analysis includes fluorescent markers for CD3, CD56, CD25, and CD69. This combination allows monitoring of changes to monocyte/macrophages, as well as activation of natural killer cells, NKT cells, and T lymphocytes.

The stained cells are analyzed by multi-parameter flow cytometry, using an acoustic dual laser Attune flow cytometer. During data analysis, the physical properties of different cell types allow electronic gating on lymphocytes versus monocytes, so that the CD69 versus CD25 expression can be analyzed on these cell types separately. In addition, the lymphocyte fraction is divided into 4 separate subpopulations, based on whether cells stain with CD3, CD56, both, or none.

Cells will be stained with the T cell marker CD3 and the CD56 markers, as well as the two activation markers CD69 and the interleukin-2 receptor CD25. This allows analysis of numbers of the following types of immune cells: CD3− CD56+ NK cells; CD3+ CD56+ NKT cells; CD3+ CD56− T lymphocytes; CD3− CD56− non-NK, non-T lymphocytes; and monocytes (identified by forward/side scatter profile). During analysis, expression levels were determined for the activation molecule CD69 and growth factor receptor CD25 on the surface of the cell populations listed above. This is based on established and published protocols for natural products research.

Methods: Expression of cytokines and growth factors: The culture supernatants from each culture are used for testing of a broad panel of pro- and anti-inflammatory cytokines, anti-viral peptides, and regenerative growth factors, using a 27-plex Luminex magnetic bead array and the MagPix© multiplexing system. The cytokine panel will include: IL-1beta, IL-1ra, IL-2, IL-4, IL-5, IL-6, IL-7, IL-8, IL-9, IL-10, IL-12 (p70), IL-13, IL-15, IL-17, eotaxin, basic FGF, G-CSF, GM-CSF, IFN-gamma, IP-10, MCP-1 (MCAF), MIP-1alpha, MIP-1beta, PDGF-BB, RANTES, TNF-alpha, and VEGF. This broad panel of cytokines is a cost-effective screening. The panel contains biomarkers with multi-faceted roles and is likely to generate results that may allow us to discuss pro-inflammatory immune-activating effects, anti-inflammatory effects, and restorative effects through growth factors acting on stem cells, such as G-CSF.

Results: It is expected that disclosed therapeutic combinations will cause measurable immune-modulating effects in exposed cells, indicating that the combinations are likely effective for treating inflammatory conditions and disorders in subjects in need thereof. Results will also provide a mechanistic understanding of anti-inflammatory effects of disclosed combinations. Results will also guide the design and implementation of additional in vitro and clinical testing, which may include, for example:
1. Determining effects on stem cells in in vitro lab testing under inflamed culture conditions
2. Continued evaluation of anti-inflammatory and immune supporting properties, and neuroendocrine panels
3. Studying the hepatotoxicity of disclosed combinations and their ingredients in liver cell models
4. Measuring anti-inflammatory and protective properties in neuronal cell models
5. Clinical studies in a study population of healthy adults, evaluating rapid and/or long-term effects. The study will be conducted as a placebo-controlled cross-over trial. Outcome measures will include stem cell mobilization, immune cell activation, serum pro- and anti-inflammatory cytokines, restorative growth factors, and serum neuroendocrine communication markers. Additionally, outcomes will be assessed by collecting questionnaire data (e.g., as described in embodiments herein) to document how subjects experience health changes.

The foregoing description, for purposes of explanation, uses specific nomenclature to provide a thorough understanding of the invention. However, it will be apparent to one skilled in the art that specific details are not required in order to practice the invention. Thus, the foregoing description of specific embodiments of the invention is presented for purposes of illustration and description. It is not intended to be exhaustive or to limit the invention to the precise forms disclosed; of course, many modifications and variations are possible in view of the above teachings. The embodiments were chosen and described in order to best explain certain key principles of the invention and its practical applications, through the elucidation of specific examples, and to thereby enable others skilled in the art to best make and utilize the invention and various embodiments with various modifications as are suited to the particular use contemplated, even when such uses are beyond the specific examples disclosed. Accordingly, the scope of the invention shall be defined solely by the following claims and their equivalents.

1-16. (canceled)

17. A method of preventing or treating an inflammatory condition or disorder, comprising administering to a subject having the inflammatory condition or disorder a therapeutic combination comprising:
a. a fungal portion, which comprises a Psilocybe cubensis extract;
b. a first plant portion, which comprises a Cannabis sativa extract;
c. a second plant portion, which comprises a Dipteryx odorata extract; and
d. an algal portion, which comprises a Pyropia yezoensis extract.

18-20. (canceled)

21. The method of claim 17, wherein the Psilocybe cubensis extract comprises psilocybin and psilocin.

22. The method of claim 21, wherein the Psilocybe cubensis extract comprises psilocybin and psilocin in a weight ratio from about 1:5 to about 5:1.

23. (canceled)

24. The method of claim 21, wherein the Psilocybe cubensis extract comprises from about 50 μg to about 500 μg of psilocybin and from about 20 μg to about 200 μg of psilocin.

25-26. (canceled)

27. The method of claim 17, wherein the Cannabis sativa extract comprises Δ⁹-tetrahydrocannabinol (THC) and cannabidiol (CBD).

28. The method of claim 27, wherein the Cannabis sativa extract comprises THC and CBD in a weight ratio from about 1:5 to about 5:1.

29. (canceled)

30. The method of claim 27, wherein the Cannabis sativa extract comprises from about 0.1 mg to about 5 mg of THC and from about 0.1 mg to about 5 mg of CBD.

31-32. (canceled)

33. The method of claim 17, wherein the Dipteryx odorata extract comprises coumarin.

34. The method of claim 17, wherein the Dipteryx odorata extract comprises from about 1 mg to about 10 mg of coumarin.

35. (canceled)

36. The method of claim 17, wherein the combination comprises from about 1 mg to about 20 mg of Pyropia yezoensis extract.

37-38. (canceled)

39. The method of claim 17, wherein the Pyropia yezoensis extract comprises porphyran.

40. The method of claim 17, wherein any one of the Psilocybe cubensis extract, Cannabis sativa extract, Dipteryx odorata extract, and Pyropia yezoensis extract is administered in a separate composition.

41. (canceled)

42. The method of claim 17, wherein the Psilocybe cubensis extract, Cannabis sativa extract, Dipteryx odorata extract, and Pyropia yezoensis extract are administered in a single composition.

43-50. (canceled)

51. The method of claim 42, wherein the single composition further comprises any of a flavorant, a colorant, and a diluent.

52. The method of claim 51, wherein the flavorant or the colorant comprises ginger or bay laurel.

53-55. (canceled)

56. The method of claim 51, wherein the diluent is water.

57-58. (canceled)

59. The method of claim 42, comprising administering between about 10 mg and about 200 mg of the single composition per single dose.

60-62. (canceled)

63. The method of claim 17, wherein the subject experiences a reduction in the severity of a symptom of the inflammatory condition or disorder.

64. (canceled)

65. The method of claim 17, wherein the inflammatory condition or disorder is any of long COVID, rheumatoid arthritis, psoriatic arthritis, reactive arthritis, tendonitis, gout, scleroderma, systemic scleroderma, localized scleroderma, CREST syndrome, sciatica, neuropathy, peripheral neuropathy, hereditary neuropathy, acquired neuropathy, motor neuropathy, sensory neuropathy, autonomic neuropathy, combination neuropathy, sciatic neuritis, myalgic encephalomyelitis, Chronic Fatigue Syndrome (CFS), fatty liver disease, endometriosis, type 1 diabetes mellitus, type 2 diabetes mellitus, inflammatory bowel disease, asthma, obesity, cancer, Kawasaki's disease, vasculitis, Uveitis, Crohn's disease, ulcerative colitis, meningitis, allergies, psoriasis, Hashimoto's Disease, Guillain Barre Syndrome, hepatitis, Celiac Disease, multiple sclerosis, fibromyalgia, lupus, and Sjogren's syndrome.

66. The method of claim 17, wherein the inflammatory condition or disorder is any of long COVID, CREST syndrome, psoriatic arthritis, Hashimoto's disease, multiple sclerosis, sciatica, peripheral neuritis, post-treatment Lyme disease (PTLD)/chronic Lyme disease (CLD) syndrome, chronic fatigue and immune dysfunction syndrome (CFIDS)/chronic fatigue syndrome (CFS), and an inflammatory skin condition.

67-210. (canceled)
